(19) United States
(12) Patent Application Publication
(10) Pub. No.: US 2015/0133426 A1
(43) Pub. Date: May 14, 2015

(54) SUBSTITUTED PYRROLOPYRIMIDINES

(71) Applicant: BAYER PHARMA AKTIENGESELLSCHAFT, Berlin (DE)

(72) Inventors: Ulrich Klar, Berlin (DE); Georg Kettschau, Berlin (DE); Detlev Sülzle, Berlin (DE); Florian Puehler, Wellesley, MA (US); Dirk Kosemund, Berlin (DE); Philip Lienau, Berlin (DE); Ulf Bömer, Glienicke (DE); Lars Wortmann, Berlin (DE)

(21) Appl. No.: 14/403,154

(22) PCT Filed: May 17, 2013
(86) PCT No.: PCT/EP2013/060232

§ 371 Date: Nov. 21, 2014

(30) Foreign Application Priority Data
May 21, 2012 (EP) 12168671.1
Feb. 4, 2013 (EP) 13153905.8

(51) Int. Cl. C07D 491/20; A61K 31/519; C07D 487/16; C07D 495/14; A61K 31/5377; C07D 487/04; C07D 487/14

(52) U.S. Cl. CPC C07D 491/20; C07D 487/04; A61K 31/519; C07D 487/14; C07D 495/14; A61K 31/5377; C07D 487/16
USPC 514/210.18; 544/250; 514/267; 544/251; 544/115; 514/232.8; 544/105; 514/230.5; 514/252.16; 540/455

(57) ABSTRACT

The present invention relates to substituted pyrrolopyrimidine compounds general formula I: in which A, X, R¹, R², m and n are as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyper-proliferative and/or angiogenesis disorder, as a sole agent or in combination with other active ingredients.

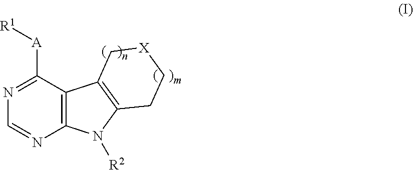

The present invention relates to substituted pyrrolopyrimidine compounds of general formula I as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyper-proliferative and/or angiogenesis disorder, as a sole agent or in combination with other active ingredients.

### BACKGROUND OF THE INVENTION

The present invention relates to chemical compounds that inhibit MKNK1 kinase (also known as MAP Kinase interacting Kinase, Mnk1) and MKNK2 kinase (also known as MAP Kinase interacting Kinase, Mnk2). Human MKNKs comprise a group of four proteins encoded by two genes (Gene symbols: MKNK1 and MKNK2) by alternative splicing. The b-forms lack a MAP kinase-binding domain situated at the C-terminus. The catalytic domains of the MKNK1 and MKNK2 are very similar and contain a unique DFD (Asp-Phe-Asp) motif in subdomain VII, which usually is DFG (Asp-Phe-Gly) in other protein kinases and suggested to alter ATP binding [Jauch et al., Structure 13, 1559-1568, 2005 and Jauch et al., EMBO J 25, 4020-4032, 2006]. MKNK1a binds to and is activated by ERK and p38 MAP Kinases, but not by JNK1. MKNK2a binds to and is activated only by ERK. MKNK1b has low activity under all conditions and MKNK2b has a basal activity independent of ERK or p38 MAP Kinase. [Buxade M et al., Frontiers in Bioscience 5359-5374, May 1, 2008]

MKNKs have been shown to phosphorylate eukaryotic initiation factor 4E (eIF4E), heterogeneous nuclear RNA-binding protein A1 (hnRNP A1), polypyrimidine-tract binding protein-associated splicing factor (PSF), cytoplasmic phospholipase A2 (cPLA2) and Sprouty 2 (hSPRY2) [Buxade M et al., Frontiers in Bioscience 5359-5374, May 1, 2008].

eIF4E is an oncogene that is amplified in many cancers and is phosphorylated exclusively by MKNKs proteins as shown by KO-mouse studies [Konicek et al., Cell Cycle 7:16, 2466-2471, 2008; Ueda et al., Mol Cell Biol 24, 6539-6549, 2004]. eIF4E has a pivotal role in enabling the translation of cellular mRNAs. eIF4E binds the 7-methylguanosine cap at the 5′ end of cellular mRNAs and delivers them to the ribosome as part of the eIF4F complex, also containing eIF4G and eIF4A. Though all capped mRNAs require eIF4E for translation, a pool of mRNAs is exceptionally dependent on elevated eIF4E activity for translation. These so-called “weak mRNAs” are usually less efficiently translated due to their long and complex 5′UTR region and they encode proteins that play significant roles in all aspects of malignancy including VEGF, FGF-2, c-Myc, cyclin D1, survivin, BCL-2, MCL-1, MMP-9, heparanase, etc. Expression and function of eIF4E is elevated in multiple human cancers and directly related to disease progression [Konicek et al., Cell Cycle 7:16, 2466-2471, 2008].

MKNK1 and MKNK2 are the only kinases known to phosphorylate eIF4E at Ser209. Overall translation rates are not affected by eIF4E phosphorylation, but it has been suggested that eIF4E phosphorylation contributes to polysome formation (i.e. multiple ribosome on a single mRNA) that ultimately enables more efficient translation of “weak mRNAs” [Buxade M et al., Frontiers in Bioscience 5359-5374, May 1, 2008]. Alternatively, phosphorylation of eIF4E by MKNK proteins might facilitate eIF4E release from the 5′ cap so that the 48S complex can move along the “weak mRNA” in order to locate the start codon [Blagden S P and Willis A E, Nat Rev Clin Oncol. 8(5):280-91, 2011]. Accordingly, increased eIF4E phosphorylation predicts poor prognosis in non-small cell lung cancer patients [Yoshizawa et al., Clin Cancer Res. 16(1):240-8, 2010]. Further data point to a functional role of MKNK1 in carcinogenesis, as overexpression of constitutively active MKNK1, but not of kinase-dead MKNK1, in mouse embryo fibroblasts accelerates tumor formation [Chrestensen C. A. et al., Genes Cells 12, 1133-1140, 2007]. Moreover, increased phosphorylation and activity of MKNK proteins correlate with overexpression of HER2 in breast cancer [Chrestensen, C. A. et al., J. Biol. Chem. 282, 4243-4252, 2007]. Constitutively active, but not kinase-dead, MKNK1 also accelerated tumor growth in a model using Ep-Myc transgenic hematopoietic stem cells to produce tumors in mice. Comparable results were achieved, when an eIF4E carrying a S209D mutation was analyzed. The S209D mutation mimicks a phosphorylation at the MKNK1 phosphorylation site. In contrast a non-phosphorylatable form of eIF4E attenuated tumor growth [Wendel H G, et al., Genes Dev. 21(24):3232-7, 2007]. A selective MKNK inhibitor that blocks eIF4E phosphorylation induces apoptosis and suppresses proliferation and soft agar growth of cancer cells in vitro. This inhibitor also suppresses outgrowth of experimental B16 melanoma pulmonary metastases and growth of subcutaneous HCT116 colon carcinoma xenograft tumors without affecting body weight [Konicek et al., Cancer Res. 71(5):1849-57, 2011]. In summary, eIF4E phosphorylation through MKNK protein activity can promote cellular proliferation and survival and is critical for malignant transformation. Inhibition of MKNK activity may provide a tractable cancer therapeutic approach.

Although pyrrolopyrimidines may be considered as common heterocyclic core, their condensation with further rings and specific substitution patterns lead to clearly distinct classes of compounds.

Typical features of compounds of the general formula I of the present invention is a condensed tricycle where a 5 or 6 or 7 membered carbocycle, optionally with one heteroatom in the ring, is fused to the 5 membered ring of pyrrolo[2,3-d]pyrimidin-4-amine and where the amino group is monosubstituted by an aromatic or heteroaromatic optionally substituted moiety while position 2 of the pyrimidine ring is unsubstituted.

WO 2010/006032 A1 (Duquesne University of the Holy Spirit) addresses tricyclic compounds as antimitotic agents for the treatment of cancer. According to the general formula of claim 1, the tricycles also comprise 5,6,7,8-tetrahydrobenzo[1]thieno[2,3-d]pyrimidines and 5,6,7,8-tetrahydrobenzo[1]pyrrolo[2,3-d]pyrimidines that may carry substituents at the carbocycle and one aromatic or heteroaromatic moiety at an optional 4-amino group. Furthermore, they may be unsubstituted at position 2 in the pyrimidine ring. However, the examples provided clearly differ from the compounds of the present invention. While the vast majority contains the C6 carbocycle completely unsaturated as aromatic ring, only two examples show the tetrahydrobenzo substructure in combination with a 4-amino group and in both cases the latter is bisubstituted by a phenyl and a methyl group. Furthermore, the compounds are with no exception pyrimidin-2-amines or 2-methyl-pyrimidines.

WO 2009134658 (National Health Research Institutes) relates to inhibitors of Aurora kinase. The invention generically covers pyrrolo[2,3-d]pyrimidin-4-amines with the third ring fused to the pyrrole subunit. However, an optional aryl or heteroaryl substituent at the 4-amino group must carry a side chain involving a carbonyl, thiocarbonyl or iminomethylene group. The vast majority of more than 250 examples is formed by bicyclic 6,7-dihydrofuro[3,2-d]pyrimidin-4-amines that show in 4 cases a direct aromatic substitution at the 4-amino group and additional substitution by two phenyl groups at the dihydrofuro subunit. None of the very few examples for tricyclic compounds show direct substitution by an aromatic moiety at the 4-amino group.

WO 1995/019970 (Warner-Lambert Company) discloses tricyclic compounds capable inhibiting tyrosine kinases of the epidermal growth factor receptor family. The middle ring of the compounds inter alia can be a pyrrolidine ring; both two rings fused to the middle pyrrolidine ring are aromatic.

WO2011056739 A1 (Glaxosmithkline LLC) addresses tricyclic compounds comprising pyrrolo[2,3-d]pyrimidin-4-amines where the third ring is fused to the pyrrole subunit and is either a tetrahydrobenzo or a tetrahydro-H-pyrido moiety that is optionally substituted. However, substituents at the 4-amino group involve optionally substituted phenyl but that is required to carry an alkylamidosulfonyl group in meta position.

WO2009033581 A1 (Bayer Healthcare AG) relates to novel compounds particularly for the treatment of cancer and comprises condensed tricycles consisting of a pyrrolo[2,3-d]pyrimidin-4-amine core with the third, one substituted nitrogen containing ring being fused to the pyrrole subunit. An unsaturated side chain is linked to the nitrogen of the third ring via a carbonyl-, sulphoxide-, sulphone- or iminomethylene bridge. The 4-amino group may be monosubstituted by an aromatic or heteroaromatic optionally substituted moiety.

However, the state of the art described above does not describe the specific pyrrolopyrimidine compounds of general formula (I) of the present invention, or a stereoisomer, a tautomer, an N-oxide, a hydrate, a solvate, or a salt thereof, or a mixture of same, as described and defined herein, and as hereinafter referred to as “compounds of the present invention”, or their pharmacological activity. It has now been found, and this constitutes the basis of the present invention, that said compounds of the present invention have surprising and advantageous properties.

In particular, said compounds of the present invention have surprisingly been found to effectively inhibit MKNK-1 kinase and may therefore be used for the treatment or prophylaxis of diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK-1 kinase, such as, for example, haemotological tumours, solid tumours, and/or metastases thereof, e.g. Leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

### SUMMARY OF THE INVENTION

The present invention covers compounds of general formula I:

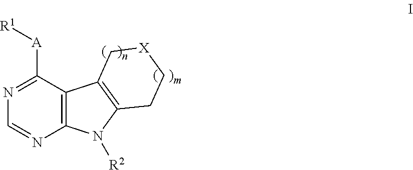

in which:
A represents —O—, —S—, —S(═O)—, —S(═O)₂—, —S(═O)(NR³)— or —NR³—;
X represents —O—, —S—, —S(═O)—, —S(═O)₂—, —S(═O)(NR^{4a})—, —NR^{4a}—, —C(O—C₁-C₆-alkyl)₂-, —C(O—CH₂—CH₂—O)—, —C(O—CH₂—CH₂—CH₂—O)—, —C(O—CH₂—C(CH₃)₂—CH₂—O)—, —C(═O)—, —C(═O)NR^{4a}—, —NR^{4a}C(═O)—, —C(═O)O—, —OC(═O)—, —C(H)(OR^{4a})—, —C(R^{4a})(OR^{4b})—, —C═NR^{4a}—, —C(H)NR^{4a}R^{4b}—, —C(R^{5a})(R^{5b})—, —C(═CR^{6a}R^{6b})— or —CH(CHR^{6a}R^{6b})—;
R¹ represents an aryl- or heteroaryl- group; wherein said aryl- or heteroaryl-group is optionally substituted, identically or differently, with 1, 2, 3, 4 or 5 R⁷ groups;
R² represents a hydrogen atom, or a C₁-C₆-alkyl- or C₃-C₆-cycloalkyl- group; wherein said C₁-C₆-alkyl- or C₃-C₆-cycloalkyl- group is optionally substituted, identically or differently, with 1, 2, 3 or 4 groups selected from:
   halogen, —OH, —CN, —C₁-C₆-alkyl, C₁-C₆-alkoxy-;
R³ represents a hydrogen atom, or a C₁-C₆-alkyl- or C₃-C₆-cycloalkyl- group; wherein said C₁-C₆-alkyl- or C₃-C₆-cycloalkyl- group is optionally substituted, identically or differently, with 1, 2, 3 or 4 groups selected from:
   halogen, —OH, —CN, —C₁-C₆-alkyl, C₁-C₆-alkoxy-;
      or
A=—NR³—; and R¹ and R³, together with the nitrogen atom they are attached to, represent a 3- to 7-membered heterocycloalkyl- or benzo fused 3- to 7-membered heterocycloalkyl- group; wherein said group is optionally substituted, identically or differently, with 1, 2, 3 or 4 R⁷ groups;
R^{4a}, R^{4b}
   represent, independently from each other, a hydrogen atom, or a C₁-C₆-alkyl-, —(CH₂)ₚ—C₂-C₆-alkenyl, —(CH₂)_{q}—C₄-C₈-cycloalkenyl, —(CH₂)ₚ—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, R^{8a}(R^{8b})N—C₁-C₆-alkyl-, halo-C₁-C₆-alkoxy-C₁-C₆-alkyl-, —C(═O)R⁸, —C(═O)N(R^{8a}R^{8b}), —C(═O)O—R⁸, —S(═O)R⁸, —S(═O)₂R⁸, —S(═O)(═NR^{8a})R^{8b} or —S(═O)₂N(R^{8a})R^{8b} group;
   said C₁-C₆-alkyl-, —(CH₂)ₚ—C₂-C₆-alkenyl, —(CH₂)_{q}—C₄-C₈-cycloalkenyl, —(CH₂)ₚ—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl- or halo-C₁-C₆-alkoxy-C₁-C₆-alkyl- group being optionally substituted, identically or differently, with 1, 2, 3, 4 or 5 R⁷ groups;
R^{5a}, R^{5b}
   represent, independently from each other, a hydrogen atom or a halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, C₁-C₆-alkoxy-, C₁-C₆-alkoxy-C₁-C₆-alkyl-, halo-C₁-C₆-alkoxy-C₁-C₆-alkyl-, —C(═O)R⁸, —C(═O)N(R^{8a}R^{8b}), —C(═O)O—R⁸, —S(═O)R⁸, —S(═O)₂R⁸, —S(═O)(═NR^{8a})R^{8b} or —S(═O)₂N(R^{8a})R^{8b} group;
      or
R^{5a} and R^{5b} together
   form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- or —O—(C₂-C₆-alkylene)-O— group;
   said C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- or —O—(C₂-C₆-alkylene)-O— group being optionally substituted, identically or differently, with 1, 2, 3, 4 or 5 R⁷ groups;
R^{6a}, R^{6b}
   represent, independently from each other, a hydrogen atom or halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C(═O)R⁸, —C(═O)N(R^{8a}R^{8b}) or —C(═O)O—R⁸ group;
      or
R^{6a} and R^{6b} together
   form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- group;
   said C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C₁-C₆-alkylene-, —(CH₂)_{q}—C₂-C₆-alkenylene-, halo-C₁-C₆-alkylene- or —(C₁-C₃-alkylene)-Q-(C₁-C₃-alkylene)- group being optionally substituted, identically or differently, with 1, 2, 3, 4 or 5 R⁷ groups;
R⁷ represents a halogen atom, or a HO—, —CN, C₁-C₆-alkoxy-, C₁-C₆-alkyl-, halo-C₁-C₆-alkyl-, R^{8a}(R^{8b})N—C₁-C₆-alkyl-, halo-C₁-C₆-alkoxy-, HO—C₁-C₆-alkyl-, HO—C₁-C₆-alkoxy-, C₁-C₆-alkoxy-C₁-C₆-alkyl-, halo-C₁-C₆-alkoxy-C₁-C₆-alkyl-, C₂-C₆-alkenyl-, C₂-C₆-alkynyl-, 3- to 7-membered heterocycloalkyl-, aryl-, heteroaryl-, —C(═O)R⁸, —C(═O)N(R^{8a})R^{8b}, —C(═O)O—R⁸, —N(R^{8a})R^{8b}, —NO₂, —N(R^{8a})C(═O)R^{8b}, —N(R^{8c})C(═O)N(R^{8a})R^{8b}, —N(R^{8a})C(═O)OR^{8b}, —N(R^{8a})S(═O)R^{8b}, —N(R^{8a})S(═O)₂R^{8b}, —N═S(═O)(R^{8a})R^{8b}, —OR⁸, —O(C═O)R⁸, —O(C═O)N(R^{8a})R^{8b}, —O(C═O)OR⁸, —SR⁸, —S(═O)R⁸, —S(═O)N(R^{8a})R^{8b}, —S(═O)₂R⁸, —S(═O)₂OR⁸, —S(═O)₂N(R^{8a})R^{8b}, —S(═O)(═NR^{8c})R⁸ or —P(═O)(R^{8a})(OR^{8b}) group;
   wherein said aryl- or heteroaryl- group is optionally substituted, identically or differently, with 1, 2 or 3 C₁-C₆-alkyl groups;
      or
      when 2 R⁷ groups are present ortho- to each other on an aryl ring, said 2 R⁷ groups together form a bridge:
   *CH═N—N(H)*, *O(CH₂)₂O*, *O(CH₂)O*, *O(CF₂)O*, *C(═O)OCH₂*, *OC(═O)C(R^{8a})═C(R^{8b})*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═S)S*, *N(R^{8a})C(═O)C(R^{8b})═C(R^{8c})*, *NHC(═O)NH*, *S(═O)ₓCH₂CH₂*, *CH₂S(═O)ₓCH₂*, *N(H)C(═O)—C(═O)N(H)*, *(CH₂)ₜ*; wherein each * represents the point of attachment to said aryl ring;
R⁸, R^{8a}, R^{8b}, R^{8c}
   represent, independently from each other, a hydrogen atom, or a C₁-C₆-alkyl-, C₃-C₆-cycloalkyl-, C₃-C₆-cycloalkyl-(CH₂)—, C₁-C₆-alkyl-aryl-, C₂-C₆-alkenyl-, 3- to 7-membered heterocycloalkyl-, aryl-, heteroaryl-, aryl-C₁-C₆-alkyl-, or heteroaryl-C₁-C₆-alkyl- group;
   said C₁-C₆-alkyl-, C₃-C₆-cycloalkyl-, C₁-C₆-alkyl-aryl-, C₂-C₆-alkenyl-,
   3- to 7-membered heterocycloalkyl-, aryl-, heteroaryl-, aryl-C₁-C₆-alkyl- or heteroaryl-C₁-C₆-alkyl- group being optionally substituted, identically or differently, with 1, 2, 3, 4 or 5 R¹⁰ groups;
      or
R^{8a} and R^{8b} together form a C₁-C₆-alkylene- or halo-C₁-C₆-alkylene- group;
Q represents a —O—, —S—, —S(═O)—, —S(═O)₂—, —S(═O)(NR⁹)—, —N(R⁹)—, —C(═O)—, —C(═O)—O— or —C(═O)—N(R⁹)— group;
R⁹, R^{9a}, R^{9b}
   represent, independently from each other, a hydrogen atom, or a C₁-C₆-alkyl-, C₃-C₆-cycloalkyl-, C₁-C₆-alkyl-aryl-, aryl- or heteroaryl- group; wherein said C₁-C₆-alkyl-, C₃-C₆-cycloalkyl-, C₁-C₆-alkyl-aryl-, aryl- or heteroaryl- group is optionally substituted, identically or differently, with 1, 2, 3, or 4 groups selected from: halogen, —OH, —CN, —C₁-C₆-alkyl, C₁-C₆-alkoxy-;
R¹⁰ represents a halogen atom or a group selected from:
   C₁-C₆-alkoxy-, C₁-C₆-alkyl-, halo-C₁-C₆-alkyl-, halo-C₁-C₆-alkoxy-, —CN, —OH, HO—C₁-C₆-alkyl-, —S(═O)_{X}(C₁-C₆-alkyl), —S(═O)_{X}(aryl), —S(═O)_{X}(C₁-C₆-alkyl-aryl), —S(═O)N(R⁹)(C₁-C₆-alkyl), —N(R^{9a})(R^{9b}), —C(═O)R⁹, —C(═O)OR⁹, —C(═O)N(R^{9a})(R^{9b});
m is an integer of 0, 1, 2, or 3;
n is an integer of 0, 1, 2, or 3;
p is an integer of 1, 2, 3, 4 or 5;
q is an integer of 0, 1, 2, 3, 4 or 5;
t is an integer of 3, 4, 5 or 6;
x is an integer of 0, 1 or 2
   or a stereoisomer, a tautomer, an N-oxide, a hydrate, a solvate, or a salt thereof, or a mixture of same.

The present invention also relates to methods of preparing said compounds, to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### DETAILED DESCRIPTION OF THE INVENTION

The terms as mentioned in the present text have preferably the following meanings:

The term “halogen atom”, “halo-” or “Hal-” is to be understood as meaning a fluorine, chlorine, bromine or iodine atom, preferably a fluorine, chlorine or bromine atom.

The term “C₁-C₆-alkyl” is to be understood as preferably meaning a linear or branched, saturated, monovalent hydrocarbon group having 1, 2, 3, 4, 5 or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group.

The term “C₂-C₆-alkylene” is to be understood as preferably meaning a linear or branched, saturated, bivalent hydrocarbon group having 2, 3, 4, 5 or 6 carbon atoms, e.g. an ethylene, n-propylene, n-butylene, n-pentylene, 2-methylbutylene, n-hexylene, 3-methylpentylene group, or an isomer thereof. Particularly, said group is linear and has 2, 3, 4 or 5 carbon atoms (“C₂-C₅-alkylene”), e.g. an ethylene, n-propylene, n-butylene, n-pentylene group, more particularly 3 or 4 carbon atoms (“C₃-C₄-alkylene”), e.g. an n-propylene or n-butylene group.

The term “halo-C₁-C₆-alkyl” is to be understood as preferably meaning a linear or branched, saturated, monovalent hydrocarbon group in which the term “C₁-C₆-alkyl” is defined supra, and in which one or more hydrogen atoms is replaced by a halogen atom, in identically or differently, i.e. one halogen atom being independent from another. Particularly, said halogen atom is F. Said halo-C₁-C₆-alkyl group is, for example, —CF₃, —CHF₂, —CH₂F, —CF₂CF₃, or —CH₂CF₃.

The term “C₁-C₆-alkoxy” is to be understood as preferably meaning a linear or branched, saturated, monovalent, hydrocarbon group of formula —O—(C₁-C₆-alkyl), in which the term “C₁-C₆-alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof.

The term “halo-C₁-C₆-alkoxy” is to be understood as preferably meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said halo-C₁-C₆-alkoxy group is, for example, —OCF₃, —OCHF₂, —OCH₂F, —OCF₂CF₃, or —OCH₂CF₃.

The term “C₁-C₆-alkoxy-C₁-C₆-alkyl” is to be understood as preferably meaning a linear or branched, saturated, monovalent C₁-C₆-alkyl group, as defined supra, in which one or more of the hydrogen atoms is replaced, in identically or differently, by a C₁-C₆-alkoxy group, as defined supra, e.g. methoxyalkyl, ethoxyalkyl, propyloxyalkyl, iso-propoxyalkyl, butoxyalkyl, iso-butoxyalkyl, tert-butoxyalkyl, sec-butoxyalkyl, pentyloxyalkyl, iso-pentyloxyalkyl, hexyloxyalkyl group, or an isomer thereof.

The term “halo-C₁-C₆-alkoxy-C₁-C₆-alkyl” is to be understood as preferably meaning a linear or branched, saturated, monovalent C₁-C₆-alkoxy-C₁-C₆-alkyl group, as defined supra, in which one or more of the hydrogen atoms is replaced, in identically or differently, by a halogen atom. Particularly, said halogen atom is F. Said halo-C₁-C₆-alkoxy-C₁-C₆-alkyl group is, for example, —CH₂CH₂OCF₃, —CH₂CH₂OCHF₂, —CH₂CH₂OCH₂F, —CH₂CH₂OCF₂CF₃, or —CH₂CH₂OCH₂CF₃.

The term “C₂-C₆-alkenyl” is to be understood as preferably meaning a linear or branched, monovalent hydrocarbon group, which contains one or more double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then said double bonds may be isolated from, or conjugated with, each other. Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, iso-propenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, or methylhexadienyl group. Particularly, said group is vinyl or allyl.

The term “C₂-C₆-alkynyl” is to be understood as preferably meaning a linear or branched, monovalent hydrocarbon group which contains one or more triple bonds, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl, or 3,3-dimethylbut-1-ynyl group. Particularly, said alkynyl group is ethynyl, prop-1-ynyl, or prop-2-ynyl.

The term “C₃-C₆-cycloalkyl” is to be understood as meaning a saturated, monovalent, monocyclic hydrocarbon ring which contains 3, 4, 5 or 6 carbon atoms (“C₃-C₆-cycloalkyl”). Said C₃-C₆-cycloalkyl group is for example a cyclopropyl, cyclobutyl, cyclopentyl, or cyclohexyl ring.

The term “C₄-C₈-cycloalkenyl” is to be understood as preferably meaning a monovalent, monocyclic hydrocarbon ring which contains 4, 5, 6, 7 or 8 carbon atoms and one, two or three double bonds, in conjugation or not, as the size of said cycloalkenyl ring allows. Said C₄-C₈-cycloalkenyl group is for example, a cyclobutenyl, cyclopentenyl, or cyclohexenyl ring.

The term “3- to 7-membered heterocycloalkyl”, is to be understood as meaning a saturated, monovalent, monocyclic hydrocarbon ring which contains 2, 3, 4, 5, or 6 carbon atoms, and one or more heteroatom-containing groups selected from C(═O), O, S, S(═O), S(═O)₂, NR^{a}, in which R^{a} represents a hydrogen atom, or a C₁-C₆-alkyl- group; it being possible for said heterocycloalkyl group to be attached to the rest of the molecule via any one of the carbon atoms or, if present, the nitrogen atom.

Particularly, said 3- to 7-membered heterocycloalkyl can contain 2, 3, 4, or 5 carbon atoms, and one or more of the above-mentioned heteroatom-containing groups (a “3- to 6-membered heterocycloalkyl”), more particularly said heterocycloalkyl can contain 4 or 5 carbon atoms, and one or more of the above-mentioned heteroatom-containing groups (a “5- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, pyrrolinyl, or a 6-membered ring, such as tetrahydropyranyl, piperidinyl, morpholinyl, dithianyl, thiomorpholinyl, piperazinyl, or trithianyl, or a 7-membered ring, such as a diazepanyl ring, for example.

The term “benzo fused 3- to 7-membered heterocycloalkyl”, is to be understood as meaning a 3- to 7-membered heterocycloalkyl group as defined above, onto which a benzene ring is fused. An example of a benzo fused 3- to 7-membered heterocycloalkyl group is

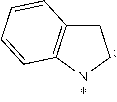

wherein * represents the point of attachment to the rest of the molecule.

The term “4- to 8-membered heterocycloalkenyl”, is to be understood as meaning an unsaturated, monovalent, monocyclic hydrocarbon ring which contains 4, 5, 6, 7 or 8 carbon atoms, and one or more heteroatom-containing groups selected from C(═O), O, S, S(═O), S(═O)₂, NR^{a}, in which R^{a} represents a hydrogen atom or a C₁-C₆-alkyl- group; it being possible for said heterocycloalkenyl group to be attached to the rest of the molecule via any one of the carbon atoms or, if present, the nitrogen atom. Examples of said heterocycloalkenyl are 4H-pyranyl, 2H-pyranyl, 3H-diazirinyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, or 4H-[1,4]thiazinyl.

The term “aryl” is to be understood as preferably meaning a monovalent, aromatic or partially aromatic, mono-, or bi- or tricyclic hydrocarbon ring having 6, 7, 8, 9, 10, 11, 12, 13 or 14 carbon atoms (a “C₆-C₁₄-aryl” group), particularly a ring having 6 carbon atoms (a “C₆-aryl” group), e.g. a phenyl group; or a biphenyl group, or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group, or a ring having 13 carbon atoms, (a “C₁₃-aryl” group), e.g. a fluorenyl group, or a ring having 14 carbon atoms, (a “C₁₄-aryl” group), e.g. an anthracenyl group. Preferably, the aryl group is a phenyl group.

The term “heteroaryl” is understood as preferably meaning a monovalent, monocyclic-, bicyclic- or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc.

In general, and unless otherwise mentioned, the heteroarylic or heteroarylenic radicals include all the possible isomeric forms thereof, e.g. the positional isomers thereof. Thus, for some illustrative non-restricting example, the term pyridinyl or pyridinylene includes pyridin-2-yl, pyridin-2-ylene, pyridin-3-yl, pyridin-3-ylene, pyridin-4-yl and pyridin-4-ylene; or the term thienyl or thienylene includes thien-2-yl, thien-2-ylene, thien-3-yl and thien-3-ylene.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅, C₁-C₆; particularly C₁-C₂, C₁-C₃, C₁-C₄, C₁-C₅, C₁-C₆; more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

Ring system substituent means a substituent attached to an aromatic or nonaromatic ring system which, for example, replaces an available hydrogen on the ring system.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

As used herein, the term “one or more times”, e.g. in the definition of the substituents of the compounds of the general formulae of the present invention, is understood as meaning “one, two, three, four or five times, particularly one, two, three or four times, more particularly one, two or three times, even more particularly one or two times”.

Where the plural form of the word compounds, salts, polymorphs, hydrates, solvates and the like, is used herein, this is taken to mean also a single compound, salt, polymorph, isomer, hydrate, solvate or the like.

The compounds of this invention may contain one or more asymmetric centre, depending upon the location and nature of the various substituents desired. Asymmetric carbon atoms may be present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

The compounds of the present invention may contain sulphur atoms which are asymmetric, such as an asymmetric sulphoxide or sulphoximine group, of structure:

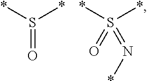

for example,
in which * indicates atoms to which the rest of the molecule can be bound.

Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, namely:

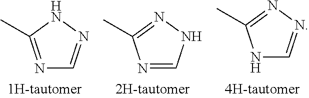

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, Lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethanesulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, Lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

As used herein, the term “in vivo hydrolysable ester” is understood as meaning an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of this invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorphs, or as a mixture of more than one polymorphs, in any ratio.

In accordance with a first aspect, the present invention covers compounds of general formula I:

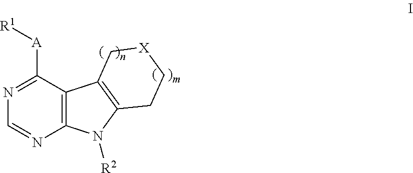

in which:
A represents —O—, —S—, —S(═O)—, —S(═O)₂—, —S(═O)(NR³)— or —NR³—;
X represents —O—, —S—, —S(═O)—, —S(═O)₂—, —S(═O)(NR^{4a})—, —NR^{4a}—, —C(O—C₁-C₆-alkyl)₂-, —C(O—CH₂—CH₂—O)—, —C(O—CH₂—CH₂—CH₂—O)—, —C(O—CH₂—C(CH₃)₂—CH₂—O)—, —C(═O)—, —C(═O)NR^{4a}—, —NR^{4a}C(═O)—, —C(═O)O—, —OC(═O)—, —C(H)(OR^{4a})—, —C(R^{4a})(OR^{4b})—, —C═NR^{4a}—, —C(H)NR^{4a}R^{4b}—, —C(R^{5a})(R^{5b})—, —C(═CR^{6a}R^{6b})— or —CH(CHR^{6a}R^{6b})—;
R¹ represents an aryl- or heteroaryl- group; wherein said aryl- or heteroaryl-group is optionally substituted, identically or differently, with 1, 2, 3, 4 or 5 R⁷ groups;
R² represents a hydrogen atom, or a C₁-C₆-alkyl- or C₃-C₆-cycloalkyl- group; wherein said C₁-C₆-alkyl- or C₃-C₆-cycloalkyl- group is optionally substituted, identically or differently, with 1, 2, 3 or 4 groups selected from:
   halogen, —OH, —CN, —C₁-C₆-alkyl, C₁-C₆-alkoxy-;
R³ represents a hydrogen atom, or a C₁-C₆-alkyl- or C₃-C₆-cycloalkyl- group; wherein said C₁-C₆-alkyl- or C₃-C₆-cycloalkyl- group is optionally substituted, identically or differently, with 1, 2, 3 or 4 groups selected from:
   halogen, —OH, —CN, —C₁-C₆-alkyl, C₁-C₆-alkoxy-;
      or
A=—NR³—; and R¹ and R³, together with the nitrogen atom they are attached to, represent a 3- to 7-membered heterocycloalkyl- or benzo fused 3- to 7-membered heterocycloalkyl- group; wherein said group is optionally substituted, identically or differently, with 1, 2, 3 or 4 R⁷ groups;
R^{4a}, R^{4b}
   represent, independently from each other, a hydrogen atom, or a C₁-C₆-alkyl-, —(CH₂)ₚ—C₂-C₆-alkenyl, —(CH₂)_{q}—C₄-C₈-cycloalkenyl, —(CH₂)ₚ—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, R^{8a}(R^{8b})N—C₁-C₆-alkyl-, halo-C₁-C₆-alkoxy-C₁-C₆-alkyl-, —C(═O)R⁸, —C(═O)N(R^{8a}R^{8b}), —C(═O)O—R⁸, —S(═O)R⁸, —S(═O)₂R⁸, —S(═O)(═NR^{8a})R^{8b} or —S(═O)₂N(R^{8a})R^{8b} group;
   said C₁-C₆-alkyl-, —(CH₂)ₚ—C₂-C₆-alkenyl, —(CH₂)_{q}—C₄-C₈-cycloalkenyl, —(CH₂)ₚ—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl- or halo-C₁-C₆-alkoxy-C₁-C₆-alkyl- group being optionally substituted, identically or differently, with 1, 2, 3, 4 or 5 R⁷ groups;
R^{5a}, R^{5b}
   represent, independently from each other, a hydrogen atom or a halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, C₁-C₆-alkoxy-, C₁-C₆-alkoxy-C₁-C₆-alkyl-, halo-C₁-C₆-alkoxy-C₁-C₆-alkyl-, —C(═O)R⁸, —C(═O)N(R^{8a}R^{8b}), —C(═O)O—R⁸, —S(═O)R⁸, —S(═O)₂R⁸, —S(═O)(═NR^{8a})R^{8b} or —S(═O)₂N(R^{8a})R^{8b} group;
   said C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl- or halo-C₁-C₆-alkoxy-C₁-C₆-alkyl- group being optionally substituted, identically or differently, with 1, 2, 3, 4 or 5 R⁷ groups;
      or
R^{5a} and R^{5b} together
   form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- or —O—(C₂-C₆-alkylene)-O— group;
   said —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- or —O—(C₂-C₆-alkylene)-O— group being optionally substituted, identically or differently, with 1, 2, 3, 4 or 5 R⁷ groups;
R^{6a}, R^{6b}
   represent, independently from each other, a hydrogen atom or halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C(═O)R⁸, —C(═O)N(R^{8a}R^{8b}) or —C(═O)O—R⁸ group;
   said C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl- or halo-C₁-C₆-alkoxy-C₁-C₆-alkyl- group being optionally substituted, identically or differently, with 1, 2, 3, 4 or 5 R⁷ groups;
      or
R^{6a} and R^{6b} together
   form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- group;
   said —C₁-C₆-alkylene-, —(CH₂)_{q}—C₂-C₆-alkenylene-, halo-C₁-C₆-alkylene- or —(C₁-C₃-alkylene)-Q-(C₁-C₃-alkylene)- group being optionally substituted, identically or differently, with 1, 2, 3, 4 or 5 R⁷ groups;
R⁷ represents a halogen atom, or a HO—, —CN, C₁-C₆-alkoxy-, C₁-C₆-alkyl-, halo-C₁-C₆-alkyl-, R^{8a}(R^{8b})N—C₁-C₆-alkyl-, halo-C₁-C₆-alkoxy-, HO—C₁-C₆-alkyl-, HO—C₁-C₆-alkoxy-, C₁-C₆-alkoxy-C₁-C₆-alkyl-, halo-C₁-C₆-alkoxy-C₁-C₆-alkyl-, C₂-C₆-alkenyl-, C₂-C₆-alkynyl-, 3- to 7-membered heterocycloalkyl-, aryl-, heteroaryl-, —C(═O)R⁸, —C(═O)N(R^{8a})R^{8b}, —C(═O)O—R⁸, —N(R^{8a})R^{8b}, —NO₂, —N(R^{8a})C(═O)R^{8b}, —N(R^{8c})C(═O)N(R^{8a})R^{8b}, —N(R^{8a})C(═O)OR^{8b}, —N(R^{8a})S(═O)R^{8b}, —N(R^{8a})S(═O)₂R^{8b}, —N═S(═O)(R^{8a})R^{8b}, —OR⁸, —O(C═O)R⁸, —O(C═O)N(R^{8a})R^{8b}, —O(C═O)OR⁸, —SR⁸, —S(═O)R⁸, —S(═O)N(R^{8a})R^{8b}, —S(═O)₂R⁸, —S(═O)₂OR⁸, —S(═O)₂N(R^{8a})R^{8b}, —S(═O)(═NR^{8c})R⁸ or —P(═O)(R^{8a})(OR^{8b}) group;
   wherein said aryl- or heteroaryl- group is optionally substituted, identically or differently, with 1, 2 or 3 C₁-C₆-alkyl groups;
      or
      when 2 R⁷ groups are present ortho- to each other on an aryl ring, said 2 R⁷ groups together form a bridge:
   *CH═N—N(H)*, *O(CH₂)₂O*, *O(CH₂)O*, *O(CF₂)O*, *C(═O)OCH₂*, *OC(═O)C(R^{8a})═C(R^{8b})*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═S)S*, *N(R^{8a})C(═O)C(R^{8b})═C(R^{8c})*, *NHC(═O)NH*, *S(═O)ₓCH₂CH₂*, *CH₂S(═O)ₓCH₂*, *N(H)C(═O)—C(═O)N(H)*, *(CH₂)ₜ*; wherein each * represents the point of attachment to said aryl ring;
R⁸, R^{8a}, R^{8b}, R^{8c}
   represent, independently from each other, a hydrogen atom, or a C₁-C₆-alkyl-, C₃-C₆-cycloalkyl-, C₃-C₆-cycloalkyl-(CH₂)—, C₁-C₆-alkyl-aryl-, C₂-C₆-alkenyl-, 3- to 7-membered heterocycloalkyl-, aryl-, heteroaryl-, aryl-C₁-C₆-alkyl-, or heteroaryl-C₁-C₆-alkyl- group;
   said C₁-C₆-alkyl-, C₃-C₆-cycloalkyl-, C₁-C₆-alkyl-aryl-, C₂-C₆-alkenyl-,
   3- to 7-membered heterocycloalkyl-, aryl-, heteroaryl-, aryl-C₁-C₆-alkyl- or heteroaryl-C₁-C₆-alkyl- group being optionally substituted, identically or differently, with 1, 2, 3, 4 or 5 R¹⁰ groups;
      or
R^{8a} and R^{8b} together form a C₁-C₆-alkylene- or halo-C₁-C₆-alkylene- group;
Q represents a —O—, —S—, —S(═O)—, —S(═O)₂—, —S(═O)(NR⁹)—, —N(R⁹)—, —C(═O)—, —C(═O)—O— or —C(═O)—N(R⁹)— group;
R⁹, R^{9a}, R^{9b}
   represent, independently from each other, a hydrogen atom, or a C₁-C₆-alkyl-, C₃-C₆-cycloalkyl-, C₁-C₆-alkyl-aryl-, aryl- or heteroaryl- group; wherein said C₁-C₆-alkyl-, C₃-C₆-cycloalkyl-, C₁-C₆-alkyl-aryl-, aryl- or heteroaryl- group is optionally substituted, identically or differently, with 1, 2, 3, or 4 groups selected from: halogen, —OH, —CN, —C₁-C₆-alkyl, C₁-C₆-alkoxy-;
R¹⁰ represents a halogen atom or a group selected from:
   C₁-C₆-alkoxy-, C₁-C₆-alkyl-, halo-C₁-C₆-alkyl-, halo-C₁-C₆-alkoxy-, —CN, —OH, HO—C₁-C₆-alkyl-, —S(═O)_{X}(C₁-C₆-alkyl), —S(═O)_{X}(aryl), —S(═O)_{X}(C₁-C₆-alkyl-aryl), —S(═O)N(R⁹)(C₁-C₆-alkyl), —N(R^{9a})(R^{9b}), —C(═O)R⁹, —C(═O)OR⁹, —C(═O)N(R^{9a})(R^{9b});
m is an integer of 0, 1, 2, or 3;
n is an integer of 0, 1, 2, or 3;
p is an integer of 1, 2, 3, 4 or 5;
q is an integer of 0, 1, 2, 3, 4 or 5;
t is an integer of 3, 4, 5 or 6;
x is an integer of 0, 1 or 2
   or a stereoisomer, a tautomer, an N-oxide, a hydrate, a solvate, or a salt thereof, or a mixture of same.

In a preferred embodiment, the invention relates to compounds of formula I, supra, wherein A represents —O—, —S— or —NR³—.

In another preferred embodiment, the invention relates to compounds of formula I, supra, wherein A represents —O— or —S—.

In another preferred embodiment, the invention relates to compounds of formula I, supra, wherein A represents —NR³—.

In another preferred embodiment, the invention relates to compounds of formula I, supra, wherein A represents —NH—.

In another preferred embodiment, the invention relates to compounds of formula I, supra, wherein R¹ and A together represent an indoline-1-yl-group; wherein said group is optionally substituted, identically or differently, with 1, 2, 3 or 4 groups selected from halogen, nitro, —OH, or —CN.

In another preferred embodiment, the invention relates to compounds of formula I, supra, wherein X represents —S—, —S(═O)—, —S(═O)₂—, —NR^{4a}—, —C(O—CH₂—CH₂—O)—, —C(R^{5a})(R^{5b})— or —CH(CHR^{6a}R^{6b})—.

In another preferred embodiment, the invention relates to compounds of formula I, supra, wherein X represents —O—.

In another preferred embodiment, the invention relates to compounds of formula I, supra, wherein X represents —S—, —S(═O)— or —S(═O)₂—.

In another preferred embodiment, the invention relates to compounds of formula I, supra, wherein X represents —NR^{4a}—.

In another preferred embodiment, the invention relates to compounds of formula I, supra, wherein X represents —C(O—CH₂—CH₂—O)—.

In another preferred embodiment, the invention relates to compounds of formula I, supra, wherein X represents —C(R^{5a})(R^{5b})—.

In another preferred embodiment, the invention relates to compounds of formula I, supra, wherein X represents a group selected from:

### —(CH₂)—, —(CF₂)—, —C(H)(C(═O)R⁸)—, —C(H)(C(═O)N(R^{8a}R^{8b}))—, —C(H)(C(═O)O—R⁸)—.

In another preferred embodiment, the invention relates to compounds of formula I, supra, wherein X represents —(CH₂)—.

In another preferred embodiment, the invention relates to compounds of formula I, supra, wherein X represents —(CF₂)—.

In another preferred embodiment, the invention relates to compounds of formula I, supra, wherein X represents —C(H)(C(═O)R⁸)—.

In another preferred embodiment, the invention relates to compounds of formula I, supra, wherein X represents —C(H)(C(═O)O—R⁸)—.

In another preferred embodiment, the invention relates to compounds of formula I, supra, wherein X represents —C(H)(C(═O)N(R^{8a}R^{8b}))—.

In another preferred embodiment, the invention relates to compounds of formula I, supra, wherein R¹ represents an aryl- or heteroaryl- group; wherein said aryl- or heteroaryl- group is optionally substituted, identically or differently, with 1, 2, 3 or 4 R⁷ groups.

In another preferred embodiment, the invention relates to compounds of formula I, supra, wherein R¹ represents an aryl- or heteroaryl- group; wherein said aryl- or heteroaryl- group is optionally substituted, identically or differently, with 1, 2 or 3 R⁷ groups.

In another preferred embodiment, the invention relates to compounds of formula I, supra, wherein R¹ represents an aryl- or heteroaryl- group; wherein said aryl- or heteroaryl- group is substituted, identically or differently, with 1, 2 or 3 R⁷ groups.

In another preferred embodiment, the invention relates to compounds of formula I, supra, wherein A represents —NR³—; and R¹ and R³, together with the nitrogen atom they are attached to, represent a 3- to 7-membered heterocycloalkyl- or benzo fused 3- to 7-membered heterocycloalkyl- group; wherein said group is optionally substituted, identically or differently, with 1, 2, 3 or 4 R⁷ groups. Preferably, the benzo fused 3- to 7-membered heterocycloalkyl- group is an indoline-1-yl-group.

In another preferred embodiment, the invention relates to compounds of formula I, supra, wherein R¹ represents

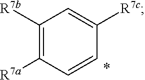

wherein * represents the point of attachment of the group to the rest of the molecule;
wherein R^{7a} and R^{7b} represent, independently from each other, a hydrogen atom or a halogen atom, or a group selected from: —CN, C₁-C₆-alkoxy-, C₁-C₆-alkyl-, halo-C₁-C₆-alkyl-, halo-C₁-C₆-alkoxy-, C₁-C₆-alkoxy-C₁-C₆-alkyl-, halo-C₁-C₆-alkoxy-C₁-C₆-alkyl-,
or
R^{7a} and R^{7b} together form a bridge:
*CH═N—N(H)*, *O(CH₂)₂O*, *O(CH₂)O*, *O(CF₂)O*, *C(═O)OCH₂*, *OC(═O)C(R^{8a})═C(R^{8b})*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═S)S*, *N(R^{8a})C(═O)C(R^{8b})═C(R^{8c})*, *NHC(═O)NH*, *S(═O)ₓCH₂CH₂*, *CH₂S(═O)ₓCH₂*, *N(H)C(═O)—C(═O)N(H)*, *(CH₂)ₜ*; wherein each * represents the point of attachment to the phenyl ring and R^{8a}, R^{8b}, R^{8c}, x, and t are as defined for general formula I, supra; and
wherein R^{7c} represents a hydrogen atom, a halogen atom or a group selected from: 3- to 7-membered heterocycloalkyl-, C₁-C₆-alkoxy-, halo-C₁-C₆-alkoxy-, HO—C₁-C₆-alkoxy-, —OR⁸; in which R⁸ represents a C₃-C₆-cycloalkyl-, C₃-C₆-cycloalkyl-(CH₂)— or 3- to 7-membered heterocycloalkyl- group.

In another preferred embodiment, the invention relates to compounds of formula I, supra, wherein R¹ represents

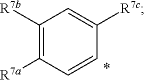

wherein * represents the point of attachment of the group to the rest of the molecule;
wherein R^{7a} and R^{7b} represent, independently from each other, a hydrogen atom or a halogen atom, or a group selected from: C₁-C₆-alkoxy-, C₁-C₆-alkyl-, halo-C₁-C₆-alkyl-;
or
R^{7a} and R^{7b} together form a bridge:
*CH═N—N(H)*, *O(CH₂)₂O*, *O(CF₂)O*, *C(═O)OCH₂*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═S)S*, *N(R^{8a})C(═O)C(R^{8b})═C(R^{8c})*, *S(═O)ₓCH₂CH₂*, *CH₂S(═O)ₓCH₂*, *N(H)C(═O)—C(═O)N(H)*, *(CH₂)ₜ*; wherein each * represents the point of attachment to the phenyl ring and R^{8a}, R^{8b}, R^{8c}, x, and t are as defined for general formula I, supra; and
wherein R^{7c} represents a hydrogen atom, a halogen atom or a group selected from: 3- to 7-membered heterocycloalkyl-, C₁-C₆-alkoxy-, halo-C₁-C₆-alkoxy-, HO—C₁-C₆-alkoxy-, —OR⁸; in which R⁸ represents a C₃-C₆-cycloalkyl-, C₃-C₆-cycloalkyl-(CH₂)— or 3- to 7-membered heterocycloalkyl- group.

In another preferred embodiment, the invention relates to compounds of formula I, supra, wherein R¹ represents

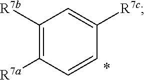

wherein * represents the point of attachment of the group to the rest of the molecule;
wherein R^{7a} and R^{7b} represent, independently from each other, a hydrogen atom or a halogen atom, or a group selected from: C₁-C₆-alkoxy-, C₁-C₆-alkyl-, halo-C₁-C₆-alkyl-;
or
R^{7a} and R^{7b} together form a bridge:
*CH═N—N(H)*, *N(H)C(═O)S*, *C(═O)OCH₂*; wherein each * represents the point of attachment to the phenyl ring; and
wherein R^{7c} represents a hydrogen atom, a halogen atom or a group selected from: 3- to 7-membered heterocycloalkyl-, C₁-C₆-alkoxy-, halo-C₁-C₆-alkoxy-, HO—C₁-C₆-alkoxy-, —OR⁸; in which R⁸ represents a C₃-C₆-cycloalkyl-, C₃-C₆-cycloalkyl-(CH₂)— or 3- to 7-membered heterocycloalkyl- group.

In another preferred embodiment, the invention relates to compounds of formula I, supra, wherein R¹ represents a group selected from:

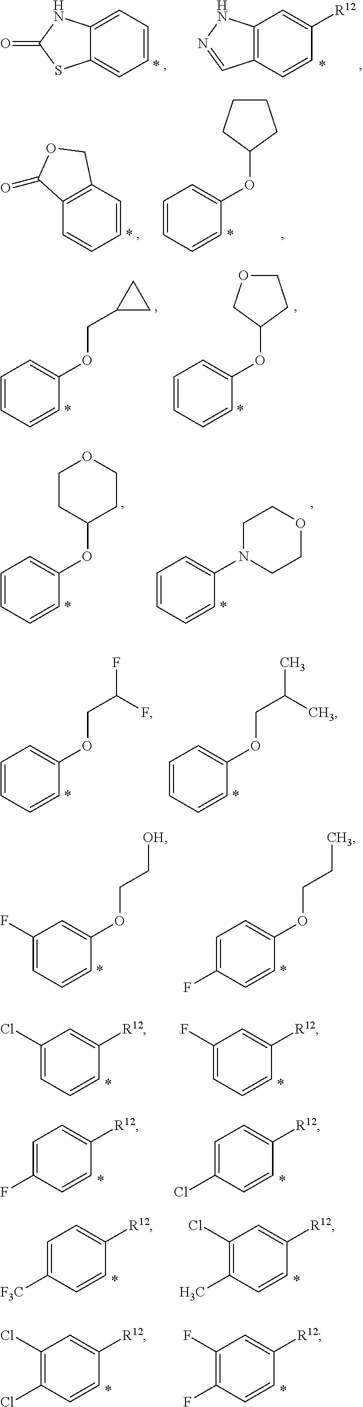

wherein * represents the point of attachment of the groups to the rest of the molecule; and R¹² represents a hydrogen atom, a halogen atom or a group selected from: C₁-C₆-alkoxy-, halo-C₁-C₆-alkoxy-, HO—C₁-C₆-alkoxy-, —OR⁸; in which R⁸ represents a C₃-C₆-cycloalkyl-, C₃-C₆-cycloalkyl-(CH₂)— or 3- to 7-membered heterocycloalkyl- group.

Preferably, R¹² represents a group selected from: C₁-C₆-alkoxy-, halo-C₁-C₆-alkoxy-, HO—C₁-C₆-alkoxy-, —OR⁸; in which R⁸ represents a C₃-C₆-cycloalkyl-, C₃-C₆-cycloalkyl-(CH₂)— or 3- to 7-membered heterocycloalkyl- group.

More preferably, R¹² represents a methoxy- or propoxy- group.

In another preferred embodiment, the invention relates to compounds of formula I, supra, wherein R¹ represents a group selected from:

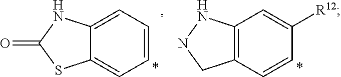

wherein * represents the point of attachment of the groups to the rest of the molecule; and R¹² represents a hydrogen atom or a group selected from: C₁-C₆-alkoxy-, halo-C₁-C₆-alkoxy-, HO—C₁-C₆-alkoxy-, —OR⁸; in which R⁸ represents a C₃-C₆-cycloalkyl-, C₃-C₆-cycloalkyl-(CH₂)— or 3- to 7-membered heterocycloalkyl-group. Preferably, R¹² represents a methoxy- or propoxy- group.

In another preferred embodiment, the invention relates to compounds of formula I, supra, wherein R² represents a hydrogen atom or a C₁-C₆-alkyl- group; wherein said C₁-C₆-alkyl- group is optionally substituted, identically or differently, with 1, 2, 3 or 4 groups selected from halogen, —OH, —CN, C₁-C₆-alkoxy-.

In another preferred embodiment, the invention relates to compounds of formula I, supra, wherein R² represents a hydrogen atom or a C₁-C₆-alkyl- group; wherein said C₁-C₆-alkyl- group is optionally substituted, identically or differently, with 1 or 2 4 groups selected from halogen, —OH, —CN.

In another preferred embodiment, the invention relates to compounds of formula I, supra, wherein R² represents a hydrogen atom or a C₁-C₆-alkyl- group.

In another preferred embodiment, the invention relates to compounds of formula I, supra, wherein R² represents a hydrogen atom.

In another preferred embodiment, the invention relates to compounds of formula I, supra, wherein R³ represents a hydrogen atom.

In another preferred embodiment, the invention relates to compounds of formula I, supra, wherein R^{4a}, R^{4b} represent, independently from each other, a hydrogen atom, or a C₁-C₆-alkyl-, aryl-, aryl-C₁-C₆-alkyl-, heteroaryl-, —C(═O)R⁸, —C(═O)N(R^{8a}R^{8b}), —C(═O)O—R⁸, —S(═O)₂R⁸ or —S(═O)₂N(R^{8a})R^{8b} group;

said group being optionally substituted, identically or differently, with 1, 2, 3, 4 or 5 R⁷ groups.

In another preferred embodiment, the invention relates to compounds of formula I, supra, wherein R^{4a}, R^{4b} represent, independently from each other, a hydrogen atom, or a C₁-C₆-alkyl-, aryl-, aryl-C₁-C₆-alkyl-, heteroaryl-, —C(═O)R⁸, —C(═O)N(R^{8a}R^{8b}), —C(═O)O—R⁸, —S(═O)₂R⁸ or —S(═O)₂N(R^{8a})R^{8b} group;

said group being optionally substituted, identically or differently, with 1, 2 or 3 R⁷ groups.

In another preferred embodiment, the invention relates to compounds of formula I, supra, wherein R^{5a}, R^{5b} represent, independently from each other, a hydrogen atom or halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
—(CH₂)_{q}-(3- to 7-membered heterocycloalkyl),
—(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C(═O)R⁸, —C(═O)N(R^{8a}R^{8b}), —C(═O)O—R⁸, —S(═O)R⁸, —S(═O)₂R⁸, —S(═O)(═NR^{8a})R^{8b} or —S(═O)₂N(R^{8a})R^{8b} group.

In another preferred embodiment, the invention relates to compounds of formula I, supra, wherein R^{5a} and R^{5b} together form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- or —O—(C₂-C₆-alkylene)-O— group;

said group being optionally substituted, identically or differently, with 1, 2, 3, 4 or 5 R⁷ groups.

In another preferred embodiment, the invention relates to compounds of formula I, supra, wherein R^{5a} and R^{5b} together form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- or —O—(C₂-C₆-alkylene)-O— group;

said being optionally substituted, identically or differently, with 1, 2 or 3 R⁷ groups.

In another preferred embodiment, the invention relates to compounds of formula I, supra, wherein R^{5a} and R^{5b} together form a —O—(C₂-C₆-alkylene)-O— group.

In another preferred embodiment, the invention relates to compounds of formula I, supra, wherein R^{5a}, R^{5b} represent, independently from each other, a hydrogen atom or a halogen atom, or a C₁-C₆-alkoxy-, C₁-C₆-alkoxy-C₁-C₆-alkyl-, —C(═O)R⁸, —C(═O)N(R^{8a}R^{8b}) or —C(═O)O—R⁸ group.

In another preferred embodiment, the invention relates to compounds of formula I, supra, wherein R^{5a} represents a hydrogen atom and R^{5b} is selected from:

### —H, —C(═O)R⁸, —C(═O)N(R^{8a}R^{8b}), —C(═O)O—R⁸.

In another preferred embodiment, the invention relates to compounds of formula I, supra, wherein each of R^{5a} and R^{5b} represents a hydrogen atom.

In another preferred embodiment, the invention relates to compounds of formula I, supra, wherein each of R^{5a} and R^{5b} represents a fluorine atom.

In another preferred embodiment, the invention relates to compounds of formula I, supra, wherein R^{5a} represents a hydrogen atom and R^{5b} represents —C(═O)R⁸.

In another preferred embodiment, the invention relates to compounds of formula I, supra, wherein R^{5a} represents a hydrogen atom and R^{5b} represents —C(═O)N(R^{8a}R^{8b}).

In another preferred embodiment, the invention relates to compounds of formula I, supra, wherein R^{5a} represents a hydrogen atom and R^{5b} represents —C(═O)O—R⁸.

In another preferred embodiment, the invention relates to compounds of formula I, supra, wherein R^{6a}, R^{6b} represent, independently from each other, a hydrogen atom or halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
—(CH₂)_{q}-(3- to 7-membered heterocycloalkyl),
—(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C(═O)R⁸, —C(═O)N(R^{8a}R^{8b}) or —C(═O)O—R⁸ group.

In another preferred embodiment, the invention relates to compounds of formula I, supra, wherein R^{6a} and R^{6b} together form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- group;

said group being optionally substituted, identically or differently, with 1, 2, 3, 4 or 5 R⁷ groups.

In another preferred embodiment, the invention relates to compounds of formula I, supra, wherein each of R^{6a} and R^{6b} represents a hydrogen atom.

In another preferred embodiment, the invention relates to compounds of formula I, supra, wherein R⁷ represents a halogen atom, or a HO—, —CN, C₁-C₆-alkoxy-, C₁-C₆-alkyl-, halo-C₁-C₆-alkyl-, R^{8a}(R^{8b})N—C₁-C₆-alkyl-, halo-C₁-C₆-alkoxy-, HO—C₁-C₆-alkyl-, HO—C₁-C₆-alkoxy, C₁-C₆-alkoxy-C₁-C₆-alkyl-, halo-C₁-C₆-alkoxy-C₁-C₆-alkyl-, C₂-C₆-alkenyl-, C₂-C₆-alkynyl-, 3- to 7-membered heterocycloalkyl-, aryl-, heteroaryl-, —O—(CH₂)_{q}—C₃-C₆-cycloalkyl, —O—(CH₂)_{q}-(3- to 7-membered heterocycloalkyl), —C(═O)R⁸, —C(═O)N(R^{8a})R^{8b}, —C(═O)O—R⁸, —N(R^{8a})R^{8b}, —NO₂, —N(R^{8a})C(═O)R^{8b}, —N(R^{8c})C(═O)N(R^{8a})R^{8b}, —N(R^{8a})C(═O)OR^{8b}, —N(R^{8a})S(═O)R^{8b}, —N(R^{8a})S(═O)₂R^{8b}, —N═S(═O)(R^{8a})R^{8b}, —OR⁸, —O(C═O)R⁸, —O(C═O)N(R^{8a})R^{8b}, —O(C═O)OR⁸, —SR⁸, —S(═O)R⁸, —S(═O)N(R^{8a})R^{8b}, —S(═O)₂R⁸, —S(═O)₂OR⁸, —S(═O)₂N(R^{8a})R^{8b}, —S(═O)(═NR^{8c})R⁸ or —P(═O)(R^{8a})(OR^{8b}) group;

or when 2 R⁷ groups are present ortho- to each other on an aryl ring, said 2 R⁷ groups together form a bridge:
*CH═N—N(H)*, *O(CH₂)₂O*, *O(CH₂)O*, *O(CF₂)O*, *C(═O)OCH₂*, *OC(═O)C(R^{8a})═C(R^{8b})*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═O)C(R^{8b})═C(R^{8c})*, *NHC(═O)NH*, *S(═O)ₓCH₂CH₂*, *CH₂S(═O)ₓCH₂*, *(CH₂)ₜ*; wherein each * represents the point of attachment to said aryl ring.

In another preferred embodiment, the invention relates to compounds of formula I, supra, wherein R⁷ represents a halogen atom, or a HO—, —CN, C₁-C₆-alkoxy-, C₁-C₆-alkyl-, halo-C₁-C₆-alkyl-, R^{8a}(R^{8b})N—C₁-C₆-alkyl-, halo-C₁-C₆-alkoxy-, HO—C₁-C₆-alkyl-, C₁-C₆-alkoxy-C₁-C₆-alkyl-, halo-C₁-C₆-alkoxy-C₁-C₆-alkyl-, C₂-C₆-alkenyl-, C₂-C₆-alkynyl-, 3- to 7-membered heterocycloalkyl-, aryl-, heteroaryl-, —C(═O)R⁸, —C(═O)N(R^{8a})R^{8b}, —C(═O)O—R⁸, —N(R^{8a})R^{8b}, —NO₂, —N(R^{8a})C(═O)R^{8b}, —N(R^{8c})C(═O)N(R^{8a})R^{8b}, —N(R^{8a})C(═O)OR^{8b}, —N(R^{8a})S(═O)R^{8b}, —N(R^{8a})S(═O)₂R^{8b}, —N═S(═O)(R^{8a})R^{8b}, —OR⁸, —O(C═O)R⁸, —O(C═O)N(R^{8a})R^{8b}, —O(C═O)OR⁸, —SR⁸, —S(═O)R⁸, —S(═O)N(R^{8a})R^{8b}, —S(═O)₂R⁸, —S(═O)₂OR⁸, —S(═O)₂N(R^{8a})R^{8b}, —S(═O)(═NR^{8c})R⁸ or —P(═O)(R^{8a})(OR^{8b}) group;

wherein said aryl- or heteroaryl- group is optionally substituted, identically or differently, with 1, 2 or 3 C₁-C₆-alkyl groups.

In another preferred embodiment, the invention relates to compounds of formula I, supra, wherein 2 R⁷ groups are present ortho- to each other on an aryl ring, said 2 R⁷ groups together form a bridge:

*CH═N—N(H)*, *O(CH₂)₂O*, *O(CH₂)O*, *O(CF₂)O*, *C(═O)OCH₂*, *OC(═O)C(R^{8a})═C(R^{8b})*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═O)C(R^{8b})═C(R^{8c})*, *NHC(═O)NH*, *S(═O)ₓCH₂CH₂*, *CH₂S(═O)ₓCH₂*, *(CH₂)ₜ*; wherein each * represents the point of attachment to said aryl ring.

In another preferred embodiment, the invention relates to compounds of formula I, supra, R⁷ represents a halogen atom, or a HO—, C₁-C₆-alkoxy-, C₁-C₆-alkyl-, halo-C₁-C₆-alkyl-, halo-C₁-C₆-alkoxy-, 3- to 7-membered heterocycloalkyl-, aryl-, heteroaryl-, —C(═O)R⁸, —C(═O)N(R^{8a})R^{8b}, —OR⁸, —SR⁸, —S(═O)R⁸, —S(═O)₂R⁸, —S(═O)₂OR⁸, —S(═O)₂N(R^{8a})R^{8b} or —P(═O)(R^{8a})(OR^{8b}) group;

wherein said aryl- or heteroaryl- group is optionally substituted, identically or differently, with 1, 2 or 3 C₁-C₆-alkyl groups;
or
when 2 R⁷ groups are present ortho- to each other on an aryl ring, said 2 R⁷ groups together form a bridge:
*CH═N—N(H)*, *O(CH₂)₂O*, *O(CF₂)O*, *C(═O)OCH₂*, *OC(═O)C(R^{8a})═C(R^{8b})*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═O)C(R^{8b})═C(R^{8c})*, *S(═O)ₓCH₂CH₂*, *CH₂S(═O)ₓCH₂*, *(CH₂)ₜ*; wherein each * represents the point of attachment to said aryl ring.

In another preferred embodiment, the invention relates to compounds of formula I, supra, R⁷ represents halogen atom, or a HO—, C₁-C₆-alkoxy-, C₁-C₆-alkyl-, halo-C₁-C₆-alkoxy-, HO—C₁-C₆-alkoxy-, 3- to 7-membered heterocycloalkyl-, heteroaryl-, —C(═O)R⁸, —C(═O)N(R^{8a})R^{8b}, —N(R^{8a})R^{8b}, —NO₂, —N(R^{8a})S(═O)₂R^{8b}, —OR⁸, —S(═O)₂OR⁸, —S(═O)₂N(R^{8a})R^{8b}, —P(═O)(R^{8a})(OR^{8b}) group; wherein said heteroaryl- group is optionally substituted, identically or differently, with 1 C₁-C₆-alkyl group.

In another preferred embodiment, the invention relates to compounds of formula I, supra, wherein 2 R⁷ groups are present ortho- to each other on a phenyl ring, said 2 R⁷ groups together form a bridge:

*CH═N—N(H)*, *O(CH₂)₂O*, *O(CF₂)O*, *C(═O)OCH₂*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═S)S*, *N(R^{8a})C(═O)C(R^{8b})═C(R^{8c})*, *S(═O)ₓCH₂CH₂*, *CH₂S(═O)ₓCH₂*, *N(H)C(═O)—C(═O)N(H)*, *(CH₂)ₜ*; wherein each * represents the point of attachment to said aryl ring.

In another preferred embodiment, the invention relates to compounds of formula I, supra, wherein 2 R⁷ groups are present ortho- to each other on a phenyl ring, said 2 R⁷ groups together form a bridge: *CH═N—N(H)*, *N(H)C(═O)S*, *C(═O)OCH₂*; wherein each * represents the point of attachment to said aryl ring.

In another preferred embodiment, the invention relates to compounds of formula I, supra, wherein R⁷ represents halogen atom, or a C₁-C₆-alkoxy-, C₁-C₆-alkyl-, halo-C₁-C₆-alkoxy-, HO—C₁-C₆-alkoxy- or —OR⁸ group.

In another preferred embodiment, the invention relates to compounds of formula I, supra, wherein R⁷ and R⁸ together form a —O—C₁-C₆-alkylene-group.

In another preferred embodiment, the invention relates to compounds of formula I, supra, wherein R⁷ and R⁹ together form a —O—C₁-C₆-alkylene-group.

In another preferred embodiment, the invention relates to compounds of formula I, supra, R⁸, R^{8a}, R^{8b}, R^{8c} represent, independently from each other, a hydrogen atom, or a C₁-C₆-alkyl-, C₃-C₆-cycloalkyl-, C₁-C₆-alkyl-aryl-, C₂-C₆-alkenyl-,

3- to 7-membered heterocycloalkyl-, —C₁-C₆-alkyl-C(═O)OR⁹—, aryl-, heteroaryl-, aryl-C₁-C₆-alkyl-, or heteroaryl-C₁-C₆-alkyl- group;
said C₁-C₆-alkyl-, C₃-C₆-cycloalkyl-, C₁-C₆-alkyl-aryl-, C₂-C₆-alkenyl-,
3- to 7-membered heterocycloalkyl-, aryl-, heteroaryl-, aryl-C₁-C₆-alkyl-, or heteroaryl-C₁-C₆-alkyl- group being optionally substituted, identically or differently, with 1, 2, 3, 4 or 5 groups selected from halogen, —OH, —CN, —C₁-C₆-alkoxy or R¹⁰ groups;
or
R^{8a} and R^{8b} together form a C₁-C₆-alkylene- or halo-C₁-C₆-alkylene- group.

In another preferred embodiment, the invention relates to compounds of formula I, supra, R⁸, R^{8a}, R^{8b}, R^{8c} represent, independently from each other, a hydrogen atom, or a C₁-C₆-alkyl-, C₃-C₆-cycloalkyl-, C₁-C₆-alkyl-aryl-,

3- to 7-membered heterocycloalkyl-, aryl-, heteroaryl-, aryl-C₁-C₆-alkyl-, or heteroaryl-C₁-C₆-alkyl- group;
said group being optionally substituted, identically or differently, with 1, 2 or 3 R¹⁰ groups;
or
R^{8a} and R^{8b} together form a C₁-C₆-alkylene- or halo-C₁-C₆-alkylene- group.

In another preferred embodiment, the invention relates to compounds of formula I, supra, R⁸, R^{8a}, R^{8b}, R^{8c} represent, independently from each other, a hydrogen atom, or a C₁-C₆-alkyl-, C₃-C₆-cycloalkyl-, C₃-C₆-cycloalkyl-(CH₂)—,

3- to 7-membered heterocycloalkyl-, aryl- group; said group being optionally substituted, identically or differently, with 1 R¹⁰ group.

In another preferred embodiment, the invention relates to compounds of formula I, supra, R^{8a} and R^{8b} together form a C₁-C₆-alkylene- or halo-C₁-C₆-alkylene- group.

In another preferred embodiment, the invention relates to compounds of formula I, supra, Q represents a —O— group.

In another preferred embodiment, the invention relates to compounds of formula I, supra, Q represents a —S—, —S(═O)— or —S(═O)₂— group.

In another preferred embodiment, the invention relates to compounds of formula I, supra, Q represents a —N(R⁹)— group.

In another preferred embodiment, the invention relates to compounds of formula I, supra, Q represents a —S(═O)(NR⁹)— group.

In another preferred embodiment, the invention relates to compounds of formula I, supra, Q represents —C(═O)—, —C(═O)—O— or —C(═O)—N(R⁹)— group.

In another preferred embodiment, the invention relates to compounds of formula I, supra, R⁹, R^{9a}, R^{9b} represent, independently from each other, a hydrogen atom, or a C₁-C₆-alkyl-, C₃-C₆-cycloalkyl-, C₁-C₆-alkyl-aryl-, aryl- or heteroaryl- group; wherein said C₁-C₆-alkyl-, C₃-C₆-cycloalkyl-, C₁-C₆-alkyl-aryl-, aryl- or heteroaryl-group is optionally substituted, identically or differently, with 1, 2 or 3 groups selected from halogen, —OH, —CN, —C₁-C₆-alkyl, C₁-C₆-alkoxy-.

In another preferred embodiment, the invention relates to compounds of formula I, supra, R⁹, R^{9a}, R^{9b} represent, independently from each other, a hydrogen atom, or a C₁-C₆-alkyl- group.

In another preferred embodiment, the invention relates to compounds of formula I, supra, R¹⁰ represents a halogen atom or a group selected from: C₁-C₆-alkyl-, halo-C₁-C₆-alkyl-, —S(═O)_{X}(C₁-C₆-alkyl), —C(═O)OR⁹.

In another preferred embodiment, the invention relates to compounds of formula I, supra, R¹⁰ represents a —S(═O)_{X}(C₁-C₆-alkyl) group.

In another preferred embodiment, the invention relates to compounds of formula I, supra, m is an integer of 0, 1 or 2.

In another preferred embodiment, the invention relates to compounds of formula I, supra, m is an integer of 0 or 1.

In another preferred embodiment, the invention relates to compounds of formula I, supra, n is an integer of 0, 1 or 2;

In another preferred embodiment, the invention relates to compounds of formula I, supra, n is an integer of 1 or 2;

In another preferred embodiment, the invention relates to compounds of formula I, supra, m is an integer of 0 and n is an integer of 1.

In another preferred embodiment, the invention relates to compounds of formula I, supra, m is an integer of 1 and n is an integer of 0.

In another preferred embodiment, the invention relates to compounds of formula I, supra, m is an integer of 1 and n is an integer of 1.

In another preferred embodiment, the invention relates to compounds of formula I, supra, p is an integer of 1, 2 or 3.

In another preferred embodiment, the invention relates to compounds of formula I, supra, q is an integer of 1, 2 or 3.

In another preferred embodiment, the invention relates to compounds of formula I, supra, t is an integer of 3, 4 or 5.

In another preferred embodiment, the invention relates to compounds of formula I, supra, t is an integer of 4.

In another preferred embodiment, the invention relates to compounds of formula I, supra x is an integer of 1 or 2.

In another preferred embodiment, the invention relates to compounds of formula I, supra x is an integer of 2.

In a further embodiment of the above-mentioned aspect, the invention relates to compounds of formula I, according to any of the above-mentioned embodiments, in the form of or a stereoisomer, a tautomer, an N-oxide, a hydrate, a solvate, or a salt thereof, or a mixture of same.

It is to be understood that the present invention relates also to any combination of the preferred embodiments described above.

Some examples of combinations are given hereinafter. However, the invention is not limited to these combinations.

In a preferred embodiment, the invention relates to compounds of formula I:

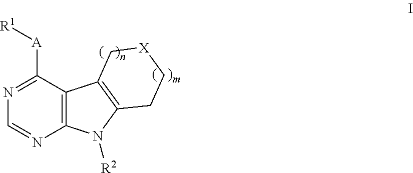

in which:
A represents —O—, —S—, —S(═O)—, —S(═O)₂— or —NR³—;
X represents —O—, —S—, —S(═O)—, —S(═O)₂—, —S(═O)(NR^{4a})—, —NR^{4a}—, —C(O—C₁-C₆-alkyl)₂-, —C(O—CH₂—CH₂—O)—, —C(O—CH₂—CH₂—CH₂—O)—, —C(O—CH₂—C(CH₃)₂—CH₂—O)—, —C(═O)—, —C(═O)NR^{4a}—, —NR^{4a}C(═O)—, —C(═O)O—, —OC(═O)—, —C(H)OR^{4a}—, —C(R^{4a})OR^{4b}—, —C═NR^{4a}—, —C(H)NR^{4a}R^{4b}—, —C(R^{5a})(R^{5b})—, —C(═CR^{6a}R^{6b})—, or —CH(CHR^{6a}R^{6b})—;
R¹ represents an aryl- or heteroaryl- group; wherein said aryl- or heteroaryl-group is optionally substituted, identically or differently, with 1, 2, 3 or 4 R⁷ groups;
R² represents a hydrogen atom, or a C₁-C₆-alkyl- or C₃-C₆-cycloalkyl- group; wherein said C₁-C₆-alkyl- or C₃-C₆-cycloalkyl- group is optionally substituted, identically or differently, with 1, 2, 3 or 4 groups selected from halogen, —OH, —CN, —C₁-C₆-alkyl, C₁-C₆-alkoxy-;
R³ represents a hydrogen atom, or a C₁-C₆-alkyl- group; wherein said C₁-C₆-alkyl- group is optionally substituted, identically or differently, with 1, 2, 3 or 4 groups selected from halogen, —OH, —CN, C₁-C₆-alkoxy-;
   or
A=—NR³—; and R¹ and R³, together with the nitrogen atom they are attached to, represent a 3- to 7-membered heterocycloalkyl- or benzo fused 3- to 7-membered heterocycloalkyl- group; wherein said group is optionally substituted, identically or differently, with 1, 2, 3 or 4 R⁷ groups;
R^{4a}, R^{4b}
   represent, independently from each other, a hydrogen atom, or a C₁-C₆-alkyl-, —(CH₂)ₚ—C₂-C₆-alkenyl, —(CH₂)_{q}—C₄-C₈-cycloalkenyl, —(CH₂)ₚ—C₂-C₆-alkynyl, —(CH₂)_{q}—C₃-C₆-cycloalkyl,
   —(CH₂)_{q}-(3- to 7-membered heterocycloalkyl), —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, R^{8a}(R^{8b})N—C₁-C₆-alkyl-, halo-C₁-C₆-alkoxy-C₁-C₆-alkyl-, —C(═O)R⁸, —C(═O)N(R^{8a}R^{8b}), —C(═O)O—R⁸, —S(═O)R⁸, —S(═O)₂R⁸, —S(═O)(═NR^{8a})R^{8b} or —S(═O)₂N(R^{8a})R^{8b} group;
   said C₁-C₆-alkyl-, —(CH₂)ₚ—C₂-C₆-alkenyl, —(CH₂)_{q}—C₄-C₈-cycloalkenyl, —(CH₂)ₚ—C₂-C₆-alkynyl, —(CH₂)_{q}—C₃-C₆-cycloalkyl, —(CH₂)_{q}-(3- to 7-membered heterocycloalkyl), —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl- group being optionally substituted, identically or differently, with 1, 2, 3, 4 or 5 R⁷ groups;
R^{5a}, R^{5b}
   represent, independently from each other, a hydrogen atom or halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl, —(CH₂)_{q}-(3- to 7-membered heterocycloalkyl), —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C(═O)R⁸, —C(═O)N(R^{8a}R^{8b}), —C(═O)O—R⁸, —S(═O)R⁸, —S(═O)₂R⁸, —S(═O)(═NR^{8a})R^{8b}, or —S(═O)₂N(R^{8a})R^{8b} group;
      or
R^{5a} and R^{5b} together
   form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- or —O—(C₂-C₆-alkylene)-O— group;
   said C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)-, —O—(C₂-C₆-alkylene)-O— group being optionally substituted, identically or differently, with 1, 2, 3, 4 or 5 R⁷ groups;
R^{6a}, R^{6b}
   represent, independently from each other, a hydrogen atom or halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C(═O)R⁸, —C(═O)N(R^{8a}R^{8b}) or —C(═O)O—R⁸ group;
      or
R^{6a} and R^{6b} together
   form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- group;
   said C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- group being optionally substituted, identically or differently, with 1, 2, 3, 4 or 5 R⁷ groups;
R⁷ represents a halogen atom, or a HO—, —CN, C₁-C₆-alkoxy-, C₁-C₆-alkyl-, halo-C₁-C₆-alkyl-, R^{8a}(R^{8b})N—C₁-C₆-alkyl-, halo-C₁-C₆-alkoxy-, HO—C₁-C₆-alkyl-, C₁-C₆-alkoxy-C₁-C₆-alkyl-, halo-C₁-C₆-alkoxy-C₁-C₆-alkyl-, C₂-C₆-alkenyl-, C₂-C₆-alkynyl-, 3- to 7-membered heterocycloalkyl-, aryl-, heteroaryl-, —C(═O)R⁸, —C(═O)N(R^{8a})R^{8b}, —C(═O)O—R⁸, —N(R^{8a})R^{8b}, —NO₂, —N(R^{8a})C(═O)R^{8b}, —N(R^{8c})C(═O)N(R^{8a})R^{8b}, —N(R^{8a})C(═O)OR^{8b}, —N(R^{8a})S(═O)R^{8b}, —N(R^{8a})S(═O)₂R^{8b}, —N═S(═O)(R^{8a})R^{8b}, —OR⁸, —O(C═O)R⁸, —O(C═O)N(R^{8a})R^{8b}, —O(C═O)OR⁸, —SR⁸, —S(═O)R⁸, —S(═O)N(R^{8a})R^{8b}, —S(═O)₂R⁸, —S(═O)₂OR⁸, —S(═O)₂N(R^{8a})R^{8b}, —S(═O)(═NR^{8c})R⁸ or —P(═O)(R^{8a})(OR^{8b}) group;
   wherein said aryl- or heteroaryl- group is optionally substituted, identically or differently, with 1, 2 or 3 C₁-C₆-alkyl groups;
      or
      when 2 R⁷ groups are present ortho- to each other on an aryl ring, said 2 R⁷ groups together form a bridge:
   *CH═N—N(H)*, *O(CH₂)₂O*, *O(CH₂)O*, *O(CF₂)O*, *C(═O)OCH₂*, *OC(═O)C(R^{8a})═C(R^{8b})*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═O)C(R^{8b})═C(R^{8c})*, *NHC(═O)NH*, *S(═O)ₓCH₂CH₂*, *CH₂S(═O)ₓCH₂*, *(CH₂)ₜ*; wherein each * represents the point of attachment to said aryl ring;
R⁸, R^{8a}, R^{8b}, R^{8c}
   represent, independently from each other, a hydrogen atom, or a C₁-C₆-alkyl-, C₃-C₆-cycloalkyl-, C₁-C₆-alkyl-aryl-, C₂-C₆-alkenyl-, 3- to 7-membered heterocycloalkyl-, aryl-, heteroaryl-, aryl-C₁-C₆-alkyl-, or heteroaryl-C₁-C₆-alkyl- group;
   said C₁-C₆-alkyl-, C₃-C₆-cycloalkyl-, C₁-C₆-alkyl-aryl-, C₂-C₆-alkenyl-, 3- to 7-membered heterocycloalkyl-, aryl-, heteroaryl-, aryl-C₁-C₆-alkyl-, or heteroaryl-C₁-C₆-alkyl- group being optionally substituted, identically or differently, with 1, 2, 3, 4 or 5 R¹⁰ groups;
      or
R^{8a} and R^{8b} together form a C₁-C₆-alkylene- or halo-C₁-C₆-alkylene- group;
Q represents a —O—, —S—, —S(═O)—, —S(═O)₂—, —S(═O)(NR⁹)—, —N(R⁹)—, —C(═O)—, —C(═O)—O— or —C(═O)—N(R⁹)— group;
R⁹, R^{9a}, R^{9b}
   represent, independently from each other, a hydrogen atom, or a C₁-C₆-alkyl-, C₃-C₆-cycloalkyl-, C₁-C₆-alkyl-aryl-, aryl- or heteroaryl- group; wherein said C₁-C₆-alkyl-, C₃-C₆-cycloalkyl-, C₁-C₆-alkyl-aryl-, aryl- or heteroaryl- group is optionally substituted, identically or differently, with 1, 2, 3, or 4 groups selected from halogen, —OH, —CN, —C₁-C₆-alkyl, C₁-C₆-alkoxy-;
R¹⁰ represents a —S(═O)_{X}(C₁-C₆-alkyl), —S(═O)_{X}(aryl), —S(═O)_{X}(C₁-C₆-alkyl-aryl), —S(═O)N(R⁹)(C₁-C₆-alkyl), —N(R^{9a})(R^{9b}), —C(═O)R⁹, —C(═O)OR⁹ or —C(═O)N(R^{9a})(R^{9b}) group;
m is an integer of 0, 1, 2, or 3;
n is an integer of 0, 1, 2, or 3;
p is an integer of 1, 2, 3, 4 or 5;
q is an integer of 0, 1, 2, 3, 4 or 5;
t is an integer of 3, 4, 5 or 6;
x is an integer of 0, 1 or 2
   or a stereoisomer, a tautomer, an N-oxide, a hydrate, a solvate, or a salt thereof, or a mixture of same.

In another preferred embodiment, the invention relates to compounds of formula I:

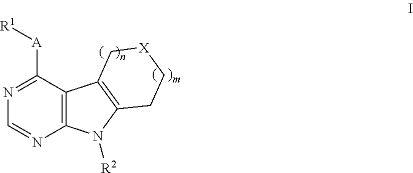

in which:
A represents —O—, —S—, —S(═O)—, —S(═O)₂— or —NR³—;
X represents —O—, —S—, —S(═O)—, —S(═O)₂—, —S(═O)(NR^{4a})—, —NR^{4a}—, —C(O—C₁-C₆-alkyl)₂-, —C(O—CH₂—CH₂—O)—, —C(O—CH₂—CH₂—CH₂—O)—, —C(O—CH₂—C(CH₃)₂—CH₂—O)—, —C(═O)—, —C(═O)NR^{4a}—, —NR^{4a}C(═O)—, —C(═O)O—, —OC(═O)—, —C(H)OR^{4a}—, —C(R^{4a})OR^{4b}—, —C═NR^{4a}—, —C(H)NR^{4a}R^{4b}—, —C(R^{5a})(R^{5b})—, —C(═CR^{6a}R^{6b})—, or —CH(CHR^{6a}R^{6b})—;
R¹ represents an aryl- or heteroaryl- group; wherein said aryl- or heteroaryl-group is optionally substituted, identically or differently, with 1, 2 or 3 R⁷ groups;
R² represents a hydrogen atom or a C₁-C₆-alkyl- group; wherein said C₁-C₆-alkyl- group is optionally substituted, identically or differently, with 1, 2 or 3 groups selected from halogen, —OH, —CN, C₁-C₆-alkoxy-;
R³ represents a hydrogen atom, or a C₁-C₆-alkyl- group; wherein said C₁-C₆-alkyl- group is optionally substituted, identically or differently, with 1, 2 or 3 groups selected from halogen, —OH, —CN, C₁-C₆-alkoxy-;
   or
A=—NR³—; and R¹ and R³, together with the nitrogen atom they are attached to, represent a 3- to 7-membered heterocycloalkyl- or benzo fused 3- to 7-membered heterocycloalkyl- group; wherein said group is optionally substituted, identically or differently, with 1, 2, 3 or 4 R⁷ groups;
R^{4a}, R^{4b}
   represent, independently from each other, a hydrogen atom, or a group selected from C₁-C₆-alkyl-, aryl-, aryl-C₁-C₆-alkyl-, heteroaryl-, —C(═O)R⁸, —C(═O)N(R^{8a}R^{8b}), —C(═O)O—R⁸, —S(═O)₂R⁸, —S(═O)₂N(R^{8a})R^{8b};
   said group being optionally substituted, identically or differently, with 1, 2, 3 or 4 R⁷ groups;
R^{5a}, R^{5b}
   represent, independently from each other, a hydrogen atom or halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl, —(CH₂)_{q}-(3- to 7-membered heterocycloalkyl), —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C(═O)R⁸, —C(═O)N(R^{8a}R^{8b}), —C(═O)O—R⁸, —S(═O)R⁸, —S(═O)₂R⁸, —S(═O)(═NR^{8a})R^{8b}, or —S(═O)₂N(R^{8a})R^{8b} group;
   or
   R^{5a} and R^{5b} together
   form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- or —O—(C₂-C₆-alkylene)-O— group;
   said C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)-, —O—(C₂-C₆-alkylene)-O— group being optionally substituted, identically or differently, with 1, 2, 3 or 4 R⁷ groups;
R^{6a}, R^{6b}
   represent, independently from each other, a hydrogen atom or halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C(═O)R⁸, —C(═O)N(R^{8a}R^{8b}) or —C(═O)O—R⁸ group;
      or
R^{6a} and R^{6b} together
   form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- group;
   said C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
   —(CH₂)_{q}-(3- to 7-membered heterocycloalkyl),
   —(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- group being optionally substituted, identically or differently, with 1, 2, 3, 4 or 5 R⁷ groups;
R⁷ represents a halogen atom, or a HO—, —CN, C₁-C₆-alkoxy-, C₁-C₆-alkyl-, halo-C₁-C₆-alkyl-, R^{8a}(R^{8b})N—C₁-C₆-alkyl-, halo-C₁-C₆-alkoxy-, HO—C₁-C₆-alkyl-, C₁-C₆-alkoxy-C₁-C₆-alkyl-, halo-C₁-C₆-alkoxy-C₁-C₆-alkyl-, C₂-C₆-alkenyl-, C₂-C₆-alkynyl-, 3- to 7-membered heterocycloalkyl-, aryl-, heteroaryl-, —C(═O)R⁸, —C(═O)N(R^{8a})R^{8b}, —C(═O)O—R⁸, —N(R^{8a})R^{8b}, —NO₂, —N(R^{8a})C(═O)R^{8b}, —N(R^{8c})C(═O)N(R^{8a})R^{8b}, —N(R^{8a})C(═O)OR^{8b}, —N(R^{8a})S(═O)R^{8b}, —N(R^{8a})S(═O)₂R^{8b}, —N═S(═O)(R^{8a})R^{8b}, —OR⁸, —O(C═O)R⁸, —O(C═O)N(R^{8a})R^{8b}, —O(C═O)OR⁸, —SR⁸, —S(═O)R⁸, —S(═O)N(R^{8a})R^{8b}, —S(═O)₂R⁸, —S(═O)₂OR⁸, —S(═O)₂N(R^{8a})R^{8b}, —S(═O)(═NR^{8c})R⁸ or —P(═O)(R^{8a})(OR^{8b}) group;
   wherein said aryl- or heteroaryl- group is optionally substituted, identically or differently, with 1, 2 or 3 C₁-C₆-alkyl groups;
      or
      when 2 R⁷ groups are present ortho- to each other on an aryl ring, said 2 R⁷ groups together form a bridge:
   *CH═N—N(H)*, *O(CH₂)₂O*, *O(CH₂)O*, *O(CF₂)O*, *C(═O)OCH₂*, *OC(═O)C(R^{8a})═C(R^{8b})*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═O)C(R^{8b})═C(R^{8c})*, *NHC(═O)NH*, *S(═O)ₓCH₂CH₂*, *CH₂S(═O)ₓCH₂*, *(CH₂)ₜ*; wherein each * represents the point of attachment to said aryl ring;
R⁸, R^{8a}, R^{8b}, R^{8c}
   represent, independently from each other, a hydrogen atom, or a C₁-C₆-alkyl-, C₃-C₆-cycloalkyl-, C₁-C₆-alkyl-aryl-, 3- to 7-membered heterocycloalkyl-, aryl-, heteroaryl-, aryl-C₁-C₆-alkyl-, or heteroaryl-C₁-C₆-alkyl-group;
   said C₁-C₆-alkyl-, C₃-C₆-cycloalkyl-, C₁-C₆-alkyl-aryl-, 3- to 7-membered heterocycloalkyl-, aryl-, heteroaryl-, aryl-C₁-C₆-alkyl-, or heteroaryl-C₁-C₆-alkyl-group being optionally substituted, identically or differently, with 1, 2, 3, 4 or 5 R¹⁰ groups;
      or
R^{8a} and R^{8b} together form a C₁-C₆-alkylene- or halo-C₁-C₆-alkylene- group;
Q represents a —O—, —S—, —S(═O)—, —S(═O)₂—, —S(═O)(NR⁹)—, —N(R⁹)—, —C(═O)—, —C(═O)—O— or —C(═O)—N(R⁹)— group;
R⁹, R^{9a}, R^{9b}
   represent, independently from each other, a hydrogen atom, or a C₁-C₆-alkyl-, C₃-C₆-cycloalkyl-, C₁-C₆-alkyl-aryl-, aryl- or heteroaryl- group; wherein said C₁-C₆-alkyl-, C₃-C₆-cycloalkyl-, C₁-C₆-alkyl-aryl-, aryl- or heteroaryl- group is optionally substituted, identically or differently, with 1, 2, 3, or 4 groups selected from halogen, —OH, —CN, —C₁-C₆-alkyl, C₁-C₆-alkoxy-;
R¹⁰ represents a —S(═O)_{X}(C₁-C₆-alkyl), —S(═O)_{X}(aryl), —S(═O)_{X}(C₁-C₆-alkyl-aryl), —S(═O)N(R⁹)(C₁-C₆-alkyl), —N(R^{9a})(R^{9b}), —C(═O)R⁹, —C(═O)OR⁹ or —C(═O)N(R^{9a})(R^{9b}) group;
m is an integer of 0, 1, 2, or 3;
n is an integer of 0, 1, 2, or 3;
p is an integer of 1, 2, 3, 4 or 5;
q is an integer of 0, 1, 2, 3, 4 or 5;
t is an integer of 3, 4, 5 or 6;
x is an integer of 0, 1 or 2
   or a stereoisomer, a tautomer, an N-oxide, a hydrate, a solvate, or a salt thereof, or a mixture of same.

In another preferred embodiment, the invention relates to compounds of formula I:

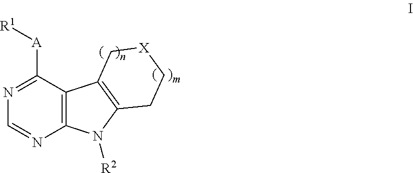

in which:
A represents —O—, —S— or —NR³—;
X represents —S—, —S(═O)—, —S(═O)₂—, —NR^{4a}—, —C(O—CH₂—CH₂—O)—, —C(R^{5a})(R^{5b})— or —CH(CHR^{6a}R^{6b})—;
R¹ represents an aryl- or heteroaryl- group; wherein said aryl- or heteroaryl-group is optionally substituted, identically or differently, with 1, 2 or 3 R⁷ groups;
R² represents a hydrogen atom or a C₁-C₆-alkyl- group;
R³ represents a hydrogen atom;
   or
A represents —NR³—; and R¹ and R³, together with the nitrogen atom they are attached to, represent a 3- to 7-membered heterocycloalkyl- or benzo fused 3- to 7-membered heterocycloalkyl- group; wherein said group is optionally substituted, identically or differently, with 1, 2, 3 or 4 R⁷ groups;
R^{4a}, R^{4b}
   represent, independently from each other, a hydrogen atom, or a C₁-C₆-alkyl-, aryl-, aryl-C₁-C₆-alkyl-, heteroaryl-, —C(═O)R⁸, —C(═O)N(R^{8a}R^{8b}), —C(═O)O—R⁸, —S(═O)₂R⁸ or —S(═O)₂N(R^{8a})R^{8b} group;
   said group being optionally substituted, identically or differently, with 1, 2 or 3 R⁷ groups;
R^{5a}, R^{5b}
   represent, independently from each other, a hydrogen atom or a halogen atom, or a C₁-C₆-alkoxy-, C₁-C₆-alkoxy-C₁-C₆-alkyl-, —C(═O)R⁸, —C(═O)N(R^{8a}R^{8b}) or —C(═O)O—R⁸ group;
      or
R^{5a} and R^{5b}
   together form a —O—(C₂-C₆-alkylene)-O— group;
R^{6a} represents a hydrogen atom;
R^{6b} represents a hydrogen atom;
R⁷ represents halogen atom, or a HO—, C₁-C₆-alkoxy-, C₁-C₆-alkyl-, halo-C₁-C₆-alkoxy-, HO—C₁-C₆-alkoxy-, 3- to 7-membered heterocycloalkyl-, heteroaryl-, —C(═O)R⁸, —C(═O)N(R^{8a})R^{8b}, —N(R^{8a})R^{8b}, —NO₂, —N(R^{8a})S(═O)₂R^{8b}, —OR⁸, —S(═O)₂OR⁸, —S(═O)₂N(R^{8a})R^{8b}, —P(═O)(R^{8a})(OR^{8b}) group;
   wherein said heteroaryl- group is optionally substituted, identically or differently, with 1 C₁-C₆-alkyl group.
      or
      when 2 R⁷ groups are present ortho- to each other on a phenyl ring, said 2 R⁷ groups together form a bridge:
   *CH═N—N(H)*, *O(CH₂)₂O*, *O(CF₂)O*, *C(═O)OCH₂*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═S)S*, *N(R^{8a})C(═O)C(R^{8b})═C(R^{8c})*, *S(═O)ₓCH₂CH₂*, *CH₂S(═O)ₓCH₂*, *N(H)C(═O)—C(═O)N(H)*, *(CH₂)ₜ*; wherein each * represents the point of attachment to said aryl ring;
R⁸, R^{8a}, R^{8b}, R^{8c}
   represent, independently from each other, a hydrogen atom, or a C₁-C₆-alkyl-, C₃-C₆-cycloalkyl-, C₃-C₆-cycloalkyl-(CH₂)—,
   3- to 7-membered heterocycloalkyl-, aryl- group; said group being optionally substituted, identically or differently, with 1 R¹⁰ group;
      or
R^{8a} and R^{8b} together form a C₁-C₆-alkylene- or halo-C₁-C₆-alkylene- group;
R⁹ represents a hydrogen atom or a C₁-C₆-alkyl- group;
R¹⁰ represents a halogen atom or a group selected from: C₁-C₆-alkyl-, halo-C₁-C₆-alkyl-, —S(═O)_{X}(C₁-C₆-alkyl), —C(═O)OR⁹;
m represents 1;
n represents 1;
t represents 3, 4 or 5;
x represents 1 or 2;
   or a stereoisomer, a tautomer, an N-oxide, a hydrate, a solvate, or a salt thereof, or a mixture of same.

In another preferred embodiment, the invention relates to compounds of formula I:

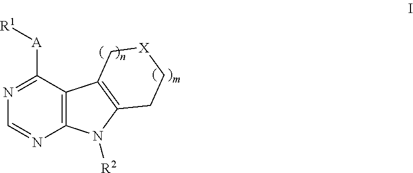

in which:
A represents —O—, —S— or —NR³—;
X represents —S—, —S(═O)—, —S(═O)₂—, —NR^{4a}—, —C(O—CH₂—CH₂—O)—, —C(R^{5a})(R^{5b})— or —CH(CHR^{6a}R^{6b})—;
R¹ represents

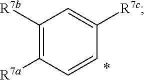

wherein * represents the point of attachment of the group to the rest of the molecule;
R² represents a hydrogen atom;
R³ represents a hydrogen atom;
   or
A represents —NR³—; and R¹ and R³, together with the nitrogen atom they are attached to, represent a 3- to 7-membered heterocycloalkyl- or benzo fused 3- to 7-membered heterocycloalkyl- group; wherein said group is optionally substituted, identically or differently, with 1, 2, 3 or 4 R⁷ groups;
R^{4a}, R^{4b}
   represent, independently from each other, a hydrogen atom, or a C₁-C₆-alkyl-, aryl-, aryl-C₁-C₆-alkyl-, heteroaryl-, —C(═O)R⁸, —C(═O)N(R^{8a}R^{8b}), —C(═O)O—R⁸, —S(═O)₂R⁸ or —S(═O)₂N(R^{8a})R^{8b} group;
   said group being optionally substituted, identically or differently, with 1, 2 or 3 R⁷ groups;
R^{5a}, R^{5b}
   represent, independently from each other, a hydrogen atom or a halogen atom, or a C₁-C₆-alkoxy-, C₁-C₆-alkoxy-C₁-C₆-alkyl-, —C(═O)R⁸, —C(═O)N(R^{8a}R^{8b}) or —C(═O)O—R⁸ group;
      or
R^{5a} and R^{5b}
   together form a —O—(C₂-C₆-alkylene)-O— group;
R^{6a} represents a hydrogen atom;
R^{6b} represents a hydrogen atom;
R⁷ represents halogen atom, or a HO—, C₁-C₆-alkoxy-, C₁-C₆-alkyl-, halo-C₁-C₆-alkoxy-, HO—C₁-C₆-alkoxy-, 3- to 7-membered heterocycloalkyl-, heteroaryl-, —C(═O)R⁸, —C(═O)N(R^{8a})R^{8b}, —N(R^{8a})R^{8b}, —NO₂, —N(R^{8a})S(═O)₂R^{8b}, —OR⁸, —S(═O)₂OR⁸, —S(═O)₂N(R^{8a})R^{8b}, —P(═O)(R^{8a})(OR^{8b}) group;
   wherein said heteroaryl- group is optionally substituted, identically or differently, with 1 C₁-C₆-alkyl group.
R^{7a}; R^{7b}
   represent, independently from each other, a hydrogen atom or a halogen atom, or a group selected from: —CN, C₁-C₆-alkoxy-, C₁-C₆-alkyl-, halo-C₁-C₆-alkyl-, halo-C₁-C₆-alkoxy-, C₁-C₆-alkoxy-C₁-C₆-alkyl-, halo-C₁-C₆-alkoxy-C₁-C₆-alkyl-;
      or
R^{7a} and R^{7b} together form a bridge:
   *CH═N—N(H)*, *O(CH₂)₂O*, *O(CH₂)O*, *O(CF₂)O*, *C(═O)OCH₂*, *OC(═O)C(R^{8a})═C(R^{8b})*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═S)S*, *N(R^{8a})C(═O)C(R^{8b})═C(R^{8c})*, *NHC(═O)NH*, *S(═O)ₓCH₂CH₂*, *CH₂S(═O)ₓCH₂*, *N(H)C(═O)—C(═O)N(H)*, *(CH₂)ₜ*;
R^{7c} represents a hydrogen atom, a halogen atom or a group selected from:
   3- to 7-membered heterocycloalkyl-, C₁-C₆-alkoxy-, halo-C₁-C₆-alkoxy-, HO—C₁-C₆-alkoxy-, —OR⁸;
R⁸ represents a C₃-C₆-cycloalkyl-, C₃-C₆-cycloalkyl-(CH₂)— or
   3- to 7-membered heterocycloalkyl- group;
R^{8a}, R^{8b}, R^{8c}
   represent, independently from each other, a hydrogen atom, or a C₁-C₆-alkyl-, C₃-C₆-cycloalkyl-, C₃-C₆-cycloalkyl-(CH₂)—,
   3- to 7-membered heterocycloalkyl-, aryl- group; said group being optionally substituted, identically or differently, with 1 R¹⁰ group;
      or
R^{8a} and R^{8b} together form a C₁-C₆-alkylene- or halo-C₁-C₆-alkylene- group;
R⁹ represents a hydrogen atom or a C₁-C₆-alkyl- group;
R¹⁰ represents a halogen atom or a group selected from: C₁-C₆-alkyl-, halo-C₁-C₆-alkyl-, —S(═O)ₓ(C₁-C₆-alkyl), —C(═O)OR⁹;
m represents 1;
n represents 1;
t represents 3, 4 or 5;
x represents 1 or 2;
   or a stereoisomer, a tautomer, an N-oxide, a hydrate, a solvate, or a salt thereof, or a mixture of same.

In another preferred embodiment, the invention relates to compounds of formula I:

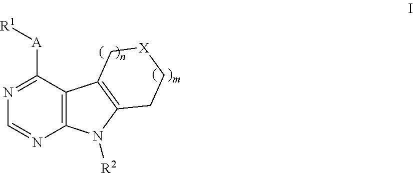

in which:
A represents —NR³—;
X represents —S—, —S(═O)—, —S(═O)₂—, —NR^{4a}—, —C(O—CH₂—CH₂—O)—, —C(R^{5a})(R^{5b})— or —CH(CHR^{6a}R^{6b})—;
R¹ represents

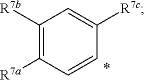

wherein * represents the point of attachment of the group to the rest of the molecule;
R² represents a hydrogen atom;
R³ represents a hydrogen atom;
   or
A represents —NR³—; and R¹ and R³, together with the nitrogen atom they are attached to, represent a 3- to 7-membered heterocycloalkyl- or benzo fused 3- to 7-membered heterocycloalkyl- group; wherein said group is optionally substituted, identically or differently, with 1, 2, 3 or 4 R⁷ groups;
R^{4a}, R^{4b}
   represent, independently from each other, a hydrogen atom, or a C₁-C₆-alkyl-, aryl-, aryl-C₁-C₆-alkyl-, heteroaryl-, —C(═O)R⁸, —C(═O)N(R^{8a}R^{8b}), —C(═O)O—R⁸, —S(═O)₂R⁸ or —S(═O)₂N(R^{8a})R^{8b} group;
   said group being optionally substituted, identically or differently, with 1, 2 or 3 R⁷ groups;
R^{5a} represents a hydrogen atom;
R^{5b} represents a group selected from:
   —H, —C(═O)R⁸, —C(═O)N(R^{8a}R^{8b}), —C(═O)O—R⁸;
      or
      each of R^{5a} and R^{5b} represents a fluorine atom;
R^{6a} represents a hydrogen atom;
R^{6b} represents a hydrogen atom;
R⁷ represents halogen atom, or a HO—, C₁-C₆-alkoxy-, C₁-C₆-alkyl-, halo-C₁-C₆-alkoxy-, HO—C₁-C₆-alkoxy-, 3- to 7-membered heterocycloalkyl-, heteroaryl-, —C(═O)R⁸, —C(═O)N(R^{8a})R^{8b}, —N(R^{8a})R^{8b}, —NO₂, —N(R^{8a})S(═O)₂R^{8b}, —OR⁸, —S(═O)₂OR⁸, —S(═O)₂N(R^{8a})R^{8b} group;
   wherein said heteroaryl- group is optionally substituted, identically or differently, with 1 C₁-C₆-alkyl group.
R^{7a}; R^{7b}
   represent, independently from each other, a hydrogen atom or a halogen atom, or a group selected from:
   C₁-C₆-alkoxy-, C₁-C₆-alkyl-, halo-C₁-C₆-alkyl-;
      or
R^{7a} and R^{7b} together form a bridge:
   *CH═N—N(H)*, *N(H)C(═O)S*, *C(═O)OCH₂*; wherein each * represents the point of attachment to the phenyl ring;
R^{7c} represents a hydrogen atom, a halogen atom or a group selected from:
   3- to 7-membered heterocycloalkyl-, C₁-C₆-alkoxy-, halo-C₁-C₆-alkoxy-, HO—C₁-C₆-alkoxy-, —OR⁸;
R⁸ represents a C₃-C₆-cycloalkyl-, C₃-C₆-cycloalkyl-(CH₂)— or
   3- to 7-membered heterocycloalkyl- group;

R^{8a}, R^{8b}, R^{8c}
represent, independently from each other, a hydrogen atom, or a C₁-C₆-alkyl-, C₃-C₆-cycloalkyl-, C₃-C₆-cycloalkyl-(CH₂)—,
3- to 7-membered heterocycloalkyl-, aryl- group; said group being optionally substituted, identically or differently, with 1 R¹⁰ group;
   or
R^{8a} and R^{8b} together form a C₁-C₆-alkylene- or halo-C₁-C₆-alkylene- group;
R⁹ represents a hydrogen atom or a C₁-C₆-alkyl- group;
R¹⁰ represents a halogen atom or a group selected from: C₁-C₆-alkyl-, halo-C₁-C₆-alkyl-, —S(═O)_{X}(C₁-C₆-alkyl), —C(═O)OR⁹;
m represents 1;
n represents 1;
t represents 3, 4 or 5;
x represents 1 or 2;
   or a stereoisomer, a tautomer, an N-oxide, a hydrate, a solvate, or a salt thereof, or a mixture of same.

In another preferred embodiment, the invention relates to compounds of formula I:

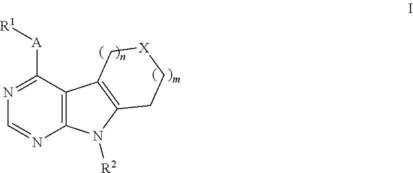

in which:
A represents —NR³—;
X represents a group selected from: —(CH₂)—, —(CF₂)—, —C(H)(C(═O)R⁸)—, —C(H)(C(═O)N(R^{8a}R^{8b}))—, —C(H)(C(═O)O—R⁸)—;
R¹ represents

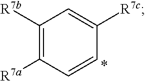

wherein * represents the point of attachment of the group to the rest of the molecule;
R² represents a hydrogen atom;
R³ represents a hydrogen atom;
   or
R¹ and R³, together with the nitrogen atom they are attached to, represent a 3- to 7-membered heterocycloalkyl- or benzo fused 3- to 7-membered heterocycloalkyl- group; wherein said group is optionally substituted, identically or differently, with 1, 2, 3 or 4 R⁷ groups;
R⁷ represents halogen atom, or a C₁-C₆-alkoxy-, C₁-C₆-alkyl-, halo-C₁-C₆-alkoxy-, HO—C₁-C₆-alkoxy- or —OR⁸ group;
R^{7a}; R^{7b}
   represent, independently from each other, a hydrogen atom or a halogen atom, or a group selected from:
   C₁-C₆-alkoxy-, C₁-C₆-alkyl-, halo-C₁-C₆-alkyl-;
      or
R^{7a} and R^{7b} together form a bridge:
   *CH═N—N(H)*, *N(H)C(═O)S*, *C(═O)OCH₂*; wherein each * represents the point of attachment to the phenyl ring;
R^{7c} represents a hydrogen atom, a halogen atom or a group selected from:
   3- to 7-membered heterocycloalkyl-, C₁-C₆-alkoxy-, halo-C₁-C₆-alkoxy-, HO—C₁-C₆-alkoxy-, —OR⁸;
R⁸ represents a C₃-C₆-cycloalkyl-, C₃-C₆-cycloalkyl-(CH₂)— or
   3- to 7-membered heterocycloalkyl- group;
R^{8a}, R^{8b}, R^{8c}
   represent, independently from each other, a hydrogen atom, or a C₁-C₆-alkyl-, C₃-C₆-cycloalkyl-, C₃-C₆-cycloalkyl-(CH₂)—,
   3- to 7-membered heterocycloalkyl-, aryl- group; said group being optionally substituted, identically or differently, with 1 R¹⁰ group;
R⁹ represents a hydrogen atom or a C₁-C₆-alkyl- group;
R¹⁰ represents a halogen atom or a group selected from: C₁-C₆-alkyl-, halo-C₁-C₆-alkyl-, —S(═O)ₓ(C₁-C₆-alkyl), —C(═O)OR⁹;
m represents 1;
n represents 1;
t represents 3, 4 or 5;
x represents 1 or 2;
   or a stereoisomer, a tautomer, an N-oxide, a hydrate, a solvate, or a salt thereof, or a mixture of same.

In another preferred embodiment, the invention relates to compounds of formula I:

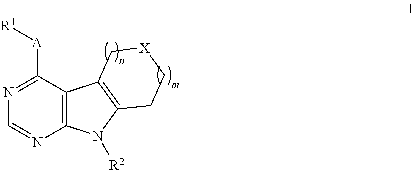

in which:
A represents —NR³—;
X represents a group selected from: —(CH₂)—, —(CF₂)—, —C(H)(C(═O)R⁸)—, —C(H)(C(═O)N(R^{8a}R^{8b}))—, —C(H)(C(═O)O—R⁸)—;
R¹ represents

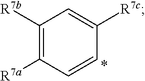

wherein * represents the point of attachment of the group to the rest of the molecule;
R² represents a hydrogen atom;
R³ represents a hydrogen atom;
R^{7a}; R^{7b}
   represent, independently from each other, a hydrogen atom or a halogen atom, or a group selected from:
   C₁-C₆-alkoxy-, C₁-C₆-alkyl-, halo-C₁-C₆-alkyl-;
      or
R^{7a} and R^{7b} together form a bridge:
   *CH═N—N(H)*, *N(H)C(═O)S*, *C(═O)OCH₂*; wherein each * represents the point of attachment to the phenyl ring;
R^{7c} represents a hydrogen atom, a halogen atom or a group selected from:
   3- to 7-membered heterocycloalkyl-, C₁-C₆-alkoxy-, halo-C₁-C₆-alkoxy-, HO—C₁-C₆-alkoxy-, —OR⁸;
R⁸ represents a C₃-C₆-cycloalkyl-, C₃-C₆-cycloalkyl-(CH₂)— or
   3- to 7-membered heterocycloalkyl- group;
R^{8a}, R^{8b}
   represent, independently from each other, a hydrogen atom, or a C₁-C₆-alkyl-, C₃-C₆-cycloalkyl-, C₃-C₆-cycloalkyl-(CH₂)—,
   3- to 7-membered heterocycloalkyl-, aryl- group; said group being optionally substituted, identically or differently, with 1 R¹⁰ group;
R⁹ represents a hydrogen atom or a C₁-C₆-alkyl- group;
R¹⁰ represents a halogen atom or a group selected from: C₁-C₆-alkyl-, halo-C₁-C₆-alkyl-, —S(═O)_{X}(C₁-C₆-alkyl), —C(═O)OR⁹;
m represents 1;
n represents 1;
t represents 3, 4 or 5;
x represents 1 or 2;
   or a stereoisomer, a tautomer, an N-oxide, a hydrate, a solvate, or a salt thereof, or a mixture of same.

It is to be understood that the present invention relates to any sub-combination within any embodiment or aspect of the present invention of compounds of general formula I, supra.

More particularly still, the present invention covers compounds of general formula I which are disclosed in the Example section of this text, infra.

In accordance with another aspect, the present invention covers methods of preparing compounds of the present invention, said methods comprising the steps as described in the Experimental Section herein.

In a preferred embodiment, the present invention relates to a method of preparing compounds of general formula I, supra, in which method an intermediate compound of general formula II:

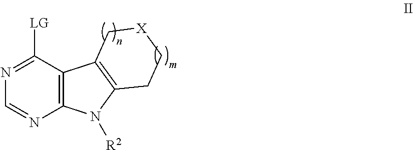

in which R², X, m, and n are as defined for the compounds of general formula I, supra, and LG represents a leaving group, such as a halogen atom or a trifluoromethylsulphonyloxy or nonafluorobutylsulphonyloxy group for example,
is allowed to react with a compound of general formula IIa:

R¹-A′ IIa

in which A′ represents a HO— or a HS— or a HNR³— group, and R¹ and R³ are as defined for the compounds of general formula I, supra;
thus providing a compound of general formula I:

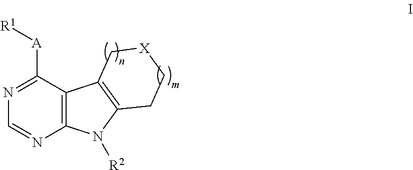

in which R¹, R², X, m, and n are as defined for the compounds of general formula I, supra.

As used herein, the term “leaving group” refers to an atom or a group of atoms that is displaced in a chemical reaction as stable species taking with it the bonding electrons. Preferably, a leaving group is selected from the group comprising: halo, in particular chloro, bromo or iodo, methanesulfonyloxy, p-toluenesulfonyloxy, trifluoromethanesulfonyloxy, nonafluorobutanesulfonyloxy, (4-bromo-benzene)sulfonyloxy, (4-nitro-benzene)sulfonyloxy, (2-nitro-benzene)-sulfonyloxy, (4-isopropyl-benzene)sulfonyloxy, (2,4,6-tri-isopropyl-benzene)-sulfonyloxy, (2,4,6-trimethyl-benzene)sulfonyloxy, (4-tertbutyl-benzene)sulfonyloxy, benzenesulfonyloxy, and (4-methoxy-benzene)sulfonyloxy.

In accordance with a further aspect, the present invention covers intermediate compounds which are useful in the preparation of compounds of the present invention of general formula I, particularly in the method described herein. In particular, the present invention covers compounds of general formula II:

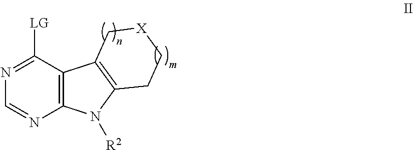

in which R², X, m, and n are as defined for the compounds of general formula I, supra, and LG represents a leaving group, such as a halogen atom or a trifluoromethylsulphonyloxy or nonafluorobutylsulphonyloxy group for example.

In accordance with yet another aspect, the present invention covers the use of the intermediate compounds of general formula II:

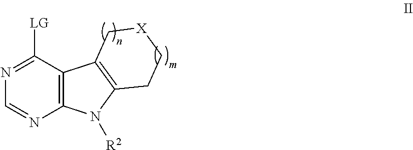

in which R², X, m, and n are as defined for the compounds of general formula I, supra, and LG represents a leaving group, such as a halogen atom or a trifluoromethylsulphonyloxy or nonafluorobutylsulphonyloxy group for example,
for the preparation of a compound of general formula I as defined supra.

### Synthesis of Compounds of General Formula I of the Present Invention

Compounds of general formula II, III, IV, V and VI wherein R¹, R², X, A, n and m have the meaning as given for general formula I and LG represents a leaving group, can be synthesized according to the procedures depicted in Scheme 1.

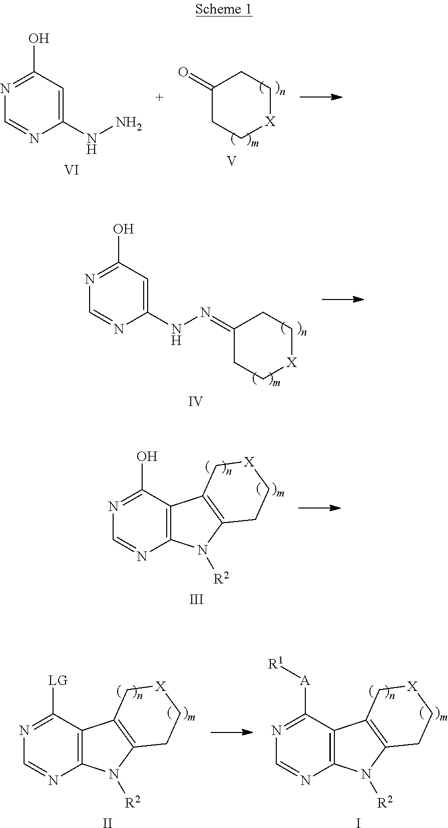

Scheme 1 exemplifies the main route that allows variations in R¹, R², X, A, n or m at different stages of the synthesis. However, also other routes may be used to synthesise the target compounds, in accordance with common general knowledge of a person skilled in the art of organic synthesis. The order of transformations exemplified in the Scheme is therefore not intended to be limiting. In addition, interconversion of any of the substituents, R¹, R², A or X can be achieved before and/or after the exemplified transformations.

These modifications can be such as the introduction of protecting groups, cleavage of protecting groups, reduction or oxidation of functional groups, halogenation, metallation, substitution or other reactions known to a person skilled in the art. These transformations include those which introduce a functionality which allows for further interconversion of substituents. Appropriate protecting groups and their introduction and cleavage are well-known to a person skilled in the art (see for example T. W. Greene and P. G. M. Wuts in Protective Groups in Organic Synthesis, 3^{rd} edition, Wiley 1999). Specific examples are described in the subsequent paragraphs. Further, it is possible that two or more successive steps may be performed without work-up being performed between said steps, e.g. a “one-pot” reaction, as it is well-known to a person skilled in the art.

Compounds of formula VI may be commercially available or can be synthesized according to procedures known to a person skilled in the art, for example applying procedures described in Journal of Organic Chemistry 1971, 36, 2462-2465.

Compounds of formula V may be commercially available or can be synthesized according to procedures known to a person skilled in the art.

Compounds of formula IV can be synthesized by reacting compound VI with carbonyl compound V in an inert solvent like, for example, ethanol or methanol at temperatures ranging from room temperature to the boiling point of the solvent, for example.

Compounds of formula III can be synthesized by heating compounds of formula IV with or without an inert additive or solvent like, for example, xylol or 1-methoxy-2-(2-methoxyethoxyl)ethane at temperatures ranging from 100° C. to 400° C. and pressures ranging from 1 atmosphere to 50 bar. Heating can be optionally performed using microwave irradiation optionally with an additive to improve the absorption of microwave radiation like, for example, an ionic liquid like, for example, 3-(triphenylphosphonio)-propane-1-sulfonate.

Compounds of formula II in which LG represents a leaving group like, for example, a halogen atom as, for example, a chlorine or bromine atom are obtained from compounds of formula III by reacting the alcohol with a halogenation agent like, for example, phosphorus trichloride or phosphorus tribromide with or without an additional inert solvent as, for example, toluene at temperatures ranging from room temperature to the boiling point of the solvent, for example.

Compounds of formula II in which LG represents a leaving group like, for example, an alkylsulfonate as, for example, methanesulfonate or trifluoromethanesulfonate or 1,1,2,2,3,3,4,4,4-nonafluorobutane-1-sulfonate or an arylsulfonate like, for example, benzenesulfonate or 4-methylbenzenesulfonate are obtained from compounds of formula III by reacting the alcohol with a suitable alkylsulfonate as, for example, methanesulfonyl chloride or trifluoromethanesulfonyl chloride or 1,1,2,2,3,3,4,4,4-nonafluorobutane-1-sulfonyl fluoride or by reacting the alcohol with a suitable arylsulfonate as, for example, benzenesulfonyl chloride or 4-methylbenzenesulfonyl chloride in an inert solvent like, for example, tetrahydrofuran or toluene or dichloromethane optionally in the presence of a suitable base like, for example, triethylamine or pyridine or N,N-dimethylpyridin-4-amine at temperatures ranging from −40° C. to the boiling point of the solvent, for example.

Compounds of formula I can be synthesized by reacting compounds of formula II with a compound of formula R¹-A′ wherein R¹ has the meaning as given for general formula I and A′ represents a HO— or a HS— or a HNR³— group, with R³ as defined for general formula I.

Acidic hydrogens in R¹—OH, R¹—SH or R¹R³NH can be removed by suitable bases, for example sodium hydride, in a suitable solvent, such as DMSO or tetrahydrofuran at temperatures ranging from room temperature to the boiling point of the solvent. The resulting nucleophiles, like, for example, R¹—O⁻, R¹—S⁻, R¹R³N⁻ or directly R¹R³NH can be used to replace LG in compounds of general formula II to form ethers, thioethers or amines to give compounds of general formula I.

Compounds of general formula II can also be reacted with amines of formula R¹HNR³ in the presence of acid like, for example, hydrochloric acid in an inert solvent like, for example, ethanol or 1,4-dioxane at temperatures ranging from room temperature to the boiling point of the solvent, for example, to give compounds of general formula I.

Compounds of general formula I containing primary or secondary amines, ethers or thioethers can also be build by Ullmann-type coupling reactions in the presence of suitable catalysts, such as, for example, copper based catalysts like copper(II)diacetate or copper(I)chloride in the presence of a suitable base, like for example, caesium carbonate starting from compounds of general formula II. Optionally, suitable ligands like N,N-dimethylglycine or phenyl hydrogen pyrrolidin-2-ylphosphonate can be added. The reaction can be performed at temperatures ranging from −40° C. to the boiling point of the solvent, for example.

In case X and/or A represent a sulfur atom in compounds of general formula I, II, III, IV or V the thioethers can be oxidized using oxidation reagents like 3-chlorobenzenecarboperoxoic acid, trifluoroethaneperoxoic acid, oxone, dimethyldioxirane or methyltrioxorhenium and hydrogen peroxide in inert solvents like dichloromethane or acetone, at temperatures ranging from −40° C. to the boiling point of the solvent. Depending on the stoichiometric ratio of oxidation reagent to the afore mentioned thioethers and the reaction conditions sulfoxides or sulfones or mixtures thereof will be obtained.

In case X and/or A represent a sulfoxide it can be transformed into sulfoximines S(O)(NR^{4a}) or S(O)(NR³) by reaction with, for example, 2,2,2-trifluoroacetamide, oxidation agents like, for example, diacetoxy(phenyl)-lambda³-iodane and magnesium oxide, a suitable catalyst like, for example, rhodium(II) diacetate in an inert solvent like, for example, dichloromethane at temperatures ranging from −40° C. to the boiling point of the solvent, for example.

In case X represents a —C(O—C₁-C₆-alkyl)₂-, —C(O—CH₂—CH₂—O)—, —C(O—CH₂—CH₂—CH₂—O)— or —C(O—CH₂—C(CH₃)₂—CH₂—O)— group, the acetal can be cleaved by methods known to a person skilled in the art to give a carbonyl group (X: —C(═O)—).

In case X represents a C(═O)— group this group can be reduced (X: —C(H)OR^{4a}), or alkylated (X: —C(R^{4a})OR^{4b}) by methods known to a person skilled in the art.

In case X represents a C(═O)— group this group can be oxydized in a Bayer-Villiger type reaction by methods known to a person skilled in the art to give lactones (X: —C(═O)O—, —OC(═O)—).

In case X represents a C(═O)— group this group can be transformed in a Beckmann type reaction by methods known to a person skilled in the art to give lactames (X: —C(═O)NR^{4a}—, —NR^{4a}C(═O)—).

In case X represents a C(═O)— group this group can be reacted in a Wittig- or Wittig-Horner- or Tebbe type reaction by methods known to a person skilled in the art to give olefines (X: —C(═CR^{6a}R^{6b})—).

In case X represents a C(═O)— group this group can be reacted with amines to give imines (X: —C═NR^{4a}—) under reaction conditions known to a person skilled in the art.

In case X represents a C(═O)— group this group can be reacted using reductive amination methods known to a person skilled in the art to give amines (X: —CH(NHR^{4a}—).

Olefins (X: —C(═CR^{6a}R^{6b})—) or imines (X: —C═NR^{4a}—) can be reduced to saturated compounds (X: —CH(CHR^{6a}R^{6b})—, —CH(NHR^{4a}—) using reaction conditions known to a person skilled in the art.

In case X represents a —C(═CR^{6a}R^{6b})— group the substitution pattern R^{6a}, R^{6b}, can be replaced using cross metathesis reaction methodologies known to a person skilled in the art.

Further, the compounds of formula I of the present invention can be converted to any salt as described herein, by any method which is known to the person skilled in the art. Similarly, any salt of a compound of formula I of the present invention can be converted into the free compound, by any method which is known to the person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallisation. In some cases, impurities may be removed by stirring using a suitable solvent. In some cases, the compounds may be purified by chromatography, particularly flash chromatography, using for example pre-packed silica gel cartridges, e.g. from Separtis such as Isolute® Flash silica gel or Isolute® Flash NH2 silica gel in combination with a suitable chromatographic system such as an Isolera system (Biotage) and eluents such as, for example, gradients of hexane/ethyl acetate or dichloromethane/methanol. In some cases, the compounds may be purified by preparative HPLC using, for example, a Waters autopurifier equipped with a diode array detector and/or on-line electrospray ionisation mass spectrometer in combination with a suitable pre-packed reverse phase column and eluents such as, for example, gradients of water and acetonitrile which may contain additives such as trifluoroacetic acid, formic acid or aqueous ammonia.

### EXAMPLES

### Example 1

### N-(4-Fluoro-2-isopropoxyphenyl)-6,7,8,9-tetrahydro-5H-pyrimido[4,5-b]indol-4-amine

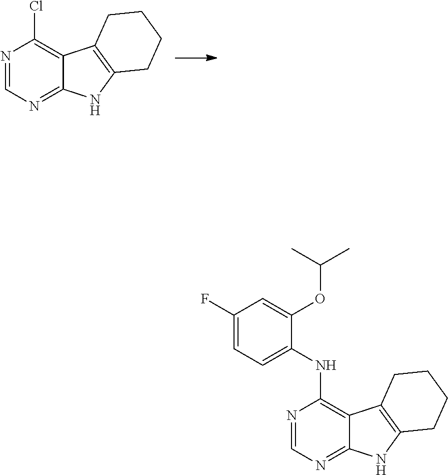 [SMILES: Clc1ncnc2[nH]c3c(c12)CCCC3 ; CC(C)Oc1cc(F)ccc1Nc1ncnc2[nH]c3c(c12)CCCC3]

A mixture comprising 17.7 mg (90 μmol) 4-chloro-6,7,8,9-tetrahydro-5H-pyrimido[4,5-b]indole which was prepared according to intermediate example 1a, 27.8 mg 4-fluoro-2-isopropoxyaniline hydrochloride, 255 μL ethanol and 4.88 μL hydrochloric acid (4M in dioxane) was reacted at 90-100° C. under microwave irradiation for 3.5 hours. The residue was solved in a mixture of ethyl acetate methanol, washed with aqueous sodium hydrogencarbonate. After removal of the solvents the residue was purified by chromatography to give 9.5 mg (29%) of the title compound.

¹H-NMR (CDCL₃): δ=1.43 (6H), 1.94 (4H), 2.78 (2H), 2.96 (2H), 4.63 (1H), 6.63-6.77 (2H), 7.76 (1H), 8.36 (1H), 8.75 (1H), 10.25 (1H) ppm.

### Example 1a

### 4-Chloro-6,7,8,9-tetrahydro-5H-pyrimido[4,5-b]indole

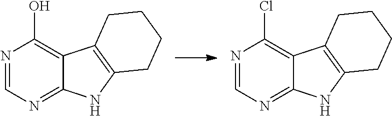 [SMILES: Oc1ncnc2[nH]c3c(c12)CCCC3 ; Clc1ncnc2[nH]c3c(c12)CCCC3]

A mixture comprising 148 mg (782 μmol) 6,7,8,9-tetrahydro-5H-pyrimido[4,5-b]indol-4-ol which was prepared according to intermediate example 1b and 5.47 mL phosphorus oxychloride was heated at 100° C. for 1 hour. The reagent was removed and the residue purified by chromatography to give 106.8 mg (62%) of the title compound.

### Example 1b

### 6,7,8,9-Tetrahydro-5H-pyrimido[4,5-b]indol-4-ol

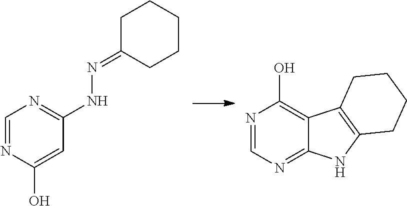 [SMILES: Oc1cc(NN=C2CCCCC2)ncn1 ; Oc1ncnc2[nH]c3c(c12)CCCC3]

A mixture comprising 192.5 mg (933 μmol) 6-(2-cyclohexylidenehydrazino)pyrimidin-4-ol which was prepared according to intermediate example 1c and 5.0 mL xylol was heated at 250° C. under microwave irradiation for 2.5 hours. The solid was filtered off and washed with diethyl ether to give 152.9 mg (87%) of the title compound.

### Example 1c

### 6-(2-Cyclohexylidenehydrazino)pyrimidin-4-ol

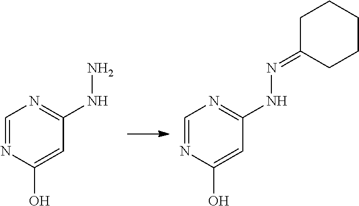 [SMILES: NNc1cc(O)ncn1 ; Oc1cc(NN=C2CCCCC2)ncn1]

A mixture comprising 240 mg (1.90 mmol) 6-hydrazinopyrimidin-4-ol/6-hydrazinopyrimidin-4(1H)-one (CAS-No: 29939-37-5), 280 mg cyclohexanone and 3.88 mL ethanol was heated under reflux for 1.5 h. After cooling to 3° C., the precipitated solid was filtered off and washed with diethyl ether to give 354.1 mg (86%) of the title compound.

### Example 2

### N-(1H-Indazol-5-yl)-6,7,8,9-tetrahydro-5H-pyrimido[4,5-b]indol-4-amine

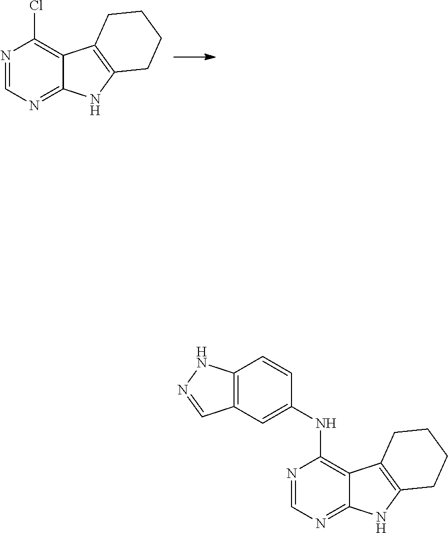 [SMILES: Clc1ncnc2[nH]c3c(c12)CCCC3 ; c1nc(Nc2ccc3[nH]ncc3c2)c2c3c([nH]c2n1)CCCC3]

20 mg (96 μmol) 4-chloro-6,7,8,9-tetrahydro-5H-pyrimido[4,5-b]indole which was prepared according to intermediate example 1a were transformed in analogy to example 1 using 1H-indazol-5-amine to give after working up and purification 9.9 mg (32%) of the title compound.

¹H-NMR (DMSO-d6): δ=1.77 (4H), 2.62 (2H), 2.89 (2H), 7.41-7.55 (2H), 7.90 (1H), 7.99 (1H), 8.06 (2H), 11.35 (1H), 12.89 (1H) ppm.

### Example 3

### N-(1H-Indazol-5-yl)-5,6,7,8,9,10-hexahydrocyclohepta[4,5]pyrrolo[2,3-d]pyrimidin-4-amine

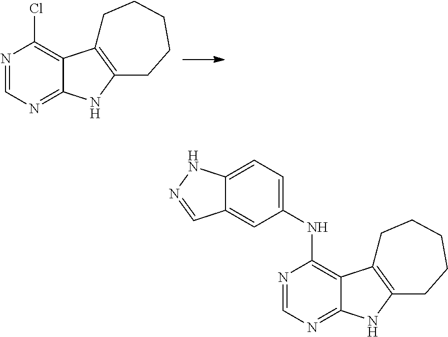 [SMILES: Clc1ncnc2[nH]c3c(c12)CCCCC3 ; c1nc(Nc2ccc3[nH]ncc3c2)c2c3c([nH]c2n1)CCCCC3]

20 mg (90 μmol) 4-chloro-5,6,7,8,9,10-hexahydrocyclohepta[4,5]pyrrolo[2,3-d]pyrimidine which was prepared according to intermediate example 3a were transformed in analogy to example 1 using 1H-indazol-5-amine to give after working up and purification 10.3 mg (34%) of the title compound.

¹H-NMR (CD₃OD): δ=1.73-1.84 (4H), 1.84-1.92 (2H), 2.4 (2H), 3.00 (2H), 7.46 (1H), 7.51 (1H), 7.86 (1H), 7.98 (2H) ppm.

### Example 3a

### 4-Chloro-5,6,7,8,9,10-hexahydrocyclohepta[4,5]pyrrolo[2,3-d]pyrimidine

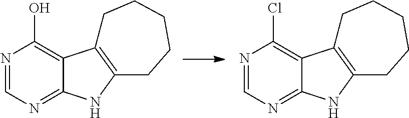 [SMILES: Oc1ncnc2[nH]c3c(c12)CCCCC3 ; Clc1ncnc2[nH]c3c(c12)CCCCC3]

188 mg (925 μmol) 5,6,7,8,9,10-hexahydrocyclohepta[4,5]pyrrolo[2,3-d]pyrimidin-4-ol which was prepared according to intermediate example 3b were transformed in analogy to intermediate example 1a to give after working up and purification 143.8 mg (67%) of the title compound.

### Example 3b

### 5,6,7,8,9,10-Hexahydrocyclohepta[4,5]pyrrolo[2,3-d]pyrimidin-4-ol

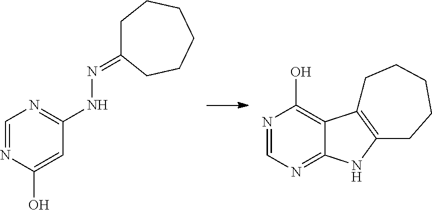 [SMILES: Oc1cc(NN=C2CCCCCC2)ncn1 ; Oc1ncnc2[nH]c3c(c12)CCCCC3]

200 mg (908 μmol) 6-(2-cycloheptylidenehydrazino)pyrimidin-4-ol which was prepared according to intermediate example 3c were transformed in analogy to intermediate example 1b to give after working up and purification 170.8 mg (88%) of the title compound.

### Example 3c

### 6-(2-Cycloheptylidenehydrazino)pyrimidin-4-ol

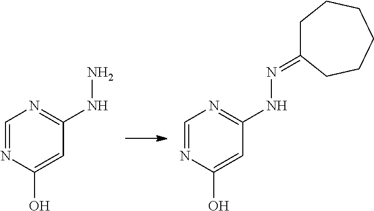 [SMILES: NNc1cc(O)ncn1 ; Oc1cc(NN=C2CCCCCC2)ncn1]

200 mg (1.59 mmol) 6-hydrazinopyrimidin-4-ol/6-hydrazinopyrimidin-4(1H)-one (CAS-No: 29939-37-5) were transformed in analogy to intermediate example 1c using cycloheptanone to give after working up and purification 289.6 mg (77%) of the title compound.

### Example 4

### N-(4-Fluoro-2-isopropoxyphenyl)-5,6,7,8,9,10-hexahydrocyclohepta[4,5]pyrrolo[2,3-d]pyrimidin-4-amine

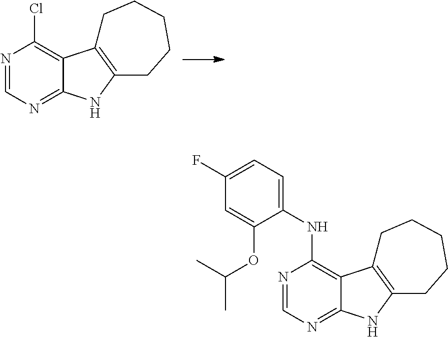 [SMILES: Clc1ncnc2[nH]c3c(c12)CCCCC3 ; CC(C)Oc1cc(F)ccc1Nc1ncnc2[nH]c3c(c12)CCCCC3]

20 mg (90 μmol) 4-chloro-5,6,7,8,9,10-hexahydrocyclohepta[4,5]pyrrolo[2,3-d]pyrimidine which was prepared according to intermediate example 3a were transformed in analogy to example 1 to give after working up and purification 5.4 mg (16%) of the title compound.

¹H-NMR (DMSO-d6): δ=1.35 (6H), 1.72 (2H), 1.77-1.94 (4H), 2.81 (2H), 3.03 (2H), 4.79 (1H), 6.79 (1H), 7.05 (1H), 7.86 (1H), 8.21 (1H), 8.74 (1H), 11.56 (1H) ppm.

### Example 5

### (RS)-Ethyl 4-(1H-indazol-5-ylamino)-6,7,8,9-tetrahydro-5H-pyrimido[4,5-b]indole-6-carboxylate

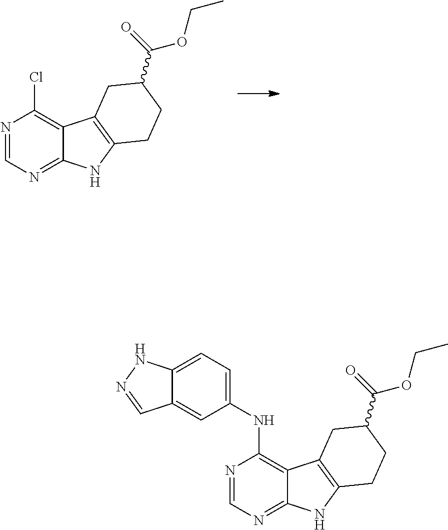 [SMILES: CCOC(=O)C1CCc2[nH]c3ncnc(Cl)c3c2C1 ; CCOC(=O)C1CCc2[nH]c3ncnc(Nc4ccc5[nH]ncc5c4)c3c2C1]

315 mg (1.13 mmol) ethyl 4-chloro-6,7,8,9-tetrahydro-5H-pyrimido[4,5-b]indole-6-carboxylate which was prepared according to intermediate example 5a were transformed in analogy to example 1 using 1H-indazol-5-amine to give after working up and purification 380.3 mg (85%) of the title compound.

¹H-NMR (DMSO-d6): δ=1.18 (3H), 1.82 (1H), 2.12 (1H), 2.63-2.81 (3H), 2.96 (1H), 3.21 (1H), 4.00-4.16 (2H), 7.43 (1H), 7.48 (1H), 7.96 (1H), 7.98 (1H), 8.03 (1H), 8.04 (1H), 11.40 (1H), 12.91 (1H) ppm.

### Example 5a

### (RS)-Ethyl 4-chloro-6,7,8,9-tetrahydro-5H-pyrimido[4,5-b]indole-6-carboxylate

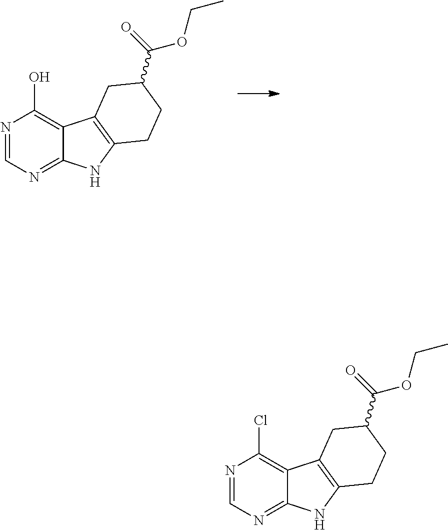 [SMILES: CCOC(=O)C1CCc2[nH]c3ncnc(O)c3c2C1 ; CCOC(=O)C1CCc2[nH]c3ncnc(Cl)c3c2C1]

412 mg (1.58 mmol) ethyl 4-hydroxy-6,7,8,9-tetrahydro-5H-pyrimido[4,5-b]indole-6-carboxylate which was prepared according to intermediate example 5b were transformed in analogy to intermediate example 1a to give after working up and purification 321.6 mg (73%) of the title compound.

### Example 5b

### (RS)-Ethyl 4-hydroxy-6,7,8,9-tetrahydro-5H-pyrimido[4,5-b]indole-6-carboxylate

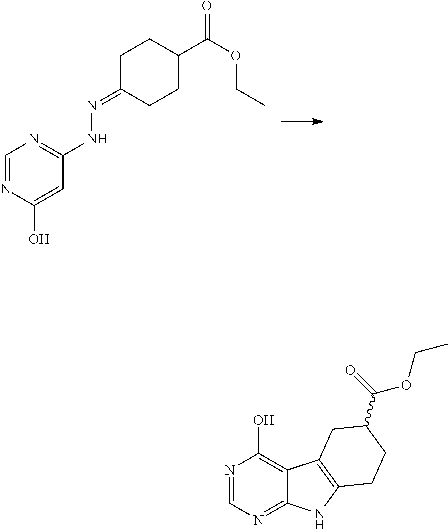 [SMILES: CCOC(=O)C1CCC(=NNc2cc(O)ncn2)CC1 ; CCOC(=O)C1CCc2[nH]c3ncnc(O)c3c2C1]

3.17 g (11.37 mmol) ethyl 4-[(6-hydroxypyrimidin-4-yl)hydrazono]cyclohexanecarboxylate which was prepared according to intermediate example 5c were transformed in analogy to intermediate example 1c to give after working up and purification 2.83 g (95%) of the title compound.

### Example 5c

### Ethyl 4-[(6-hydroxypyrimidin-4-yl)hydrazono]cyclohexanecarboxylate

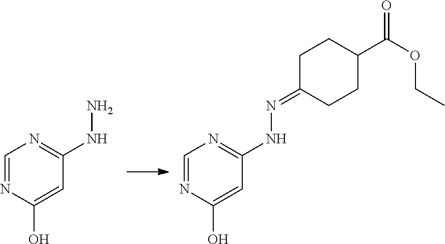 [SMILES: NNc1cc(O)ncn1 ; CCOC(=O)C1CCC(=NNc2cc(O)ncn2)CC1]

4.29 g (34.0 mmol) 6-hydrazinopyrimidin-4-ol/6-hydrazinopyrimidin-4(1H)-one (CAS-No: 29939-37-5) were transformed in analogy to intermediate example 1c using ethyl 4-oxocyclohexanecarboxylate to give after working up and purification 6.83 g (72%) of the title compound.

### Example 6

### (RS)-4-(1H-Indazol-5-ylamino)-6,7,8,9-tetrahydro-5H-pyrimido[4,5-b]indole-6-carboxylic acid

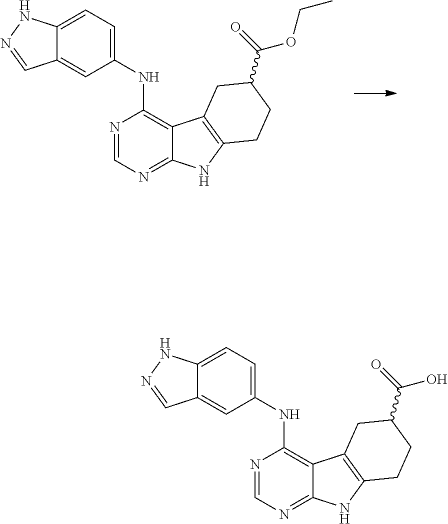 [SMILES: CCOC(=O)C1CCc2[nH]c3ncnc(Nc4ccc5[nH]ncc5c4)c3c2C1 ; O=C(O)C1CCc2[nH]c3ncnc(Nc4ccc5[nH]ncc5c4)c3c2C1]

A mixture comprising 317.2 mg (843 μmol) (RS)-ethyl 4-(1H-indazol-5-ylamino)-6,7,8,9-tetrahydro-5H-pyrimido[4,5-b]indole-6-carboxylate which was prepared according to example 5, 5.06 mL lithium hydroxide solution (1M in water), 14.6 mL tetrahydrofuran and 3.9 mL methanol was stirred at 23° C. over night. Hydrochloric acid was added and the solvents were removed. The residue was solved in ethanol, ether was added until the product started to precipitate. After filtration 293 mg (99%) of the title compound were obtained.

¹H-NMR (DMSO-d6): δ=1.82 (1H), 2.15 (1H), 2.67-2.81 (3H), 2.93 (1H), 3.22 (1H), 7.36 (1H), 7.68 (1H), 7.87 (1H), 8.06 (1H), 8.14 (1H), 10.03 (1H), 12.66 (1H), 13.33 (1H) ppm.

### Example 7

### (RS)—N-Ethyl-4-(1H-indazol-5-ylamino)-6,7,8,9-tetrahydro-5H-pyrimido[4,5-b]indole-6-carboxamide

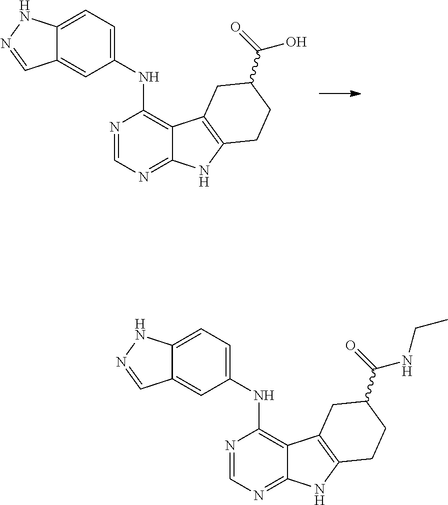 [SMILES: O=C(O)C1CCc2[nH]c3ncnc(Nc4ccc5[nH]ncc5c4)c3c2C1 ; CCNC(=O)C1CCc2[nH]c3ncnc(Nc4ccc5[nH]ncc5c4)c3c2C1]

A mixture comprising 50 mg (144 μmol) (RS)-4-(1H-indazol-5-ylamino)-6,7,8,9-tetrahydro-5H-pyrimido[4,5-b]indole-6-carboxylic acid which was prepared according to example 6, 215 μL ethanamine solution in tetrahydrofuran (2M), 81.9 mg N-[(dimethylamino)(3H-[1,2,3]triazolo[4,5-b]pyridin-3-yloxy)methylene]-N-methylmethanaminium hexafluorophosphate, 26.3 mg N,N-dimethylpyridin-4-amine and 2.75 mL N,N-dimethylformamide was stirred at 23° C. overnight. The solvent was removed and the residue purified by chromatography to give 5.0 mg (8%) of the title compound.

¹H-NMR (DMSO-d6): δ=1.01 (3H), 1.77 (1H), 1.97 (1H), 2.61-2.71 (2H), 2.93 (1H), 3.02-3.16 (4H), 7.43 (1H), 7.49 (1H), 7.87 (1H), 7.93-8.01 (3H), 8.04 (1H), 11.36 (1H), 12.91 (1H) ppm.

### Example 8

### 6-Benzyl-N-(4-fluoro-2-isopropoxyphenyl)-6,7,8,9-tetrahydro-5H-pyrido[3′,4′:4,5]pyrrolo[2,3-d]pyrimidin-4-amine

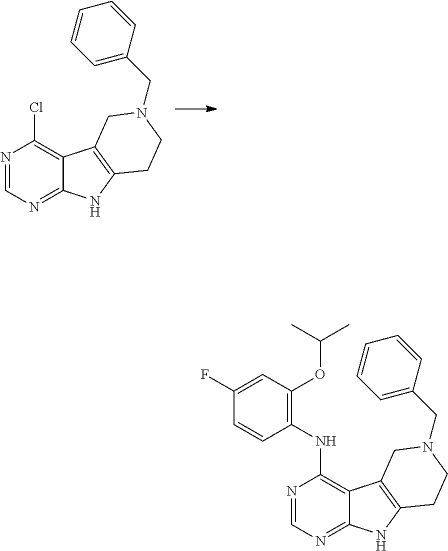 [SMILES: Clc1ncnc2[nH]c3c(c12)CN(Cc1ccccc1)CC3 ; CC(C)Oc1cc(F)ccc1Nc1ncnc2[nH]c3c(c12)CN(Cc1ccccc1)CC3]

23 mg (77 μmol) 6-benzyl-4-chloro-6,7,8,9-tetrahydro-5H-pyrido[3′,4′:4,5]pyrrolo[2,3-d]pyrimidine hydrochloride which was prepared according to intermediate example 8a were transformed in analogy to example 1 to give after working up and purification 2.1 mg (6%) of the title compound.

¹H-NMR (DMSO-d6): δ=1.18 (6H), 2.66-2.84 (4H), 3.76 (2H), 3.88 (2H), 4.67 (1H), 6.73 (1H), 6.97 (1H), 7.20-7.39 (6H), 8.20 (1H), 8.63 (1H), 11.64 (1H) ppm.

### Example 8a

### 6-Benzyl-4-chloro-6,7,8,9-tetrahydro-5H-pyrido[3′,4′:4,5]pyrrolo[2,3-d]pyrimidine hydrochloride

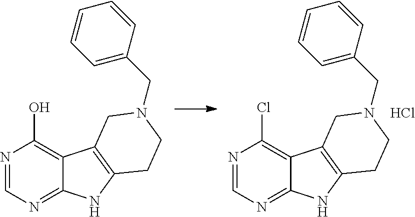 [SMILES: Oc1ncnc2[nH]c3c(c12)CN(Cc1ccccc1)CC3 ; Clc1ncnc2[nH]c3c(c12)CN(Cc1ccccc1)CC3 ; Cl]

144 mg (514 μmol) 6-benzyl-6,7,8,9-tetrahydro-5H-pyrido[3′,4′:4,5]pyrrolo[2,3-d]pyrimidin-4-ol which was prepared according to intermediate example 8b were transformed in analogy to intermediate example 1a to give after working up and purification 131 mg (85%) of the title compound.

### Example 8b

### 6-Benzyl-6,7,8,9-tetrahydro-5H-pyrido[3′,4′:4,5]pyrrolo[2,3-d]pyrimidin-4-ol

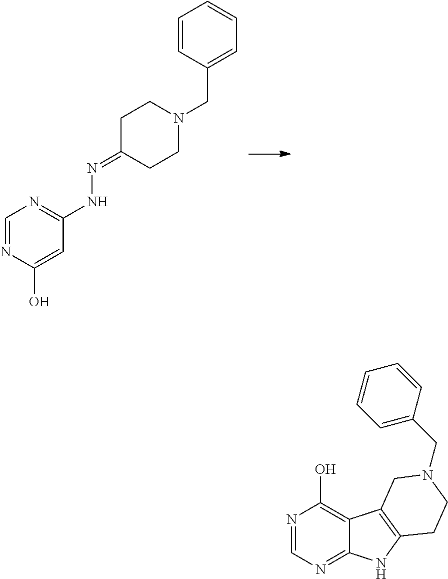 [SMILES: Oc1cc(NN=C2CCN(Cc3ccccc3)CC2)ncn1 ; Oc1ncnc2[nH]c3c(c12)CN(Cc1ccccc1)CC3]

190 mg (639 μmol) 6-[2-(1-benzylpiperidin-4-ylidene)hydrazino]pyrimidin-4-ol which was prepared according to intermediate example 8c were transformed in analogy to intermediate example 1b to give after working up and purification 148.2 mg (79%) of the title compound.

### Example 8c

### 6-[2-(1-Benzylpiperidin-4-ylidene)hydrazino]pyrimidin-4-ol

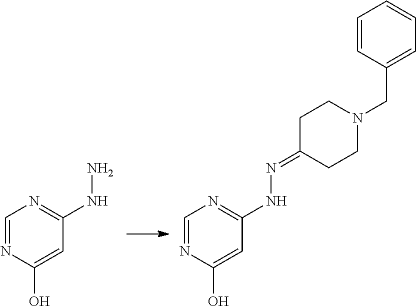 [SMILES: NNc1cc(O)ncn1 ; Oc1cc(NN=C2CCN(Cc3ccccc3)CC2)ncn1]

100 mg (793 μmol) 6-hydrazinopyrimidin-4-ol/6-hydrazinopyrimidin-4(1H)-one (CAS-No: 29939-37-5) were transformed in analogy to intermediate example 1c using 1-benzylpiperidin-4-one to give after working up and purification 194.8 mg (78%) of the title compound.

### Example 9

### 6,6-Difluoro-N-(4-fluoro-2-isopropoxyphenyl)-6,7,8,9-tetrahydro-5H-pyrimido[4,5-b]indol-4-amine

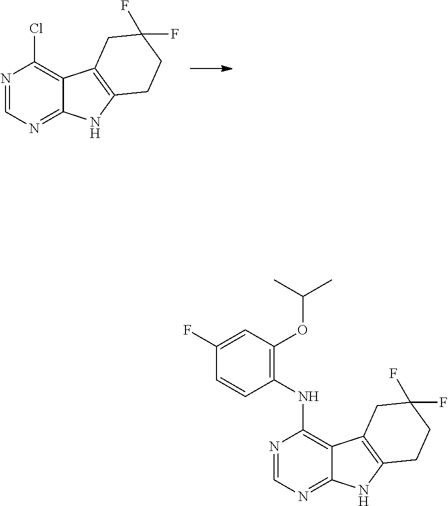 [SMILES: FC1(F)CCc2[nH]c3ncnc(Cl)c3c2C1 ; CC(C)Oc1cc(F)ccc1Nc1ncnc2[nH]c3c(c12)CC(F)(F)CC3]

23 mg (94 μmol) 4-chloro-6,6-difluoro-6,7,8,9-tetrahydro-5H-pyrimido[4,5-b]indole which was prepared according to intermediate example 9a were transformed in analogy to example 1 to give after working up and purification 16.9 mg (48%) of the title compound.

¹H-NMR (DMSO-d6): δ=1.32 (6H), 2.26-2.39 (2H), 2.86 (2H), 3.48 (2H), 4.75 (1H), 6.75 (1H), 7.01 (1H), 7.55 (1H), 8.21 (1H), 8.58 (1H), 11.71 (1H) ppm.

### Example 9a

### 4-Chloro-6,6-difluoro-6,7,8,9-tetrahydro-5H-pyrimido[4,5-b]indole

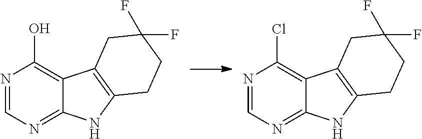 [SMILES: Oc1ncnc2[nH]c3c(c12)CC(F)(F)CC3 ; FC1(F)CCc2[nH]c3ncnc(Cl)c3c2C1]

118 mg (524 μmol) 6,6-difluoro-6,7,8,9-tetrahydro-5H-pyrimido[4,5-b]indol-4-ol which was prepared according to intermediate example 9b were transformed in analogy to intermediate example 1a to give after working up and purification 71.3 mg (56%) of the title compound.

### Example 9b

### 6,6-Difluoro-6,7,8,9-tetrahydro-5H-pyrimido[4,5-b]indol-4-ol

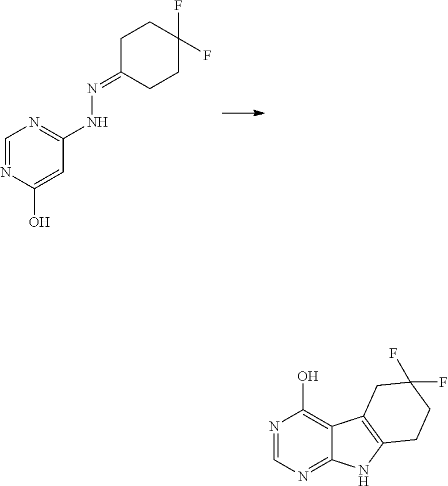 [SMILES: Oc1cc(NN=C2CCC(F)(F)CC2)ncn1 ; Oc1ncnc2[nH]c3c(c12)CC(F)(F)CC3]

163 mg (673 μmol) 6-[2-(4,4-difluorocyclohexylidene)hydrazino]pyrimidin-4-ol which was prepared according to intermediate example 9c were transformed in analogy to intermediate example 1b to give after working up and purification 122.5 mg (77%) of the title compound.

### Example 9c

### 6-[2-(4,4-Difluorocyclohexylidene)hydrazino]pyrimidin-4-ol

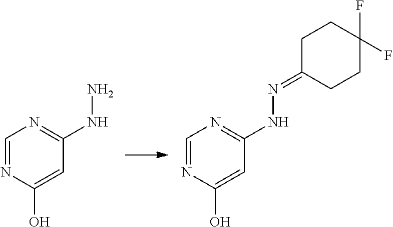 [SMILES: NNc1cc(O)ncn1 ; Oc1cc(NN=C2CCC(F)(F)CC2)ncn1]

100 mg (793 μmol) 6-hydrazinopyrimidin-4-ol/6-hydrazinopyrimidin-4(1H)-one (CAS-No: 29939-37-5) were transformed in analogy to intermediate example 1c using 4,4-difluorocyclohexanone to give after working up and purification 167.2 mg (83%) of the title compound.

### Example 10

### 6,6-Difluoro-N-(1H-indazol-5-yl)-6,7,8,9-tetrahydro-5H-pyrimido[4,5-b]indol-4-amine

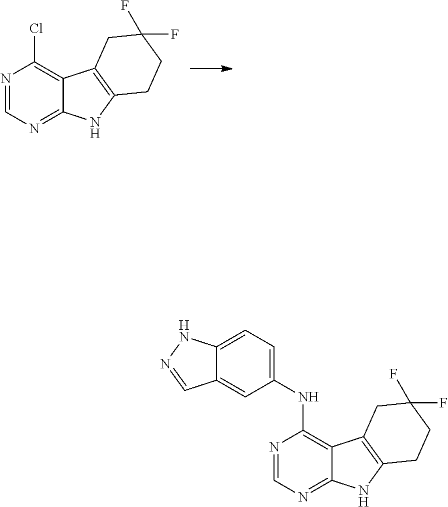 [SMILES: FC1(F)CCc2[nH]c3ncnc(Cl)c3c2C1 ; FC1(F)CCc2[nH]c3ncnc(Nc4ccc5[nH]ncc5c4)c3c2C1]

23 mg (94 μmol) 4-chloro-6,6-difluoro-6,7,8,9-tetrahydro-5H-pyrimido[4,5-b]indole which was prepared according to intermediate example 9a were transformed in analogy to example 1 using 1H-indazol-5-amine to give after working up and purification 11 mg (34%) of the title compound.

¹H-NMR (DMSO-d6): δ=2.22-2.35 (2H), 2.85 (2H), 3.52 (2H), 7.45 (1H), 7.51 (1H), 7.99 (2H), 8.09 (2H), 11.58 (1H), 12.92 (1H) ppm.

### Example 11

### N-(4-Fluoro-2-isopropoxyphenyl)-5,7,8,9-tetrahydrothiopyrano[3′,4′:4,5]pyrrolo[2,3-d]pyrimidin-4-amine

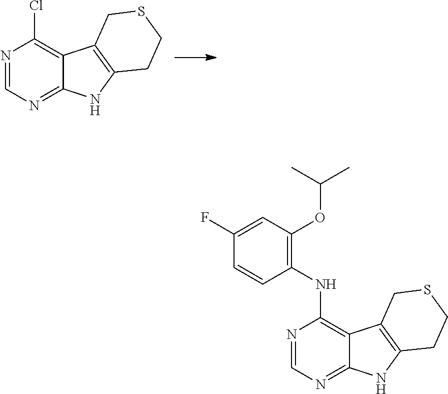 [SMILES: Clc1ncnc2[nH]c3c(c12)CSCC3 ; CC(C)Oc1cc(F)ccc1Nc1ncnc2[nH]c3c(c12)CSCC3]

23 mg (102 μmol) 4-chloro-5,7,8,9-tetrahydrothiopyrano[3′,4′:4,5]pyrrolo[2,3-d]pyrimidine which was prepared according to intermediate example 11a were transformed in analogy to example 1 to give after working up and purification 13 mg (36%) of the title compound.

¹H-NMR (DMSO-d6): δ=1.30 (6H), 2.86-2.98 (4H), 4.05 (2H), 4.72 (1H), 6.74 (1H), 6.99 (1H), 7.59 (1H), 8.19 (1H), 8.59 (1H), 11.64 (1H) ppm.

### Example 11a

### 4-Chloro-5,7,8,9-tetrahydrothiopyrano[3′,4′:4,5]pyrrolo[2,3-d]pyrimidine

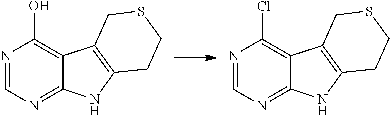 [SMILES: Oc1ncnc2[nH]c3c(c12)CSCC3 ; Clc1ncnc2[nH]c3c(c12)CSCC3]

104 mg (502 μmol) 5,7,8,9-tetrahydrothiopyrano[3′,4′:4,5]pyrrolo[2,3-d]pyrimidin-4-ol which was prepared according to intermediate example 11b were transformed in analogy to intermediate example 1a to give after working up and purification 46.7 mg (41%) of the title compound.

### Example 11b

### 5,7,8,9-Tetrahydrothiopyrano[3′,4′:4,5]pyrrolo[2,3-d]pyrimidin-4-ol

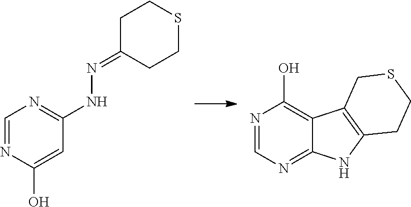 [SMILES: Oc1cc(NN=C2CCSCC2)ncn1 ; Oc1ncnc2[nH]c3c(c12)CSCC3]

192.5 mg (585 μmol) 6-[2-(tetrahydro-4H-thiopyran-4-ylidene)hydrazino]pyrimidin-4-ol which was prepared according to intermediate example 11c were transformed in analogy to intermediate example 1b to give after working up and purification 107.2 mg (57%) of the title compound.

### Example 11c

### 6-[2-(Tetrahydro-4H-thiopyran-4-ylidene)hydrazino]pyrimidin-4-ol

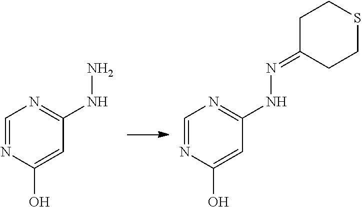 [SMILES: NNc1cc(O)ncn1 ; Oc1cc(NN=C2CCSCC2)ncn1]

100 mg (793 μmol) 6-hydrazinopyrimidin-4-ol/6-hydrazinopyrimidin-4(1H)-one (CAS-No: 29939-37-5) were transformed in analogy to intermediate example 1c using tetrahydro-4H-thiopyran-4-one to give after working up and purification 182.5 mg (97%) of the title compound.

### Example 12

### N-(4,5-Dichloro-2-methoxyphenyl)-5,6,7,8,9,10-hexahydrocyclohepta[4,5]pyrrolo[2,3-d]pyrimidin-4-amine

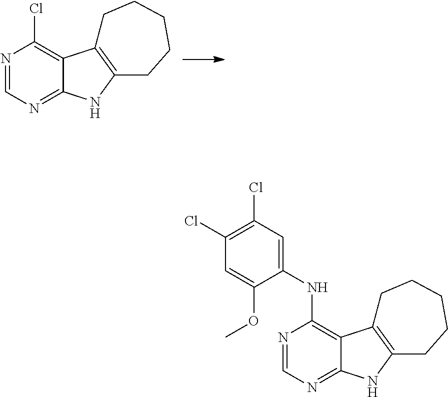 [SMILES: Clc1ncnc2[nH]c3c(c12)CCCCC3 ; COc1cc(Cl)c(Cl)cc1Nc1ncnc2[nH]c3c(c12)CCCCC3]

20 mg (90 μmol) 4-chloro-5,6,7,8,9,10-hexahydrocyclohepta[4,5]pyrrolo[2,3-d]pyrimidine which was prepared according to intermediate example 3a were transformed in analogy to example 1 using 4,5-dichloro-2-methoxyaniline to give after working up and purification 11.7 mg (33%) of the title compound.

¹H-NMR (DMSO-d6): δ=1.63-1.85 (6H), 2.77 (2H), 2.93 (2H), 3.93 (3H), 7.30 (1H), 7.85 (1H), 8.23 (1H), 8.88 (1H), 11.58 (1H) ppm.

### Example 13

### (RS)-Ethyl 4-[(4,5-dichloro-2-methoxyphenyl)amino]-6,7,8,9-tetrahydro-5H-pyrimido[4,5-b]indole-6-carboxylate

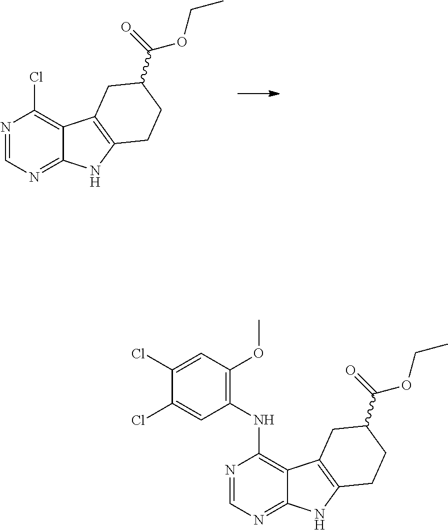 [SMILES: CCOC(=O)C1CCc2[nH]c3ncnc(Cl)c3c2C1 ; CCOC(=O)C1CCc2[nH]c3ncnc(Nc4cc(Cl)c(Cl)cc4OC)c3c2C1]

100 mg (357 μmol) (RS)-Ethyl 4-chloro-6,7,8,9-tetrahydro-5H-pyrimido[4,5-b]indole-6-carboxylate which was prepared according to intermediate example 5a were transformed in analogy to intermediate example 1a using 4,5-dichloro-2-methoxyaniline to give after working up and purification 131 mg (84%) of the title compound.

¹H-NMR (DMSO-d6): δ=1.19 (3H), 1.83 (1H), 2.16 (1H), 2.71-2.87 (3H), 2.93 (1H), 3.15 (1H), 3.82 (3H), 4.04-4.16 (2H), 7.46 (1H), 8.09 (1H), 8.23 (1H), 9.20 (1H), 12.47 (1H) ppm.

### Example 14

### (RS)-4-(1H-Indazol-5-ylamino)-N-[3-(methylsulfonyl)propyl]-6,7,8,9-tetrahydro-5H-pyrimido[4,5-b]indole-6-carboxamide

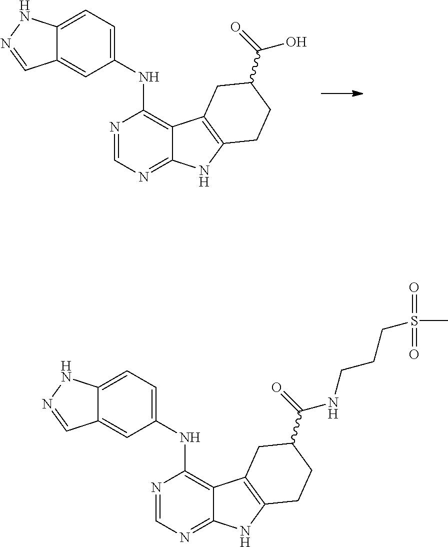 [SMILES: O=C(O)C1CCc2[nH]c3ncnc(Nc4ccc5[nH]ncc5c4)c3c2C1 ; CS(=O)(=O)CCCNC(=O)C1CCc2[nH]c3ncnc(Nc4ccc5[nH]ncc5c4)c3c2C1]

A mixture comprising 25 mg (72 μmol) (RS)-4-(1H-indazol-5-ylamino)-6,7,8,9-tetrahydro-5H-pyrimido[4,5-b]indole-6-carboxylic acid which was prepared according to example 6, 0.8 mL N,N-dimethylformamide, 37.4 mg 3-(methylsulfonyl)propan-1-amine hydrochloride, 42.7 μL 2,4,6-tripropyl-1,3,5,2,4,6-trioxatriphosphinane 2,4,6-trioxide solution (50% in ethyl acetate) and 37.5 μL N-ethyl-N-isopropylpropan-2-amine was stirred at 23° C. for 3 hours. Water was added, the precipitate filtered off, washed with water and dried to give 5.4 mg (16%) of the title compound.

¹H-NMR (DMSO-d6): δ=1.72-1.90 (3H), 2.01 (1H), 2.51 (1H), 2.60-2.75 (2H), 2.94 (3H), 2.96 (1H), 3.06-3.25 (5H), 7.43 (1H), 7.51 (1H), 7.93-8.02 (4H), 8.05 (1H), 11.36 (1H), 12.89 (1H) ppm.

### Example 15

### (RS)—N—Cyclopropyl-4-(1H-indazol-5-ylamino)-6,7,8,9-tetrahydro-5H-pyrimido[4,5-b]indole-6-carboxamide

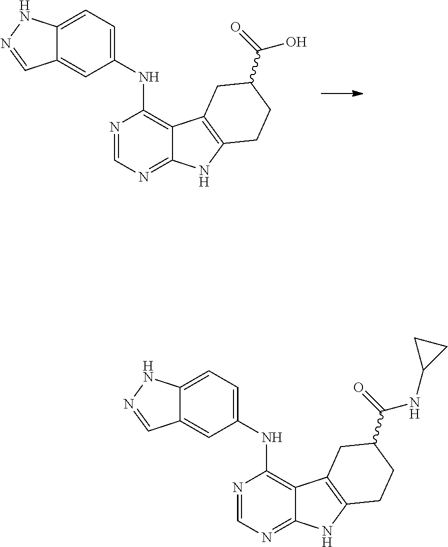 [SMILES: O=C(O)C1CCc2[nH]c3ncnc(Nc4ccc5[nH]ncc5c4)c3c2C1 ; O=C(NC1CC1)C1CCc2[nH]c3ncnc(Nc4ccc5[nH]ncc5c4)c3c2C1]

25 mg (72 μmol) (RS)-4-(1H-indazol-5-ylamino)-6,7,8,9-tetrahydro-5H-pyrimido[4,5-b]indole-6-carboxylic acid which was prepared according to example 6 were transformed in analogy to example 14 using cyclopropanamine to give after working up and purification 6.0 mg (28%) of the title compound.

¹H-NMR (DMSO-d6): δ=0.38 (2H), 0.59 (2H), 1.76 (1H), 1.95 (1H), 2.42 (1H), 2.58-2.72 (3H), 2.92 (1H), 3.09 (1H), 7.44 (1H), 7.49 (1H), 7.92 (1H), 7.95-8.00 (3H), 8.04 (1H), 11.35 (1H), 12.90 (1H) ppm.

### Example 16

### (RS)-4-(1H-Indazol-5-ylamino)-N-phenyl-6,7,8,9-tetrahydro-5H-pyrimido[4,5-b]indole-6-carboxamide

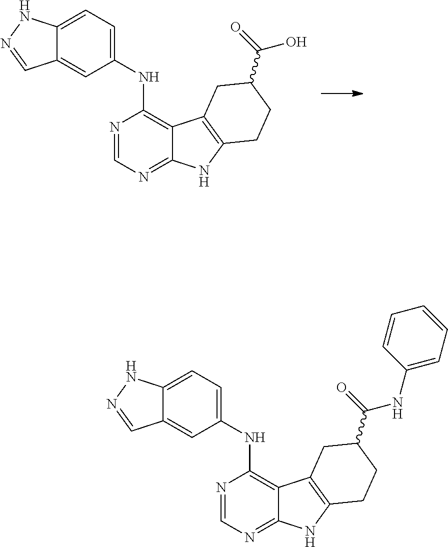 [SMILES: O=C(O)C1CCc2[nH]c3ncnc(Nc4ccc5[nH]ncc5c4)c3c2C1 ; O=C(Nc1ccccc1)C1CCc2[nH]c3ncnc(Nc4ccc5[nH]ncc5c4)c3c2C1]

25 mg (72 μmol) (RS)-4-(1H-indazol-5-ylamino)-6,7,8,9-tetrahydro-5H-pyrimido[4,5-b]indole-6-carboxylic acid which was prepared according to example 6 were transformed in analogy to example 14 using aniline to give after working up and purification 14.6 mg (48%) of the title compound.

¹H-NMR (DMSO-d6): δ=1.88 (1H), 2.11 (1H), 2.65-2.83 (4H), 3.04 (1H), 7.00 (1H), 7.27 (2H), 7.42 (1H), 7.49 (1H), 7.62 (2H), 7.91-8.07 (4H), 9.96 (1H), 11.39 (1H), 12.88 (1H) ppm.

### Example 17

### (RS)-(3,3-Difluoroazetidin-1-yl)[4-(1H-indazol-5-ylamino)-6,7,8,9-tetrahydro-5H-pyrimido[4,5-b]indol-6-yl]methanone

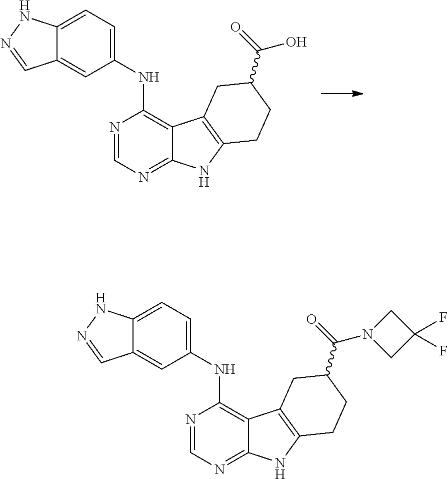 [SMILES: O=C(O)C1CCc2[nH]c3ncnc(Nc4ccc5[nH]ncc5c4)c3c2C1 ; O=C(C1CCc2[nH]c3ncnc(Nc4ccc5[nH]ncc5c4)c3c2C1)N1CC(F)(F)C1]

25 mg (72 μmol) (RS)-4-(1H-indazol-5-ylamino)-6,7,8,9-tetrahydro-5H-pyrimido[4,5-b]indole-6-carboxylic acid which was prepared according to example 6 were transformed in analogy to example 14 using 3,3-difluoroazetidine to give after working up and purification 6.3 mg (19%) of the title compound.

¹H-NMR (DMSO-d6): δ=1.75 (1H), 1.99 (1H), 2.65-2.75 (3H), 2.88 (1H), 3.15 (1H), 4.28 (2H), 4.71 (2H), 7.44 (1H), 7.50 (1H), 7.93-8.01 (3H), 8.05 (1H), 11.36 (1H), 12.89 (1H) ppm.

### Example 18

### (RS)-4-[(4,5-Dichloro-2-methoxyphenyl)amino]-6,7,8,9-tetrahydro-5H-pyrimido[4,5-b]indole-6-carboxylic acid

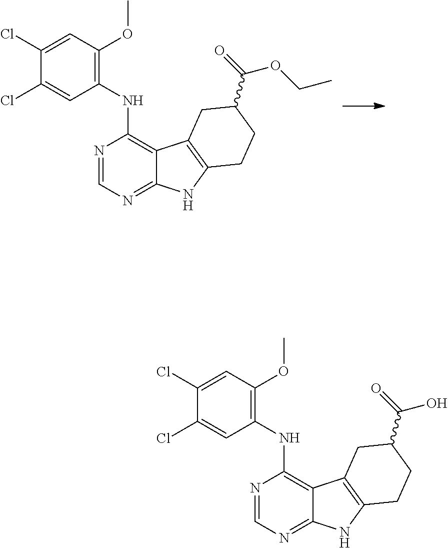 [SMILES: CCOC(=O)C1CCc2[nH]c3ncnc(Nc4cc(Cl)c(Cl)cc4OC)c3c2C1 ; COc1cc(Cl)c(Cl)cc1Nc1ncnc2[nH]c3c(c12)CC(C(=O)O)CC3]

107 mg (246 μmol) (RS)-Ethyl 4-[(4,5-dichloro-2-methoxyphenyl)amino]-6,7,8,9-tetrahydro-5H-pyrimido[4,5-b]indole-6-carboxylate which was prepared according to example 13 were transformed in analogy to example 6 to give after working up and purification 93.5 mg (93%) of the title compound.

¹H-NMR (DMSO-d6): δ=1.80 (1H), 2.14 (1H), 2.64-2.74 (3H), 2.98 (1H), 3.11 (1H), 3.91 (3H), 7.27 (1H), 7.73 (1H), 8.25 (1H), 8.88 (1H), 11.57 (1H) ppm.

### Example 19

### N-(4,5-Dichloro-2-methoxyphenyl)-6,6-difluoro-6,7,8,9-tetrahydro-5H-pyrimido[4,5-b]indol-4-amine

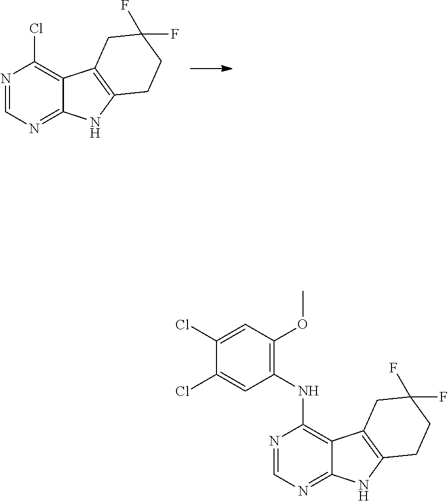 [SMILES: FC1(F)CCc2[nH]c3ncnc(Cl)c3c2C1 ; COc1cc(Cl)c(Cl)cc1Nc1ncnc2[nH]c3c(c12)CC(F)(F)CC3]

22.3 mg (92 μmol) 4-chloro-6,6-difluoro-6,7,8,9-tetrahydro-5H-pyrimido[4,5-b]indole which was prepared according to intermediate example 9a were transformed in analogy to example 1 using 4,5-dichloro-2-methoxyaniline to give after working up and purification 29.3 mg (72%) of the title compound.

¹H-NMR (DMSO-d6): δ=2.29 (2H), 2.87 (2H), 3.46 (2H), 3.91 (3H), 7.31 (1H), 7.66 (1H), 8.26 (1H), 8.72 (1H), 11.78 (1H) ppm.

### Example 20

### (RS)—N-Cyclopropyl-4-[(4,5-dichloro-2-methoxyphenyl)amino]-6,7,8,9-tetrahydro-5H-pyrimido[4,5-b]indole-6-carboxamide

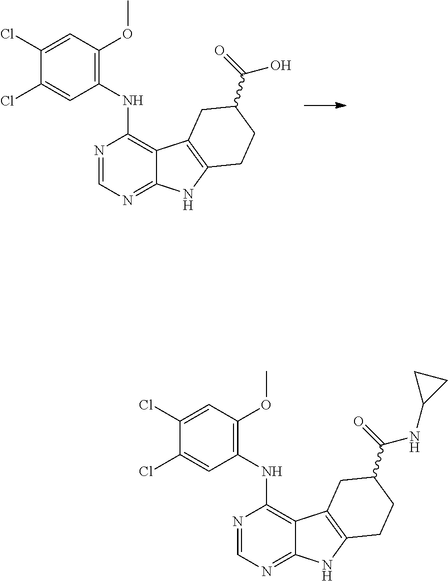 [SMILES: COc1cc(Cl)c(Cl)cc1Nc1ncnc2[nH]c3c(c12)CC(C(=O)O)CC3 ; COc1cc(Cl)c(Cl)cc1Nc1ncnc2[nH]c3c(c12)CC(C(=O)NC1CC1)CC3]

25 mg (61 μmol) 4-[(4,5-dichloro-2-methoxyphenyl)amino]-6,7,8,9-tetrahydro-5H-pyrimido[4,5-b]indole-6-carboxylic acid which was prepared according to example 18 were transformed in analogy to example 14 using cyclopropanamine to give after working up and purification 13.6 mg (47%) of the title compound.

¹H-NMR (DMSO-d6): δ=0.39 (2H), 0.61 (2H), 1.76 (1H), 1.99 (1H), 2.52 (1H), 2.60-2.74 (3H), 2.85-3.03 (2H), 3.90 (3H), 7.30 (1H), 7.70 (1H), 7.99 (1H), 8.25 (1H), 8.85 (1H), 11.60 (1H) ppm.

### Example 21

### (RS)-4-[(4,5-Dichloro-2-methoxyphenyl)amino]-N-ethyl-6,7,8,9-tetrahydro-5H-pyrimido[4,5-b]indole-6-carboxamide

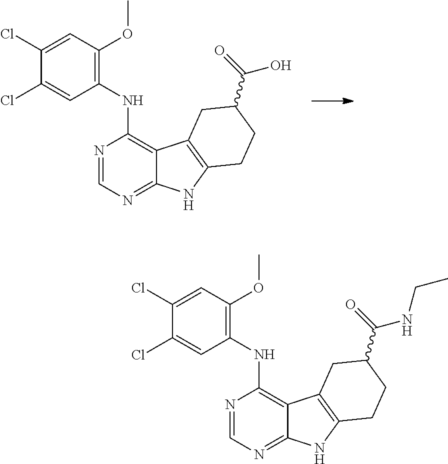 [SMILES: COc1cc(Cl)c(Cl)cc1Nc1ncnc2[nH]c3c(c12)CC(C(=O)O)CC3 ; CCNC(=O)C1CCc2[nH]c3ncnc(Nc4cc(Cl)c(Cl)cc4OC)c3c2C1]

25 mg (61 μmol) 4-[(4,5-dichloro-2-methoxyphenyl)amino]-6,7,8,9-tetrahydro-5H-pyrimido[4,5-b]indole-6-carboxylic acid which was prepared according to example 18 were transformed in analogy to example 14 using ethanamine to give after working up and purification 11.0 mg (39%) of the title compound.

¹H-NMR (DMSO-d6): δ=1.03 (3H), 1.78 (1H), 2.01 (1H), 2.56 (1H), 2.69 (2H), 2.85-3.18 (4H), 3.89 (3H), 7.29 (1H), 7.70 (1H), 7.90 (1H), 8.25 (1H), 8.86 (1H), 11.59 (1H) ppm.

### Example 22

### (RS)-4-[(4,5-Dichloro-2-methoxyphenyl)amino]-N-phenyl-6,7,8,9-tetrahydro-5H-pyrimido[4,5-b]indole-6-carboxamide

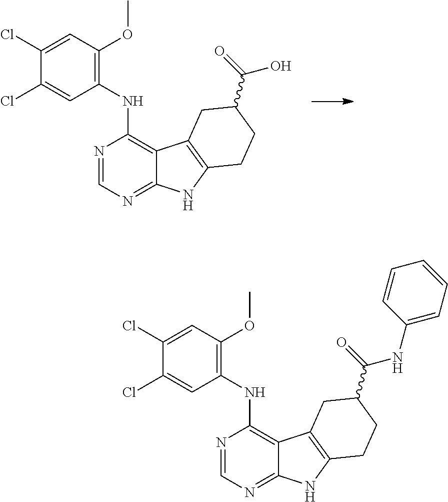 [SMILES: COc1cc(Cl)c(Cl)cc1Nc1ncnc2[nH]c3c(c12)CC(C(=O)O)CC3 ; COc1cc(Cl)c(Cl)cc1Nc1ncnc2[nH]c3c(c12)CC(C(=O)Nc1ccccc1)CC3]

25 mg (61 μmol) 4-[(4,5-dichloro-2-methoxyphenyl)amino]-6,7,8,9-tetrahydro-5H-pyrimido[4,5-b]indole-6-carboxylic acid which was prepared according to example 18 were transformed in analogy to example 14 using aniline to give after working up and purification 8.9 mg (29%) of the title compound.

¹H-NMR (DMSO-d6): δ=1.87 (1H), 2.16 (1H), 2.70-2.91 (3H), 3.04 (1H), 3.18 (1H), 3.83 (3H), 7.01 (1H), 7.24-7.33 (3H), 7.62 (2H), 7.74 (1H), 8.26 (1H), 8.82 (1H), 10.01 (1H), 11.62 (1H) ppm.

### Example 23

### N-(4,5-Dichloro-2-methoxyphenyl)-6,7,8,9-tetrahydro-5H-pyrimido[4,5-b]indol-4-amine

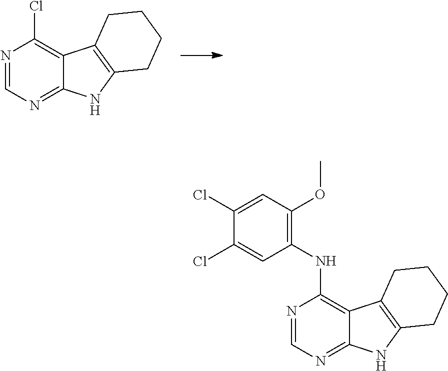 [SMILES: Clc1ncnc2[nH]c3c(c12)CCCC3 ; COc1cc(Cl)c(Cl)cc1Nc1ncnc2[nH]c3c(c12)CCCC3]

15 mg (72 μmol) 4-chloro-6,7,8,9-tetrahydro-5H-pyrimido[4,5-b]indole which was prepared according to intermediate example 1a were transformed in analogy to example 1 using 4,5-dichloro-2-methoxyaniline to give after working up and purification 11.8 mg (43%) of the title compound.

¹H-NMR (DMSO-d6): δ=1.74-1.87 (4H), 2.63 (2H), 2.84 (2H), 3.93 (3H), 7.29 (1H), 7.66 (1H), 8.25 (1H), 8.95 (1H), 11.54 (1H) ppm.

### Example 24

### 6-Benzyl-N-(1H-indazol-5-yl)-6,7,8,9-tetrahydro-5H-pyrido[3′,4′:4,5]pyrrolo[2,3-d]pyrimidin-4-amine

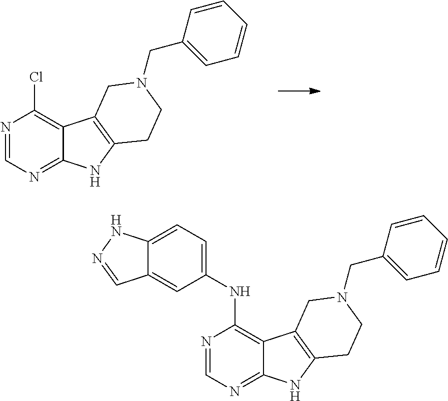 [SMILES: Clc1ncnc2[nH]c3c(c12)CN(Cc1ccccc1)CC3 ; c1ccc(CN2CCc3[nH]c4ncnc(Nc5ccc6[nH]ncc6c5)c4c3C2)cc1]

23 mg (77 μmol) 6-benzyl-4-chloro-6,7,8,9-tetrahydro-5H-pyrido[3′,4′:4,5]pyrrolo[2,3-d]pyrimidine which was prepared according to intermediate example 8a were transformed in analogy to example 1 using 1H-indazol-5-amine to give after working up and purification 5.8 mg (18%) of the title compound.

¹H-NMR (DMSO-d6): δ=2.67 (4H), 3.67 (2H), 3.82 (2H), 7.22 (1H), 7.27-7.34 (4H), 7.44 (2H), 7.91 (1H), 7.94 (1H), 7.98 (1H), 8.06 (1H), 11.44 (1H), 12.91 (1H) ppm.

### Example 25

### (RS)-4-[(4-Fluoro-2-isopropoxyphenyl)amino]-9-methyl-N-[3-(methylsulfonyl)propyl]-6,7,8,9-tetrahydro-5H-pyrimido[4,5-b]indole-6-carboxamide

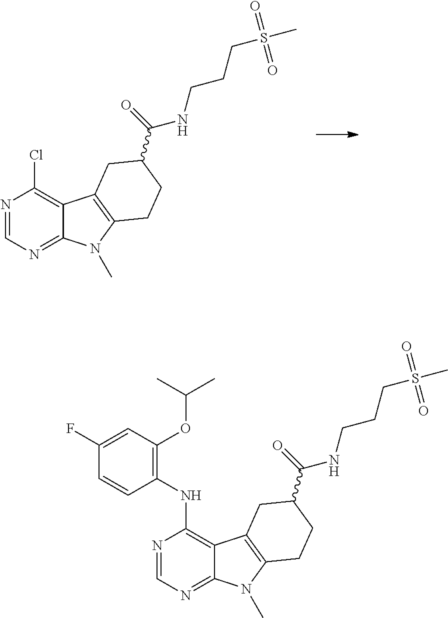 [SMILES: Cn1c2c(c3c(Cl)ncnc31)CC(C(=O)NCCCS(C)(=O)=O)CC2 ; CC(C)Oc1cc(F)ccc1Nc1ncnc2c1c1c(n2C)CCC(C(=O)NCCCS(C)(=O)=O)C1]

23 mg (60 μmol) (RS)-4-chloro-9-methyl-N-[3-(methylsulfonyl)propyl]-6,7,8,9-tetrahydro-5H-pyrimido[4,5-b]indole-6-carboxamide which was prepared according to intermediate example 25a were transformed in analogy to example 1 to give after working up and purification 14.6 mg (45%) of the title compound.

¹H-NMR (DMSO-d6): δ=1.21-1.36 (6H), 1.83 (2H), 2.08 (1H), 2.60 (1H), 2.69 (1H), 2.83 (1H), 2.94 (3H), 2.98-3.23 (7H), 3.58 (3H), 4.74 (1H), 6.75 (1H), 7.01 (1H), 7.68 (1H), 8.09 (1H), 8.25 (1H), 8.73 (1H) ppm.

### Example 25a

### (RS)-4-Chloro-9-methyl-N-[3-(methylsulfonyl)propyl]-6,7,8,9-tetrahydro-5H-pyrimido[4,5-b]indole-6-carboxamide

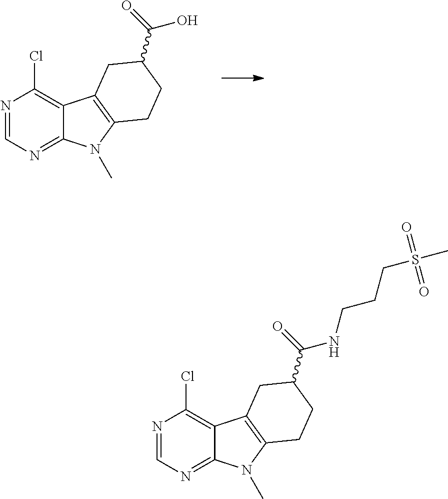 [SMILES: Cn1c2c(c3c(Cl)ncnc31)CC(C(=O)O)CC2 ; Cn1c2c(c3c(Cl)ncnc31)CC(C(=O)NCCCS(C)(=O)=O)CC2]

135 mg (508 μmol) (RS)-4-chloro-9-methyl-6,7,8,9-tetrahydro-5H-pyrimido[4,5-b]indole-6-carboxylic acid which was prepared according to intermediate example 25b were transformed in analogy to example 14 to give after working up and purification 49.4 mg (25%) of the title compound.

### Example 25b

### (RS)-4-Chloro-9-methyl-6,7,8,9-tetrahydro-5H-pyrimido[4,5-b]indole-6-carboxylic acid

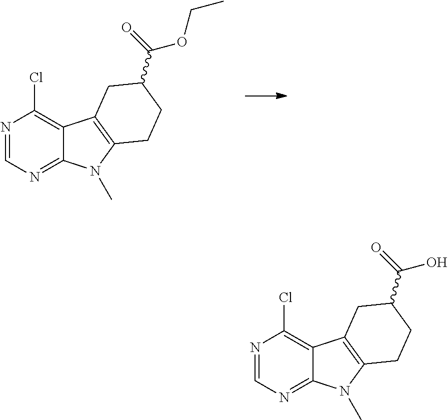 [SMILES: CCOC(=O)C1CCc2c(c3c(Cl)ncnc3n2C)C1 ; Cn1c2c(c3c(Cl)ncnc31)CC(C(=O)O)CC2]

180 mg (613 μmol) (RS)-ethyl 4-chloro-9-methyl-6,7,8,9-tetrahydro-5H-pyrimido[4,5-b]indole-6-carboxylate which was prepared according to intermediate example 25c were transformed in analogy to example 6 to give after working up and purification 138.9 mg (81%) of the title compound.

### Example 25c

### (RS)-Ethyl 4-chloro-9-methyl-6,7,8,9-tetrahydro-5H-pyrimido[4,5-b]indole-6-carboxylate

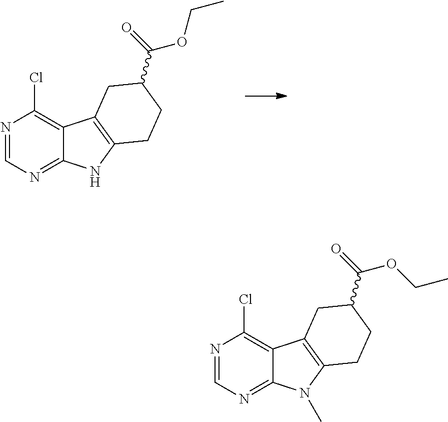 [SMILES: CCOC(=O)C1CCc2[nH]c3ncnc(Cl)c3c2C1 ; CCOC(=O)C1CCc2c(c3c(Cl)ncnc3n2C)C1]

A mixture comprising 250 mg (894 μmol) (RS)-ethyl 4-chloro-6,7,8,9-tetrahydro-5H-pyrimido[4,5-b]indole-6-carboxylate which was prepared according to intermediate example 5a, 111 μL methyl iodide, 437 mg cesium carbonate and 2.13 mL N,N-dimethylformamide was stirred at 23° C. overnight. The mixture was poured into water and extracted with dichloromethane. The organic layer was washed with water, brine and dried over sodium sulfate. After filtration and removal of the solvent the residue was purified by chromatography to give 184.1 mg (67%) of the title compound.

### Example 26

### (RS)-4-(1H-Indazol-5-ylamino)-9-methyl-N-[3-(methylsulfonyl)propyl]-6,7,8,9-tetrahydro-5H-pyrimido[4,5-b]indole-6-carboxamide

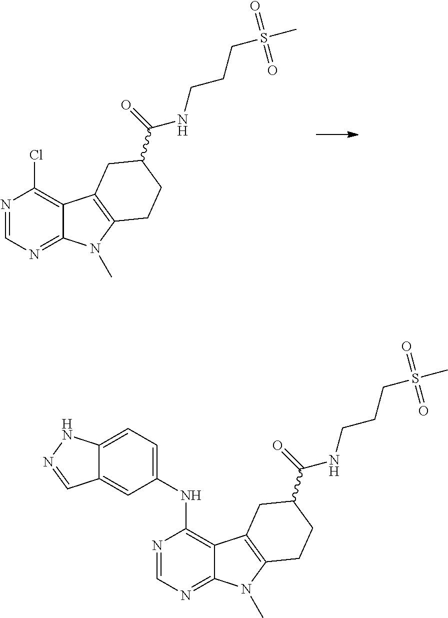 [SMILES: Cn1c2c(c3c(Cl)ncnc31)CC(C(=O)NCCCS(C)(=O)=O)CC2 ; Cn1c2c(c3c(Nc4ccc5[nH]ncc5c4)ncnc31)CC(C(=O)NCCCS(C)(=O)=O)CC2]

23 mg (60 μmol) (RS)-4-chloro-9-methyl-N-[3-(methylsulfonyl)propyl]-6,7,8,9-tetrahydro-5H-pyrimido[4,5-b]indole-6-carboxamide which was prepared according to intermediate example 25a were transformed in analogy to example 1 using 1H-indazol-5-amine to give after working up and purification 20.7 mg (68%) of the title compound.

¹H-NMR (DMSO-d6): δ=1.72-1.89 (2H), 2.07 (1H), 2.67 (1H), 2.83 (1H), 2.94 (3H), 2.99 (1H), 3.05-3.23 (7H), 3.57 (3H), 7.43 (1H), 7.51 (1H), 7.94-8.06 (4H), 8.10 (1H), 12.91 (1H) ppm.

### Example 27

### N-(1H-Indazol-5-yl)-5′,7′,8′,9′-tetrahydrospiro[1,3-dioxolane-2,6′-pyrimido[4,5-b]indol]-4′-amine

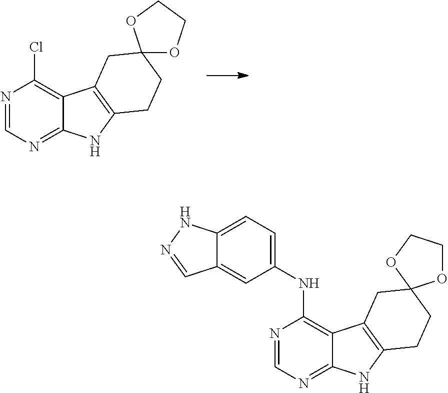 [SMILES: Clc1ncnc2[nH]c3c(c12)CC1(CC3)OCCO1 ; c1nc(Nc2ccc3[nH]ncc3c2)c2c3c([nH]c2n1)CCC1(C3)OCCO1]

40 mg (151 μmol) 4′-Chloro-5′,7′,8′,9′-tetrahydrospiro[1,3-dioxolane-2,6′-pyrimido[4,5-b]indole] which was prepared according to intermediate example 27a were transformed in analogy to example 1 using 1H-indazol-5-amine to give after working up and purification 4.7 mg (8%) of the title compound.

¹H-NMR (DMSO-d6): δ=1.89 (2H), 2.75 (2H), 3.09 (2H), 3.89-3.97 (4H), 7.44 (1H), 7.51 (1H), 7.92 (1H), 7.96-8.02 (2H), 8.05 (1H), 11.39 (1H), 12.92 (1H) ppm.

### Example 27a

### 4′-Chloro-5′,7′,8′,9′-tetrahydrospiro[1,3-dioxolane-2,6′-pyrimido[4,5-b]indole]

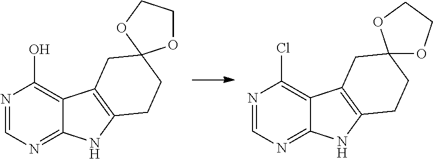 [SMILES: Oc1ncnc2[nH]c3c(c12)CC1(CC3)OCCO1 ; Clc1ncnc2[nH]c3c(c12)CC1(CC3)OCCO1]

387 mg (1.57 mmol) 5′,7′,8′,9′-Tetrahydrospiro[1,3-dioxolane-2,6′-pyrimido[4,5-b]indol]-4′-ol which was prepared according to intermediate example 27b were transformed in analogy to intermediate example 1a to give after working up and purification 241.6 mg (70%) of the title compound.

### Example 27b

### 5′,7′,8′,9′-Tetrahydrospiro[1,3-dioxolane-2,6′-pyrimido[4,5-b]indol]-4′-ol

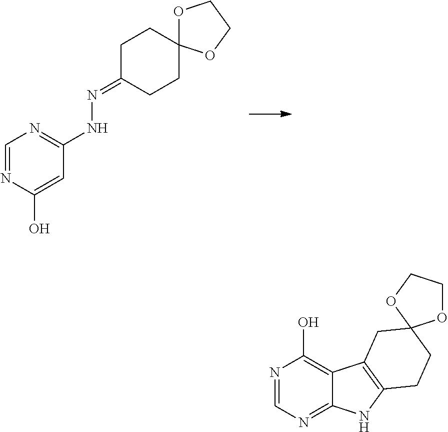 [SMILES: Oc1cc(NN=C2CCC3(CC2)OCCO3)ncn1 ; Oc1ncnc2[nH]c3c(c12)CC1(CC3)OCCO1]

511 mg (1.93 mmol) 6-[2-(1,4-Dioxaspiro[4.5]dec-8-ylidene)hydrazino]pyrimidin-4-ol which was prepared according to intermediate example 27c were transformed in analogy to intermediate example 1b to give after working up and purification 469 mg (93%) of the title compound.

### Example 27c

### 6-[2-(1,4-Dioxaspiro[4.5]dec-8-ylidene)hydrazino]pyrimidin-4-ol

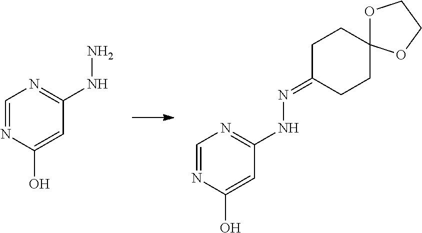 [SMILES: NNc1cc(O)ncn1 ; Oc1cc(NN=C2CCC3(CC2)OCCO3)ncn1]

350 mg (2.78 mmol) 6-hydrazinopyrimidin-4-ol/6-hydrazinopyrimidin-4(1H)-one (CAS-No: 29939-37-5) were transformed in analogy to intermediate example 1c using 1,4-dioxaspiro[4.5]decan-8-one to give after working up and purification 639 mg (87%) of the title compound.

### Example 28

### (RS)-4-{[4-Fluoro-2-(propan-2-yloxy)phenyl]amino}-N-[3-(methylsulfonyl)propyl]-6,7,8,9-tetrahydro-5H-pyrimido[4,5-b]indole-6-carboxamide

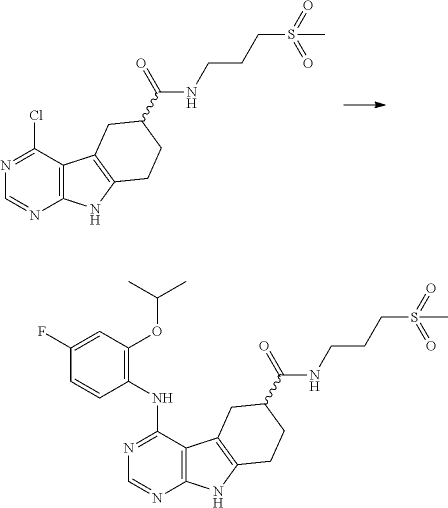 [SMILES: CS(=O)(=O)CCCNC(=O)C1CCc2[nH]c3ncnc(Cl)c3c2C1 ; CC(C)Oc1cc(F)ccc1Nc1ncnc2[nH]c3c(c12)CC(C(=O)NCCCS(C)(=O)=O)CC3]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 to give after working up and purification 14.3 mg (33%) of the title compound.

¹H-NMR (DMSO-d6): δ=1.28 (6H), 1.83 (3H), 2.01 (1H), 2.60 (1H), 2.70 (1H), 2.94 (3H), 2.97-3.25 (7H), 4.74 (1H), 6.74 (1H), 7.01 (1H), 7.65 (1H), 8.07 (1H), 8.20 (1H), 8.74 (1H), 11.52 (1H) ppm.

### Example 28a

### (RS)-4-Chloro-N-[3-(methylsulfonyl)propyl]-6,7,8,9-tetrahydro-5H-pyrimido[4,5-b]indole-6-carboxamide

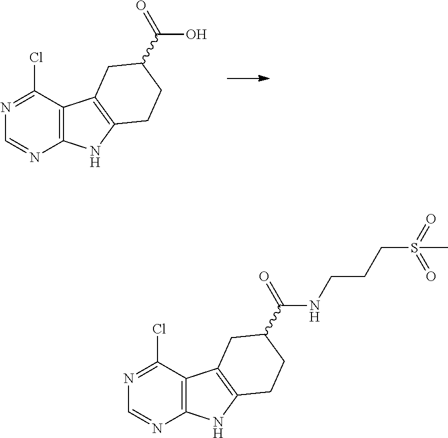 [SMILES: O=C(O)C1CCc2[nH]c3ncnc(Cl)c3c2C1 ; CS(=O)(=O)CCCNC(=O)C1CCc2[nH]c3ncnc(Cl)c3c2C1]

3.09 g (12.29 mmol) (RS)-4-chloro-6,7,8,9-tetrahydro-5H-pyrimido[4,5-b]indole-6-carboxylic acid which was prepared according to intermediate example 28b were transformed in analogy to example 14 to give after working up and purification 3.35 mg (70%) of the title compound.

### Example 28b

### (RS)-4-Chloro-6,7,8,9-tetrahydro-5H-pyrimido[4,5-b]indole-6-carboxylic acid

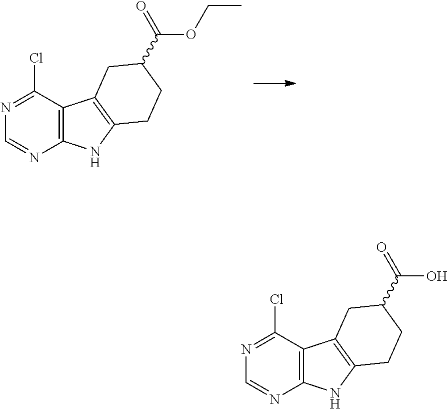 [SMILES: CCOC(=O)C1CCc2[nH]c3ncnc(Cl)c3c2C1 ; O=C(O)C1CCc2[nH]c3ncnc(Cl)c3c2C1]

4.56 g (16.3 mmol) (RS)-ethyl 4-chloro-6,7,8,9-tetrahydro-5H-pyrimido[4,5-b]indole-6-carboxylate which was prepared according to intermediate example 5a were transformed in analogy to example 6 to give after working up and purification 3.57 g (83%) of the title compound.

### Example 29

### (RS)—N-[3-(Methylsulfonyl)propyl]-4-[(2-oxo-2,3-dihydro-1,3-benzothiazol-6-yl)amino]-6,7,8,9-tetrahydro-5H-pyrimido[4,5-b]indole-6-carboxamide

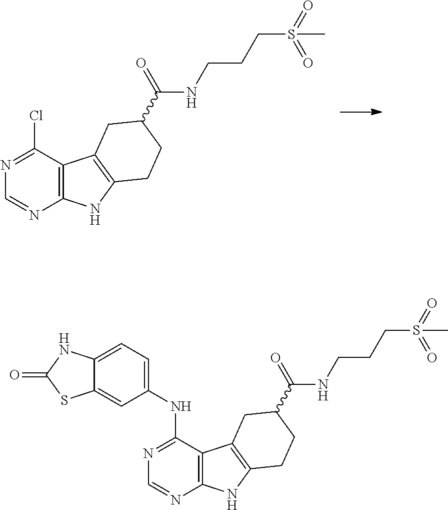 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc5[nH]c(=O)sc5c4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 6-amino-1,3-benzothiazol-2(3H)-one to give after working up and purification 10.7 mg (25%) of the title compound.

¹H-NMR (DMSO-d6): δ=1.68-1.91 (3H), 2.01 (1H), 2.70 (2H), 2.84-2.99 (4H), 3.03-3.24 (6H), 7.12 (1H), 7.39 (1H), 7.77 (1H), 8.02-8.14 (2H), 8.93 (1H), 11.94 (1H), 12.01 (1H) ppm.

### Example 30

### (RS)—N-[3-(Methylsulfonyl)propyl]-4-{[2-(morpholin-4-yl)phenyl]amino}-6,7,8,9-tetrahydro-5H-pyrimido[4,5-b]indole-6-carboxamide

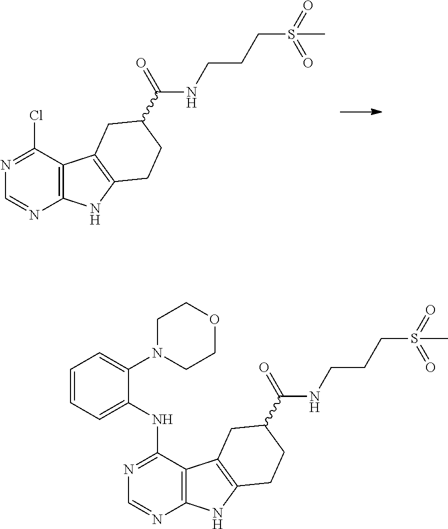 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccccc4N4CCOCC4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2-(morpholin-4-yl)aniline to give after working up and purification 9.0 mg (21%) of the title compound.

¹H-NMR (DMSO-d6): δ=1.77-1.92 (3H), 2.03 (1H), 2.57-2.86 (7H), 2.93 (3H), 3.06-3.24 (6H), 3.73 (4H), 6.95 (1H), 7.15 (1H), 7.34 (1H), 8.10 (1H), 8.23 (1H), 8.66 (1H), 8.86 (1H), 11.52 (1H) ppm.

### Example 31

### (RS)-4-{[4-(Difluoromethoxy)phenyl]amino}-N-[3-(methylsulfonyl)propyl]-6,7,8,9-tetrahydro-5H-pyrimido[4,5-b]indole-6-carboxamide

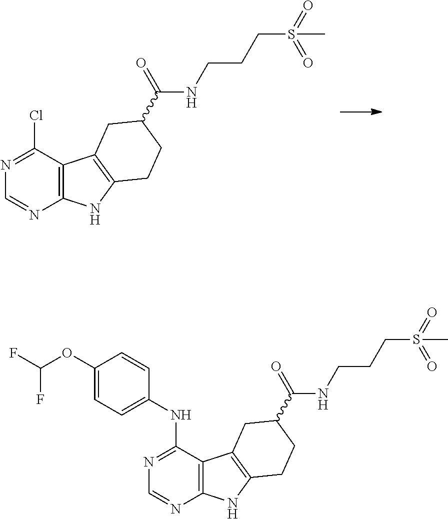 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc(OC(F)F)cc4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 4-(difluoromethoxy)aniline to give after working up and purification 10.1 mg (24%) of the title compound.

¹H-NMR (DMSO-d6): δ=1.75-1.89 (3H), 2.02 (1H), 2.53 (1H), 2.71 (1H), 2.92-3.02 (4H), 3.08-3.27 (6H), 7.11 (2H), 7.13 (1H), 7.69 (2H), 8.03 (1H), 8.05 (1H), 8.11 (1H), 11.46 (1H) ppm.

### Example 32

### (RS)—N-[3-(Methylsulfonyl)propyl]-4-{[2-(trifluoromethoxy)phenyl]amino}-6,7,8,9-tetrahydro-5H-pyrimido[4,5-b]indole-6-carboxamide

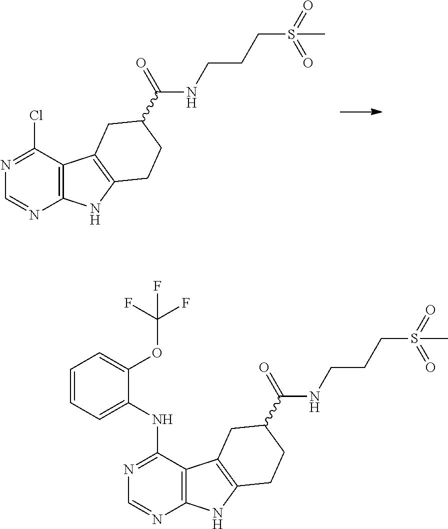 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccccc4OC(F)(F)F)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2-(trifluoromethoxy)aniline to give after working up and purification 5.5 mg (13%) of the title compound.

¹H-NMR (DMSO-d6): δ=1.77-1.90 (3H), 2.03 (1H), 2.59 (1H), 2.72 (1H), 2.95 (3H), 2.92-3.26 (7H), 7.17 (1H), 7.35-7.41 (2H), 7.76 (1H), 8.04 (1H), 8.15 (1H), 8.31 (1H), 11.55 (1H) ppm.

### Example 33

### (RS)-4-[(4-Methyl-2-oxo-2H-chromen-7-yl)amino]-N-[3-(methylsulfonyl)propyl]-6,7,8,9-tetrahydro-5H-pyrimido[4,5-b]indole-6-carboxamide

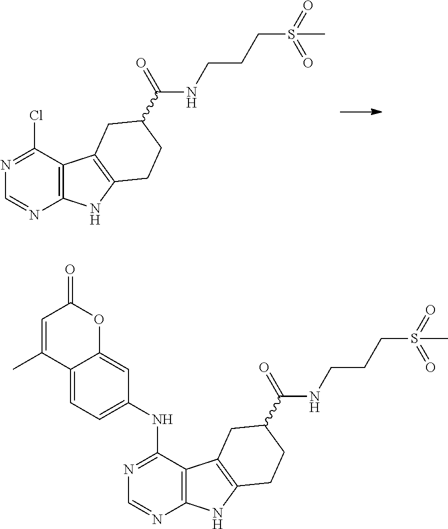

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 7-amino-4-methyl-2H-chromen-2-one to give after working up and purification 10.4 mg (24%) of the title compound.

¹H-NMR (DMSO-d6): δ=1.77-1.89 (3H), 2.04 (1H), 2.40 (3H), 2.55 (1H), 2.74 (1H), 2.95-3.25 (7H), 2.97 (3H), 6.19 (1H), 7.63 (1H), 7.67 (1H), 7.97 (1H), 8.05 (1H), 8.27 (1H), 8.52 (1H), 11.64 (1H) ppm.

### Example 34

### (RS)-4-[(4-Fluoro-3-methoxyphenyl)amino]-N-[3-(methylsulfonyl)propyl]-6,7,8,9-tetrahydro-5H-pyrimido[4,5-b]indole-6-carboxamide

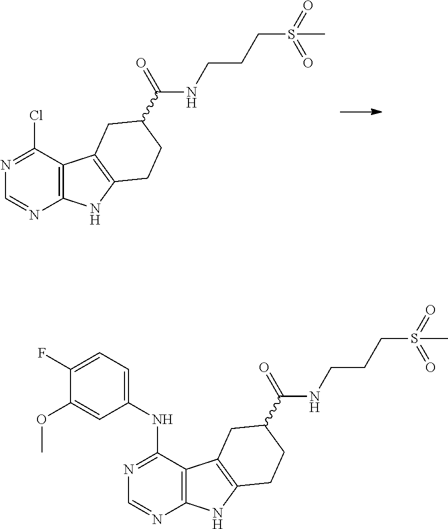 [SMILES: CS(=O)(=O)CCCNC(=O)C1CCc2[nH]c3ncnc(Cl)c3c2C1 ; COc1cc(Nc2ncnc3[nH]c4c(c23)CC(C(=O)NCCCS(C)(=O)=O)CC4)ccc1F]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 4-fluoro-3-methoxyaniline to give after working up and purification 21.3 mg (53%) of the title compound.

¹H-NMR (DMSO-d6): δ=1.73-1.87 (3H), 2.00 (1H), 2.51 (1H), 2.66 (1H), 2.89-3.00 (1H), 2.94 (3H), 3.06-3.24 (6H), 3.79 (3H), 7.08 (1H), 7.25 (1H), 7.46 (1H), 7.92 (1H), 8.00 (1H), 8.09 (1H), 11.43 (1H) ppm.

### Example 35

### (RS)—N-[3-(Methylsulfonyl)propyl]-4-{[4-(propan-2-yloxy)phenyl]amino}-6,7,8,9-tetrahydro-5H-pyrimido[4,5-b]indole-6-carboxamide

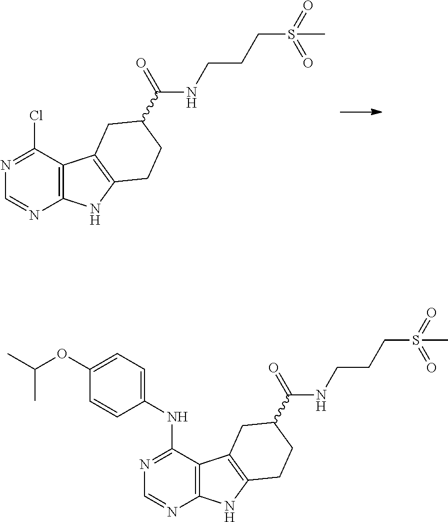

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 4-isopropoxyaniline to give after working up and purification 20.3 mg (49%) of the title compound.

¹H-NMR (DMSO-d6): δ=1.22 (6H), 1.69-1.88 (3H), 1.99 (1H), 2.51 (1H), 2.67 (2H), 2.91 (1H), 2.94 (3H), 3.04-3.25 (5H), 4.51 (1H), 6.82 (2H), 7.46 (2H), 7.79 (1H), 8.00 (1H), 8.02 (1H), 11.36 (1H) ppm.

### Example 36

### (RS)-4-[(2,2-Difluoro-1,3-benzodioxol-5-yl)amino]-N-[3-(methylsulfonyl)propyl]-6,7,8,9-tetrahydro-5H-pyrimido[4,5-b]indole-6-carboxamide

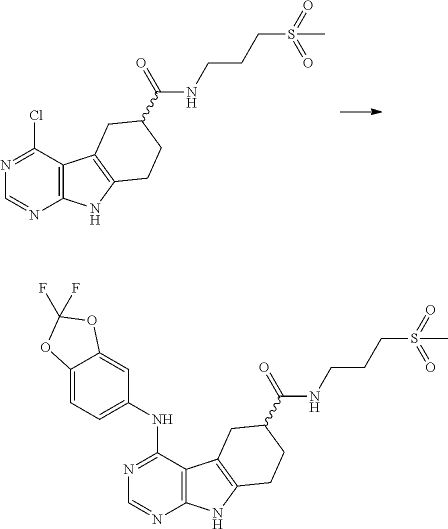 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc5c(c4)OC(F)(F)O5)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2,2-difluoro-1,3-benzodioxol-5-amine to give after working up and purification 16.2 mg (37%) of the title compound.

¹H-NMR (DMSO-d6): δ=1.72-1.88 (3H), 2.00 (1H), 2.52 (1H), 2.63-2.75 (2H), 2.87-3.03 (1H), 2.94 (3H), 3.05-3.23 (5H), 7.28 (1H), 7.36 (1H), 7.79 (1H), 8.00 (1H), 8.11 (1H), 8.13 (1H), 11.47 (1H) ppm.

### Example 37

### (RS)-4-[(4-Fluoro-2-methoxyphenyl)amino]-N-[3-(methylsulfonyl)propyl]-6,7,8,9-tetrahydro-5H-pyrimido[4,5-b]indole-6-carboxamide

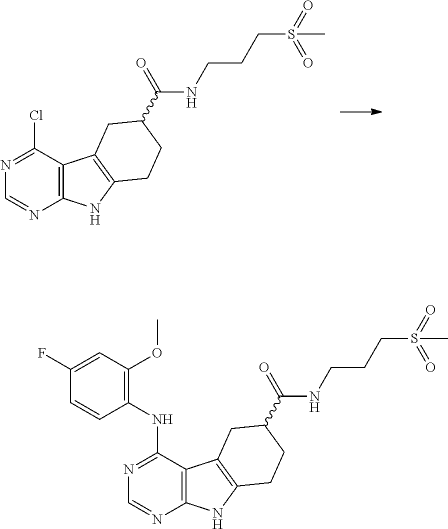 [SMILES: CS(=O)(=O)CCCNC(=O)C1CCc2[nH]c3ncnc(Cl)c3c2C1 ; COc1cc(F)ccc1Nc1ncnc2[nH]c3c(c12)CC(C(=O)NCCCS(C)(=O)=O)CC3]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 4-fluoro-2-methoxyaniline to give after working up and purification 6.9 mg (17%) of the title compound.

¹H-NMR (DMSO-d6): δ=1.70-1.91 (3H), 2.02 (1H), 2.58 (1H), 2.69 (2H), 2.88-3.26 (6H), 2.94 (3H), 3.84 (3H), 6.76 (1H), 6.98 (1H), 7.55 (1H), 8.05 (1H), 8.12 (1H), 8.35 (1H), 11.48 (1H) ppm.

### Example 38

### (RS)—N-[3-(Methylsulfonyl)propyl]-4-{[4-(pyrrolidin-1-ylcarbonyl)phenyl]amino}-6,7,8,9-tetrahydro-5H-pyrimido[4,5-b]indole-6-carboxamide

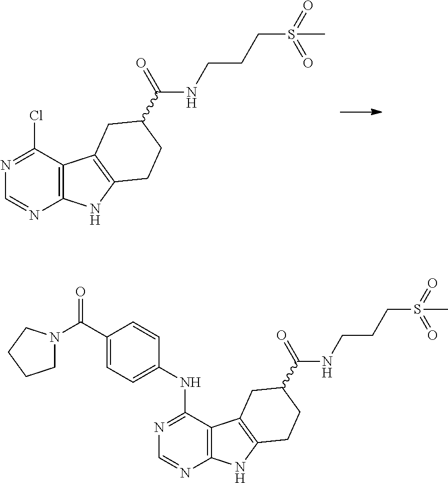 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc(C(=O)N5CCCC5)cc4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using (4-aminophenyl)(pyrrolidin-1-yl)methanone to give after working up and purification 19.1 mg (43%) of the title compound.

¹H-NMR (DMSO-d6): δ=1.70-1.90 (7H), 2.00 (1H), 2.52 (1H), 2.69 (2H), 2.90-3.24 (6H), 2.94 (3H), 3.43 (4H), 7.45 (2H), 7.72 (2H), 8.00 (1H), 8.16 (1H), 8.17 (1H), 11.51 (1H) ppm.

### Example 39

### (RS)-4-[(5-Fluoro-2-methoxyphenyl)amino]-N-[3-(methylsulfonyl)propyl]-6,7,8,9-tetrahydro-5H-pyrimido[4,5-b]indole-6-carboxamide

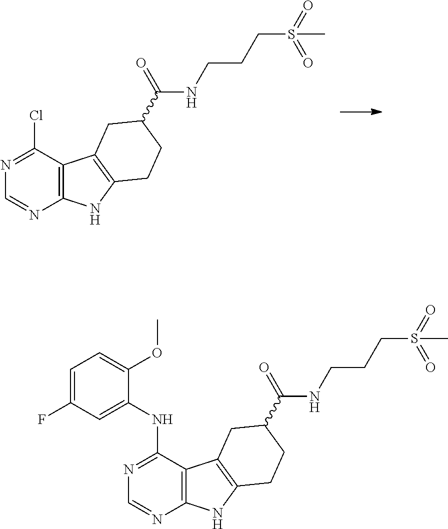 [SMILES: CS(=O)(=O)CCCNC(=O)C1CCc2[nH]c3ncnc(Cl)c3c2C1 ; COc1ccc(F)cc1Nc1ncnc2[nH]c3c(c12)CC(C(=O)NCCCS(C)(=O)=O)CC3]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 5-fluoro-2-methoxyaniline to give after working up and purification 8.1 mg (20%) of the title compound.

¹H-NMR (DMSO-d6): δ=1.72-1.91 (3H), 2.03 (1H), 2.54-2.77 (3H), 2.91-3.25 (6H), 2.94 (3H), 3.85 (3H), 6.74 (1H), 7.03 (1H), 7.78 (1H), 8.09 (1H), 8.25 (1H), 8.55 (1H), 11.61 (1H) ppm.

### Example 40

### (RS)-4-[(2,2-Dioxido-1,3-dihydro-2-benzothiophen-5-yl)amino]-N-[3-(methylsulfonyl)propyl]-6,7,8,9-tetrahydro-5H-pyrimido[4,5-b]indole-6-carboxamide

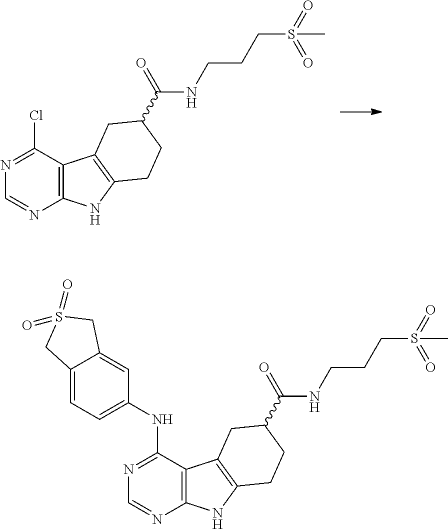 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc5c(c4)CS(=O)(=O)C5)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 1,3-dihydro-2-benzothiophen-5-amine 2,2-dioxide to give after working up and purification 12.7 mg (29%) of the title compound.

¹H-NMR (DMSO-d6): δ=1.76-1.89 (3H), 2.02 (1H), 2.53 (1H), 2.67-2.81 (1H), 2.77 (1H), 2.94-3.04 (1H), 2.97 (3H), 3.08-3.25 (5H), 4.41 (2H), 4.47 (2H), 7.28 (1H), 7.64 (1H), 7.77 (1H), 8.02 (1H), 8.10 (1H), 8.15 (1H), 11.50 (1H) ppm.

### Example 41

### (RS)-4-{[4-(Methylsulfamoyl)phenyl]amino}-N-[3-(methylsulfonyl)propyl]-6,7,8,9-tetrahydro-5H-pyrimido[4,5-b]indole-6-carboxamide

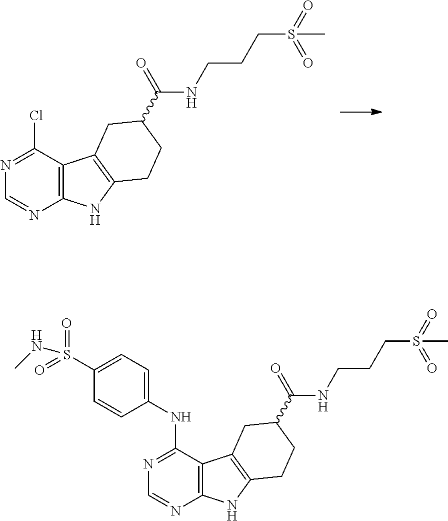

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 4-amino-N-methylbenzenesulfonamide to give after working up and purification 17.5 mg (39%) of the title compound.

¹H-NMR (DMSO-d6): δ=1.73-1.87 (3H), 2.01 (1H), 2.37 (3H), 2.51 (1H), 2.65-2.77 (2H), 2.94 (3H), 2.95-3.03 (1H), 3.07-3.24 (5H), 7.20 (1H), 7.64 (2H), 7.86 (2H), 8.00 (1H), 8.20 (1H), 8.43 (1H), 11.57 (1H) ppm.

### Example 42

### (RS)-4-[(2,5-Dimethoxyphenyl)amino]-N-[3-(methylsulfonyl)propyl]-6,7,8,9-tetrahydro-5H-pyrimido[4,5-b]indole-6-carboxamide

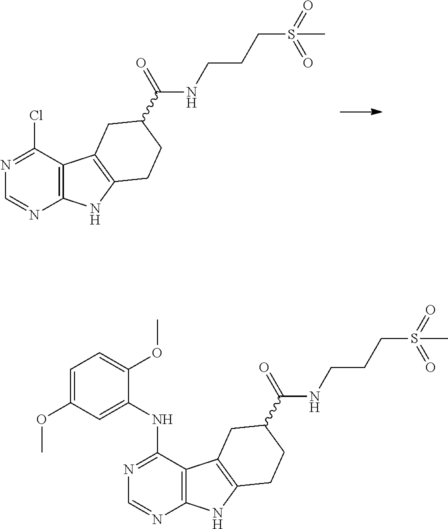 [SMILES: CS(=O)(=O)CCCNC(=O)C1CCc2[nH]c3ncnc(Cl)c3c2C1 ; COc1ccc(OC)c(Nc2ncnc3[nH]c4c(c23)CC(C(=O)NCCCS(C)(=O)=O)CC4)c1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2,5-dimethoxyaniline to give after working up and purification 19.1 mg (46%) of the title compound.

¹H-NMR (DMSO-d6): δ=1.77-1.91 (3H), 2.06 (1H), 2.63 (1H), 2.72 (2H), 2.94-3.17 (4H), 2.96 (3H), 3.23 (2H), 3.72 (3H), 3.83 (3H), 6.52 (1H), 6.96 (1H), 7.74 (1H), 8.08 (1H), 8.24 (1H), 8.36 (1H), 11.54 (1H) ppm.

### Example 43

### (RS)-4-{[4-Fluoro-3-(trifluoromethoxy)phenyl]amino}-N-[3-(methylsulfonyl)propyl]-6,7,8,9-tetrahydro-5H-pyrimido[4,5-b]indole-6-carboxamide

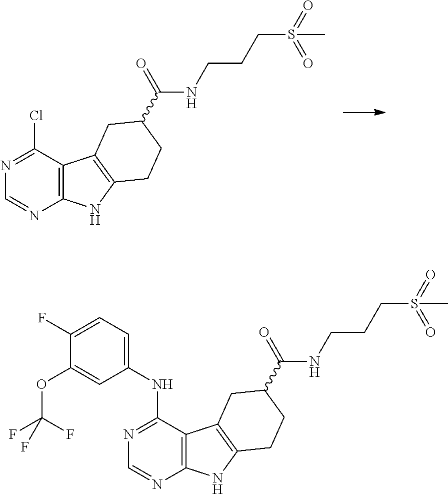 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc(F)c(OC(F)(F)F)c4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 4-fluoro-3-(trifluoromethoxy)aniline to give after working up and purification 15.5 mg (34%) of the title compound.

¹H-NMR (DMSO-d6): δ=1.73-1.87 (3H), 2.00 (1H), 2.51 (1H), 2.69 (2H), 2.91-3.01 (1H), 2.94 (3H), 3.06-3.25 (5H), 7.38 (1H), 7.71 (1H), 7.94 (1H), 8.02 (1H), 8.13 (1H), 8.22 (1H), 11.52 (1H) ppm.

### Example 44

### (RS)-4-[(4,5-Dichloro-2-methoxyphenyl)amino]-N-[3-(methylsulfonyl)propyl]-6,7,8,9-tetrahydro-5H-pyrimido[4,5-b]indole-6-carboxamide

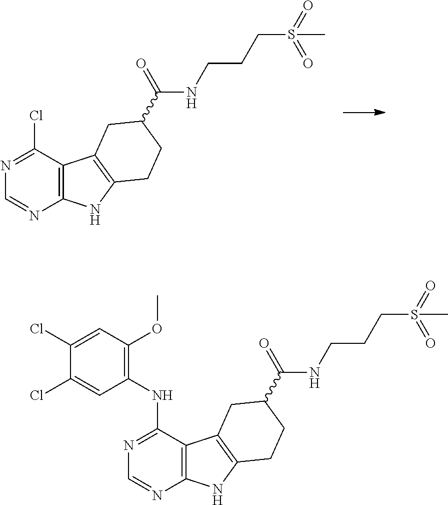 [SMILES: CS(=O)(=O)CCCNC(=O)C1CCc2[nH]c3ncnc(Cl)c3c2C1 ; COc1cc(Cl)c(Cl)cc1Nc1ncnc2[nH]c3c(c12)CC(C(=O)NCCCS(C)(=O)=O)CC3]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 4,5-dichloro-2-methoxyaniline to give after working up and purification 12.2 mg (27%) of the title compound.

¹H-NMR (DMSO-d6): δ=1.74-1.87 (3H), 2.03 (1H), 2.61 (1H), 2.69 (2H), 2.91-3.15 (4H), 2.94 (3H), 3.16-3.24 (2H), 3.89 (3H), 7.30 (1H), 7.70 (1H), 8.06 (1H), 8.26 (1H), 8.86 (1H), 11.62 (1H) ppm.

### Example 45

### (RS)—N-[3-(Methylsulfonyl)propyl]-4-[(1-oxo-1,3-dihydro-2-benzofuran-5-yl)amino]-6,7,8,9-tetrahydro-5H-pyrimido[4,5-b]indole-6-carboxamide

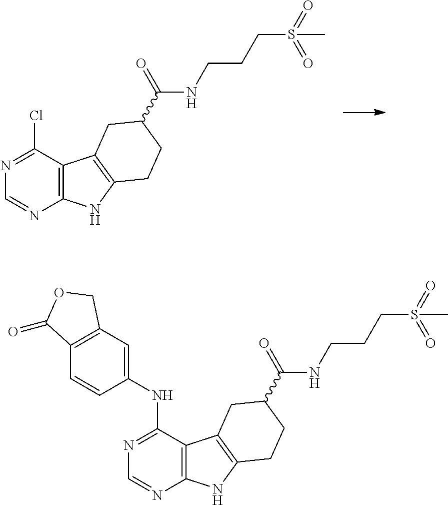 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc5c(c4)COC5=O)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 5-amino-2-benzofuran-1(3H)-one to give after working up and purification 17.4 mg (42%) of the title compound.

¹H-NMR (DMSO-d6): δ=1.77-1.89 (3H), 2.03 (1H), 2.54 (1H), 2.69-2.81 (2H), 2.97 (3H), 2.99-3.06 (1H), 3.10-3.26 (5H), 5.35 (2H), 7.73 (1H), 7.82 (1H), 8.03 (1H), 8.08 (1H), 8.27 (1H), 8.62 (1H), 11.65 (1H) ppm.

### Example 46

### (RS)—N-[3-(Methylsulfonyl)propyl]-4-[(3-oxo-1,3-dihydro-2-benzofuran-5-yl)amino]-6,7,8,9-tetrahydro-5H-pyrimido[4,5-b]indole-6-carboxamide

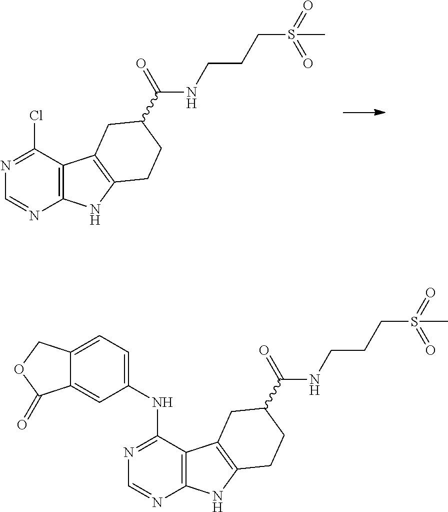 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc5c(c4)C(=O)OC5)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 6-amino-2-benzofuran-1(3H)-one to give after working up and purification 17.9 mg (43%) of the title compound.

¹H-NMR (DMSO-d6): δ=1.72-1.87 (3H), 2.00 (1H), 2.52 (1H), 2.69 (2H), 2.93-3.26 (6H), 2.94 (3H), 5.34 (2H), 7.55 (1H), 7.99 (1H), 8.04 (1H), 8.17 (1H), 8.26 (1H), 8.32 (1H), 11.54 (1H) ppm.

### Example 47

### (RS)—N-[3-(Methylsulfonyl)propyl]-4-{[3-(1H-tetrazol-1-yl)phenyl]amino}-6,7,8,9-tetrahydro-5H-pyrimido[4,5-b]indole-6-carboxamide

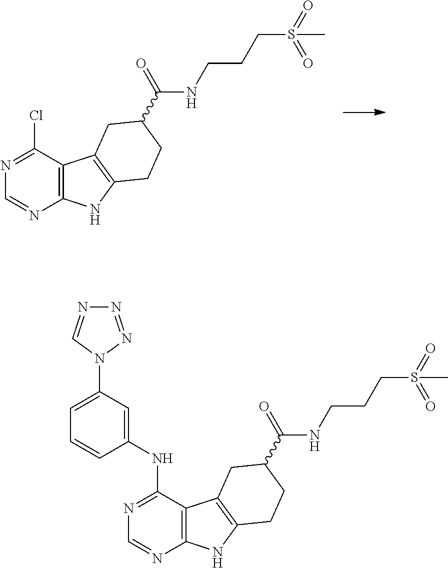 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4cccc(-n5cnnn5)c4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 3-(1H-tetrazol-1-yl)aniline to give after working up and purification 14.1 mg (33%) of the title compound.

¹H-NMR (DMSO-d6): δ=1.73-1.87 (3H), 2.00 (1H), 2.52 (1H), 2.70 (2H), 2.92-3.25 (6H), 2.93 (3H), 7.44 (1H), 7.52 (1H), 7.86 (1H), 8.03 (1H), 8.18 (1H), 8.31 (1H), 8.38 (1H), 10.05 (1H), 11.56 (1H) ppm.

### Example 48

### (RS)—N-[3-(Methylsulfonyl)propyl]-4-[(4-sulfamoylphenyl)amino]-6,7,8,9-tetrahydro-5H-pyrimido[4,5-b]indole-6-carboxamide

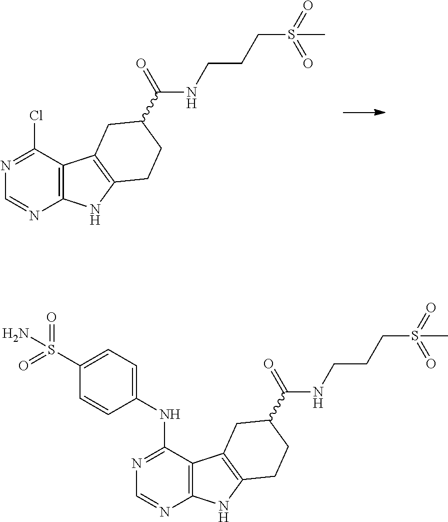 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc(S(N)(=O)=O)cc4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 4-aminobenzenesulfonamide to give after working up and purification 10.1 mg (23%) of the title compound.

¹H-NMR (DMSO-d6): δ=1.71-1.89 (3H), 2.00 (1H), 2.52 (1H), 2.70 (2H), 2.93-3.23 (6H), 2.94 (3H), 7.16 (2H), 7.68 (2H), 7.83 (2H), 8.03 (1H), 8.19 (1H), 8.36 (1H). 11.58 (1H) ppm.

### Example 49

### (RS)-4-(1,3-Benzothiazol-6-ylamino)-N-[3-(methylsulfonyl)propyl]-6,7,8,9-tetrahydro-5H-pyrimido[4,5-b]indole-6-carboxamide

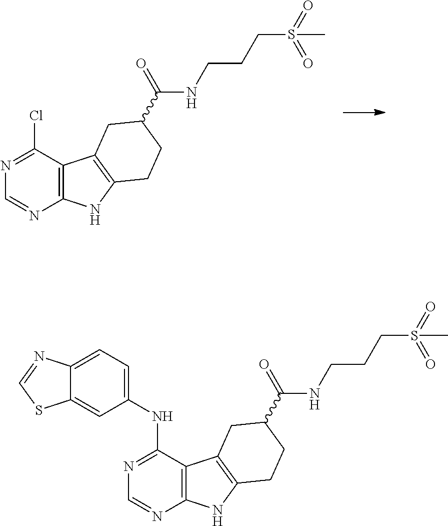 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc5ncsc5c4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 1,3-benzothiazol-6-amine to give after working up and purification 17.9 mg (43%) of the title compound.

¹H-NMR (DMSO-d6): δ=1.73-1.87 (3H), 2.00 (1H), 2.52 (1H), 2.69 (2H), 2.93-3.25 (6H), 2.94 (3H), 7.76 (1H), 7.96 (1H), 8.03 (1H), 8.15 (1H), 8.23 (1H), 8.51 (1H), 9.19 (1H), 11.51 (1H) ppm.

### Example 50

### (RS)-4-[(2-Methyl-1,3-benzothiazol-5-yl)amino]-N-[3-(methylsulfonyl)propyl]-6,7,8,9-tetrahydro-5H-pyrimido[4,5-b]indole-6-carboxamide

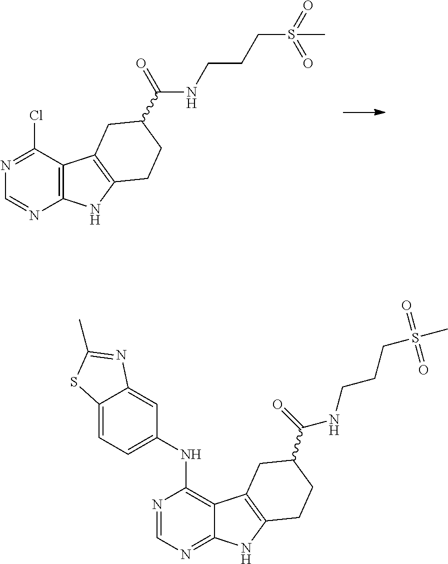 [SMILES: CS(=O)(=O)CCCNC(=O)C1CCc2[nH]c3ncnc(Cl)c3c2C1 ; Cc1nc2cc(Nc3ncnc4[nH]c5c(c34)CC(C(=O)NCCCS(C)(=O)=O)CC5)ccc2s1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2-methyl-1,3-benzothiazol-5-amine to give after working up and purification 16.3 mg (38%) of the title compound.

¹H-NMR (DMSO-d6): δ=1.74-1.89 (3H), 2.01 (1H), 2.52 (1H), 2.70 (2H), 2.75 (3H), 2.94 (3H), 2.96-3.22 (6H), 7.62 (1H), 7.86 (1H), 7.99 (1H), 8.13 (1H), 8.19 (1H), 8.30 (1H), 11.49 (1H) ppm.

### Example 51

### (RS)—N-[3-(Methylsulfonyl)propyl]-4-[(3-oxo-3,4-dihydro-2H-1,4-benzoxazin-7-yl)amino]-6,7,8,9-tetrahydro-5H-pyrimido[4,5-b]indole-6-carboxamide

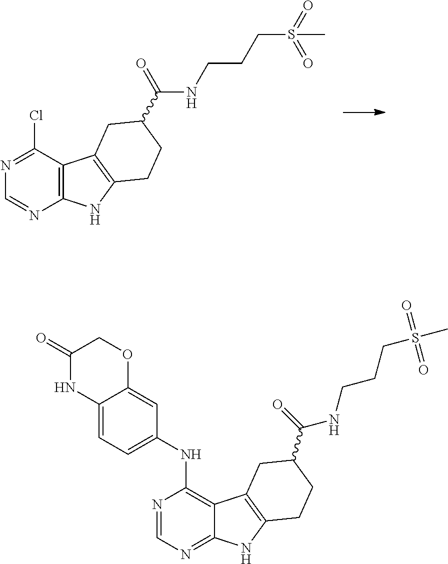 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc5c(c4)OCC(=O)N5)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 7-amino-2H-1,4-benzoxazin-3(4H)-one to give after working up and purification 7.3 mg (17%) of the title compound.

¹H-NMR (DMSO-d6): δ=1.69-1.88 (3H), 1.99 (1H), 2.52 (1H), 2.69 (2H), 2.93 (3H), 2.86-3.23 (6H), 4.51 (2H), 6.79 (1H), 7.15 (1H), 7.36 (1H), 7.90-8.06 (2H), 8.08 (1H), 10.57 (1H), 11.48 (1H) ppm.

### Example 52

### (RS)-4-[(1,1-Dioxido-2,3-dihydro-1-benzothiophen-5-yl)amino]-N-[3-(methylsulfonyl)propyl]-6,7,8,9-tetrahydro-5H-pyrimido[4,5-b]indole-6-carboxamide

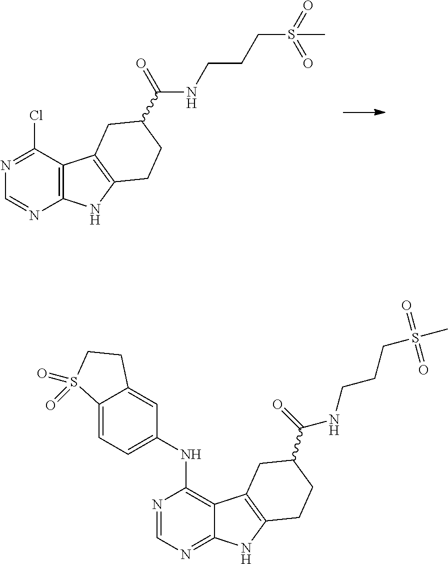 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc5c(c4)CCS5(=O)=O)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2,3-dihydro-1-benzothiophen-5-amine 1,1-dioxide to give after working up and purification 12.4 mg (42%) of the title compound.

¹H-NMR (DMSO-d6): δ=1.73-1.89 (3H), 2.01 (1H), 2.52 (1H), 2.70 (2H), 2.94 (3H), 2.97-3.36 (8H), 3.51 (2H), 7.59 (1H), 7.80 (1H), 7.85 (1H), 8.01 (1H), 8.22 (1H), 8.44 (1H), 11.59 (1H) ppm.

### Example 53

### (RS)-4-[(4-Acetylphenyl)amino]-N-[3-(methylsulfonyl)propyl]-6,7,8,9-tetrahydro-5H-pyrimido[4,5-b]indole-6-carboxamide

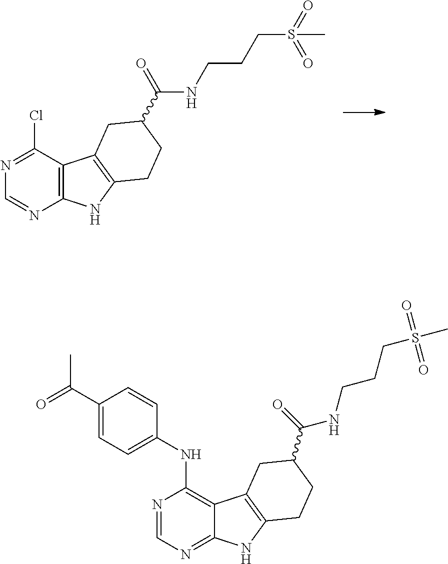 [SMILES: CS(=O)(=O)CCCNC(=O)C1CCc2[nH]c3ncnc(Cl)c3c2C1 ; CC(=O)c1ccc(Nc2ncnc3[nH]c4c(c23)CC(C(=O)NCCCS(C)(=O)=O)CC4)cc1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 1-(4-aminophenyl)ethanone to give after working up and purification 16.5 mg (41%) of the title compound.

¹H-NMR (DMSO-d6): δ=1.74-1.87 (3H), 2.00 (1H), 2.49 (3H), 2.52 (1H), 2.71 (2H), 2.94 (3H), 3.01 (1H), 3.07-3.24 (5H), 7.81 (2H), 7.87 (2H), 8.00 (1H), 8.22 (1H), 8.41 (1H), 11.57 (1H) ppm.

### Example 54

### (RS)-4-[(2-Hydroxy-4-methylphenyl)amino]-N-[3-(methylsulfonyl)propyl]-6,7,8,9-tetrahydro-5H-pyrimido[4,5-b]indole-6-carboxamide

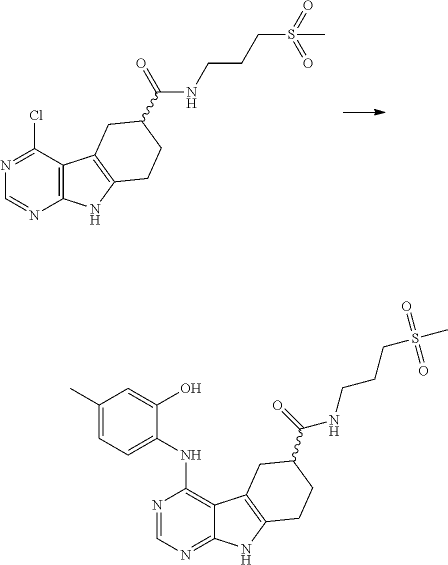

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2-amino-5-methylphenol to give after working up and purification 12.4 mg (30%) of the title compound.

¹H-NMR (DMSO-d6): δ=1.73-1.88 (3H), 2.02 (1H), 2.18 (3H), 2.58 (1H), 2.68 (2H), 2.94 (3H), 2.96-3.26 (6H), 6.59 (1H), 6.65 (1H), 7.59 (1H), 8.02 (1H), 8.14 (1H), 8.20 (1H), 10.07 (1H), 11.44 (1H) ppm.

### Example 55

### (RS)—N-[3-(Methylsulfonyl)propyl]-4-{[3-(1,3-oxazol-5-yl)phenyl]amino}-6,7,8,9-tetrahydro-5H-pyrimido[4,5-b]indole-6-carboxamide

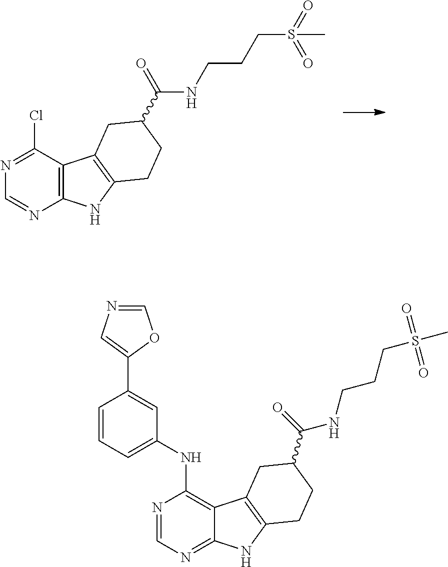 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4cccc(-c5cnco5)c4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 3-(1,3-oxazol-5-yl)aniline to give after working up and purification 8.1 mg (20%) of the title compound.

¹H-NMR (DMSO-d6): δ=1.74-1.88 (3H), 2.01 (1H), 2.52 (1H), 2.70 (2H), 2.94 (3H), 2.98 (1H), 3.08-3.25 (5H), 7.31-7.39 (2H), 7.59 (1H), 7.70 (1H), 7.98-8.05 (2H), 8.12 (1H), 8.14 (1H), 8.41 (1H), 11.47 (1H) ppm.

### Example 56

### (RS)-4-(1H-Indazol-5-yloxy)-N-[3-(methylsulfonyl)propyl]-6,7,8,9-tetrahydro-5H-pyrimido[4,5-b]indole-6-carboxamide

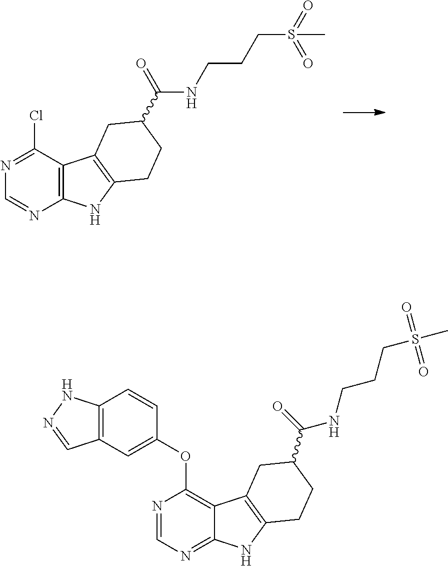 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Oc4ccc5[nH]ncc5c4)c3c2C1]

A mixture comprising 50 mg (135 μmol) (6RS)-4-chloro-N-[3-(methylsulfonyl) propyl]-6,7,8,9-tetrahydro-5H-pyrimido[4,5-b]indole-6-carboxamide which was prepared according to intermediate example 28a, 54.3 mg 1H-indazol-5-ol 132 mg caesium carbonate, 5.6 mg N,N-dimethylglycine, 5.3 mg copper(I)chloride and 1.6 mL 1,4-dioxane was heated at 160° C. using microwave irradiation for 3 hours. Dichloromethane and methanol were added, the mixture was filtered, the filtrate evaporated and the residue purified by chromatography to give 11.3 mg (16%) of the title compound.

¹H-NMR (DMSO-d6): δ=1.75-1.90 (3H), 2.05 (1H), 2.58 (1H), 2.64-2.94 (4H), 2.91 (3H), 2.98-3.22 (5H), 7.17 (1H), 7.51-7.57 (2H), 8.01 (1H), 8.03 (1H), 8.09 (1H), 11.80 (1H), 13.14 (1H) ppm.

### Example 57

### (RS)-4-(4-Fluoro-2-methoxyphenoxy)-N-[3-(methylsulfonyl)propyl]-6,7,8,9-tetrahydro-5H-pyrimido[4,5-b]indole-6-carboxamide

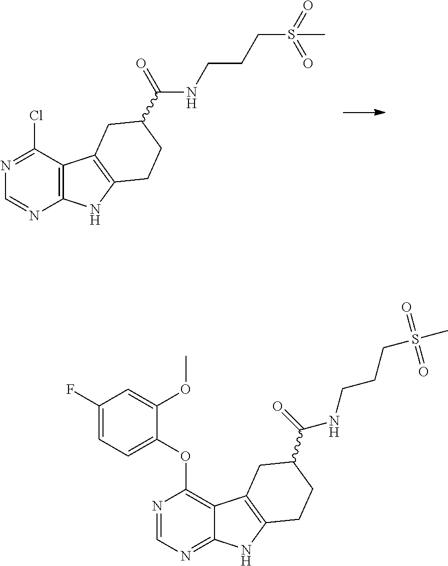 [SMILES: CS(=O)(=O)CCCNC(=O)C1CCc2[nH]c3ncnc(Cl)c3c2C1 ; COc1cc(F)ccc1Oc1ncnc2[nH]c3c(c12)CC(C(=O)NCCCS(C)(=O)=O)CC3]

50 mg (135 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 56 using 4-fluoro-2-methoxyphenol to give after working up and purification 7.8 mg (16%) of the title compound.

¹H-NMR (DMSO-d6): δ=1.73-1.87 (3H), 2.04 (1H), 2.55 (1H), 2.69-2.84 (3H), 2.92 (3H), 2.96 (1H), 3.03-3.20 (4H), 3.66 (3H), 6.77 (1H), 7.04 (1H), 7.17 (1H), 8.00 (1H), 8.06 (1H), 11.76 (1H) ppm.

### Example 58

### (RS)—N-[3-(Methylsulfonyl)propyl]-4-(5,6,7,8-tetrahydronaphthalen-1-ylamino)-6,7,8,9-tetrahydro-5H-pyrimido[4,5-b]indole-6-carboxamide

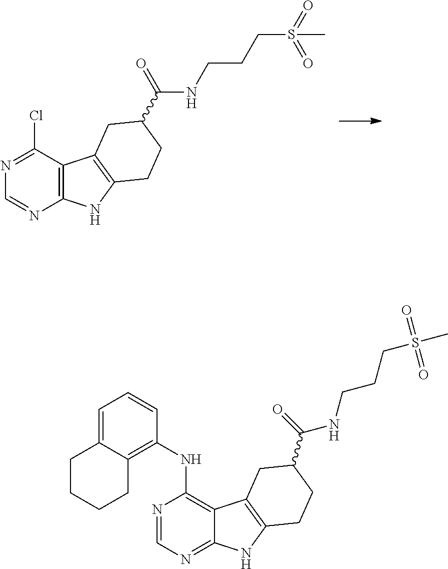 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4cccc5c4CCCC5)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 5,6,7,8-tetrahydronaphthalen-1-amine to give after working up and purification 8.8 mg (21%) of the title compound.

¹H-NMR (DMSO-d6): δ=1.60-1.88 (7H), 1.99 (1H), 2.51-2.75 (7H), 2.83 (1H), 2.92 (3H), 2.99-3.23 (5H), 6.86 (1H), 7.04 (1H), 7.36 (1H), 7.51 (1H), 7.96 (2H), 11.31 (1H) ppm.

### Example 59

### (RS)-2-Methyl-5-[(6-{[3-(methylsulfonyl)propyl]carbamoyl}-6,7,8,9-tetrahydro-5H-pyrimido[4,5-b]indol-4-yl)amino]benzenesulfonic acid

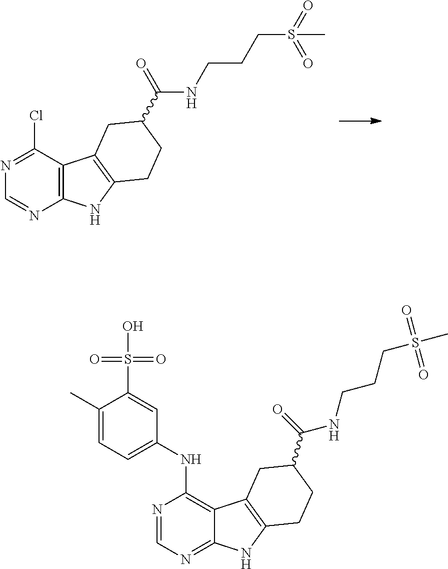

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 5-amino-2-methylbenzenesulfonic acid to give after working up and purification 3.6 mg (8%) of the title compound.

¹H-NMR (DMSO-d6): δ=1.68-1.92 (3H), 2.02 (1H), 2.23 (3H), 2.54 (1H), 2.67 (3H), 2.87 (1H), 2.97 (3H), 3.05-3.22 (5H), 7.09 (1H), 7.47 (1H), 7.83 (1H), 8.11 (1H), 8.38 (1H), 9.53 (1H), 11.35 (1H) ppm.

### Example 60

### (RS)-4-[(2,2-Difluoro-1,3-benzodioxol-4-yl)amino]-N-[3-(methylsulfonyl)propyl]-6,7,8,9-tetrahydro-5H-pyrimido[4,5-b]indole-6-carboxamide

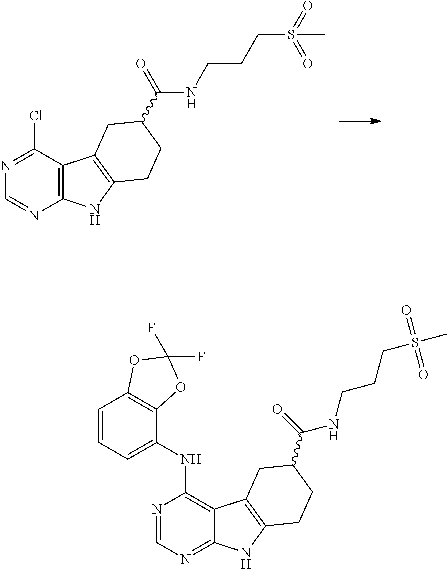 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4cccc5c4OC(F)(F)O5)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2,2-difluoro-1,3-benzodioxol-4-amine to give after working up and purification 14.6 mg (34%) of the title compound.

¹H-NMR (DMSO-d6): δ=1.73-1.87 (3H), 1.99 (1H), 2.52 (1H), 2.65-2.77 (2H), 2.87-2.97 (1H), 2.93 (3H), 3.03-3.24 (5H), 7.08-7.16 (2H), 7.26 (1H), 8.01 (1H), 8.05 (1H), 8.47 (1H), 11.49 (1H) ppm.

### Example 61

### (RS)-4-[(6-Fluoro-1H-indazol-5-yl)amino]-N-[3-(methylsulfonyl)propyl]-6,7,8,9-tetrahydro-5H-pyrimido[4,5-b]indole-6-carboxamide

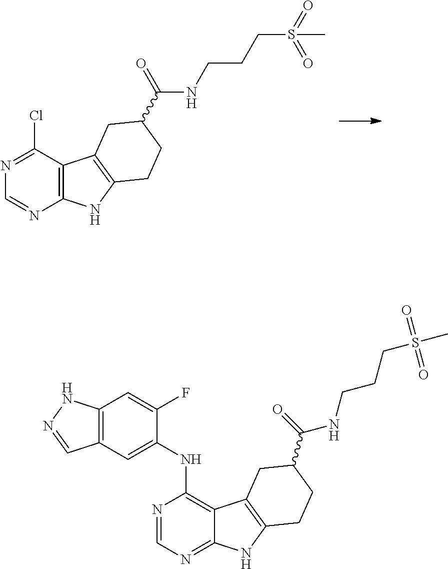 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4cc5cn[nH]c5cc4F)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 6-fluoro-1H-indazol-5-amine to give after working up and purification 9.6 mg (23%) of the title compound.

¹H-NMR (DMSO-d6): δ=1.73-1.87 (3H), 2.01 (1H), 2.54 (1H), 2.65-2.75 (2H), 2.87-2.98 (1H), 2.93 (3H), 3.05-3.25 (5H), 7.36 (1H), 7.90 (1H), 7.97-8.06 (4H), 11.38 (1H), 13.04 (1H) ppm.

### Example 62

### (RS)-4-{[2-Fluoro-5-(trifluoromethoxy)phenyl]amino}-N-[3-(methylsulfonyl)propyl]-6,7,8,9-tetrahydro-5H-pyrimido[4,5-b]indole-6-carboxamide

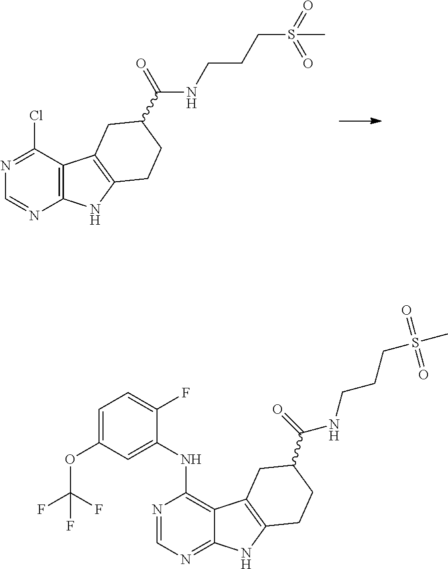 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4cc(OC(F)(F)F)ccc4F)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2-fluoro-5-(trifluoromethoxy)aniline to give after working up and purification 4.6 mg (10%) of the title compound.

¹H-NMR (DMSO-d6): δ=1.72-1.88 (3H), 2.01 (1H), 2.54 (1H), 2.65-2.77 (2H), 2.89-3.00 (1H), 2.93 (3H), 3.03-3.24 (5H), 7.08 (1H), 7.34 (1H), 7.99 (1H), 8.02-8.09 (2H), 8.12 (1H), 11.56 (1H) ppm.

### Example 63

### (RS)—N-[3-(Methylsulfonyl)propyl]-4-{[4-(morpholin-4-yl)phenyl]amino}-6,7,8,9-tetrahydro-5H-pyrimido[4,5-b]indole-6-carboxamide

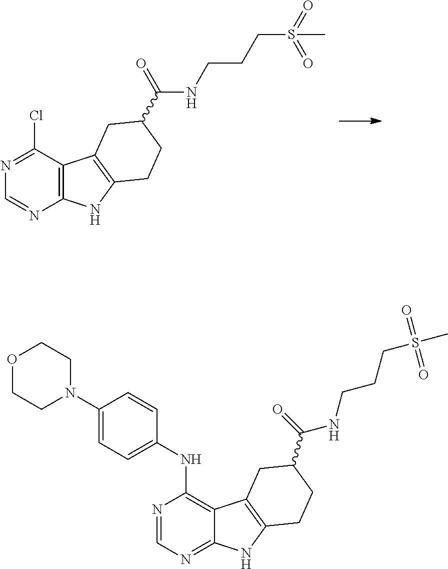 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc(N5CCOCC5)cc4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 4-(morpholin-4-yl)aniline to give after working up and purification 22.5 mg (52%) of the title compound.

¹H-NMR (DMSO-d6): δ=1.72-1.87 (3H), 1.99 (1H), 2.50 (1H), 2.58-2.76 (2H), 2.91 (1H), 2.94 (3H), 3.02 (4H), 3.05-3.23 (5H), 3.71 (4H), 6.87 (2H), 7.46 (2H), 7.73 (1H), 7.98 (1H), 8.02 (1H), 11.32 (1H) ppm.

### Example 64

### (RS)-Methyl methyl{4-[(6-{[3-(methylsulfonyl)propyl]carbamoyl}-6,7,8,9-tetrahydro-5H-pyrimido[4,5-b]indol-4-yl)amino]phenyl}phosphinate

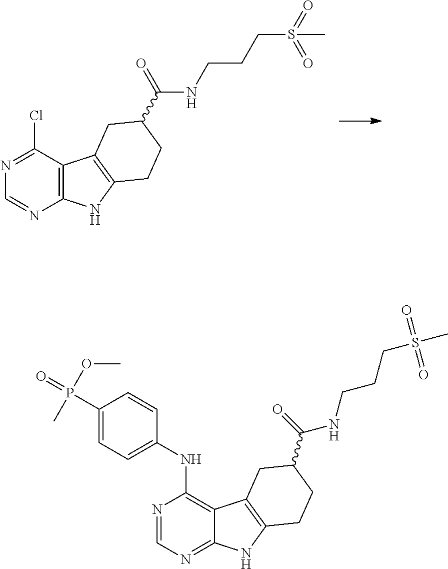 [SMILES: CS(=O)(=O)CCCNC(=O)C1CCc2[nH]c3ncnc(Cl)c3c2C1 ; COP(C)(=O)c1ccc(Nc2ncnc3[nH]c4c(c23)CC(C(=O)NCCCS(C)(=O)=O)CC4)cc1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using methyl(4-aminophenyl)methylphosphinate to give after working up and purification 14.3 mg (32%) of the title compound.

¹H-NMR (DMSO-d6): δ=1.58 (3H), 1.73-1.87 (3H), 2.00 (1H), 2.51 (1H), 2.70 (2H), 2.94 (3H), 2.98 (1H), 3.06-3.24 (5H), 3.44 (3H), 7.61 (2H), 7.82 (2H), 8.00 (1H), 8.19 (1H), 8.32 (1H), 11.55 (1H) ppm.

### Example 65

### (RS)-4-{[7-(Methylsulfanyl)-2,3-dihydro-1,4-benzodioxin-6-yl]amino}-N-[3-(methylsulfonyl)propyl]-6,7,8,9-tetrahydro-5H-pyrimido[4,5-b]indole-6-carboxamide

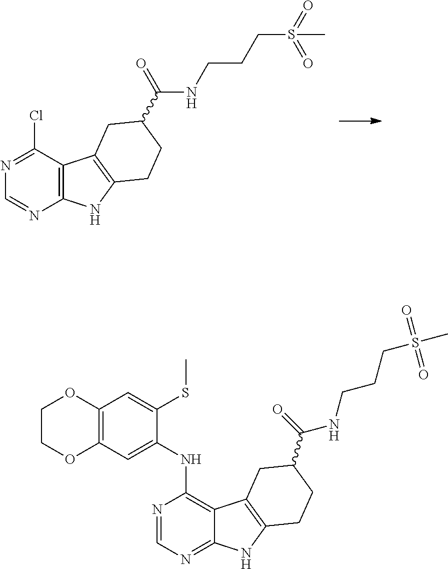 [SMILES: CS(=O)(=O)CCCNC(=O)C1CCc2[nH]c3ncnc(Cl)c3c2C1 ; CSc1cc2c(cc1Nc1ncnc3[nH]c4c(c13)CC(C(=O)NCCCS(C)(=O)=O)CC4)OCCO2]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 7-(methylsulfanyl)-2,3-dihydro-1,4-benzodioxin-6-amine to give after working up and purification 10.1 mg (22%) of the title compound.

¹H-NMR (DMSO-d6): δ=1.73-1.87 (3H), 2.01 (1H), 2.27 (3H), 2.54-2.73 (3H), 2.93 (3H), 2.98 (1H), 3.06-3.23 (5H), 4.17-4.26 (4H), 6.98 (1H), 7.97 (1H), 7.99 (1H), 8.02 (1H), 8.12 (1H), 11.47 (1H) ppm.

### Example 66

### N-(1H-Indazol-5-yl)-5,7,8,9-tetrahydrothiopyrano[3′,4′:4,5]pyrrolo[2,3-d]pyrimidin-4-amine

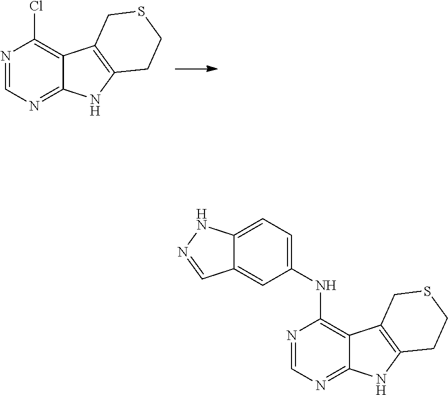 [SMILES: Clc1ncnc2[nH]c3c(c12)CSCC3 ; c1nc(Nc2ccc3[nH]ncc3c2)c2c3c([nH]c2n1)CCSC3]

30 mg (133 μmol) 4-chloro-5,7,8,9-tetrahydrothiopyrano[3′,4′:4,5]pyrrolo[2,3-d]pyrimidine which was prepared according to intermediate example 11a were transformed in analogy to example 1 using 1H-indazol-5-amine to give after working up and purification 42 mg (98%) of the title compound.

¹H-NMR (DMSO-d6): δ=2.94 (4H), 4.04 (2H), 7.40 (1H), 7.65 (1H), 7.91 (1H), 8.09 (1H), 8.12 (1H), 9.65 (1H), 12.55 (1H), 13.27 (1H) ppm.

### Example 67

### (RS)-4-{[2-Methoxy-5-(trifluoromethyl)phenyl]amino}-N-[3-(methylsulfonyl)propyl]-6,7,8,9-tetrahydro-5H-pyrimido[4,5-b]indole-6-carboxamide

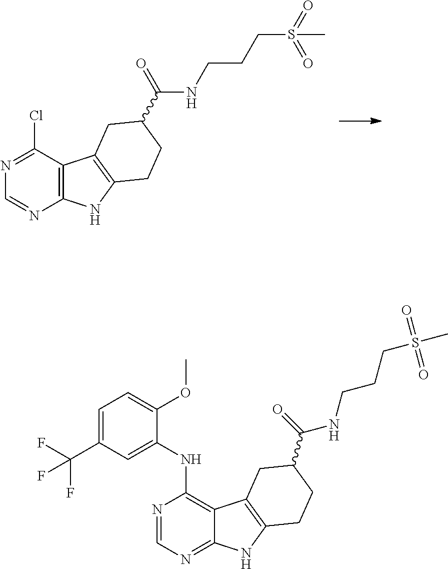 [SMILES: CS(=O)(=O)CCCNC(=O)C1CCc2[nH]c3ncnc(Cl)c3c2C1 ; COc1ccc(C(F)(F)F)cc1Nc1ncnc2[nH]c3c(c12)CC(C(=O)NCCCS(C)(=O)=O)CC3]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2-methoxy-5-(trifluoromethyl)aniline to give after working up and purification 23.4 mg (55%) of the title compound.

¹H-NMR (DMSO-d6): δ=1.74-1.91 (3H), 2.04 (1H), 2.56-2.76 (3H), 2.94 (3H), 2.97-3.23 (6H), 3.93 (3H), 7.23 (1H), 7.36 (1H), 7.94 (1H), 8.05 (1H), 8.25 (1H), 8.89 (1H), 11.66 (1H) ppm.

### Example 68

### (RS)-4-[(5-Methoxy-2-methyl-1,3-benzothiazol-6-yl)amino]-N-[3-(methylsulfonyl)propyl]-6,7,8,9-tetrahydro-5H-pyrimido[4,5-b]indole-6-carboxamide

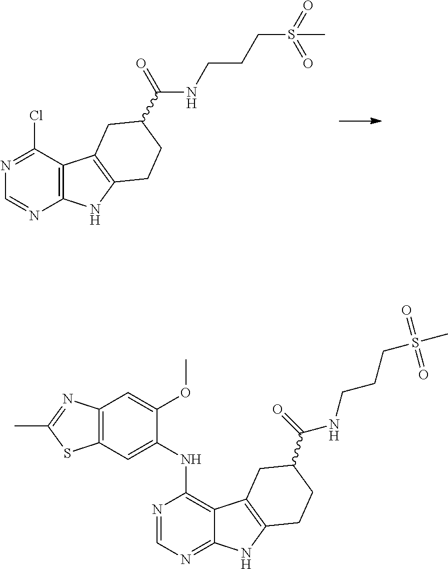

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 5-methoxy-2-methyl-1,3-benzothiazol-6-amine to give after working up and purification 19.1 mg (45%) of the title compound.

¹H-NMR (DMSO-d6): δ=1.75-1.90 (3H), 2.05 (1H), 2.57-2.75 (3H), 2.73 (3H), 2.94 (3H), 2.95-3.24 (6H), 3.94 (3H), 7.56 (1H), 7.98 (1H), 8.06 (1H), 8.24 (1H), 9.09 (1H), 11.61 (1H) ppm.

### Example 69

### N-(1H-Indazol-5-yl)-5,6,7,8-tetrahydrocyclopenta[4,5]pyrrolo[2,3-d]pyrimidin-4-amine

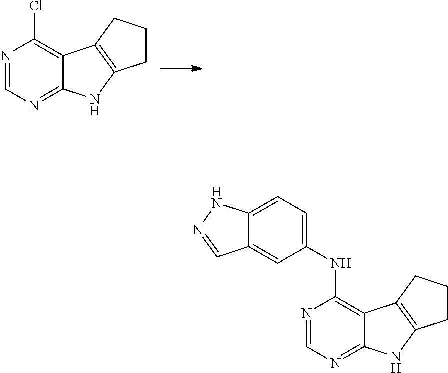 [SMILES: Clc1ncnc2[nH]c3c(c12)CCC3 ; c1nc(Nc2ccc3[nH]ncc3c2)c2c3c([nH]c2n1)CCC3]

30 mg (155 μmol) 4-chloro-5,6,7,8-tetrahydrocyclopenta[4,5]pyrrolo[2,3-d]pyrimidine which was prepared according to intermediate example 69a were transformed in analogy to example 1 using 1H-indazol-5-amine to give after working up and purification 18.6 mg (39%) of the title compound.

¹H-NMR (DMSO-d6): δ=2.39 (2H), 2.83 (2H), 2.92 (2H), 6.58 (2H), 7.50 (1H), 7.62 (1H), 7.86 (1H), 7.95 (1H), 8.11 (1H), 13.20 (1H) ppm.

### Example 69a

### 4-Chloro-5,6,7,8-tetrahydrocyclopenta[4,5]pyrrolo[2,3-d]pyrimidine

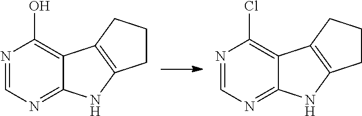 [SMILES: Oc1ncnc2[nH]c3c(c12)CCC3 ; Clc1ncnc2[nH]c3c(c12)CCC3]

332 mg (1.89 mmol) 5,6,7,8-tetrahydrocyclopenta[4,5]pyrrolo[2,3-d]pyrimidin-4-ol which was prepared according to intermediate example 69b were transformed in analogy to intermediate example 1a to give after working up and purification 203 mg (55%) of the title compound.

### Example 69b

### 5,6,7,8-Tetrahydrocyclopenta[4,5]pyrrolo[2,3-d]pyrimidin-4-ol

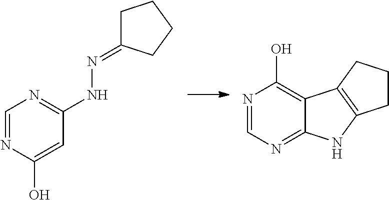 [SMILES: Oc1cc(NN=C2CCCC2)ncn1 ; Oc1ncnc2[nH]c3c(c12)CCC3]

602 mg (3.14 mmol) 6-(2-cyclopentylidenehydrazino)pyrimidin-4-ol which was prepared according to intermediate example 69c were transformed in analogy to intermediate example 1b to give after working up and purification 339 mg (62%) of the title compound.

### Example 69c

### 6-(2-Cyclopentylidenehydrazino)pyrimidin-4-ol

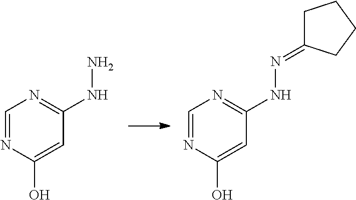 [SMILES: NNc1cc(O)ncn1 ; Oc1cc(NN=C2CCCC2)ncn1]

1.0 g (7.93 mmol) 6-hydrazinopyrimidin-4-ol/6-hydrazinopyrimidin-4(1H)-one (CAS-No: 29939-37-5) were transformed in analogy to intermediate example 1c using cyclopentanone to give after working up and purification 1.34 g (88%) of the title compound.

### Example 70

### N-(4-Fluoro-2-isopropoxyphenyl)-5,6,7,8-tetrahydrocyclopenta[4,5]pyrrolo[2,3-d]pyrimidin-4-amine

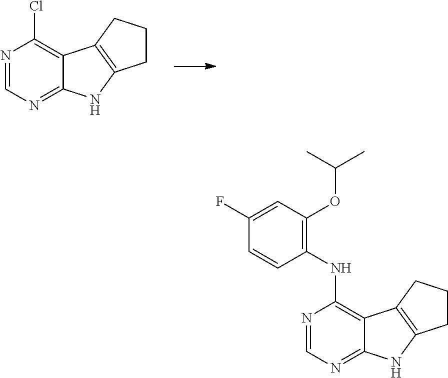 [SMILES: Clc1ncnc2[nH]c3c(c12)CCC3 ; CC(C)Oc1cc(F)ccc1Nc1ncnc2[nH]c3c(c12)CCC3]

30 mg (155 μmol) 4-chloro-5,6,7,8-tetrahydrocyclopenta[4,5]pyrrolo[2,3-d]pyrimidine which was prepared according to intermediate example 69a were transformed in analogy to example 1 to give after working up and purification 20.2 mg (38%) of the title compound.

¹H-NMR (DMSO-d6): δ=1.29 (6H), 2.44 (2H), 2.78 (2H), 2.83 (2H), 4.71 (1H), 6.75 (1H), 7.00 (1H), 7.55 (1H), 8.17 (1H), 8.55 (1H), 11.61 (1H) ppm.

### Example 71

### (RS)-4-{[2-Methoxy-4-(morpholin-4-yl)phenyl]amino}-N-[3-(methylsulfonyl)propyl]-6,7,8,9-tetrahydro-5H-pyrimido[4,5-b]indole-6-carboxamide

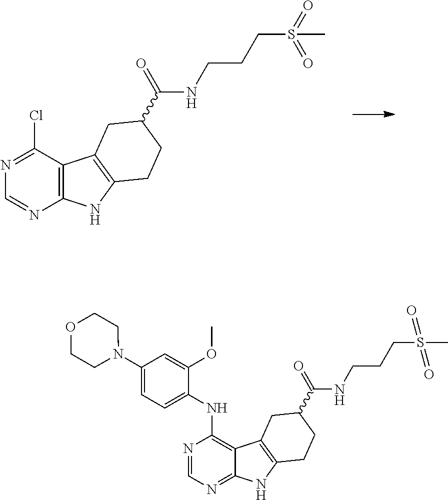 [SMILES: CS(=O)(=O)CCCNC(=O)C1CCc2[nH]c3ncnc(Cl)c3c2C1 ; COc1cc(N2CCOCC2)ccc1Nc1ncnc2[nH]c3c(c12)CC(C(=O)NCCCS(C)(=O)=O)CC3]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2-methoxy-4-(morpholin-4-yl)aniline to give after working up and purification 24.3 mg (53%) of the title compound.

¹H-NMR (DMSO-d6): δ=1.71-1.89 (3H), 2.02 (1H), 2.53-2.73 (3H), 2.87-3.23 (10H), 2.93 (3H), 3.65-3.77 (4H), 3.81 (3H), 6.49 (1H), 6.65 (1H), 7.43 (1H), 8.03 (1H), 8.08 (1H), 8.17 (1H), 11.38 (1H) ppm.

### Example 72

### (RS)-4-[(2,6-Dimethoxyphenyl)amino]-N-[3-(methylsulfonyl)propyl]-6,7,8,9-tetrahydro-5H-pyrimido[4,5-b]indole-6-carboxamide

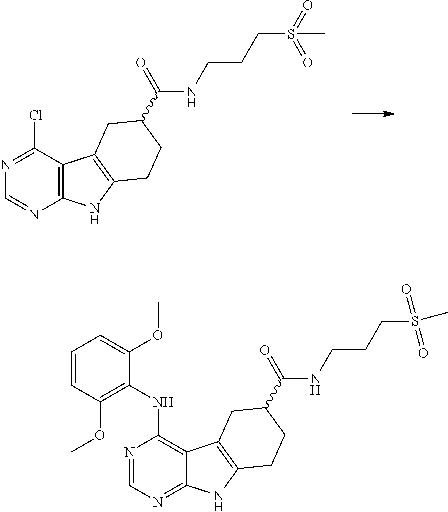 [SMILES: CS(=O)(=O)CCCNC(=O)C1CCc2[nH]c3ncnc(Cl)c3c2C1 ; COc1cccc(OC)c1Nc1ncnc2[nH]c3c(c12)CC(C(=O)NCCCS(C)(=O)=O)CC3]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2,6-dimethoxyaniline to give after working up and purification 12.1 mg (28%) of the title compound.

¹H-NMR (DMSO-d6): δ=1.70-1.88 (3H), 2.01 (1H), 2.61-2.84 (3H), 2.92 (3H), 2.99-3.23 (5H), 3.35 (1H), 3.65 (6H), 6.66 (2H), 7.02 (1H), 7.16 (1H), 7.81 (1H), 7.95 (1H), 11.18 (1H) ppm.

### Example 73

### (RS)-4-[(2,4-Dimethoxyphenyl)amino]-N-[3-(methylsulfonyl)propyl]-6,7,8,9-tetrahydro-5H-pyrimido[4,5-b]indole-6-carboxamide

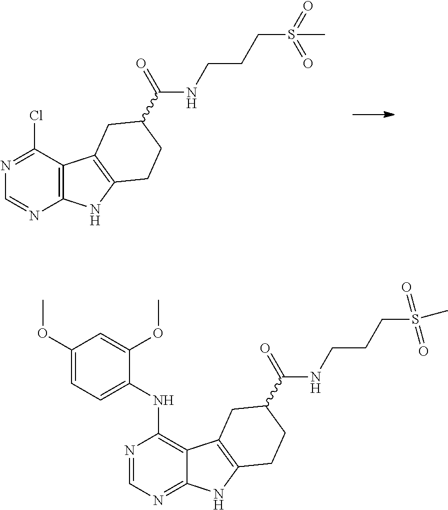 [SMILES: CS(=O)(=O)CCCNC(=O)C1CCc2[nH]c3ncnc(Cl)c3c2C1 ; COc1ccc(Nc2ncnc3[nH]c4c(c23)CC(C(=O)NCCCS(C)(=O)=O)CC4)c(OC)c1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2,4-dimethoxyaniline to give after working up and purification 21.1 mg (51%) of the title compound.

¹H-NMR (DMSO-d6): δ=1.71-1.90 (3H), 2.02 (1H), 2.53-2.73 (3H), 2.87-3.24 (6H), 2.93 (3H), 3.72 (3H), 3.81 (3H), 6.51 (1H), 6.63 (1H), 7.44 (1H), 8.03 (1H), 8.08 (1H), 8.18 (1H), 11.39 (1H) ppm.

### Example 74

### (RS)-4-[(2,3-Dimethoxyphenyl)amino]-N-[3-(methylsulfonyl)propyl]-6,7,8,9-tetrahydro-5H-pyrimido[4,5-b]indole-6-carboxamide

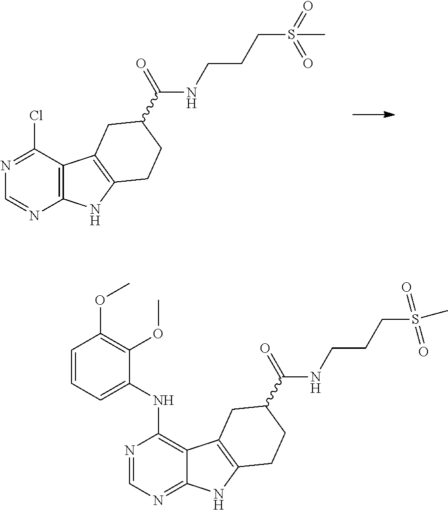 [SMILES: CS(=O)(=O)CCCNC(=O)C1CCc2[nH]c3ncnc(Cl)c3c2C1 ; COc1cccc(Nc2ncnc3[nH]c4c(c23)CC(C(=O)NCCCS(C)(=O)=O)CC4)c1OC]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2,3-dimethoxyaniline to give after working up and purification 11.2 mg (27%) of the title compound.

¹H-NMR (DMSO-d6): δ=1.73-1.89 (3H), 2.04 (1H), 2.56-2.76 (3H), 2.95 (3H), 2.99-3.24 (6H), 3.79 (6H), 6.69 (1H), 7.02 (1H), 7.78 (1H), 8.05 (1H), 8.20 (1H), 8.27 (1H), 11.52 (1H) ppm.

### Example 75

### (RS)-4-[(2-Methoxy-6-methylphenyl)amino]-N-[3-(methylsulfonyl)propyl]-6,7,8,9-tetrahydro-5H-pyrimido[4,5-b]indole-6-carboxamide

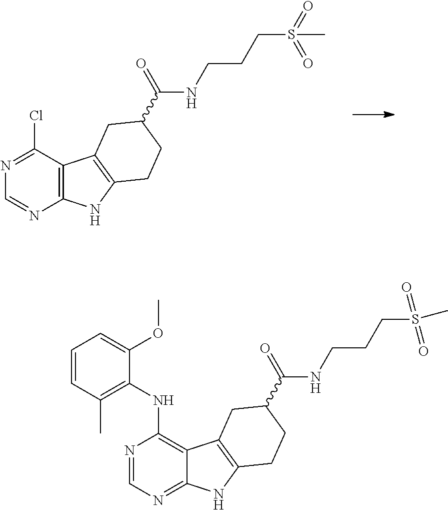 [SMILES: CS(=O)(=O)CCCNC(=O)C1CCc2[nH]c3ncnc(Cl)c3c2C1 ; COc1cccc(C)c1Nc1ncnc2[nH]c3c(c12)CC(C(=O)NCCCS(C)(=O)=O)CC3]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2-methoxy-6-methylaniline to give after working up and purification 11.1 mg (28%) of the title compound.

¹H-NMR (DMSO-d6): δ=1.68-1.89 (3H), 2.01 (1H), 2.09 (3H), 2.52 (1H), 2.68 (2H), 2.82 (1H), 2.92 (3H), 3.02-3.25 (5H), 3.65 (3H), 6.83 (2H), 7.11 (1H), 7.27 (1H), 7.83 (1H), 7.99 (1H), 11.25 (1H) ppm.

### Example 76

### (RS)-4-{[2-Methoxy-4-(4-methylpiperazin-1-yl)phenyl]amino}-N-[3-(methylsulfonyl)propyl]-6,7,8,9-tetrahydro-5H-pyrimido[4,5-b]indole-6-carboxamide

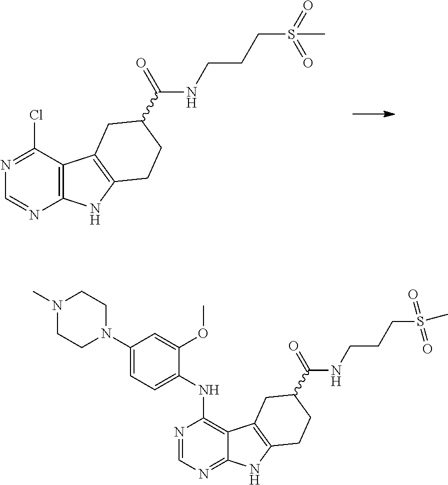

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2-methoxy-4-(4-methylpiperazin-1-yl)aniline to give after working up and purification 10.2 mg (22%) of the title compound.

¹H-NMR (DMSO-d6): δ=1.70-1.89 (3H), 2.02 (1H), 2.19 (3H), 2.42 (4H), 2.58 (1H), 2.68 (2H), 2.85-3.24 (10H), 2.94 (3H), 3.80 (3H), 6.47 (1H), 6.63 (1H), 7.42 (1H), 8.05 (1H), 8.07 (1H), 8.14 (1H), 11.39 (1H) ppm.

### Example 77

### (RS)-4-[(2,3-Difluorophenyl)amino]-N-[3-(methylsulfonyl)propyl]-6,7,8,9-tetrahydro-5H-pyrimido[4,5-b]indole-6-carboxamide

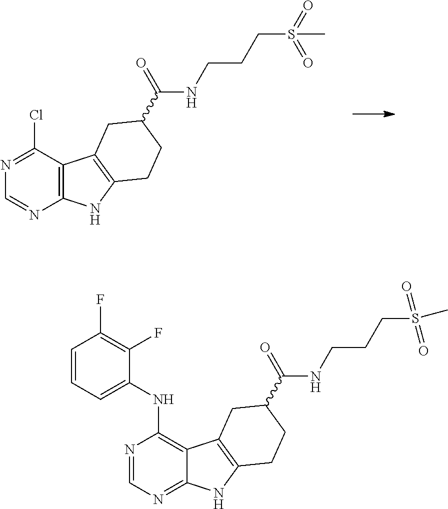 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4cccc(F)c4F)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2,3-difluoroaniline to give after working up and purification 10.1 mg (27%) of the title compound.

¹H-NMR (DMSO-d6): δ=1.73-1.87 (3H), 2.00 (1H), 2.52 (1H), 2.69 (2H), 2.87-3.00 (1H), 2.93 (3H), 3.03-3.25 (5H), 7.13 (2H), 7.51 (1H), 8.01 (1H), 8.06 (1H), 8.13 (1H), 11.47 (1H) ppm.

### Example 78

### (RS)-4-{[2-(Cyclopentyloxy)phenyl]amino}-N-[3-(methylsulfonyl)propyl]-6,7,8,9-tetrahydro-5H-pyrimido[4,5-b]indole-6-carboxamide

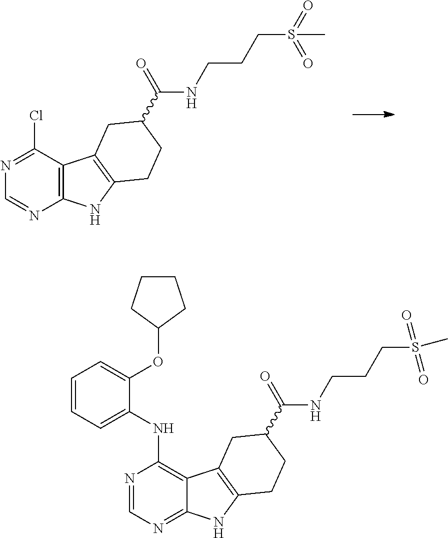 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccccc4OC4CCCC4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2-(cyclopentyloxy)aniline to give after working up and purification 10 mg (24%) of the title compound.

¹H-NMR (DMSO-d6): δ=1.49-1.59 (2H), 1.62-1.96 (9H), 2.00 (1H), 2.60 (1H), 2.65-2.73 (2H), 2.94 (3H), 2.98-3.15 (5H), 3.28 (1H), 4.92 (1H), 6.87-6.93 (2H), 6.99 (1H), 7.69 (1H), 8.06 (1H), 8.21 (1H), 8.79 (1H), 11.52 (1H) ppm.

### Example 79

### (RS)—N-[3-(Methylsulfonyl)propyl]-4-{[2-(tetrahydro-2H-pyran-4-yloxy)phenyl]amino}-6,7,8,9-tetrahydro-5H-pyrimido[4,5-b]indole-6-carboxamide

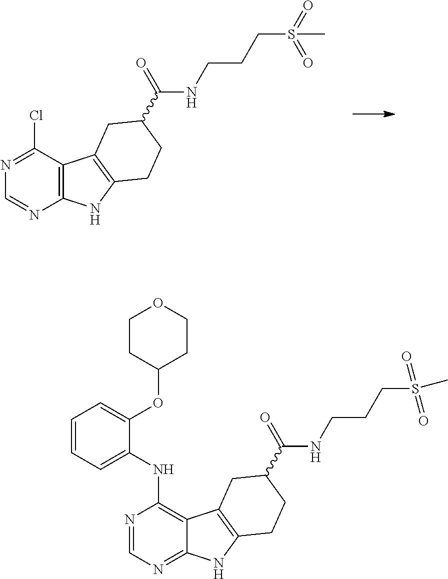 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccccc4OC4CCOCC4)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2-(tetrahydro-2H-pyran-4-yloxy)aniline to give after working up and purification 8.8 mg (21%) of the title compound.

¹H-NMR (DMSO-d6): δ=1.59 (2H), 1.76-1.88 (3H), 1.94-2.05 (3H), 2.60 (1H), 2.66-2.73 (2H), 2.93 (3H), 2.98-3.14 (5H), 3.38-3.47 (3H), 3.83 (2H), 4.63 (1H), 6.88-6.95 (2H), 7.11 (1H), 7.75 (1H), 8.06 (1H), 8.22 (1H), 8.77 (1H), 11.52 (1H) ppm.

### Example 80

### (RS)-4-[(2,2-Dimethyl-2,3-dihydro-1-benzofuran-7-yl)amino]-N-[3-(methylsulfonyl)propyl]-6,7,8,9-tetrahydro-5H-pyrimido[4,5-b]indole-6-carboxamide

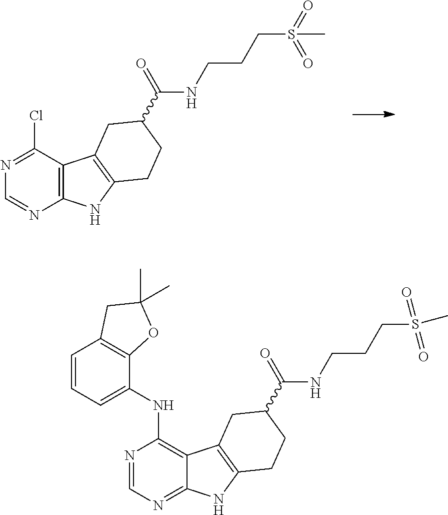 [SMILES: CS(=O)(=O)CCCNC(=O)C1CCc2[nH]c3ncnc(Cl)c3c2C1 ; CC1(C)Cc2cccc(Nc3ncnc4[nH]c5c(c34)CC(C(=O)NCCCS(C)(=O)=O)CC5)c2O1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 2,2-dimethyl-2,3-dihydro-1-benzofuran-7-amine to give after working up and purification 19.1 mg (47%) of the title compound.

¹H-NMR (DMSO-d6): δ=1.38 (6H), 1.73-1.87 (3H), 2.01 (1H), 2.56 (1H), 2.64-2.73 (2H), 2.90 (1H), 2.93 (3H), 2.96-3.05 (3H), 3.07-3.25 (4H), 6.75 (1H), 6.86 (1H), 7.32 (1H), 7.79 (1H), 8.02 (1H), 8.08 (1H), 11.41 (1H) ppm.

### Example 81

### (RS)—N-[3-(Methylsulfonyl)propyl]-4-[(1-oxo-2,3-dihydro-1H-isoindol-4-yl)amino]-6,7,8,9-tetrahydro-5H-pyrimido[4,5-b]indole-6-carboxamide

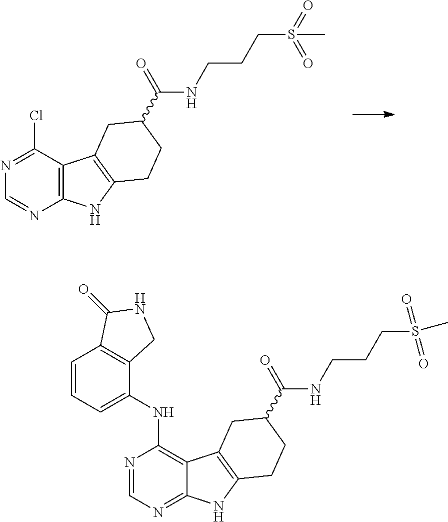 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4cccc5c4CNC5=O)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 4-aminoisoindolin-1-one to give after working up and purification 6.4 mg (16%) of the title compound.

¹H-NMR (DMSO-d6): δ=1.72-1.88 (3H), 2.01 (1H), 2.51 (1H), 2.69 (2H), 2.88-2.99 (1H), 2.93 (3H), 3.05-3.25 (5H), 4.25 (2H), 7.38-7.46 (2H), 7.84 (1H), 7.99 (1H), 8.04 (1H), 8.07 (1H), 8.41 (1H), 11.46 (1H) ppm.

### Example 82

### (RS)-4-[(8-Fluoro-2-oxo-1,2-dihydroquinolin-5-yl)amino]-N-[3-(methylsulfonyl)propyl]-6,7,8,9-tetrahydro-5H-pyrimido[4,5-b]indole-6-carboxamide

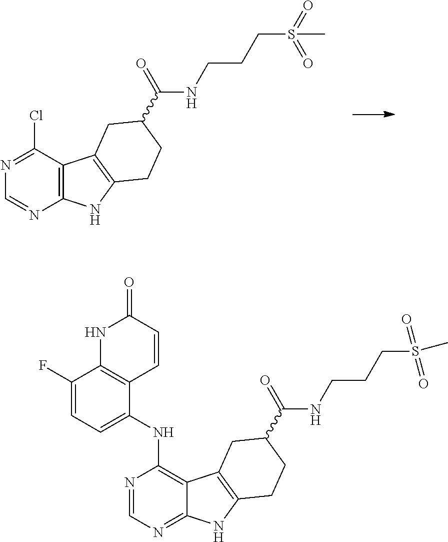 [SMILES: CS(=O)(=O)CCCNC(=O)C1CCc2[nH]c3ncnc(Cl)c3c2C1 ; CS(=O)(=O)CCCNC(=O)C1CCc2[nH]c3ncnc(Nc4ccc(F)c5[nH]c(=O)ccc45)c3c2C1]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 5-amino-8-fluoroquinolin-2(1H)-one to give after working up and purification 3.2 mg (8%) of the title compound.

¹H-NMR (DMSO-d6): δ=1.71-1.89 (3H), 2.00 (1H), 2.50 (1H), 2.60-2.75 (2H), 2.89 (1H), 3.92 (3H), 3.03-3.23 (5H), 6.43 (1H), 7.07 (1H), 7.35 (1H), 7.67 (1H), 7.89 (1H), 8.00 (1H), 8.27 (1H), 11.41 (1H), 11.67 (1H) ppm.

### Example 83

### N-(1H-Indazol-5-yl)-5,7,8,9-tetrahydrothiopyrano[3′,4′:4,5]pyrrolo[2,3-d]pyrimidin-4-amine 6,6-dioxide

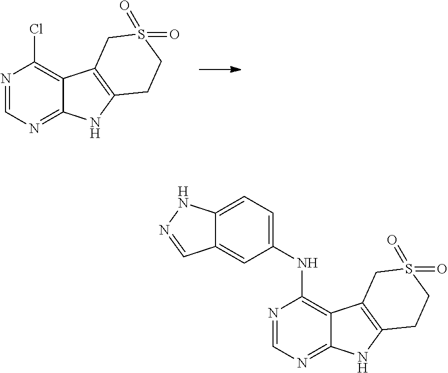 [SMILES: O=S1(=O)CCc2[nH]c3ncnc(Cl)c3c2C1 ; O=S1(=O)CCc2[nH]c3ncnc(Nc4ccc5[nH]ncc5c4)c3c2C1]

30 mg (116 μmol) 4-chloro-5,7,8,9-tetrahydrothiopyrano[3′,4′:4,5]pyrrolo[2,3-d]pyrimidine 6,6-dioxide which was prepared according to intermediate example 83a, compound A, were transformed in analogy to example 1 using 1H-indazol-5-amine to give after working up and purification 30.3 mg (70%) of the title compound.

¹H-NMR (DMSO-d6): δ=3.26 (2H), 3.50 (2H), 4.76 (2H), 7.41 (1H), 7.64 (1H), 7.91 (1H), 8.12 (1H), 8.13 (1H), 9.67 (1H), 12.77 (1H), 13.23 (1H) ppm.

### Example 83a

### 4-Chloro-5,7,8,9-tetrahydrothiopyrano[3′,4′:4,5]pyrrolo[2,3-d]pyrimidine 6,6-dioxide (A) and (RS)-4-chloro-5,7,8,9-tetrahydrothiopyrano[3′,4′:4,5]pyrrolo[2,3-d]pyrimidine 6-oxide (B)

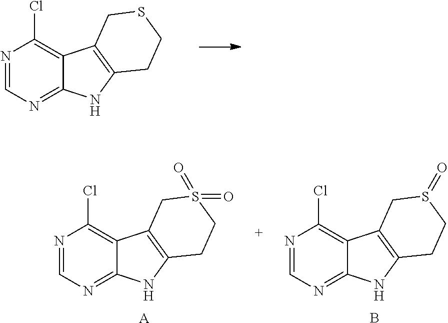

To a suspension of 227 mg (1.01 mmol) 4-chloro-5,7,8,9-tetrahydrothiopyrano[3′,4′:4,5]pyrrolo[2,3-d]pyrimidine which was prepared according to intermediate example 11a in 8 mL trichloromethane were added 498 mg 3-chlorobenzenecarboperoxoic acid (77%) and the mixture was stirred at 23° C. for 3 hours. The solvent was removed and the residue purified by chromatography to give 134 mg (49%) of title compound A and 64.3 mg (21%) of title compound B.

### Example 84

### N-(4-Fluoro-2-isopropoxyphenyl)-5,7,8,9-tetrahydrothiopyrano[3′,4′:4,5]pyrrolo[2,3-d]pyrimidin-4-amine 6,6-dioxide

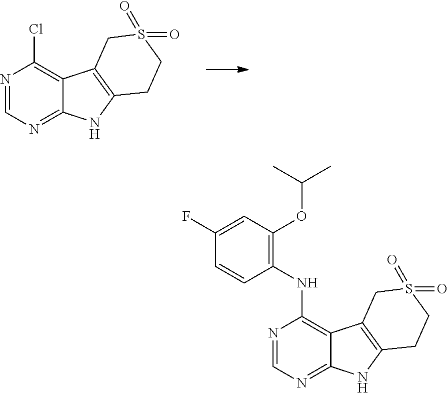 [SMILES: O=S1(=O)CCc2[nH]c3ncnc(Cl)c3c2C1 ; CC(C)Oc1cc(F)ccc1Nc1ncnc2[nH]c3c(c12)CS(=O)(=O)CC3]

30 mg (116 μmol) 4-chloro-5,7,8,9-tetrahydrothiopyrano[3′,4′:4,5]pyrrolo[2,3-d]pyrimidine 6,6-dioxide which was prepared according to intermediate example 83a, compound A, were transformed in analogy to example 1 to give after working up and purification 6.6 mg (14%) of the title compound.

¹H-NMR (DMSO-d6): δ=1.29 (6H), 3.20 (2H), 3.47 (2H), 4.67 (2H), 4.70 (1H), 6.74 (1H), 6.99 (1H), 7.57 (1H), 8.19 (1H), 8.32 (1H), 11.90 (1H) ppm.

### Example 85

### (RS)—N-(1H-Indazol-5-yl)-5,7,8,9-tetrahydrothiopyrano[3′,4′:4,5]pyrrolo[2,3-d]pyrimidin-4-amine 6-oxide

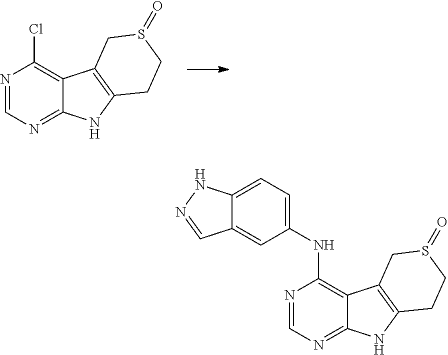 [SMILES: O=S1CCc2[nH]c3ncnc(Cl)c3c2C1 ; O=S1CCc2[nH]c3ncnc(Nc4ccc5[nH]ncc5c4)c3c2C1]

30 mg (124 μmol) (RS)-4-chloro-5,7,8,9-tetrahydrothiopyrano[3′,4′:4,5]pyrrolo[2,3-d]pyrimidine 6-oxide which was prepared according to intermediate example 83a, compound B, were transformed in analogy to example 1 using 1H-indazol-5-amine to give after working up and purification 18.2 mg (41%) of the title compound.

¹H-NMR (DMSO-d6): δ=2.93-3.16 (3H), 3.26 (1H), 4.36 (2H), 7.45 (1H), 7.51 (1H), 7.98 (2H), 8.10 (1H), 8.18 (1H), 11.70 (1H), 12.92 (1H) ppm.

### Example 86

### (RS)—N-(4-Fluoro-2-isopropoxyphenyl)-5,7,8,9-tetrahydrothiopyrano[3′,4′:4,5]pyrrolo[2,3-d]pyrimidin-4-amine 6-oxide

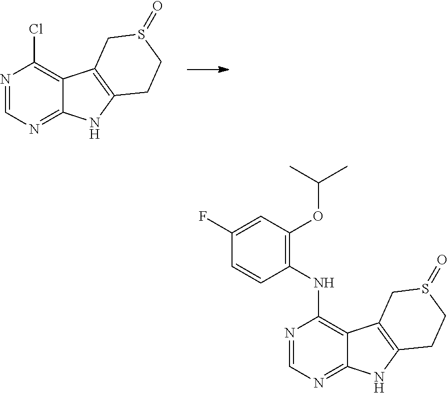 [SMILES: O=S1CCc2[nH]c3ncnc(Cl)c3c2C1 ; CC(C)Oc1cc(F)ccc1Nc1ncnc2[nH]c3c(c12)CS(=O)CC3]

30 mg (124 μmol) (RS)-4-chloro-5,7,8,9-tetrahydrothiopyrano[3′,4′:4,5]pyrrolo[2,3-d]pyrimidine 6-oxide which was prepared according to intermediate example 83a, compound B, were transformed in analogy to example 1 to give after working up and purification 15.8 mg (32%) of the title compound.

¹H-NMR (DMSO-d6): δ=1.31 (6H), 2.98 (1H), 3.03-3.17 (2H), 3.31 (1H), 4.12 (1H), 4.44 (1H), 4.72 (1H), 6.75 (1H), 7.00 (1H), 7.61 (1H), 8.20 (1H), 8.44 (1H), 11.81 (1H) ppm.

### Example 87

### (RS)-Ethyl 4-[(2-oxo-2,3-dihydro-1,3-benzothiazol-6-yl)amino]-6,7,8,9-tetrahydro-5H-pyrimido[4,5-b]indole-6-carboxylate

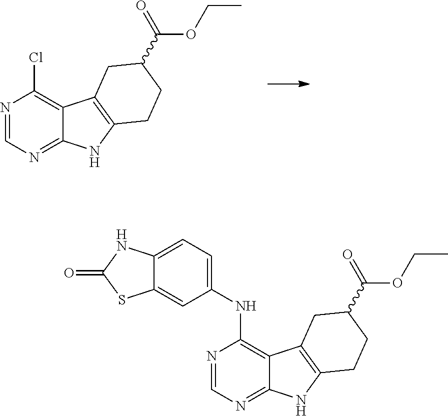 [SMILES: CCOC(=O)C1CCc2[nH]c3ncnc(Cl)c3c2C1 ; CCOC(=O)C1CCc2[nH]c3ncnc(Nc4ccc5[nH]c(=O)sc5c4)c3c2C1]

2.50 g (8.94 mmol) ethyl 4-chloro-6,7,8,9-tetrahydro-5H-pyrimido[4,5-b]indole-6-carboxylate which was prepared according to intermediate example 5a were transformed in analogy to example 1 using 6-amino-1,3-benzothiazol-2(3H)-one to give after working up and purification 3.57 g (97%) of the title compound.

¹H-NMR (DMSO-d6): δ=1.18 (3H), 1.82 (1H), 2.13 (1H), 2.69-2.94 (4H), 3.14 (1H), 4.08 (2H), 7.21 (1H), 7.35 (1H), 7.73 (1H), 8.13 (1H), 9.67 (1H), 12.13 (1H), 12.50 (1H) ppm.

### Example 88

### (RS)-4-[(2-Oxo-2,3-dihydro-1,3-benzothiazol-6-yl)amino]-6,7,8,9-tetrahydro-5H-pyrimido[4,5-b]indole-6-carboxylic acid

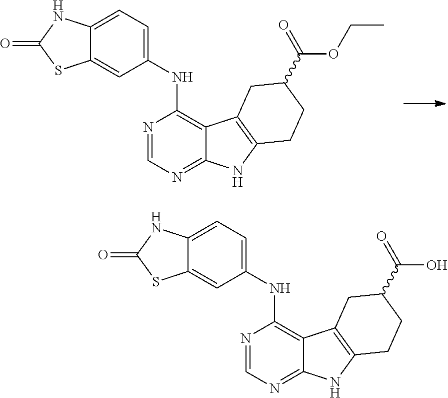 [SMILES: CCOC(=O)C1CCc2[nH]c3ncnc(Nc4ccc5[nH]c(=O)sc5c4)c3c2C1 ; O=C(O)C1CCc2[nH]c3ncnc(Nc4ccc5[nH]c(=O)sc5c4)c3c2C1]

3.56 g (8.69 mmol) (RS)-ethyl 4-[(2-oxo-2,3-dihydro-1,3-benzothiazol-6-yl)amino]-6,7,8,9-tetrahydro-5H-pyrimido[4,5-b]indole-6-carboxylate which was prepared according to example 87 were transformed in analogy to example 6 to give after working up and purification 2.70 g (77%) of the title compound.

¹H-NMR (DMSO-d6): δ=1.82 (1H), 2.13 (1H), 2.64-2.77 (3H), 2.92 (1H), 3.19 (1H), 7.14 (1H), 7.38 (1H), 7.76 (1H), 8.10 (1H), 9.08 (1H), 11.96 (1H), 12.09 (1H), 12.34 (1H) ppm.

### Example 89

### (RS)—N,N-Dimethyl-4-[(2-oxo-2,3-dihydro-1,3-benzothiazol-6-yl)amino]-6,7,8,9-tetrahydro-5H-pyrimido[4,5-b]indole-6-carboxamide

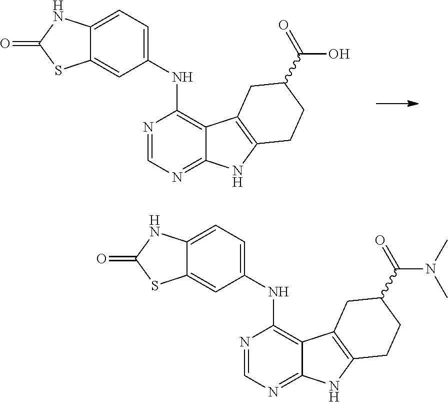 [SMILES: O=C(O)C1CCc2[nH]c3ncnc(Nc4ccc5[nH]c(=O)sc5c4)c3c2C1 ; CN(C)C(=O)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using N-methylmethanamine to give after working up and purification 38.6 mg (60%) of the title compound.

¹H-NMR (DMSO-d6): δ=1.76 (1H), 1.91 (1H), 2.61-3.11 (5H), 2.84 (3H), 3.05 (3H), 7.02 (1H), 7.45 (1H), 7.83 (1H), 7.98 (1H), 8.06 (1H), 10.78 (1H), 11.40 (1H) ppm.

### Example 90

### (RS)-4-[(2-Oxo-2,3-dihydro-1,3-benzothiazol-6-yl)amino]-N-[3-(trifluoromethyl)benzyl]-6,7,8,9-tetrahydro-5H-pyrimido[4,5-b]indole-6-carboxamide

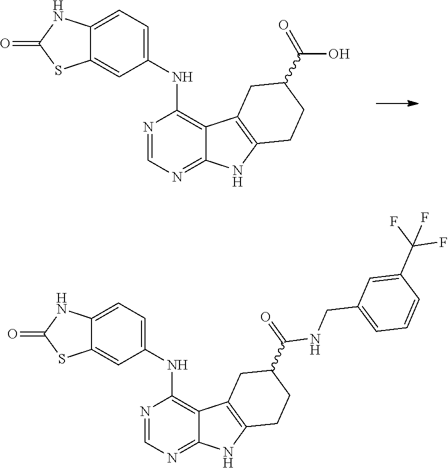 [SMILES: O=C(O)C1CCc2[nH]c3ncnc(Nc4ccc5[nH]c(=O)sc5c4)c3c2C1 ; O=C(NCc1cccc(C(F)(F)F)c1)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using 1-[3-(trifluoromethyl)phenyl]methanamine to give after working up and purification 7.2 mg (9%) of the title compound.

¹H-NMR (DMSO-d6): δ=1.83 (1H), 2.03 (1H), 2.55-2.76 (3H), 3.00 (1H), 3.14 (1H), 4.38 (2H), 7.01 (1H), 7.45 (1H), 7.51-7.64 (4H), 7.84 (1H), 7.98 (1H), 8.07 (1H), 8.53 (1H), 11.42 (1H), 11.68 (1H) ppm.

### Example 91

### (RS)—N-(3-Fluorobenzyl)-4-[(2-oxo-2,3-dihydro-1,3-benzothiazol-6-yl)amino]-6,7,8,9-tetrahydro-5H-pyrimido[4,5-b]indole-6-carboxamide

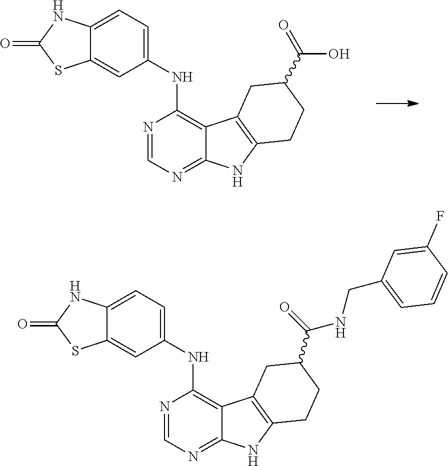 [SMILES: O=C(O)C1CCc2[nH]c3ncnc(Nc4ccc5[nH]c(=O)sc5c4)c3c2C1 ; O=C(NCc1cccc(F)c1)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using 1-(3-fluorophenyl)methanamine to give after working up and purification 16.3 mg (21%) of the title compound.

¹H-NMR (DMSO-d6): δ=1.82 (1H), 2.03 (1H), 2.54-2.74 (3H), 2.99 (1H), 3.14 (1H), 4.31 (2H), 6.99-7.13 (4H), 7.34 (1H), 7.46 (1H), 7.85 (1H), 8.00 (1H), 8.07 (1H), 8.46 (1H), 11.42 (1H), 11.64 (1H) ppm.

### Example 92

### (RS)—N-Benzyl-4-[(2-oxo-2,3-dihydro-1,3-benzothiazol-6-yl)amino]-6,7,8,9-tetrahydro-5H-pyrimido[4,5-b]indole-6-carboxamide

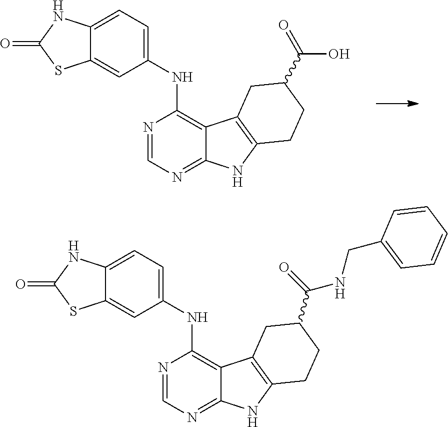 [SMILES: O=C(O)C1CCc2[nH]c3ncnc(Nc4ccc5[nH]c(=O)sc5c4)c3c2C1 ; O=C(NCc1ccccc1)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using 1-phenylmethanamine to give after working up and purification 6.4 mg (9%) of the title compound.

¹H-NMR (DMSO-d6): δ=1.82 (1H), 2.02 (1H), 2.53-2.76 (3H), 3.00 (1H), 3.13 (1H), 4.20-4.39 (2H), 7.02 (1H), 7.17-7.33 (5H), 7.46 (1H), 7.84 (1H), 8.00 (1H), 8.07 (1H), 8.41 (1H), 11.41 (1H), 11.61 (1H) ppm.

### Example 93

### (RS)-4-[(2-Oxo-2,3-dihydro-1,3-benzothiazol-6-yl)amino]-N-phenyl-6,7,8,9-tetrahydro-5H-pyrimido[4,5-b]indole-6-carboxamide

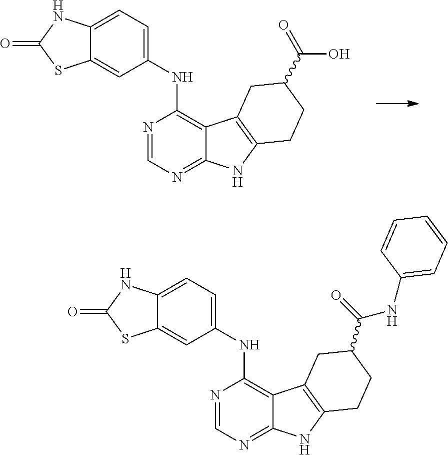 [SMILES: O=C(O)C1CCc2[nH]c3ncnc(Nc4ccc5[nH]c(=O)sc5c4)c3c2C1 ; O=C(Nc1ccccc1)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using aniline to give after working up and purification 8.6 mg (12%) of the title compound.

¹H-NMR (DMSO-d6): δ=1.87 (1H), 2.10 (1H), 2.66-2.81 (3H), 3.02 (1H), 3.23 (1H), 6.97-7.05 (2H), 7.27 (2H), 7.44 (1H), 7.62 (2H), 7.83 (1H), 8.01 (1H), 8.07 (1H), 9.98 (1H), 11.44 (1H), 11.63 (1H) ppm.

### Example 94

### (RS)-4-[(2-Oxo-2,3-dihydro-1,3-benzothiazol-6-yl)amino]-N-(3,3,3-trifluoropropyl)-6,7,8,9-tetrahydro-5H-pyrimido[4,5-b]indole-6-carboxamide

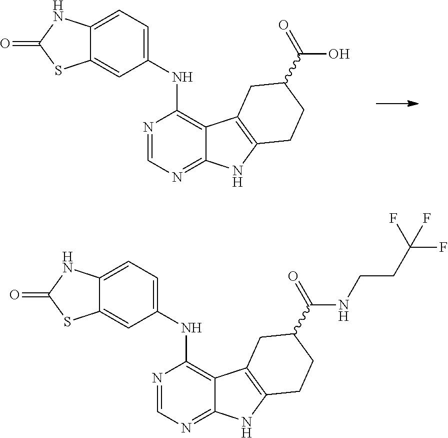 [SMILES: O=C(O)C1CCc2[nH]c3ncnc(Nc4ccc5[nH]c(=O)sc5c4)c3c2C1 ; O=C(NCCC(F)(F)F)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using 3,3,3-trifluoropropan-1-amine to give after working up and purification 5.6 mg (7%) of the title compound.

¹H-NMR (DMSO-d6): δ=1.76 (1H), 1.97 (1H), 2.41 (2H), 2.53 (1H), 2.68 (2H), 2.92 (1H), 3.07 (1H), 3.18-3.41 (2H), 7.02 (1H), 7.44 (1H), 7.84 (1H), 7.97 (1H), 8.06 (1H), 8.13 (1H), 11.41 (2H) ppm.

### Example 95

### (RS)-4-[(2-Oxo-2,3-dihydro-1,3-benzothiazol-6-yl)amino]-N-(2,2,2-trifluoroethyl)-6,7,8,9-tetrahydro-5H-pyrimido[4,5-b]indole-6-carboxamide

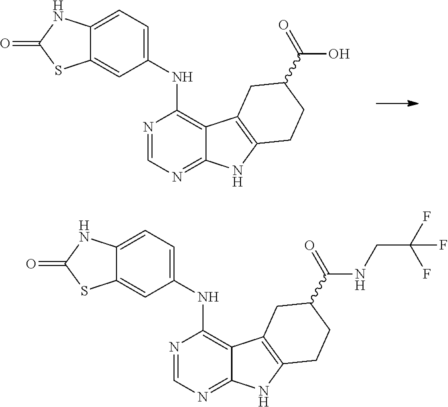 [SMILES: O=C(O)C1CCc2[nH]c3ncnc(Nc4ccc5[nH]c(=O)sc5c4)c3c2C1 ; O=C(NCC(F)(F)F)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using 2,2,2-trifluoroethanamine to give after working up and purification 15.6 mg (21%) of the title compound.

¹H-NMR (DMSO-d6): δ=1.79 (1H), 1.99 (1H), 2.58-2.73 (3H), 2.96 (1H), 3.09 (1H), 3.80-4.02 (2H), 7.02 (1H), 7.43 (1H), 7.83 (1H), 7.99 (1H), 8.06 (1H), 8.56 (1H), 11.41 (1H), 11.54 (1H) ppm.

### Example 96

### (RS)—N-(Cyclopropylmethyl)-4-[(2-oxo-2,3-dihydro-1,3-benzothiazol-6-yl)amino]-6,7,8,9-tetrahydro-5H-pyrimido[4,5-b]indole-6-carboxamide

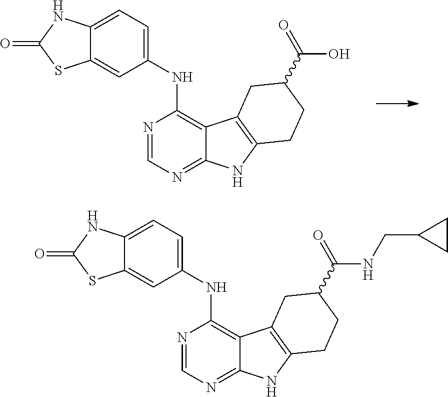 [SMILES: O=C(O)C1CCc2[nH]c3ncnc(Nc4ccc5[nH]c(=O)sc5c4)c3c2C1 ; O=C(NCC1CC1)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using 1-cyclopropylmethanamine to give after working up and purification 12.0 mg (18%) of the title compound.

¹H-NMR (DMSO-d6): δ=0.13 (2H), 0.38 (2H), 0.88 (1H), 1.78 (1H), 1.97 (1H), 2.52 (1H), 2.59-2.73 (2H), 2.87-3.10 (4H), 7.02 (1H), 7.44 (1H), 7.83 (1H), 7.93 (1H), 7.97 (1H), 8.06 (1H), 11.38 (1H), 11.64 (1H) ppm.

### Example 97

### (RS)—N-Isobutyl-4-[(2-oxo-2,3-dihydro-1,3-benzothiazol-6-yl)amino]-6,7,8,9-tetrahydro-5H-pyrimido[4,5-b]indole-6-carboxamide

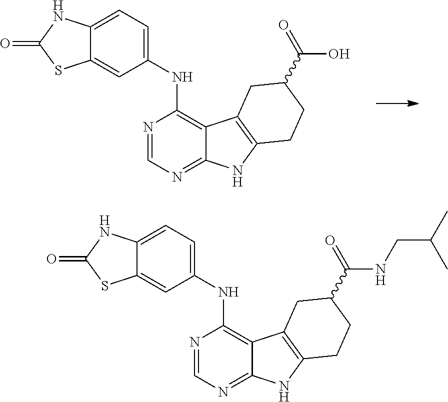 [SMILES: O=C(O)C1CCc2[nH]c3ncnc(Nc4ccc5[nH]c(=O)sc5c4)c3c2C1 ; CC(C)CNC(=O)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using 2-methylpropan-1-amine to give after working up and purification 16.8 mg (24%) of the title compound.

¹H-NMR (DMSO-d6): δ=0.83 (6H), 1.68 (1H), 1.78 (1H), 1.97 (1H), 2.53 (1H), 2.60-2.73 (2H), 2.79-3.00 (3H), 3.06 (1H), 7.02 (1H), 7.43 (1H), 7.80-7.86 (2H), 7.97 (1H), 8.06 (1H), 11.38 (1H), 11.67 (1H) ppm.

### Example 98

### (RS)—N-Isopropyl-4-[(2-oxo-2,3-dihydro-1,3-benzothiazol-6-yl)amino]-6,7,8,9-tetrahydro-5H-pyrimido[4,5-b]indole-6-carboxamide

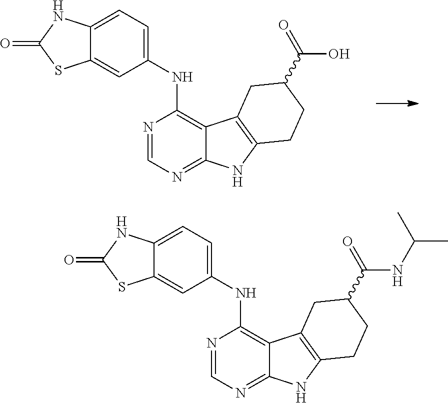 [SMILES: O=C(O)C1CCc2[nH]c3ncnc(Nc4ccc5[nH]c(=O)sc5c4)c3c2C1 ; CC(C)NC(=O)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using propan-2-amine to give after working up and purification 7.7 mg (12%) of the title compound.

¹H-NMR (DMSO-d6): δ=1.05 (6H), 1.76 (1H), 1.94 (1H), 2.45 (1H), 2.58-2.75 (2H), 2.92 (1H), 3.03 (1H), 3.84 (1H), 7.02 (1H), 7.44 (1H), 7.70 (1H), 7.83 (1H), 7.98 (1H), 8.06 (1H), 11.38 (1H), 11.52 (1H) ppm.

### Example 99

### (RS)—N-Ethyl-4-[(2-oxo-2,3-dihydro-1,3-benzothiazol-6-yl)amino]-6,7,8,9-tetrahydro-5H-pyrimido[4,5-b]indole-6-carboxamide

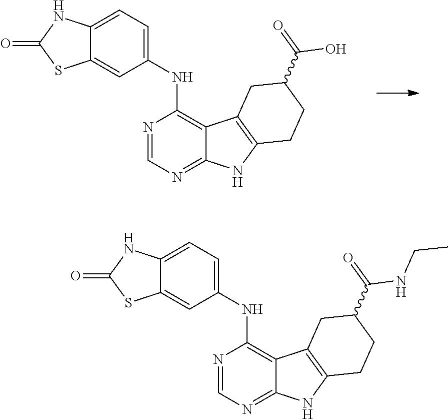 [SMILES: O=C(O)C1CCc2[nH]c3ncnc(Nc4ccc5[nH]c(=O)sc5c4)c3c2C1 ; CCNC(=O)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using ethanamine to give after working up and purification 9.8 mg (15%) of the title compound.

¹H-NMR (DMSO-d6): δ=1.01 (3H), 1.77 (1H), 1.96 (1H), 2.49 (1H), 2.57-2.73 (2H), 2.91 (1H), 3.02-3.14 (3H), 7.02 (1H), 7.45 (1H), 7.81-7.87 (2H), 7.96 (1H), 8.06 (1H), 11.38 (1H), 11.52 (1H) ppm.

### Example 100

### (RS)—N-Methyl-4-[(2-oxo-2,3-dihydro-1,3-benzothiazol-6-yl)amino]-6,7,8,9-tetrahydro-5H-pyrimido[4,5-b]indole-6-carboxamide

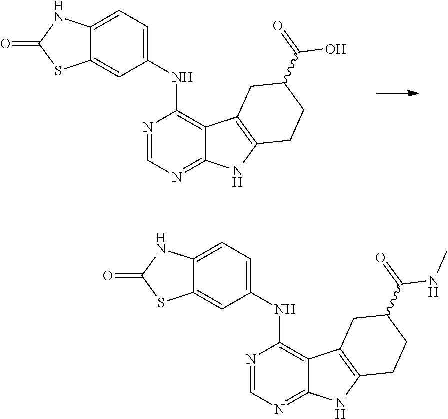 [SMILES: O=C(O)C1CCc2[nH]c3ncnc(Nc4ccc5[nH]c(=O)sc5c4)c3c2C1 ; CNC(=O)C1CCc2[nH]c3ncnc(Nc4ccc5[nH]c(=O)sc5c4)c3c2C1]

60 mg (157 μmol) (RS)-4-[(2-Oxo-2,3-dihydro-1,3-benzothiazol-6-yl)amino]-6,7,8,9-tetrahydro-5H-pyrimido[4,5-b]indole-6-carboxylic acid which was prepared according to example 88 were transformed in analogy to example 14 using methanamine to give after working up and purification 7.3 mg (12%) of the title compound.

¹H-NMR (DMSO-d6): δ=1.77 (1H), 1.97 (1H), 2.49 (1H), 2.56-2.74 (2H), 2.59 (3H), 2.91 (1H), 3.09 (1H), 7.02 (1H), 7.46 (1H), 7.80 (1H), 7.85 (1H), 7.94 (1H), 8.06 (1H), 11.38 (2H) ppm.

### Example 101

### (RS)-Ethyl 4-{[2-methoxy-5-(trifluoromethyl)phenyl]amino}-6,7,8,9-tetrahydro-5H-pyrimido[4,5-b]indole-6-carboxylate

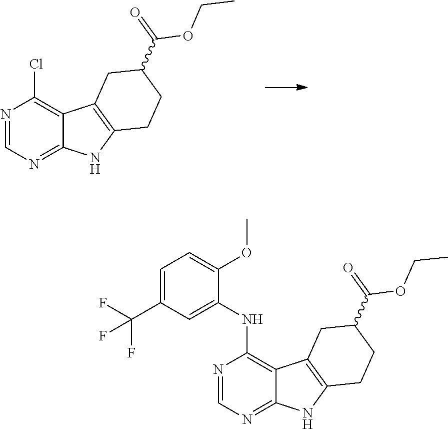 [SMILES: CCOC(=O)C1CCc2[nH]c3ncnc(Cl)c3c2C1 ; CCOC(=O)C1CCc2[nH]c3ncnc(Nc4cc(C(F)(F)F)ccc4OC)c3c2C1]

1.00 g (3.58 mmol) ethyl 4-chloro-6,7,8,9-tetrahydro-5H-pyrimido[4,5-b]indole-6-carboxylate which was prepared according to intermediate example 5a were transformed in analogy to example 1 using 2-methoxy-5-(trifluoromethyl)aniline to give after working up and purification 964 mg (62%) of the title compound.

¹H-NMR (DMSO-d6): δ=1.20 (3H), 1.84 (1H), 2.18 (1H), 2.69-2.88 (3H), 2.96 (1H), 3.17 (1H), 3.89 (3H), 4.05-4.17 (2H), 7.32 (1H), 7.59 (1H), 8.23 (1H), 8.36 (1H), 8.91 (1H), 12.25 (1H) ppm.

### Example 102

### (RS)-4-[(4-Chloro-2-methoxybenzyl)amino]-N-[3-(methylsulfonyl)propyl]-6,7,8,9-tetrahydro-5H-pyrimido[4,5-b]indole-6-carboxamide

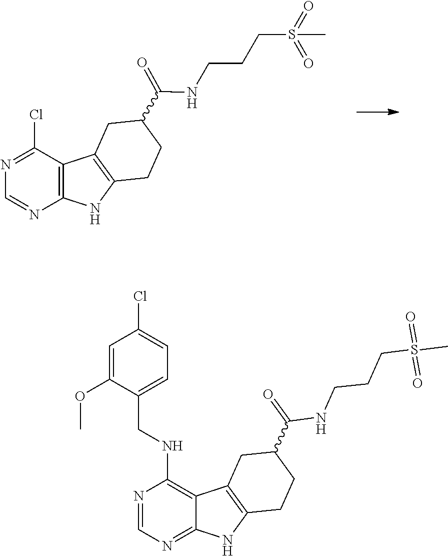 [SMILES: CS(=O)(=O)CCCNC(=O)C1CCc2[nH]c3ncnc(Cl)c3c2C1 ; COc1cc(Cl)ccc1CNc1ncnc2[nH]c3c(c12)CC(C(=O)NCCCS(C)(=O)=O)CC3]

30 mg (81 μmol) (6RS)-4-chloro-N-[3-(methylsulfonyl)propyl]-6,7,8,9-tetrahydro-5H-pyrimido[4,5-b]indole-6-carboxamide which was prepared according to intermediate example 28a were transformed in analogy to example 1 using 1-(4-chloro-2-methoxyphenyl)methanamine to give after working up and purification 18.0 mg (44%) of the title compound.

¹H-NMR (DMSO-d6): δ=1.70-1.87 (3H), 1.98 (1H), 2.51 (1H), 2.63 (2H), 2.85 (1H), 2.93 (3H), 2.99-3.24 (5H), 3.83 (3H), 4.55 (2H), 6.68 (1H), 6.87 (1H), 7.01 (1H), 7.06 (1H), 7.89 (1H), 7.99 (1H), 11.18 (1H) ppm.

### Example 103

### (RS)-4-{[2-Methoxy-5-(trifluoromethyl)phenyl]amino}-6,7,8,9-tetrahydro-5H-pyrimido[4,5-b]indole-6-carboxylic acid

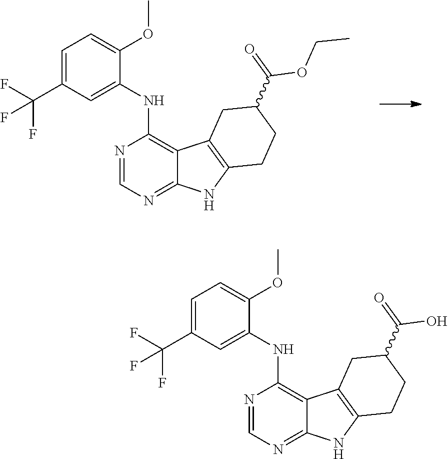 [SMILES: CCOC(=O)C1CCc2[nH]c3ncnc(Nc4cc(C(F)(F)F)ccc4OC)c3c2C1 ; COc1ccc(C(F)(F)F)cc1Nc1ncnc2[nH]c3c(c12)CC(C(=O)O)CC3]

938 mg (2.16 mmol) (RS)-ethyl 4-{[2-methoxy-5-(trifluoromethyl)phenyl]amino}-6,7,8,9-tetrahydro-5H-pyrimido[4,5-b]indole-6-carboxylate which was prepared according to example 101 were transformed in analogy to example 6 to give after working up and purification 785 mg (85%) of the title compound.

¹H-NMR (DMSO-d6): δ=1.83 (1H), 2.16 (1H), 2.70-2.82 (3H), 2.96 (1H), 3.18 (1H), 3.88 (3H), 7.33 (1H), 7.60 (1H), 8.22 (1H), 8.28 (1H), 9.01 (1H), 12.29 (1H) ppm.

### Example 104

### (RS)—N-Cyclopropyl-4-{[2-methoxy-5-(trifluoromethyl)phenyl]amino}-6,7,8,9-tetrahydro-5H-pyrimido[4,5-b]indole-6-carboxamide

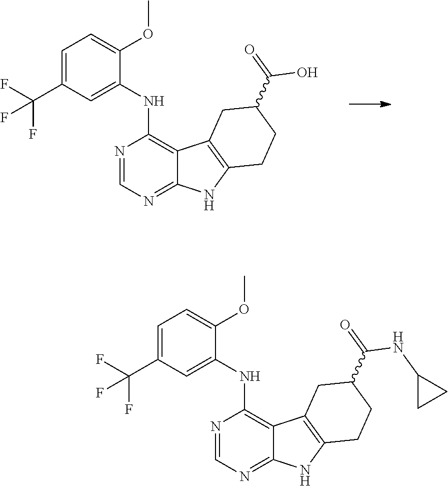 [SMILES: COc1ccc(C(F)(F)F)cc1Nc1ncnc2[nH]c3c(c12)CC(C(=O)O)CC3 ; COc1ccc(C(F)(F)F)cc1Nc1ncnc2[nH]c3c(c12)CC(C(=O)NC1CC1)CC3]

60 mg (148 μmol) (RS)-4-{[2-methoxy-5-(trifluoromethyl)phenyl]amino}-6,7,8,9-tetrahydro-5H-pyrimido[4,5-b]indole-6-carboxylic acid which was prepared according to example 103 were transformed in analogy to example 14 using cyclopropanamine to give after working up and purification 20.6 mg (31%) of the title compound.

¹H-NMR (DMSO-d6): δ=0.39 (2H), 0.62 (2H), 1.76 (1H), 2.00 (1H), 2.49-2.75 (4H), 2.86-3.10 (2H), 3.94 (3H), 7.21 (1H), 7.33 (1H), 7.82 (1H), 8.00 (1H), 8.25 (1H), 8.96 (1H), 11.58 (1H) ppm.

### Example 105

### (RS)—N-Ethyl-4-{[2-methoxy-5-(trifluoromethyl)phenyl]amino}-6,7,8,9-tetrahydro-5H-pyrimido[4,5-b]indole-6-carboxamide

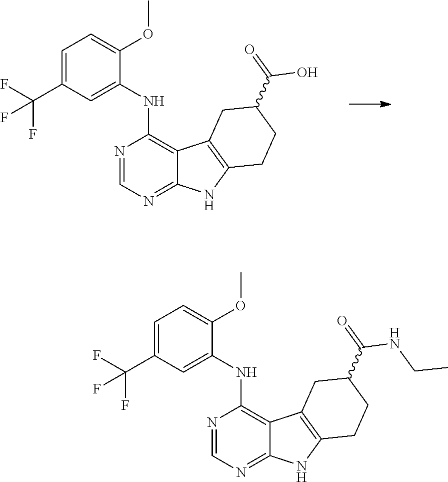 [SMILES: COc1ccc(C(F)(F)F)cc1Nc1ncnc2[nH]c3c(c12)CC(C(=O)O)CC3 ; CCNC(=O)C1CCc2[nH]c3ncnc(Nc4cc(C(F)(F)F)ccc4OC)c3c2C1]

60 mg (148 μmol) (RS)-4-{[2-methoxy-5-(trifluoromethyl)phenyl]amino}-6,7,8,9-tetrahydro-5H-pyrimido[4,5-b]indole-6-carboxylic acid which was prepared according to example 103 were transformed in analogy to example 14 using ethanamine to give after working up and purification 39.9 mg (59%) of the title compound.

¹H-NMR (DMSO-d6): δ=1.03 (3H), 1.80 (1H), 2.01 (1H), 2.57 (1H), 2.69 (2H), 2.91-3.19 (4H), 3.93 (3H), 7.21 (1H), 7.32 (1H), 7.83 (1H), 7.94 (1H), 8.25 (1H), 8.97 (1H), 11.59 (1H) ppm.

### Example 106

### (RS)-4-{[2-Methoxy-5-(trifluoromethyl)phenyl]amino}-N-methyl-6,7,8,9-tetrahydro-5H-pyrimido[4,5-b]indole-6-carboxamide

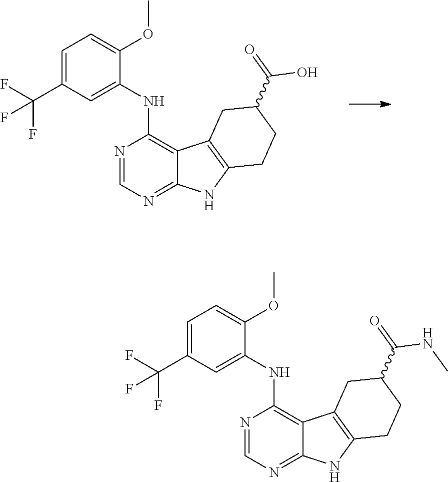 [SMILES: COc1ccc(C(F)(F)F)cc1Nc1ncnc2[nH]c3c(c12)CC(C(=O)O)CC3 ; CNC(=O)C1CCc2[nH]c3ncnc(Nc4cc(C(F)(F)F)ccc4OC)c3c2C1]

60 mg (148 μmol) (RS)-4-{[2-methoxy-5-(trifluoromethyl)phenyl]amino}-6,7,8,9-tetrahydro-5H-pyrimido[4,5-b]indole-6-carboxylic acid which was prepared according to example 103 were transformed in analogy to example 14 using methanamine to give after working up and purification 32.7 mg (50%) of the title compound.

¹H-NMR (DMSO-d6): δ=1.79 (1H), 2.02 (1H), 2.49-2.74 (3H), 2.62 (3H), 2.91-3.10 (2H), 3.93 (3H), 7.21 (1H), 7.33 (1H), 7.83 (1H), 7.88 (1H), 8.24 (1H), 8.94 (1H), 11.58 (1H) ppm.

### Example 107

### (RS)-4-{[2-Methoxy-5-(trifluoromethyl)phenyl]amino}-N,N-dimethyl-6,7,8,9-tetrahydro-5H-pyrimido[4,5-b]indole-6-carboxamide

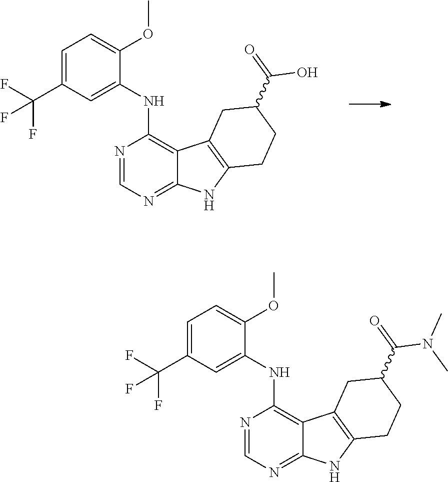 [SMILES: COc1ccc(C(F)(F)F)cc1Nc1ncnc2[nH]c3c(c12)CC(C(=O)O)CC3 ; COc1ccc(C(F)(F)F)cc1Nc1ncnc2[nH]c3c(c12)CC(C(=O)N(C)C)CC3]

60 mg (148 μmol) (RS)-4-{[2-methoxy-5-(trifluoromethyl)phenyl]amino}-6,7,8,9-tetrahydro-5H-pyrimido[4,5-b]indole-6-carboxylic acid which was prepared according to example 103 were transformed in analogy to example 14 using N-methylmethanamine to give after working up and purification 16.3 mg (25%) of the title compound.

¹H-NMR (DMSO-d6): δ=1.75 (1H), 1.98 (1H), 2.62-2.81 (2H), 2.87 (3H), 2.93-3.17 (3H), 3.08 (3H), 3.92 (3H), 7.21 (1H), 7.32 (1H), 7.83 (1H), 8.24 (1H), 8.95 (1H), 11.59 (1H) ppm.

### Examples 108-130

Compounds of examples 108-130 were prepared in analogy to example 1 from 4-chloro-6,7,8,9-tetrahydro-5H-pyrimido[4,5-b]indole (prepared according to intermediate example 1a) as starting material and the reagent given.

### Example 108

### N-(5-Fluoro-2-methoxyphenyl)-6,7,8,9-tetrahydro-5H-pyrimido[4,5-(B]indol-4-amine

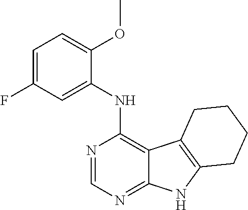 [SMILES: COc1ccc(F)cc1Nc1ncnc2[nH]c3c(c12)CCCC3]

Starting material: Reagent: Product:

40 mg (193 μmol) 5-fluoro-2-methoxyaniline 47.3 mg (75%)

¹H-NMR (DMSO-d6): δ=1.79 (4H), 2.66 (2H), 2.82 (2H), 3.80 (3H), 7.06 (1H), 7.15 (1H), 7.89 (1H), 8.22 (1H), 8.85 (1H), 12.23 (1H) ppm.

### Example 109

### 4-Fluoro-5-nitro-N-(6,7,8,9-tetrahydro-5H-pyrimido[4,5-(B]indol-4-yl)(Benzene-1,2-diamine

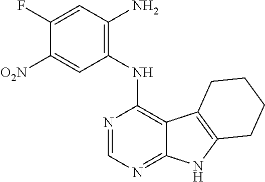 [SMILES: Nc1cc(F)c([N+](=O)[O-])cc1Nc1ncnc2[nH]c3c(c12)CCCC3]

Starting material: Reagent: Product:

100 mg (482 μmol) 4-fluoro-5-nitrobenzene-1,2-diamine 33.1 mg (20%)

¹H-NMR (DMSO-d6): δ=1.76 (4H), 2.61 (2H), 2.85 (2H), 6.55 (1H), 6.69 (2H), 7.45 (1H), 7.92 (1H), 7.96 (1H), 11.29 (1H) ppm.

### Example 110

### 6-(6,7,8,9-Tetrahydro-5H-pyrimido[4,5-(B]indol-4-ylamino)-1,4-dihydroquinoxaline-2,3-dione

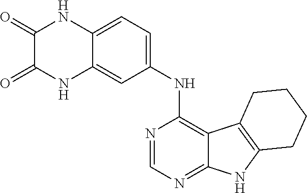 [SMILES: O=c1[nH]c2ccc(Nc3ncnc4[nH]c5c(c34)CCCC5)cc2[nH]c1=O]

Starting material: Reagent: Product:

50 mg (241 μmol) 6-amino-1,4-dihydroquinoxaline-2,3-dione 32.2 mg (38%)

¹H-NMR (DMSO-d6): δ=1.76 (4H), 2.61 (2H), 2.86 (2H), 7.01 (1H), 7.24 (1H), 7.59 (1H), 7.96 (1H), 8.07 (1H), 11.39 (1H), 11.82 (1H), 11.87 (1H) ppm.

### Example 111

### 1-(6,7,8,9-Tetrahydro-5H-pyrimido[4,5-(B]indol-4-yl)-2,3-dihydro-1H-indol-6-amine

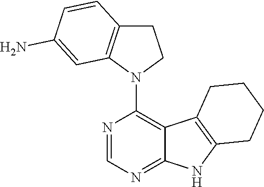 [SMILES: Nc1ccc2c(c1)N(c1ncnc3[nH]c4c(c13)CCCC4)CC2]

Starting material: Reagent: Product:

58 mg (156 μmol) indolin-6-amine 22.3 mg (47%)

¹H-NMR (DMSO-d6): δ=1.62 (2H), 1.77 (2H), 2.53 (2H), 2.66 (2H), 2.86 (2H), 4.00 (2H), 4.75 (2H), 6.01 (1H), 6.33 (1H), 6.80 (1H), 8.22 (1H), 11.53 (1H) ppm.

### Example 112

### N-(6-Methoxy-1H-indazol-5-yl)-6,7,8,9-tetrahydro-5H-pyrimido[4,5-(B]indol-4-amine

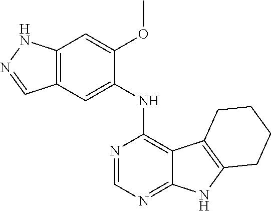 [SMILES: COc1cc2[nH]ncc2cc1Nc1ncnc2[nH]c3c(c12)CCCC3]

Starting material: Reagent: Product:

200 mg (963 μmol) 6-methoxy-1H-indazol-5-amine 167 mg (52%)

¹H-NMR (DMSO-d6): δ=1.82 (4H), 2.63 (2H), 2.89 (2H), 3.97 (3H), 7.04 (1H), 7.73 (1H), 7.94 (1H), 8.21 (1H), 8.89 (1H), 11.40 (1H), 12.73 (1H) ppm.

### Example 113

### 1-(6,7,8,9-Tetrahydro-5H-pyrimido[4,5-(B]indol-4-yl)-2,3-dihydro-1H-indol-5-amine

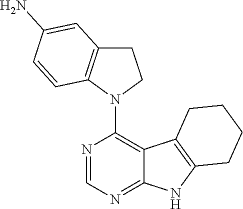 [SMILES: Nc1ccc2c(c1)CCN2c1ncnc2[nH]c3c(c12)CCCC3]

Starting material: Reagent: Product:

25 mg (75 μmol) indolin-5-amine 2.7 mg (11%)

¹H-NMR (DMSO-d6): δ=1.59-1.70 (2H), 1.70-1.81 (2H), 2.59 (2H), 2.64 (2H), 2.93 (2H), 4.03 (2H), 4.67 (2H), 6.28 (1H), 6.47 (1H), 7.08 (1H), 8.08 (1H), 11.43 (1H) ppm.

### Example 114

### N-(3,4-Dichlorophenyl)-6,7,8,9-tetrahydro-5H-pyrimido[4,5-(B]indol-4-amine

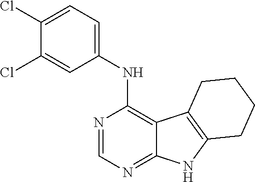 [SMILES: Clc1ccc(Nc2ncnc3[nH]c4c(c23)CCCC4)cc1Cl]

Starting material: Reagent: Product:

50 mg (241 μmol) 3,4-dichloroaniline 30.4 mg (36%)

¹H-NMR (DMSO-d6): δ=1.77 (4H), 2.63 (2H), 2.89 (2H), 7.49 (1H), 7.73 (1H), 8.08 (2H), 8.18 (1H), 11.47 (1H) ppm.

### Example 115

### 5-(6,7,8,9-Tetrahydro-5H-pyrimido[4,5-(B]indol-4-ylamino)-1,3-dihydro-2H-(Benzimidazol-2-one

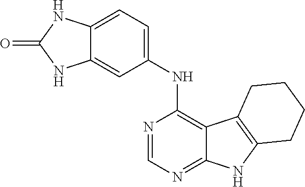 [SMILES: O=c1[nH]c2ccc(Nc3ncnc4[nH]c5c(c34)CCCC5)cc2[nH]1]

Starting material: Reagent: Product:

50 mg (241 μmol) 5-amino-1,3-dihydro-2H-benzimidazo-2- 9.7 mg (12%) one

¹H-NMR (DMSO-d6): δ=1.75 (4H), 2.60 (2H), 2.86 (2H), 6.80 (1H), 7.08 (1H), 7.42 (1H), 7.66 (1H), 8.04 (1H), 10.49 (1H), 10.43 (1H), 11.30 (1H) ppm.

### Example 116

### N-[2-(Cyclopentyloxy)phenyl]-6,7,8,9-tetrahydro-5H-pyrimido[4,5-(B]indol-4-amine

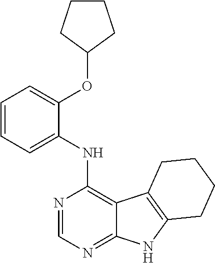 [SMILES: c1ccc(OC2CCCC2)c(Nc2ncnc3[nH]c4c(c23)CCCC4)c1]

Starting material: Reagent: Product:

40 mg (193 μmol) 5-amino-1,3-dihydro-2H-benzimidazo-2-one 53.2 mg (75%)

¹H-NMR (DMSO-d6): δ=1.31-1.65 (6H), 1.71-1.86 (6H), 2.66 (2H), 2.78 (2H), 4.87 (1H), 7.01 (1H), 7.12 (1H), 7.25 (1H), 7.79 (1H), 8.16 (1H), 9.04 (1H), 12.29 (1H) ppm.

### Example 117

### 6-(6,7,8,9-Tetrahydro-5H-pyrimido[4,5-(B]indol-4-ylamino)-1,3-(Benzothiazol-2(3H)-one

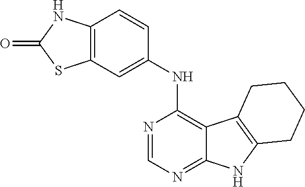 [SMILES: O=c1[nH]c2ccc(Nc3ncnc4[nH]c5c(c34)CCCC5)cc2s1]

Starting material: Reagent: Product:

100 mg (482 μmol) 6-amino-1,3-benzothiazol-2(3H)-one 159 mg (93%)

¹H-NMR (DMSO-d6): δ=1.68-1.88 (4H), 2.66 (2H), 2.82 (2H), 7.23 (1H), 7.35 (1H), 7.72 (1H), 8.12 (1H), 9.69 (1H), 12.20 (1H), 12.53 (1H) ppm.

### Example 118

### N-[2-(Morpholin-4-yl)phenyl]-6,7,8,9-tetrahydro-5H-pyrimido[4,5-(B]indol-4-amine

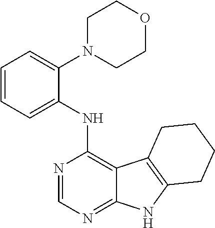 [SMILES: c1ccc(N2CCOCC2)c(Nc2ncnc3[nH]c4c(c23)CCCC4)c1]

Starting material: Reagent: Product:

40 mg (193 μmol) 2-(morpholin-4-yl)aniline 40.4 mg (57%)

¹H-NMR (DMSO-d6): δ=1.81 (4H), 2.67 (2H), 2.77-2.96 (8H), 3.74 (2H), 7.02 (1H), 7.12-7.24 (2H), 7.29 (1H), 8.19 (1H), 9.18 (1H), 12.15 (1H) ppm.

### Example 119

### N-[2-Methoxy-5-(trifluoromethyl)phenyl]-6,7,8,9-tetrahydro-5H-pyrimido[4,5-(B]indol-4-amine

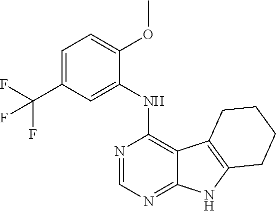 [SMILES: COc1ccc(C(F)(F)F)cc1Nc1ncnc2[nH]c3c(c12)CCCC3]

Starting material: Reagent: Product:

40 mg (193 μmol) 2-methoxy-5-(trifluoromethyl)aniline 52.8 mg (72%)

¹H-NMR (DMSO-d6): δ=2.67 (4H), 2.84 (4H), 3.87 (3H), 7.34 (1H), 7.64 (1H), 8.22 (2H), 9.06 (1H), 12.33 (1H) ppm.

### Example 120

### 4-(2,3-Dihydro-1H-indol-1-yl)-6,7,8,9-tetrahydro-5H-pyrimido[4,5-(B]indole

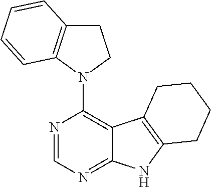 [SMILES: c1ccc2c(c1)CCN2c1ncnc2[nH]c3c(c12)CCCC3]

Starting material: Reagent: Product:

100 mg (482 μmol) indoline 64.6 mg (44%)

¹H-NMR (DMSO-d6): δ=1.62 (2H), 1.76 (2H), 2.54 (2H), 2.66 (2H), 3.06 (2H), 4.10 (2H), 6.79 (1H), 6.99 (1H), 7.09 (1H), 7.18 (1H), 8.22 (1H), 11.59 (1H) ppm.

### Example 121

### 4-(6-Nitro-2,3-dihydro-1H-indol-1-yl)-6,7,8,9-tetrahydro-5H-pyrimido[4,5-(B]indole hydrochloride (1:1)

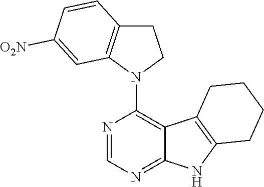 [SMILES: O=[N+]([O-])c1ccc2c(c1)N(c1ncnc3[nH]c4c(c13)CCCC4)CC2]

Starting material: Reagent: Product:

100 mg (482 μmol) 6-nitroindoline 153.7 mg (95%)

¹H-NMR (DMSO-d6): δ=1.64 (2H), 1.77 (2H), 2.56 (2H), 2.72 (2H), 3.27 (2H), 4.34 (2H), 7.49 (1H), 7.79 (1H), 8.08 (1H), 8.43 (1H), 12.31 (1H) ppm.

### Example 122

### 4-(5-Chloro-2,3-dihydro-1H-indol-1-yl)-6,7,8,9-tetrahydro-5H-pyrimido[4,5-(B]indole hydrochloride (1:1)

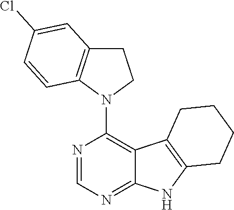 [SMILES: Clc1ccc2c(c1)CCN2c1ncnc2[nH]c3c(c12)CCCC3]

Starting material: Reagent: Product:

100 mg (482 μmol) 5-chloroindoline 142 mg (82%)

¹H-NMR (DMSO-d6): δ=1.63 (2H), 1.77 (2H), 2.53 (2H), 2.70 (2H), 3.15 (2H), 4.29 (2H), 7.17 (1H), 7.34 (2H), 8.39 (1H), 12.47 (1H) ppm.

### Example 123

### 4-(5-Nitro-2,3-dihydro-1H-indol-1-yl)-6,7,8,9-tetrahydro-5H-pyrimido[4,5-(B]indole

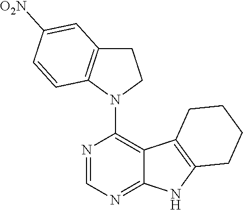 [SMILES: O=[N+]([O-])c1ccc2c(c1)CCN2c1ncnc2[nH]c3c(c12)CCCC3]

Starting material: Reagent: Product:

100 mg (482 μmol) 5-nitroindoline 10.6 mg (6%)

¹H-NMR (DMSO-d6): δ=1.62 (2H), 1.77 (2H), 2.50 (2H), 2.69 (2H), 3.21 (2H), 4.26 (2H), 6.99 (1H), 7.97 (1H), 8.04 (1H), 8.37 (1H), 11.84 (1H) ppm.

### Example 124

### N-[5-Chloro-2-(propan-2-yloxy)phenyl]-6,7,8,9-tetrahydro-5H-pyrimido[4,5-(B]indol-4-amine

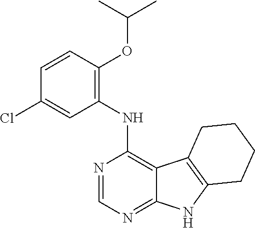 [SMILES: CC(C)Oc1ccc(Cl)cc1Nc1ncnc2[nH]c3c(c12)CCCC3]

Starting material: Reagent: Product:

50 mg (241 μmol) 5-chloro-2-isopropoxyaniline 2.9 mg (3%)

¹H-NMR (DMSO-d6): δ=1.32 (6H), 1.82 (4H), 2.84 (2H), 2.90 (2H), 4.74 (1H), 6.93 (1H), 7.06 (1H), 7.83 (1H), 8.26 (1H), 8.89 (1H), 11.52 (1H) ppm.

### Example 125

### N-(5-Chloro-2-methoxyphenyl)-6,7,8,9-tetrahydro-5H-pyrimido[4,5-(B]indol-4-amine

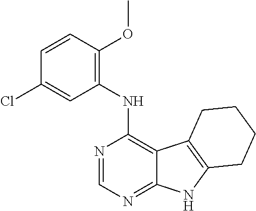 [SMILES: COc1ccc(Cl)cc1Nc1ncnc2[nH]c3c(c12)CCCC3]

Starting material: Reagent: Product:

100 mg (482 μmol) 5-chloro-2-methoxyaniline 113.5 mg (68%)

¹H-NMR (DMSO-d6): δ=1.81 (4H), 2.63 (2H), 2.85 (2H), 3.90 (3H), 6.97 (1H), 7.04 (1H), 7.71 (1H), 8.25 (1H), 8.79 (1H), 11.53 (1H) ppm.

### Example 126

### N-[6-(Propan-2-yloxy)-1H-indazol-5-yl]-6,7,8,9-tetrahydro-5H-pyrimido[4,5-(B]indol-4-amine hydrochloride (1:1)

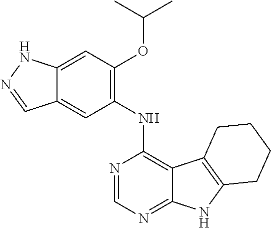 [SMILES: CC(C)Oc1cc2[nH]ncc2cc1Nc1ncnc2[nH]c3c(c12)CCCC3]

Starting material: Reagent: Product:

68.8 mg (331 μmol) 6-isopropoxy-1H-indazol-5-amine 94.2 mg (68%)

¹H-NMR (DMSO-d6): δ=1.15 (6H), 1.79 (4H), 2.68 (2H), 2.84 (2H), 4.68 (1H), 7.14 (1H), 7.87-8.24 (1H), 8.03 (1H), 8.12 (1H), 9.37 (1H), 12.46 (1H), 13.02 (1H) ppm.

### Example 127

### N-[5-(Bromo-2-(propan-2-yloxy)phenyl]-6,7,8,9-tetrahydro-5H-pyrimido[4,5-(B]indol-4-amine

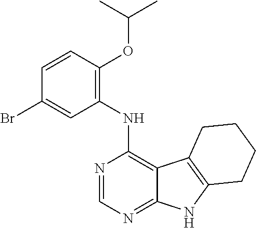 [SMILES: CC(C)Oc1ccc(Br)cc1Nc1ncnc2[nH]c3c(c12)CCCC3]

Starting material: Reagent: Product:

100 mg (482 μmol) 5-bromo-2-isopropoxyaniline 127 mg (62%)

¹H-NMR (DMSO-d6): δ=1.32 (6H), 1.82 (4H), 2.64 (2H), 2.90 (2H), 4.73 (1H), 7.01 (1H), 7.06 (1H), 7.82 (1H), 8.26 (1H), 9.01 (1H), 11.52 (1H) ppm.

### Example 128

### N-(5-(Bromo-2-methoxyphenyl)-6,7,8,9-tetrahydro-5H-pyrimido[4,5-(B]indol-4-amine

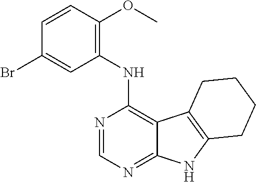 [SMILES: COc1ccc(Br)cc1Nc1ncnc2[nH]c3c(c12)CCCC3]

Starting material: Reagent: Product:

50 mg (241 μmol) 5-bromo-2-methoxyaniline 4.9 mg (4%)

¹H-NMR (DMSO-d6): δ=1.81 (4H), 2.63 (2H), 2.86 (2H), 3.90 (3H), 7.00 (1H), 7.10 (1H), 7.70 (1H), 8.25 (1H), 8.91 (1H), 11.51 (1H) ppm.

### Example 129

### N-[3-Methoxy-4-(6,7,8,9-tetrahydro-5H-pyrimido[4,5-(B]indol-4-ylamino)phenyl]methanesulfonamide

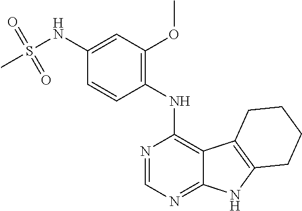 [SMILES: COc1cc(NS(C)(=O)=O)ccc1Nc1ncnc2[nH]c3c(c12)CCCC3]

Starting material: Reagent: Product:

100 mg (482 μmol) N-(4-amino-3-methoxyphenyl)methanesulfonamide 149.3 mg (76%)

¹H-NMR (DMSO-d6): δ=1.81 (4H), 2.62 (2H), 2.85 (2H), 2.92 (3H), 3.27 (3H), 6.81 (1H), 6.90 (1H), 7.54 (1H), 8.14 (1H), 8.48 (1H), 9.45 (1H), 11.40 (1H) ppm.

### Example 130

### N-[2-(tetrahydro-2H-pyran-4-yloxy)phenyl]-6,7,8,9-tetrahydro-5H-pyrimido[4,5-b]indol-4-amine N-[3-Methoxy-4-(6,7,8,9-tetrahydro-5H-pyrimido[4,5-(B]indol-4-ylamino)phenyl]methanesulfonamide

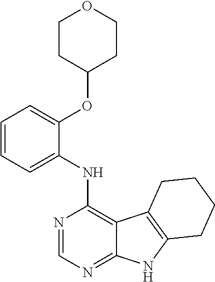 [SMILES: c1ccc(OC2CCOCC2)c(Nc2ncnc3[nH]c4c(c23)CCCC4)c1]

Starting material: Reagent: Product:

40 mg (193 μmol) 2-(tetrahydro-2H-pyran-4-yloxy)aniline 47.2 mg (64%)

¹H-NMR (DMSO-d6): δ=1.44 (2H), 1.76-1.96 (6H), 2.71 (2H), 2.83 (2H), 3.38-3.60 (4H), 4.68 (1H), 7.08 (1H), 7.24-7.39 (2H), 7.79 (1H), 8.21 (1H), 9.23 (1H), 12.42 (1H) ppm.

### Example 131

### (RS)-5-Chloro-6-{[6-methoxy-6-(methoxymethyl)-6,7,8,9-tetrahydro-5H-pyrimido[4,5-(B]indol-4-yl]amino}-1,3-dihydro-2H-(Benzimidazol-2-one

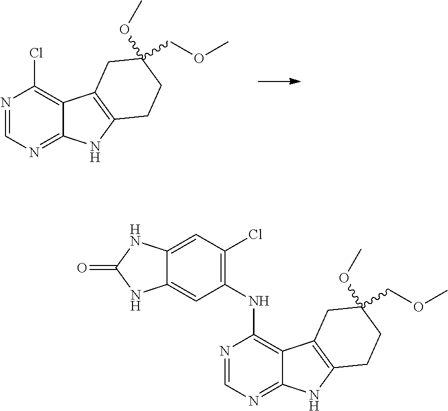

60 mg (213 μmol) (RS)-4-chloro-6-methoxy-6-(methoxymethyl)-6,7,8,9-tetrahydro-5H-pyrimido[4,5-b]indole (prepared according to intermediate example 131a) were transformed in analogy to example 1 using 5-amino-6-chloro-1,3-dihydro-2H-benzimidazol-2-one to give after working up and purification 6.9 mg (7%) of the title compound.

¹H-NMR (DMSO-d6): δ=1.76-1.99 (2H), 2.40-2.67 (4H), 2.85 (1H), 3.00 (1H), 3.16 (3H), 3.41 (3H), 7.00 (1H), 7.64 (1H), 7.78 (1H), 8.05 (1H), 10.66 (2H), 11.42 (1H) ppm.

### Example 131a

### (RS)-4-Chloro-6-methoxy-6-(methoxymethyl)-6,7,8,9-tetrahydro-5H-pyrimido[4,5-b]indole

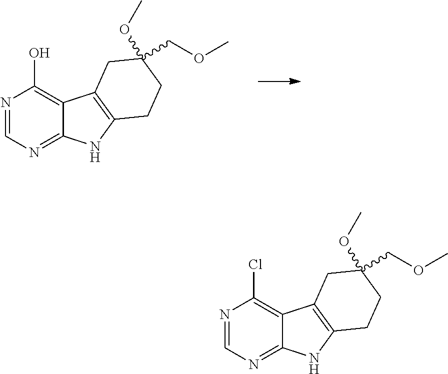

250 mg (949 mmol) (RS)-6-methoxy-6-(methoxymethyl)-6,7,8,9-tetrahydro-5H-pyrimido[4,5-b]indol-4-ol (prepared according to intermediate example 131b) were transformed in analogy to intermediate example 1a to give after working up and purification 170.3 mg (60%) of the title compound.

### Example 131b

### (RS)-6-Methoxy-6-(methoxymethyl)-6,7,8,9-tetrahydro-5H-pyrimido[4,5-b]indol-4-ol

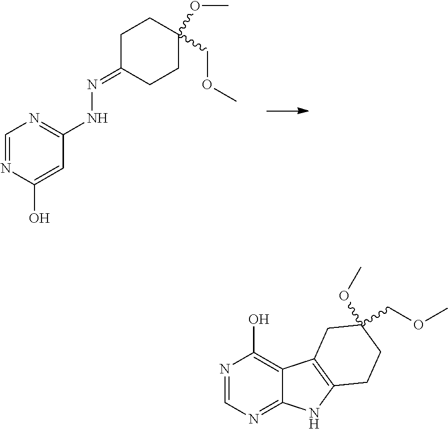

300 mg (1.07 mmol) (RS)-6-{2-[4-methoxy-4-(methoxymethyl)cyclohexylidene]hydrazino}pyrimidin-4-ol (prepared according to intermediate example 131c) were transformed in analogy to intermediate example 1b to give after working up and purification 254.9 mg (86%) of the title compound.

### Example 131c

### (RS)-6-{2-[4-Methoxy-4-(methoxymethyl)cyclohexylidene]hydrazino}pyrimidin-4-ol

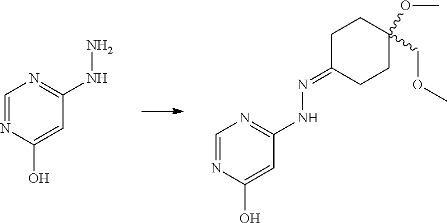

3.00 g (23.79 mmol) 6-hydrazinopyrimidin-4-ol/6-hydrazinopyrimidin-4(1H)-one (CAS-No: 29939-37-5) were transformed in analogy to intermediate example 1c using 4-methoxy-4-(methoxymethyl)cyclohexanone to give after working up and purification 4.46 g (64%) of the title compound.

### Examples 132-140

Compounds of examples 132-140 were prepared in analogy to example 1 from (RS)-4-chloro-6-methoxy-6-(methoxymethyl)-6,7,8,9-tetrahydro-5H-pyrimido[4,5-b]indole (prepared according to intermediate example 131a) as starting material and the reagent given.

### Example 132

### (RS)—N-[5-Chloro-2-(propan-2-yloxy)phenyl]-6-methoxy-6-(methoxymethyl)-6,7,8,9-tetrahydro-5H-pyrimido[4,5-(B]indol-4-amine

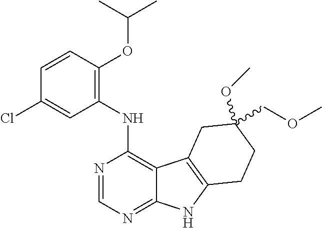

Starting material: Reagent: Product:

60 mg (213 μmol) 5-chloro-2-isopropoxyaniline 23.3 mg (24%)

¹H-NMR (DMSO-d6): δ=1.34 (6H), 1.78 (1H), 2.00 (1H), 2.54-2.71 (2H), 2.93 (1H), 3.03 (1H), 3.17 (3H), 3.29 (3H), 3.42 (1H), 3.49 (1H), 4.75 (1H), 6.93 (1H), 7.08 (1H), 7.76 (1H), 8.27 (1H), 8.91 (1H), 11.59 (1H) ppm.

### Example 133

### (RS)—N-(5-Chloro-2-methoxyphenyl)-6-methoxy-6-(methoxymethyl)-6,7,8,9-tetrahydro-5H-pyrimido[4,5-(B]indol-4-amine

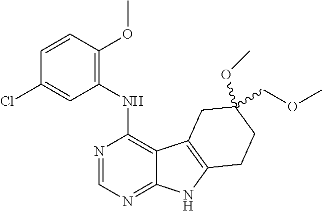

Starting material: Reagent: Product:

60 mg (213 μmol) 5-chloro-2-methoxyaniline 24.9 mg (28%)

¹H-NMR (DMSO-d6): δ=1.74-1.82 (1H), 1.94-2.01 (1H), 2.54-2.69 (2H), 2.91 (1H), 3.01 (1H), 3.18 (3H), 3.33 (3H), 3.43 (1H), 3.50 (1H), 3.90 (3H), 6.97 (1H), 7.05 (1H), 7.76 (1H), 8.25 (1H), 8.76 (1H), 11.56 (1H) ppm.

### Example 134

### (RS)—N-[2-(Cyclopentyloxy)-4-fluorophenyl]-6-methoxy-6-(methoxymethyl)-6,7,8,9-tetrahydro-5H-pyrimido[4,5-(B]indol-4-amine

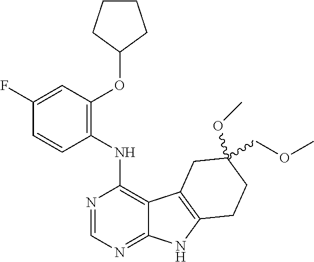

Starting material: Reagent: Product:

60 mg (213 μmol) 2-(cyclopentyloxy)-4-fluoroaniline 38.9 mg (39%)

¹H-NMR (DMSO-d6): δ=1.53-1.87 (7H), 1.91-2.05 (3H), 2.49-2.67 (2H), 2.88 (1H), 2.98 (1H), 3.16 (3H), 3.30 (3H), 3.43 (2H), 4.95 (1H), 6.74 (1H), 6.93 (1H), 7.47 (1H), 8.17 (1H), 8.70 (1H), 11.51 (1H) ppm.

### Example 135

### (RS)—N-(1H-Indazol-5-yl)-6-methoxy-6-(methoxymethyl)-6,7,8,9-tetrahydro-5H-pyrimido[4,5-(B]indol-4-amine

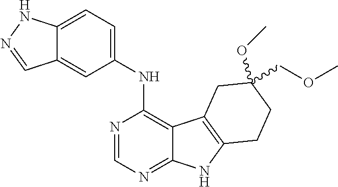

Starting material: Reagent: Product:

40 mg (142 μmol) 1H-indazol-5-amine 50.5 mg (89%)

¹H-NMR (DMSO-d6): δ=1.83 (1H), 2.03 (1H), 2.63-2.77 (2H), 2.82 (1H), 3.06 (1H), 3.19 (3H), 3.31 (3H), 3.43 (2H), 7.42 (1H), 7.71 (1H), 7.93 (1H), 8.10 (1H), 8.18 (1H), 9.92 (1H), 12.59 (1H), 13.36 (1H) ppm.

### Example 136

### (RS)-6-Methoxy-N-(6-methoxy-1H-indazol-5-yl)-6-(methoxymethyl)-6,7,8,9-tetrahydro-5H-pyrimido[4,5-(B]indol-4-amine

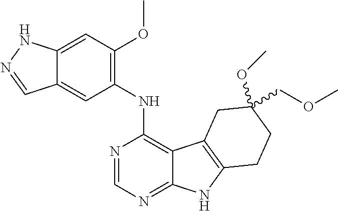

Starting material: Reagent: Product:

40 mg (142 μmol) 6-methoxy-1H-indazol-5-amine 50.8 mg (83%)

¹H-NMR (DMSO-d6): δ=1.83 (1H), 2.04 (1H), 2.64-2.76 (2H), 2.84 (1H), 3.06 (1H), 3.20 (3H), 3.33 (3H), 3.45 (2H), 3.86 (3H), 7.20 (1H), 7.99 (1H), 8.08 (1H), 8.11 (1H), 9.64 (1H), 12.56 (1H), 13.14 (1H) ppm.

### Example 137

### (RS)—N-[4-Fluoro-2-(propan-2-yloxy)phenyl]-6-methoxy-6-(methoxymethyl)-6,7,8,9-tetrahydro-5H-pyrimido[4,5-(B]indol-4-amine

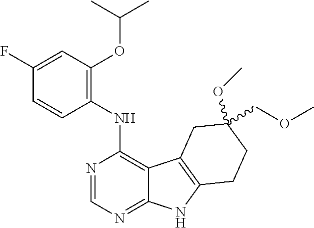

Starting material: Reagent: Product:

40 mg (142 μmol) 4-fluoro-2-isopropoxyaniline 33.3 mg (54%)

¹H-NMR (DMSO-d6): δ=1.17 (6H), 1.83 (1H), 2.03 (1H), 2.63-2.76 (2H), 2.82 (1H), 3.04 (1H), 3.19 (3H), 3.33 (3H), 3.46 (2H), 4.71 (1H), 6.91 (1H), 7.18 (1H), 7.77 (1H), 8.21 (1H), 9.30 (1H), 12.50 (1H) ppm.

### Example 138

### (RS)-6-{[6-Methoxy-6-(methoxymethyl)-6,7,8,9-tetrahydro-5H-pyrimido[4,5-(B]indol-4-yl]amino}-1,3-(Benzothiazol-2(3H)-one

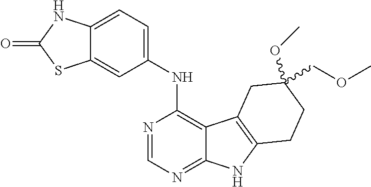

Starting material: Reagent: Product:

40 mg (142 μmol) 6-amino-1,3-benzothiazol-2(3H)-one 53 mg (86%)

¹H-NMR (DMSO-d6): δ=1.83 (1H), 2.02 (1H), 2.63-2.75 (2H), 2.80 (1H), 3.03 (1H), 3.19 (3H), 3.32 (3H), 3.42 (1H), 3.46 (1H), 7.27 (1H), 7.40 (1H), 7.77 (1H), 8.17 (1H), 9.77 (1H), 12.18 (1H), 12.56 (1H) ppm.

### Example 139

### (RS)—N-(6-Fluoro-1H-indazol-5-yl)-6-methoxy-6-(methoxymethyl)-6,7,8,9-tetrahydro-5H-pyrimido[4,5-(B]indol-4-amine

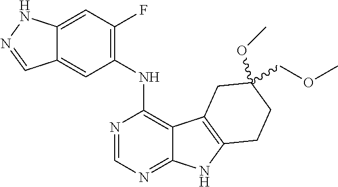

Starting material: Reagent: Product:

60 mg (213 μmol) 6-fluoro-1H-indazol-5-amine 17.7 mg (20%)

¹H-NMR (DMSO-d6): δ=1.74-1.88 (1H), 1.88-1.98 (1H), 2.51-2.69 (2H), 2.85 (1H), 3.01 (1H), 3.17 (3H), 3.28 (3H), 3.38 (1H), 3.44 (1H), 7.37 (1H), 7.89 (1H), 7.98 (1H), 8.03 (1H), 8.04 (1H), 11.36 (1H), 13.03 (1H) ppm.

### Example 140

### (RS)—N-(4,5-Dichloro-2-methoxyphenyl)-6-methoxy-6-(methoxymethyl)-6,7,8,9-tetrahydro-5H-pyrimido[4,5-(B]indol-4-amine

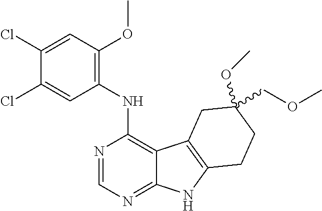

Starting material: Reagent: Product:

60 mg (213 μmol) 4,5-dichloro-2-methoxyaniline 10.2 mg (10%)

¹H-NMR (DMSO-d6): δ=1.77 (1H), 1.97 (1H), 2.53-2.69 (2H), 2.89 (1H), 3.01 (1H), 3.18 (3H), 3.33 (3H), 3.41 (1H), 3.49 (1H), 3.93 (3H), 7.30 (1H), 7.71 (1H), 8.25 (1H), 8.90 (1H), 11.58 (1H) ppm.

### Examples 141-145

Compounds of examples 141-145 were prepared from 4-chloro-6,6-difluoro-6,7,8,9-tetrahydro-5H-pyrimido[4,5-b]indole (prepared according to intermediate example 9a) as starting material in analogy to example 1 and the reagent given.

### Example 141

### 6,6-Difluoro-N-(6-methoxy-1H-indazol-5-yl)-6,7,8,9-tetrahydro-5H-pyrimido[4,5-(B]indol-4-amine

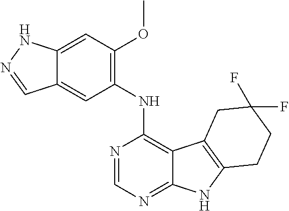 [SMILES: COc1cc2[nH]ncc2cc1Nc1ncnc2[nH]c3c(c12)CC(F)(F)CC3]

Starting material: Reagent: Product:

60 mg (246 μmol) 6-methoxy-1H-indazol-5-amine 85.6 mg (89%)

¹H-NMR (DMSO-d6): δ=2.38 (2H), 2.91 (2H), 3.46 (2H), 3.81 (3H), 7.16 (1H), 7.94 (1H), 8.05 (1H), 8.11 (1H), 9.64 (1H), 12.72 (1H), 13.09 (1H) ppm.

### Example 142

### 6-[(6,6-Difluoro-6,7,8,9-tetrahydro-5H-pyrimido[4,5-(B]indol-4-yl)amino]-1,3-(Benzothiazol-2(3H)-one

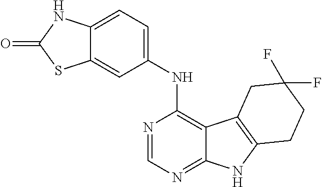 [SMILES: O=c1[nH]c2ccc(Nc3ncnc4[nH]c5c(c34)CC(F)(F)CC5)cc2s1]

Starting material: Reagent: Product:

60 mg (246 μmol) 6-amino-1,3-benzothiazol-2(3H)-one 83.2 mg (86%)

¹H-NMR (DMSO-d6): δ=2.24-2.45 (2H), 2.90 (2H), 3.47 (2H), 7.20 (1H), 7.38 (1H), 7.75 (1H), 8.16 (1H), 9.57 (1H), 12.08 (1H), 12.58 (1H) ppm.

### Example 143

### N-[5-Chloro-2-(propan-2-yloxy)phenyl]-6,6-difluoro-6,7,8,9-tetrahydro-5H-pyrimido[4,5-(B]indol-4-amine

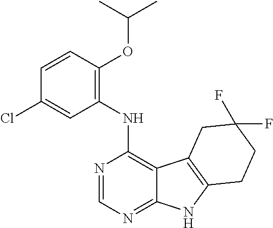 [SMILES: CC(C)Oc1ccc(Cl)cc1Nc1ncnc2[nH]c3c(c12)CC(F)(F)CC3]

Starting material: Reagent: Product:

60 mg (246 μmol) 5-chloro-2-isopropoxyaniline 40.3 mg (40%)

¹H-NMR (DMSO-d6): δ=1.33 (6H), 2.32 (2H), 2.87 (2H), 3.50 (2H), 4.74 (1H), 6.95 (1H), 7.07 (1H), 7.72 (1H), 8.31 (1H), 8.82 (1H), 11.82 (1H) ppm.

### Example 144

### N-(5-Chloro-2-methoxyphenyl)-6,6-difluoro-6,7,8,9-tetrahydro-5H-pyrimido[4,5-(B]indol-4-amine

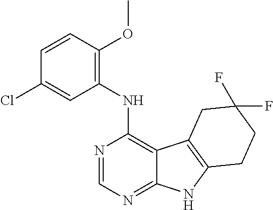 [SMILES: COc1ccc(Cl)cc1Nc1ncnc2[nH]c3c(c12)CC(F)(F)CC3]

Starting material: Reagent: Product:

60 mg (246 μmol) 5-chloro-2-methoxyaniline 20.5 mg (22%)

¹H-NMR (DMSO-d6): δ=2.31 (2H), 2.87 (2H), 3.47 (2H), 3.88 (3H), 7.01 (1H), 7.06 (1H), 7.66 (1H), 8.26 (1H), 8.59 (1H), 11.77 (1H) ppm.

### Example 145

### 6,6-Difluoro-N-[2-methoxy-5-(trifluoromethyl)phenyl]-6,7,8,9-tetrahydro-5H-pyrimido[4,5-(B]indol-4-amine

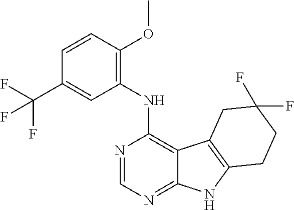 [SMILES: COc1ccc(C(F)(F)F)cc1Nc1ncnc2[nH]c3c(c12)CC(F)(F)CC3]

Starting material: Reagent: Product:

60 mg (246 μmol) 2-methoxy-5-(trifluoromethyl)aniline 31.2 mg (30%)

¹H-NMR (DMSO-d6): δ=2.31 (2H), 2.87 (2H), 3.48 (2H), 3.94 (3H), 7.22 (1H), 7.36 (1H), 7.77 (1H), 8.25 (1H), 8.80 (1H), 11.79 (1H) ppm.

### Examples 146-169

Compounds of examples 146-169 were prepared in analogy to example 1 from (RS)-4-chloro-N-[3-(methylsulfonyl)propyl]-6,7,8,9-tetrahydro-5H-pyrimido[4,5-b]indole-6-carboxamide (prepared according to intermediate example 28a) as starting material and the reagent given.

### Example 146

### (RS)-4-(1H-Indazol-5-ylsulfanyl)-N-[3-(methylsulfonyl)propyl]-6,7,8,9-tetrahydro-5H-pyrimido[4,5-b]indole-6-carboxamide

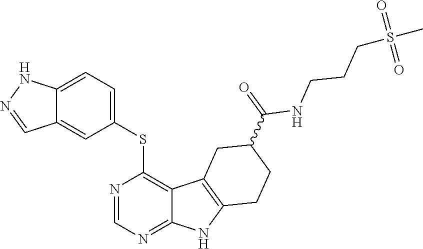 [SMILES: CS(=O)(=O)CCCNC(=O)C1CCc2[nH]c3ncnc(Sc4ccc5[nH]ncc5c4)c3c2C1]

Starting material: Reagent: Product:

60 mg (162 μmol) 1H-indazole-5-thiol 21.7 mg (26%)

¹H-NMR (DMSO-d6): δ=1.73-1.89 (3H), 2.03 (1H), 2.57 (1H), 2.78 (2H), 2.90 (1H), 2.94 (3H), 3.05-3.27 (5H), 7.42 (1H), 7.58 (1H), 8.00 (1H), 8.05 (1H), 8.09 (1H), 8.17 (1H), 11.81 (1H), 13.26 (1H) ppm.

### Example 147

### (RS)-4-[(4-Fluoro-2-methoxyphenyl)sulfanyl]-N-[3-(methylsulfonyl)propyl]-6,7,8,9-tetrahydro-5H-pyrimido[4,5-b]indole-6-carboxamide

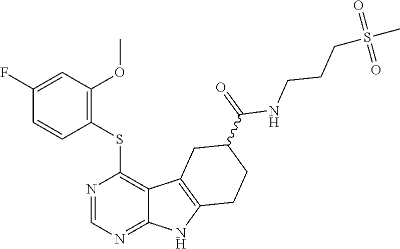 [SMILES: COc1cc(F)ccc1Sc1ncnc2[nH]c3c(c12)CC(C(=O)NCCCS(C)(=O)=O)CC3]

Starting material: Reagent: Product:

60 mg (162 μmol) 4-fluoro-2-methoxybenzenethio 7.9 mg (9%)

¹H-NMR (DMSO-d6): δ=1.75-1.90 (3H), 2.03 (1H), 2.56 (1H), 2.72 (2H), 2.86 (1H), 2.93 (3H), 3.04-3.24 (5H), 3.70 (3H), 6.83 (1H), 7.03 (1H), 7.51 (1H), 8.03 (1H), 8.18 (1H), 11.76 (1H) ppm.

### Example 148

### (RS)-4-[(5-Chloro-2-methoxyphenyl)sulfanyl]-N-[3-(methylsulfonyl)propyl]-6,7,8,9-tetrahydro-5H-pyrimido[4,5-b]indole-6-carboxamide

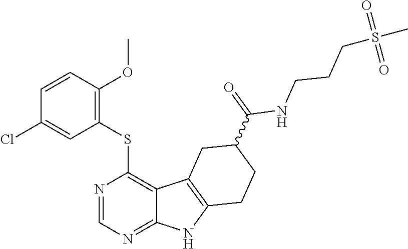 [SMILES: COc1ccc(Cl)cc1Sc1ncnc2[nH]c3c(c12)CC(C(=O)NCCCS(C)(=O)=O)CC3]

Starting material: Reagent: Product:

60 mg (162 μmol) 4-chloro-2-methoxybenzenethiol 12.6 mg (15%)

¹H-NMR (DMSO-d6): δ=1.74-1.90 (3H), 2.03 (1H), 2.56 (1H), 2.73 (2H), 2.85 (1H), 2.93 (3H), 3.02-3.23 (5H), 3.70 (3H), 7.13 (1H), 7.47 (1H), 7.51 (1H), 8.03 (1H), 8.24 (1H), 11.82 (1H) ppm.

### Example 149

### (RS)-4-[(5-Chloro-2-methoxyphenyl)amino]-N-[3-(methylsulfonyl)propyl]-6,7,8,9-tetrahydro-5H-pyrimido[4,5-b]indole-6-carboxamide

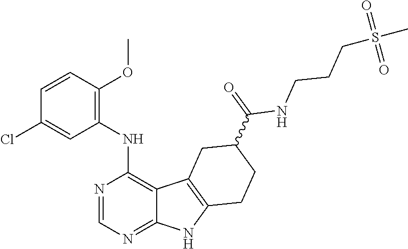 [SMILES: COc1ccc(Cl)cc1Nc1ncnc2[nH]c3c(c12)CC(C(=O)NCCCS(C)(=O)=O)CC3]

Starting material: Reagent: Product:

50 mg (135 μmol) 4-chloro-2-methoxyaniline 4.9 mg (7%)

¹H-NMR (DMSO-d6): δ=1.74-1.89 (3H), 2.03 (1H), 2.61 (1H), 2.69 (2H), 2.92-3.13 (4H), 2.94 (3H), 3.21 (2H), 3.86 (3H), 6.97 (1H), 7.05 (1H), 7.74 (1H), 8.05 (1H), 8.26 (1H), 8.72 (1H), 11.57 (1H) ppm.

### Example 150

### (RS)-4-{[5-Chloro-2-(propan-2-yloxy)phenyl]amino}-N-[3-(methylsulfonyl)propyl]-6,7,8,9-tetrahydro-5H-pyrimido[4,5-b]indole-6-carboxamide

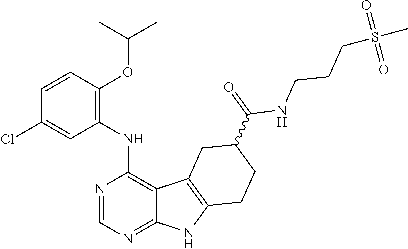 [SMILES: CC(C)Oc1ccc(Cl)cc1Nc1ncnc2[nH]c3c(c12)CC(C(=O)NCCCS(C)(=O)=O)CC3]

Starting material: Reagent: Product:

50 mg (135 μmol) 4-chloro-2-isopropoxyaniline 5.0 mg (7%)

¹H-NMR (DMSO-d6): δ=1.28 (6H), 1.75-1.88 (3H), 2.03 (1H), 2.56-2.66 (1H), 2.71 (2H), 2.94 (3H), 2.96-3.15 (5H), 3.23 (1H), 4.72 (1H), 6.93 (1H), 7.07 (1H), 7.83 (1H), 8.07 (1H), 8.28 (1H), 8.91 (1H), 11.60 (1H) ppm.

### Example 151

### (RS)-4-{[2-(Cyclopropylmethoxy)phenyl]amino}-N-[3-(methylsulfonyl)propyl]-6,7,8,9-tetrahydro-5H-pyrimido[4,5-b]indole-6-carboxamide

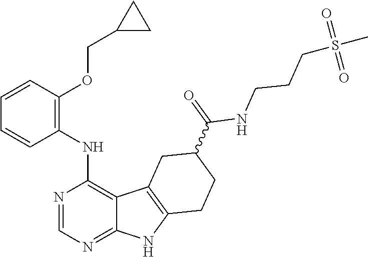 [SMILES: CS(=O)(=O)CCCNC(=O)C1CCc2[nH]c3ncnc(Nc4ccccc4OCC4CC4)c3c2C1]

Starting material: Reagent: Product:

50 mg (135 μmol) 2-(cyclopropylmethoxy)aniline 15.1 mg (21%)

¹H-NMR (DMSO-d6): δ=0.28 (2H), 0.50-0.63 (2H), 1.50 (1H), 1.75-1.87 (3H), 2.00 (1H), 2.61 (1H), 2.70 (2H), 2.94 (3H). 2.97-3.13 (5H), 3.21 (1H), 3.82-3.90 (2H), 6.86-6.95 (3H), 7.87 (1H), 8.06 (1H), 8.22 (1H), 8.75 (1H), 11.48 (1H) ppm.

### Example 152

### (RS)-4-{[2-(2,2-Difluoroethoxy)phenyl]amino}-N-[3-(methylsulfonyl)propyl]-6,7,8,9-tetrahydro-5H-pyrimido[4,5-b]indole-6-carboxamide

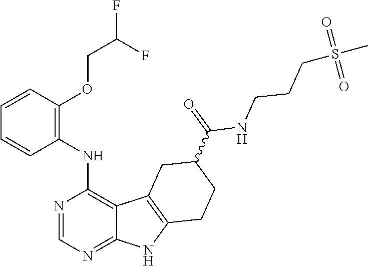 [SMILES: CS(=O)(=O)CCCNC(=O)C1CCc2[nH]c3ncnc(Nc4ccccc4OCC(F)F)c3c2C1]

Starting material: Reagent: Product:

50 mg (135 μmol) 2-(2,2-difluoroethoxy)aniline 53.5 mg (74%)

¹H-NMR (DMSO-d6): δ=1.72-1.92 (3H), 2.02 (1H), 2.59 (1H), 2.69 (2H), 2.93 (3H), 3.06-3.27 (4H), 3.40 (1H), 4.38 (2H), 4.42-4.43 (1H), 6.35 (1H), 6.97 (2H), 7.10 (1H), 7.69 (1H), 8.03 (1H), 8.21 (1H), 8.71 (1H), 11.55 (1H) ppm.

### Example 153

### (RS)-4-{[2-(2-Methylpropoxy)phenyl]amino}-N-[3-(methylsulfonyl)propyl]-6,7,8,9-tetrahydro-5H-pyrimido[4,5-b]indole-6-carboxamide

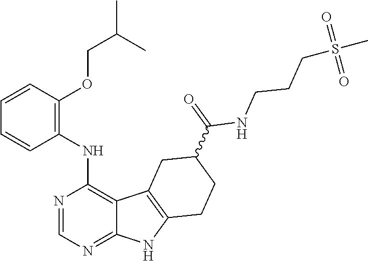 [SMILES: CC(C)COc1ccccc1Nc1ncnc2[nH]c3c(c12)CC(C(=O)NCCCS(C)(=O)=O)CC3]

Starting material: Reagent: Product:

50 mg (135 μmol) 2-isobutoxyaniline 16.8 mg (24%)

¹H-NMR (DMSO-d6): δ=0.95 (6H), 1.74-1.90 (3H), 1.95-2.08 (2H), 2.59 (1H), 2.69 (2H), 2.94 (3H), 2.97-3.18 (5H), 3.29-3.34 (1H), 3.81 (2H), 6.88-6.94 (2H), 6.99 (1H), 7.70 (1H), 8.04 (1H), 8.21 (1H), 8.75 (1H), 11.52 (1H) ppm.

### Example 154

### (RS)-4-{[2-(Difluoromethoxy)phenyl]amino}-N-[3-(methylsulfonyl)propyl]-6,7,8,9-tetrahydro-5H-pyrimido[4,5-b]indole-6-carboxamide

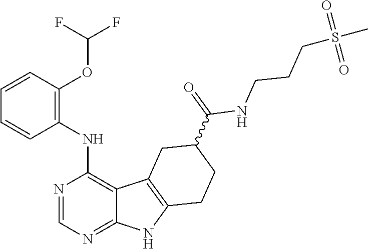 [SMILES: CS(=O)(=O)CCCNC(=O)C1CCc2[nH]c3ncnc(Nc4ccccc4OC(F)F)c3c2C1]

Starting material: Reagent: Product:

50 mg (135 μmol) 2-(difluoromethoxy)aniline 11.4 mg (16%)

¹H-NMR (DMSO-d6): δ=1.77-1.86 (3H), 2.00 (1H), 2.57 (1H), 2.65-2.73 (2H), 2.90-2.98 (1H), 2.93 (3H), 3.01-3.25 (6H), 7.08 (1H), 7.12 (1H), 7.19-7.26 (2H), 7.67 (1H), 8.02 (1H), 8.14 (1H), 8.36 (1H) ppm.

### Example 155

### (RS)—N-[3-(Methylsulfonyl)propyl]-4-[(2-thioxo-2,3-dihydro-1,3-benzothiazol-6-yl)amino]-6,7,8,9-tetrahydro-5H-pyrimido[4,5-b]indole-6-carboxamide

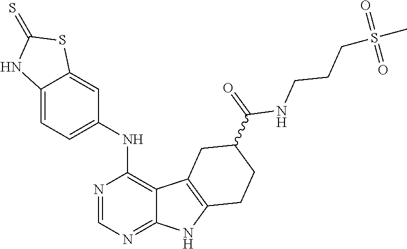 [SMILES: CS(=O)(=O)CCCNC(=O)C1CCc2[nH]c3ncnc(Nc4ccc5[nH]c(=S)sc5c4)c3c2C1]

Starting material: Reagent: Product:

40 mg (108 μmol) 6-amino-1,3-benzothiazole-2(3H)-thione 3.3 mg (5%)

¹H-NMR (DMSO-d6): δ=1.72-1.87 (3H), 1.99 (1H), 2.48-2.54 (1H), 2.60-2.75 (2H), 2.89-2.99 (1H), 2.94 (3H), 3.06-3.25 (5H), 7.21 (1H), 7.58 (1H), 7.97-8.05 (2H), 8.10 (2H), 11.46 (1H), 13.58 (1H) ppm.

### Example 156

### (RS)-4-{[2-(Difluoromethoxy)-4-methylphenyl]amino}-N-[3-(methylsulfonyl)propyl]-6,7,8,9-tetrahydro-5H-pyrimido[4,5-b]indole-6-carboxamide

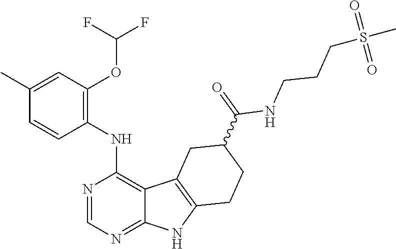

Starting material: Reagent: Product:

50 mg (135 μmol) 2-(difluoromethoxy)-4-methylaniline 14.2 mg (20%)

¹H-NMR (DMSO-d6): δ=1.74-1.86 (3H), 1.99 (1H), 2.28 (3H), 2.56 (1H), 2.62-2.73 (2H), 2.88-2.96 (1H), 2.93 (3H), 3.00-3.26 (5H), 7.03 (1H), 7.04 (1H), 7.08 (1H), 7.60 (1H), 8.02 (1H), 8.10 (1H), 8.12 (1H), 11.48 (1H) ppm.

### Example 157

### (RS)-4-[(5-Chloro-4-fluoro-2-methoxyphenyl)amino]-N-[3-(methylsulfonyl)propyl]-6,7,8,9-tetrahydro-5H-pyrimido[4,5-b]indole-6-carboxamide

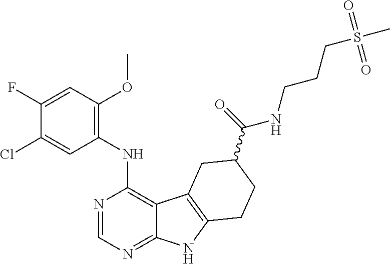 [SMILES: COc1cc(F)c(Cl)cc1Nc1ncnc2[nH]c3c(c12)CC(C(=O)NCCCS(C)(=O)=O)CC3]

Starting material: Reagent: Product:

50 mg (135 μmol) 5-chloro-4-fluoro-2-methoxyaniline 35.4 mg (49%)

¹H-NMR (DMSO-d6): δ=1.70-1.89 (3H), 2.00 (1H), 2.51 (1H), 2.69 (2H), 2.90 (1H), 2.93 (3H), 3.01-3.26 (5H), 3.84 (3H), 7.14 (1H), 7.68 (1H), 7.84 (1H), 7.96-8.05 (2H), 11.39 (1H) ppm.

### Example 158

### (RS)-4-{[5-(Dimethylamino)-2-methoxyphenyl]amino}-N-[3-(methylsulfonyl)propyl]-6,7,8,9-tetrahydro-5H-pyrimido[4,5-b]indole-6-carboxamide

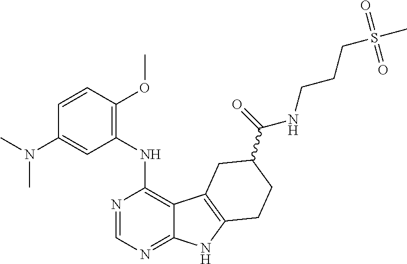

Starting material: Reagent: Product:

50 mg (135 μmol) 4-methoxy-N¹,N¹-dimethylbenzene-1,3-diamine 25.5 mg (36%)

¹H-NMR (DMSO-d6): δ=1.72-1.87 (3H), 2.02 (1H), 2.57 (1H), 2.67 (2H), 2.85 (6H), 2.89 (1H), 2.94 (3H), 3.02 (1H), 3.10 (2H), 3.20 (2H), 3.80 (3H), 6.29 (1H), 6.41 (1H), 7.36 (1H), 8.00-8.07 (3H), 11.35 (1H) ppm.

### Example 159

### (RS)-4-{[4-Fluoro-2-(2-hydroxyethoxy)phenyl]amino}-N-[3-(methylsulfonyl)propyl]-6,7,8,9-tetrahydro-5H-pyrimido[4,5-b]indole-6-carboxamide

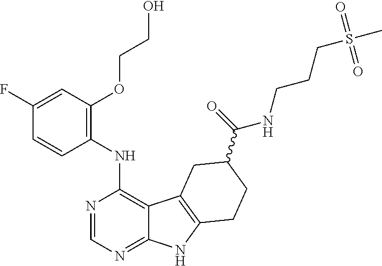 [SMILES: CS(=O)(=O)CCCNC(=O)C1CCc2[nH]c3ncnc(Nc4ccc(F)cc4OCCO)c3c2C1]

Starting material: Reagent: Product:

50 mg (135 μmol) 2-(2-amino-5-fluorophenoxy)ethanol 31.6 mg (44%)

¹H-NMR (DMSO-d6): δ=1.70-1.91 (3H), 2.04 (1H), 2.60 (1H), 2.69 (2H), 2.94 (3H), 2.99-3.25 (6H), 3.74 (2H), 4.08 (2H), 4.80 (1H), 6.76 (1H), 6.97 (1H), 7.70 (1H), 8.03 (1H), 8.18 (1H), 8.63 (1H), 11.48 (1H) ppm.

### Example 160

### (RS)-4-[(5-Fluoro-2-propoxyphenyl)amino]-N-[3-(methylsulfonyl)propyl]-6,7,8,9-tetrahydro-5H-pyrimido[4,5-b]indole-6-carboxamide

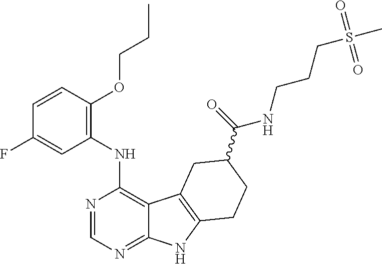 [SMILES: CCCOc1ccc(F)cc1Nc1ncnc2[nH]c3c(c12)CC(C(=O)NCCCS(C)(=O)=O)CC3]

Starting material: Reagent: Product:

50 mg (135 μmol) 5-fluoro-2-propoxyaniline 25.6 mg (36%)

¹H-NMR (DMSO-d6): δ=0.93 (3H), 1.68-1.89 (5H), 2.01 (1H), 2.58 (1H), 2.69 (2H), 2.94 (3H), 2.96-3.34 (6H), 4.00 (2H), 6.75 (1H), 6.94 (1H), 7.59 (1H), 8.03 (1H), 8.18 (1H), 8.66 (1H), 11.50 (1H) ppm.

### Example 161

### (RS)-4-[(4,5-Difluoro-2-methoxyphenyl)amino]-N-[3-(methylsulfonyl)propyl]-6,7,8,9-tetrahydro-5H-pyrimido[4,5-b]indole-6-carboxamide

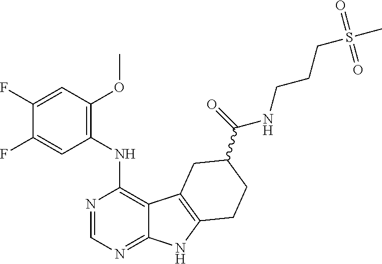 [SMILES: COc1cc(F)c(F)cc1Nc1ncnc2[nH]c3c(c12)CC(C(=O)NCCCS(C)(=O)=O)CC3]

Starting material: Reagent: Product:

50 mg (135 μmol) 4,5-difluoro-2-methoxyaniline 9.2 mg (13%)

¹H-NMR (DMSO-d6): δ=1.74-1.87 (3H), 2.03 (1H), 2.59 (1H), 2.69 (2H), 2.94 (3H), 2.95 (1H), 3.02 (1H), 3.10 (2H), 3.20 (2H), 3.85 (3H), 7.25 (1H), 7.62 (1H), 8.06 (1H), 8.21 (1H), 8.63 (1H), 11.57 (1H) ppm.

### Example 162

### (RS)-4-[(4-Chloro-2-methoxy-5-methylphenyl)amino]-N-[3-(methylsulfonyl)propyl]-6,7,8,9-tetrahydro-5H-pyrimido[4,5-b]indole-6-carboxamide

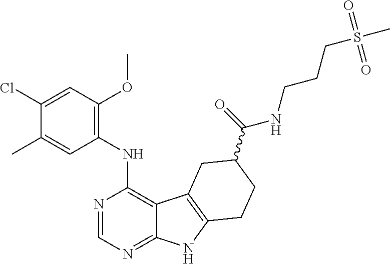

Starting material: Reagent: Product:

50 mg (135 μmol) 4-chloro-2-methoxy-5-methylaniline 15.5 mg (22%)

¹H-NMR (DMSO-d6): δ=1.73-1.88 (3H), 2.03 (1H), 2.26 (3H), 2.59 (1H), 2.68 (2H), 2.91-2.97 (1H), 2.94 (3H), 3.03 (1H), 3.10 (2H), 3.20 (2H), 3.84 (3H), 7.08 (1H), 7.62 (1H), 8.05 (1H), 8.19 (1H), 8.47 (1H), 11.52 (1H) ppm.

### Example 163

### (RS)-4-[(6-Methoxy-1H-indazol-5-yl)amino]-N-[3-(methylsulfonyl)propyl]-6,7,8,9-tetrahydro-5H-pyrimido[4,5-b]indole-6-carboxamide

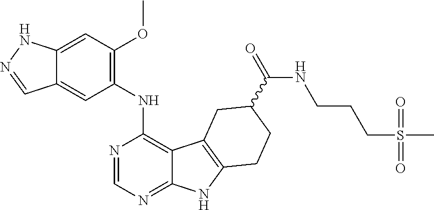 [SMILES: COc1cc2[nH]ncc2cc1Nc1ncnc2[nH]c3c(c12)CC(C(=O)NCCCS(C)(=O)=O)CC3]

Starting material: Reagent: Product:

75 mg (202 μmol) 6-methoxy-1H-indazol-5-amine 36.5 mg (34%)

¹H-NMR (DMSO-d6): δ=1.73-1.88 (3H), 2.05 (1H), 2.61 (1H), 2.69 (2H), 2.91-3.14 (4H), 2.94 (3H), 3.20 (2H), 3.93 (3H), 7.03 (1H), 7.76 (1H), 7.94 (1H), 8.05 (1H), 8.21 (1H), 8.61 (1H), 11.45 (1H), 12.74 (1H) ppm.

### Example 164

### (RS)-4-[(6-Methoxy-2-oxo-2,3-dihydro-1H-benzimidazol-5-yl)amino]-N-[3-(methylsulfonyl)propyl]-6,7,8,9-tetrahydro-5H-pyrimido[4,5-b]indole-6-carboxamide

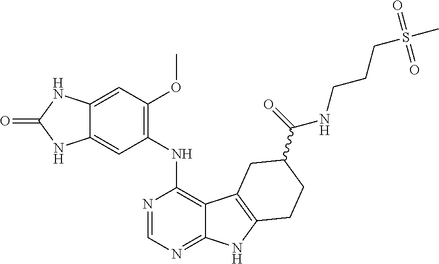 [SMILES: COc1cc2[nH]c(=O)[nH]c2cc1Nc1ncnc2[nH]c3c(c12)CC(C(=O)NCCCS(C)(=O)=O)CC3]

Starting material: Reagent: Product:

100 mg (270 μmol) 5-amino-6-methoxy-1,3-dihydro-2H-benzimidazol-2-one 87.8 mg (60%)

¹H-NMR (DMSO-d6): δ=1.72-1.89 (3H), 2.03 (1H), 2.57 (1H), 2.72 (2H), 2.93 (4H), 3.02-3.23 (6H), 3.72 (3H), 6.74 (1H), 7.26 (1H), 8.03-8.12 (2H), 10.53 (1H), 10.69 (1H), 12.30 (1H) ppm.

### Example 165

### (RS)-4-[(4-Chloro-2-ethoxyphenyl)amino]-N-[3-(methylsulfonyl)propyl]-6,7,8,9-tetrahydro-5H-pyrimido[4,5-b]indole-6-carboxamide

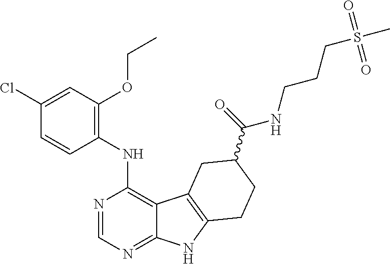 [SMILES: CCOc1cc(Cl)ccc1Nc1ncnc2[nH]c3c(c12)CC(C(=O)NCCCS(C)(=O)=O)CC3]

Starting material: Reagent: Product:

50 mg (135 μmol) 4-chloro-2-ethoxyaniline 43.1 mg (60%)

¹H-NMR (DMSO-d6): δ=1.35 (3H), 1.73-1.89 (3H), 2.01 (1H), 2.59 (1H), 2.69 (2H), 2.94 (3H), 2.96-3.25 (6H), 4.11 (2H), 6.99 (1H), 7.06 (1H), 7.73 (1H), 8.04 (1H), 8.22 (1H), 8.74 (1H), 11.54 (1H) ppm.

### Example 166

### (RS)-4-[(3,4-Dichlorophenyl)amino]-N-[3-(methylsulfonyl)propyl]-6,7,8,9-tetrahydro-5H-pyrimido[4,5-b]indole-6-carboxamide

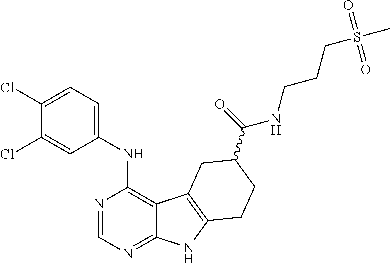 [SMILES: CS(=O)(=O)CCCNC(=O)C1CCc2[nH]c3ncnc(Nc4ccc(Cl)c(Cl)c4)c3c2C1]

Starting material: Reagent: Product:

50 mg (135 μmol) 3,4-dichloroanitine 26 mg (37%)

¹H-NMR (DMSO-d6): δ=1.71-1.89 (3H), 1.99 (1H), 2.50 (1H), 2.69 (2H), 2.94 (3H), 2.96 (1H), 3.05-3.27 (5H), 7.49 (1H), 7.70 (1H), 8.01 (1H), 8.06 (1H), 8.16 (1H), 8.22 (1H), 11.56 (1H) ppm.

### Example 167

### (RS)—N-[3-(Methylsulfonyl)propyl]-4-{[2-(tetrahydrofuran-3-yloxy)phenyl]amino}-6,7,8,9-tetrahydro-5H-pyrimido[4,5-b]indole-6-carboxamide

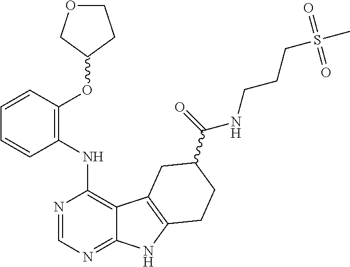 [SMILES: CS(=O)(=O)CCCNC(=O)C1CCc2[nH]c3ncnc(Nc4ccccc4OC4CCOC4)c3c2C1]

Starting material: Reagent: Product:

50 mg (135 μmol) (RS)-2-(tetrahydrofuran-3-yloxy)aniline 61.2 mg (84%)

¹H-NMR (DMSO-d6): δ=1.74-1.90 (3H), 1.95-2.08 (2H), 2.23 (1H), 2.59 (1H), 2.69 (2H), 2.94 (3H), 2.97-3.25 (6H), 3.68-3.92 (4H), 5.12 (1H), 6.88-7.02 (3H), 7.71 (1H), 8.03 (1H), 8.21 (1H), 8.78 (1H), 11.53 (1H) ppm.

### Example 168

### (RS)-4-(2,3-Dihydro-1H-indol-1-yl)-N-[3-(methylsulfonyl)propyl]-6,7,8,9-tetrahydro-5H-pyrimido[4,5-b]indole-6-carboxamide

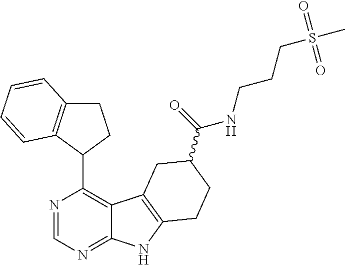 [SMILES: CS(=O)(=O)CCCNC(=O)C1CCc2[nH]c3ncnc(C4CCc5ccccc54)c3c2C1]

Starting material: Reagent: Product:

100 mg (270 μmol) indoline 67 mg (52%)

¹H-NMR (DMSO-d6): δ=1.69-1.87 (3H), 1.97 (1H), 2.36 (1H), 2.62-2.81 (4H), 2.90 (3H), 2.95-3.20 (6H), 3.99 (1H), 4.20 (1H), 6.80 (1H), 7.01 (1H), 7.13 (1H), 7.19 (1H), 7.90 (1H), 8.22 (1H), 11.68 (1H) ppm.

### Example 169

### (RS)-4-[(2-Methyl-1H-benzimidazol-4-yl)amino]-N-[3-(methylsulfonyl)propyl]-6,7,8,9-tetrahydro-5H-pyrimido[4,5-b]indole-6-carboxamide

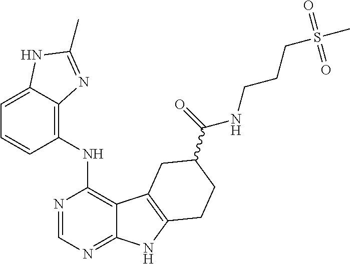 [SMILES: Cc1nc2c(Nc3ncnc4[nH]c5c(c34)CC(C(=O)NCCCS(C)(=O)=O)CC5)cccc2[nH]1]

Starting material: Reagent: Product:

60 mg (162 μmol) 2-methyl-1H-benzimidazo-4-amine 3.4 mg (4%)

¹H-NMR (DMSO-d6): δ=1.76-1.92 (3H), 2.02 (1H), 2.46 (3H), 2.57-2.76 (3H), 2.93 (3H), 3.03-3.25 (6H), 6.99-7.08 (2H), 8.12 (2H), 8.23 (1H), 8.30 (1H), 11.54 (1H), 12.30 (1H) ppm.

### Examples 170-179

Compounds of examples 170-179 were prepared in analogy to example 1 from (RS)-Ethyl 4-chloro-6,7,8,9-tetrahydro-5H-pyrimido[4,5-b]indole-6-carboxylate (prepared according to intermediate example 5a) as starting material and the reagent given.

### Example 170

### (RS)-Ethyl 4-{[2-(cyclopentyloxy)phenyl]amino}-6,7,8,9-tetrahydro-5H-pyrimido[4,5-b]indole-6-carboxylate

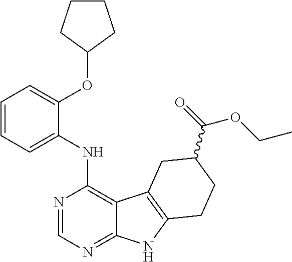 [SMILES: CCOC(=O)C1CCc2[nH]c3ncnc(Nc4ccccc4OC4CCCC4)c3c2C1]

Starting material: Reagent: Product:

500 mg (1.78 mmol) 2-(cyclopentyloxy)aniline 542 mg (69%)

¹H-NMR (DMSO-d6): δ=1.19 (3H), 1.53-1.98 (9H), 2.13 (1H), 2.71 (2H), 2.85 (1H), 3.04 (1H), 3.15 (1H), 4.00-4.18 (2H), 4.92 (1H), 6.87-7.02 (3H), 7.67 (1H), 8.21 (1H), 8.76 (1H), 11.56 (1H) ppm.

### Example 171

### (RS)-Ethyl 4-[(5-fluoro-2-methoxyphenyl)amino]-6,7,8,9-tetrahydro-5H-pyrimido[4,5-b]indole-6-carboxylate

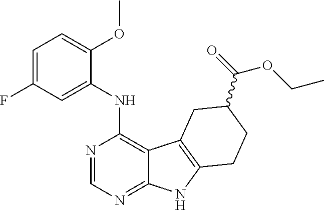 [SMILES: CCOC(=O)C1CCc2[nH]c3ncnc(Nc4cc(F)ccc4OC)c3c2C1]

Starting material: Reagent: Product:

500 mg (1.79 mmol) 5-fluoro-2-methoxyaniline 601 mg (83%)

¹H-NMR (DMSO-d6): δ=1.19 (3H), 1.84 (1H), 2.16 (1H), 2.68-2.97 (4H), 3.14 (1H), 3.78 (3H), 4.02-4.18 (2H), 7.06-7.21 (2H), 7.76 (1H), 8.22 (1H), 9.17 (1H), 12.45 (1H) ppm.

### Example 172

### (RS)-Ethyl 4-{[2-(morpholin-4-yl)phenyl]amino}-6,7,8,9-tetrahydro-5H-pyrimido[4,5-b]indole-6-carboxylate

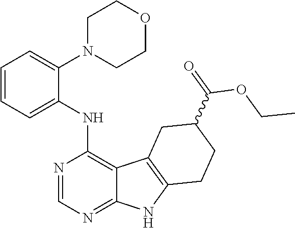 [SMILES: CCOC(=O)C1CCc2[nH]c3ncnc(Nc4ccccc4N4CCOCC4)c3c2C1]

Starting material: Reagent: Product:

500 mg (1.79 mmol) 2-(morpholin-4-yl)aniline 392 mg (49%)

¹H-NMR (DMSO-d6): δ=1.19 (3H), 1.86 (1H), 2.15 (1H), 2.71-2.89 (6H), 2.96 (1H), 3.19 (1H), 3.41 (4H), 3.75 (1H), 3.99-4.18 (2H), 7.12-7.32 (3H), 7.93 (1H), 8.22 (1H), 9.41 (1H), 12.40 (1H) ppm.

### Example 173

### (RS)-Ethyl 4-(2,3-dihydro-1H-indol-1-yl)-6,7,8,9-tetrahydro-5H-pyrimido[4,5-b]indole-6-carboxylate

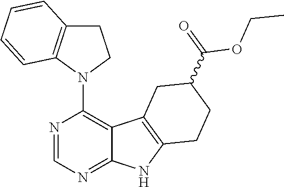 [SMILES: CCOC(=O)C1CCc2[nH]c3ncnc(N4CCc5ccccc54)c3c2C1]

Starting material: Reagent: Product:

500 mg (1.79 mmol) indole 554 mg (85%)

¹H-NMR (DMSO-d6): δ=1.10 (3H), 1.86 (1H), 2.10 (1H), 2.58-2.87 (5H), 2.99-3.16 (2H), 3.95-4.21 (4H), 6.80 (1H), 7.00 (1H), 7.09 (1H), 7.19 (1H), 8.24 (1H), 11.68 (1H) ppm.

### Example 174

### (RS)-Ethyl 4-{[4-fluoro-2-(propan-2-yloxy)phenyl]amino}-6,7,8,9-tetrahydro-5H-pyrimido[4,5-b]indole-6-carboxylate

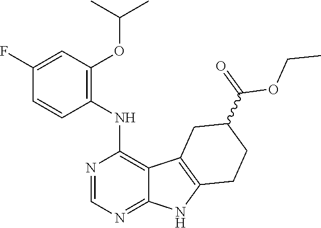 [SMILES: CCOC(=O)C1CCc2[nH]c3ncnc(Nc4ccc(F)cc4OC(C)C)c3c2C1]

Starting material: Reagent: Product:

75 mg (268 μmol) 4-fluoro-2-isopropoxyaniline 90.5 mg (78%)

¹H-NMR (DMSO-d6): δ=1.20 (3H), 1.28-1.33 (6H), 1.84 (1H), 2.17 (1H), 2.72 (2H), 2.85 (1H), 3.03 (1H), 3.17 (1H), 4.02-4.17 (2H), 4.75 (1H), 6.75 (1H), 7.02 (1H), 7.62 (1H), 8.20 (1H), 8.73 (1H), 11.54 (1H) ppm.

### Example 175

### (RS)-Ethyl 4-[(5-chloro-2-methoxyphenyl)amino]-6,7,8,9-tetrahydro-5H-pyrimido[4,5-b]indole-6-carboxylate

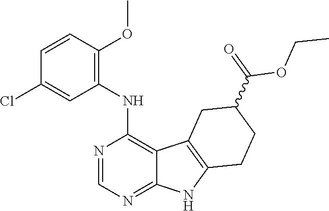 [SMILES: CCOC(=O)C1CCc2[nH]c3ncnc(Nc4cc(Cl)ccc4OC)c3c2C1]

Starting material: Reagent: Product:

100 mg (357 μmol) 5-chloro-2-methoxyaniline 16.7 mg (12%)

¹H-NMR (DMSO-d6): δ=1.21 (3H), 1.83 (1H), 2.19 (1H), 2.65-2.78 (2H), 2.86 (1H), 3.01 (1H), 3.17 (1H), 3.89 (3H), 4.13 (2H), 6.97 (1H), 7.05 (1H), 7.76 (1H), 8.26 (1H), 8.75 (1H), 11.59 (1H) ppm.

### Example 176

### (RS)-Ethyl 4-{[4-fluoro-2-(2-hydroxyethoxy)phenyl]amino}-6,7,8,9-tetrahydro-5H-pyrimido[4,5-b]indole-6-carboxylate

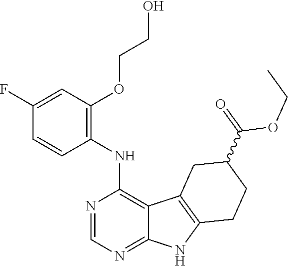 [SMILES: CCOC(=O)C1CCc2[nH]c3ncnc(Nc4ccc(F)cc4OCCO)c3c2C1]

Starting material: Reagent: Product:

500 mg (1.79 mmol) 2-(2-amino-5-fluorophenoxy)ethanol 599 mg (77%)

¹H-NMR (DMSO-d6): δ=1.20 (3H), 1.84 (1H), 2.14 (1H), 2.70 (2H), 2.81 (1H), 3.04 (1H), 3.20 (1H), 3.75 (2H), 4.02-4.19 (4H), 4.82 (1H), 6.76 (1H), 6.99 (1H), 7.69 (1H), 8.18 (1H), 8.63 (1H), 11.52 (1H) ppm.

### Example 177

### (RS)-Ethyl 4-{[5-chloro-2-(propan-2-yloxy)phenyl]amino}-6,7,8,9-tetrahydro-5H-pyrimido[4,5-b]indole-6-carboxylate

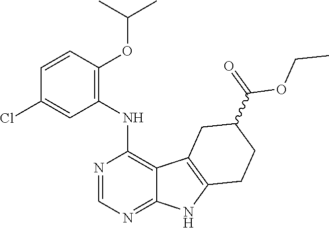 [SMILES: CCOC(=O)C1CCc2[nH]c3ncnc(Nc4cc(Cl)ccc4OC(C)C)c3c2C1]

Starting material: Reagent: Product:

100 mg (357 μmol) 5-chloro-2-isopropoxyaniline 32.3 mg (20%)

¹H-NMR (DMSO-d6): δ=1.20 (3H), 1.33 (6H), 1.84 (1H), 2.17 (1H), 2.72 (2H), 2.86 (1H), 3.04 (1H), 3.17 (1H), 4.01-4.17 (2H), 4.73 (1H), 6.93 (1H), 7.07 (1H), 7.79 (1H), 8.28 (1H), 8.90 (1H), 11.62 (1H) ppm.

### Example 178

### (RS)-Ethyl 4-[(3,4-dichlorophenyl)amino]-6,7,8,9-tetrahydro-5H-pyrimido[4,5-b]indole-6-carboxylate

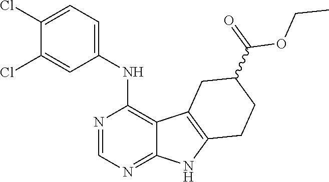 [SMILES: CCOC(=O)C1CCc2[nH]c3ncnc(Nc4ccc(Cl)c(Cl)c4)c3c2C1]

Starting material: Reagent: Product:

100 mg (357 μmol) 3,4-dichloroaniline 46.7 mg (31%)

¹H-NMR (DMSO-d6): δ=1.19 (3H), 1.83 (1H), 2.12 (1H), 2.67-2.79 (3H), 2.98 (1H), 3.22 (1H), 4.03-4.15 (2H), 7.49 (1H), 7.68 (1H), 8.04 (1H), 8.18 (1H), 8.26 (1H), 11.56 (1H) ppm.

### Example 179

### (RS)-Ethyl 4-[(6-methoxy-1H-indazol-5-yl)amino]-6,7,8,9-tetrahydro-5H-pyrimido[4,5-b]indole-6-carboxylate

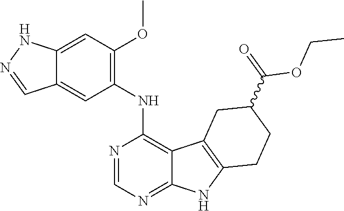 [SMILES: CCOC(=O)C1CCc2[nH]c3ncnc(Nc4cc5cn[nH]c5cc4OC)c3c2C1]

Starting material: Reagent: Product:

606 mg (2.17 mmol) 6-methoxy-1H-indazol-5-amine 948 mg (99%)

¹H-NMR (DMSO-d6): δ=1.22 (3H), 1.84 (1H), 2.19 (1H), 2.65-2.79 (2H), 2.87 (1H), 3.04 (1H), 3.21 (1H), 3.96 (3H), 4.14 (2H), 7.05 (1H), 7.79 (1H), 7.94 (1H), 8.22 (1H), 8.86 (1H), 11.48 (1H), 12.75 (1H) ppm.

### Examples 180-186

Compounds of examples 180-186 were prepared in analogy to example 6 from the starting materials given.

### Example 180

### (RS)-4-{[2-(Cyclopentyloxy)phenyl]amino}-6,7,8,9-tetrahydro-5H-pyrimido[4,5-b]indole-6-carboxylic acid

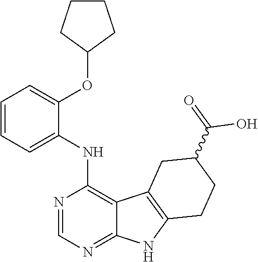 [SMILES: O=C(O)C1CCc2[nH]c3ncnc(Nc4ccccc4OC4CCCC4)c3c2C1]

Starting material: Product:

532 mg (1.27 mmol) (RS)-Ethyl 4-{[2-(cyclopentyloxy)phenyl]amino}-6,7,8,9-tetrahydro-5H-pyrimido[4,5-b]indole-6-carboxylate (prepared according to example 170) 473 mg (91%)

¹H-NMR (DMSO-d6): δ=1.48-1.99 (8H), 2.14 (1H), 2.23 (1H), 2.65-2.81 (3H), 3.02 (1H), 3.15 (1H), 4.92 (1H), 6.86-7.02 (3H), 7.70 (1H), 8.21 (1H), 8.78 (1H), 11.53 (1H), 12.41 (1H) ppm.

### Example 181

### (RS)-4-[(5-Fluoro-2-methoxyphenyl)amino]-6,7,8,9-tetrahydro-5H-pyrimido[4,5-b]indole-6-carboxylic acid

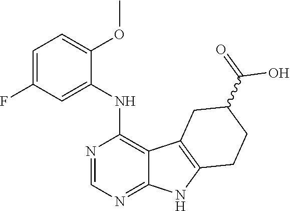 [SMILES: COc1ccc(F)cc1Nc1ncnc2[nH]c3c(c12)CC(C(=O)O)CC3]

Starting material: Product:

591 mg (1.54 mmol) (RS)-Ethyl 4-[(5-fluoro-2-methoxyphenyl)amino]-6,7,8,9-tetrahydro-5H-pyrimido[4,5-b]indole-6-carboxylate (prepared according to example 171) 487 mg (84%)

¹H-NMR (DMSO-d6): δ=1.83 (1H), 2.17 (1H), 2.71 (2H), 2.79 (1H), 3.01 (1H), 3.16 (1H), 3.88 (3H), 6.74 (1H), 7.03 (1H), 7.82 (1H), 8.25 (1H), 8.56 (1H), 11.57 (1H), 12.37 (1H) ppm.

### Example 182

### (RS)-4-{[4-Fluoro-2-(2-hydroxyethoxy)phenyl]amino}-6,7,8,9-tetrahydro-5H-pyrimido[4,5-b]indole-6-carboxylic acid

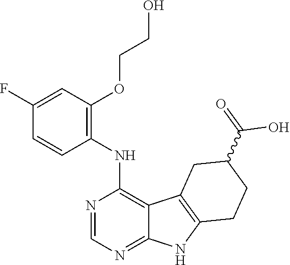 [SMILES: O=C(O)C1CCc2[nH]c3ncnc(Nc4ccc(F)cc4OCCO)c3c2C1]

Starting material: Product:

592 mg (1.43 mmol) (RS)-Ethyl 4-{[4-fluoro-2-(2-hydroxyethoxy)phenyl]amino}-6,7,8,9-tetrahydro-5H-pyrimido[4,5-b]indole-6-carboxylate (prepared according to example 176) 501 mg (86%)

¹H-NMR (DMSO-d6): δ=1.83 (1H), 2.15 (1H), 2.68-2.80 (3H), 2.99 (1H), 3.18 (1H), 3.65 (2H), 4.07 (2H), 4.73 (1H), 6.83 (1H), 7.07 (1H), 8.15 (2H), 8.46 (1H), 11.99 (1H), 12.39 (1H) ppm.

### Example 183

### (RS)-4-{[4-Fluoro-2-(propan-2-yloxy)phenyl]amino}-6,7,8,9-tetrahydro-5H-pyrimido[4,5-b]indole-6-carboxylic acid

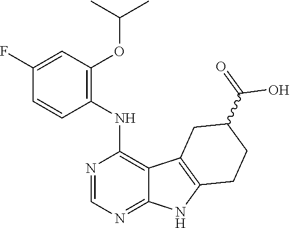 [SMILES: CC(C)Oc1cc(F)ccc1Nc1ncnc2[nH]c3c(c12)CC(C(=O)O)CC3]

Starting material: Product:

79.8 mg (193 μmol) (RS)-Ethyl 4-{[4-fluoro-2-(propan-2-yloxy)phenyl]amino}-6,7,8,9-tetrahydro-5H-pyrimido[4,5-b]indole-6-carboxylate (prepared according to example 174) 15.2 mg (20%)

¹H-NMR (DMSO-d6): δ=1.32 (6H), 1.81 (1H), 2.17 (1H), 2.66-2.85 (3H), 3.02 (1H), 3.17 (1H), 4.75 (1H), 6.75 (1H), 7.02 (1H), 7.65 (1H), 8.20 (1H), 8.75 (1H), 11.51 (1H), 12.36 (1H) ppm.

### Example 184

### (RS)-4-[(6-Methoxy-1H-indazol-5-yl)amino]-6,7,8,9-tetrahydro-5H-pyrimido[4,5-b]indole-6-carboxylic acid hydrochloride (1:1)

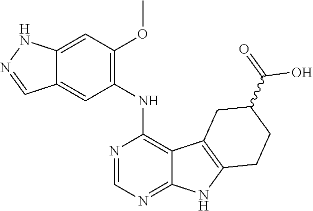 [SMILES: COc1cc2[nH]ncc2cc1Nc1ncnc2[nH]c3c(c12)CC(C(=O)O)CC3]

Starting material: Product:

1.70 g (3.84 mmol) (RS)-Ethyl 4-[(6-methoxy-1H-indazol-5-yl)amino]-6,7,8,9-tetrahydro-5H-pyrimido[4,5-b]indole-6-carboxylate (prepared according to example 179) 1.18 g (74%)

¹H-NMR (DMSO-d6): δ=1.83 (1H), 2.15 (1H), 2.69-2.81 (3H), 2.95 (1H), 3.21 (1H), 3.81 (3H), 7.15 (1H), 7.94 (1H), 8.03 (1H), 8.07 (1H), 9.60 (1H), 12.54 (1H), 13.05 (1H) ppm.

### Example 185

### (RS)-4-(2,3-Dihydro-1H-indol-1-yl)-6,7,8,9-tetrahydro-5H-pyrimido[4,5-b]indole-6-carboxylic acid

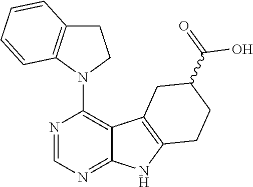 [SMILES: O=C(O)C1CCc2[nH]c3ncnc(N4CCc5ccccc54)c3c2C1]

Starting material: Product:

540 mg (1.49 mmol) (RS)-Ethyl 4-(2,3-dihydro-1H-indol-1-yl)-6,7,8,9-tetrahydro-5H-pyrimido[4,5-b]indole-6-carboxylate (prepared according to example 173) 540 mg (98%)

¹H-NMR (DMSO-d6): δ=1.77 (1H), 2.04 (1H), 2.23-2.44 (1H), 2.62-2.78 (4H), 2.98-3.16 (2H), 4.03 (1H), 4.16 (1H), 6.78 (1H), 6.98 (1H), 7.08 (1H), 7.17 (1H), 8.22 (1H), 11.67 (1H) ppm.

### Example 186

### (RS)-4-{[2-(Morpholin-4-yl)phenyl]amino}-6,7,8,9-tetrahydro-5H-pyrimido[4,5-b]indole-6-carboxylic acid

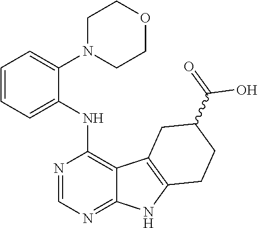 [SMILES: O=C(O)C1CCc2[nH]c3ncnc(Nc4ccccc4N4CCOCC4)c3c2C1]

Starting material: Product:

382 mg (906 μmol) (RS)-Ethyl 4-{[2-(morpholin-4-yl)phenyl]amino}-6,7,8,9-tetrahydro-5H-pyrimido[4,5-b]indole-6-carboxylate (prepared according to example 172) 259 mg (69%)

¹H-NMR (DMSO-d6): δ=1.83 (1H), 2.20 (1H), 2.67-2.88 (8H), 3.14 (1H), 3.72-3.88 (4H), 6.96 (1H), 7.15 (1H), 7.33 (1H), 8.22 (1H), 8.59 (1H), 8.84 (1H), 11.54 (1H), 12.49 (1H) ppm.

### Example 187

### (RS)—N-[(4-{[4-Fluoro-2-(2-hydroxyethoxy)phenyl]amino}-6,7,8,9-tetrahydro-5H-pyrimido[4,5-b]indol-6-yl)carbonyl]glycine

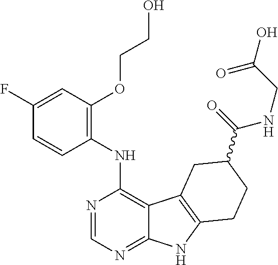 [SMILES: O=C(O)CNC(=O)C1CCc2[nH]c3ncnc(Nc4ccc(F)cc4OCCO)c3c2C1]

92 mg (195 μmol) (RS)-Ethyl N-[(4-{[4-fluoro-2-(2-hydroxyethoxy)phenyl]amino}-6,7,8,9-tetrahydro-5H-pyrimido[4,5-b]indol-6-yl)carbonyl]glycinate (prepared according to intermediate example 187a) were transformed in analogy to example 6 to give after working up and purification 42.2 mg (46%) of the title compound.

¹H-NMR (DMSO-d6): δ=1.81 (1H), 2.05 (1H), 2.62-2.78 (3H), 2.93-3.15 (2H), 3.68-3.76 (3H), 3.86 (1H), 4.08 (2H), 4.76 (1H), 6.77 (1H), 6.99 (1H), 7.73 (1H), 8.18 (1H), 8.29 (1H), 8.60 (1H), 11.54 (1H), 12.53 (1H) ppm.

### Example 187a

### (RS)-Ethyl N-[(4-{[4-fluoro-2-(2-hydroxyethoxy)phenyl]amino}-6,7,8,9-tetrahydro-5H-pyrimido[4,5-b]indol-6-yl)carbonyl]glycinate

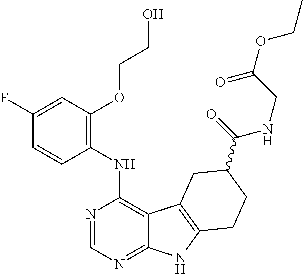 [SMILES: CCOC(=O)CNC(=O)C1CCc2[nH]c3ncnc(Nc4ccc(F)cc4OCCO)c3c2C1]

200 mg (518 μmol) (RS)-4-{[4-fluoro-2-(2-hydroxyethoxy)phenyl]amino}-6,7,8,9-tetrahydro-5H-pyrimido[4,5-b]indole-6-carboxylic acid (prepared according to example 182) were transformed in analogy to example 14 using ethyl glycinate to give after working up and purification 117.7 mg (48%) of the title compound.

### Examples 188-193

Compounds of examples 188-193 were prepared in analogy to example 14 from (RS)-4-{[2-(cyclopentyloxy)phenyl]amino}-6,7,8,9-tetrahydro-5H-pyrimido[4,5-b]indole-6-carboxylic acid (prepared according to example 180) as starting material and the reagent given.

### Example 188

### (RS)-4-{[2-(Cyclopentyloxy)phenyl]amino}-N,N-dimethyl-6,7,8,9-tetrahydro-5H-pyrimido[4,5-b]indole-6-carboxamide

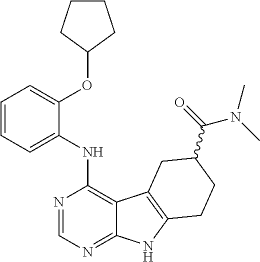 [SMILES: CN(C)C(=O)C1CCc2[nH]c3ncnc(Nc4ccccc4OC4CCCC4)c3c2C1]

Starting material: Reagent: Product:

60 mg (153 μmol) N-methylmethanamine 25.8 mg (38%)

¹H-NMR (DMSO-d6): δ=1.49-1.98 (10H), 2.65-2.79 (2H), 2.85 (3H), 2.93 (1H), 3.02 (1H), 3.08 (3H), 3.12 (1H), 4.91 (1H), 6.87-6.92 (2H), 6.98 (1H), 7.70 (1H), 8.21 (1H), 8.80 (1H), 11.51 (1H) ppm.

### Example 189

### (RS)-4-{[2-(Cyclopentyloxy)phenyl]amino}-N-methyl-6,7,8,9-tetrahydro-5H-pyrimido[4,5-b]indole-6-carboxamide

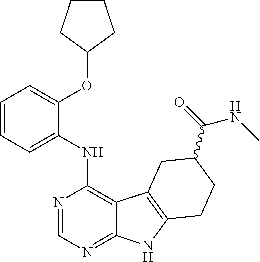 [SMILES: CNC(=O)C1CCc2[nH]c3ncnc(Nc4ccccc4OC4CCCC4)c3c2C1]

Starting material: Reagent: Product:

60 mg (153 μmol) methanamine 46.2 mg (71%)

¹H-NMR (DMSO-d6): δ=1.49-1.99 (10H), 2.54-2.73 (3H), 2.60 (3H), 2.95 (1H), 3.04 (1H), 4.92 (1H), 6.87-6.92 (2H), 6.99 (1H), 7.69 (1H), 7.88 (1H), 8.21 (1H), 8.80 (1H), 11.52 (1H) ppm.

### Example 190

### (RS)-4-{[2-(Cyclopentyloxy)phenyl]amino}-N-ethyl-6,7,8,9-tetrahydro-5H-pyrimido[4,5-b]indole-6-carboxamide

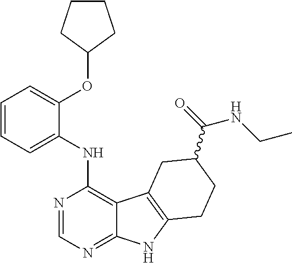 [SMILES: CCNC(=O)C1CCc2[nH]c3ncnc(Nc4ccccc4OC4CCCC4)c3c2C1]

Starting material: Reagent: Product:

60 mg (153 μmol) ethanamine 30.6 mg (45%)

¹H-NMR (DMSO-d6): δ=1.03 (3H), 1.48-1.99 (10H), 2.57 (1H), 2.69 (2H), 2.94-3.20 (4H), 4.92 (1H), 6.87-6.92 (2H), 6.99 (1H), 7.69 (1H), 7.91 (1H), 8.21 (1H), 8.79 (1H), 11.51 (1H) ppm.

### Example 191

### (RS)-4-{[2-(Cyclopentyloxy)phenyl]amino}-N-cyclopropyl-6,7,8,9-tetrahydro-5H-pyrimido[4, 5-b]indole-6-carboxamide

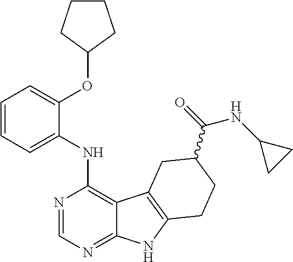 [SMILES: O=C(NC1CC1)C1CCc2[nH]c3ncnc(Nc4ccccc4OC4CCCC4)c3c2C1]

Starting material: Reagent: Product:

60 mg (153 μmol) cyclopropanamine 44.1 mg (64%)

¹H-NMR (DMSO-d6): δ=0.33-0.45 (2H), 0.61 (2H), 1.48-1.99 (10H), 2.49-2.56 (1H), 2.59-2.74 (3H), 2.99 (2H), 4.92 (1H), 6.87-6.92 (2H), 6.99 (1H), 7.69 (1H), 7.98 (1H), 8.21 (1H), 8.79 (1H), 11.51 (1H) ppm.

### Example 192

### (RS)-(4-{[2-(Cyclopentyloxy)phenyl]amino}-6,7,8,9-tetrahydro-5H-pyrimido[4,5-b]indol-6-yl)(4-methylpiperazin-1-yl)methanone

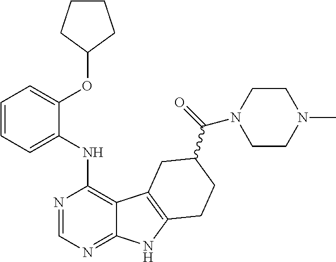 [SMILES: CN1CCN(C(=O)C2CCc3[nH]c4ncnc(Nc5ccccc5OC5CCCC5)c4c3C2)CC1]

Starting material: Reagent: Product:

40 mg (102 μmol) 1-methylpiperazine 16.5 mg (32%)

¹H-NMR (DMSO-d6): δ=1.46-1.99 (10H), 2.15 (3H), 2.20-2.35 (3H), 2.62-2.85 (2H), 2.87-3.64 (8H), 4.91 (1H), 6.86-7.01 (3H), 7.68 (1H), 8.21 (1H), 8.77 (1H), 11.52 (1H) ppm.

### Example 193

### (RS)-4-{[2-(Cyclopentyloxy)phenyl]amino}-N-phenyl-6,7,8,9-tetrahydro-5H-pyrimido[4,5-b]indole-6-carboxamide

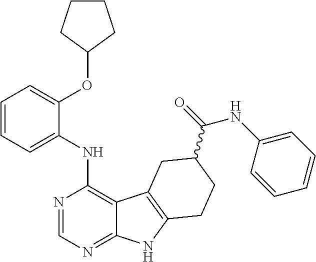 [SMILES: O=C(Nc1ccccc1)C1CCc2[nH]c3ncnc(Nc4ccccc4OC4CCCC4)c3c2C1]

Starting material: Reagent: Product:

60 mg (153 μmol) aniline 48.7 mg (65%)

¹H-NMR (DMSO-d6): δ=1.20-1.35 (2H), 1.42-1.54 (2H), 1.62-1.86 (4H), 1.91 (1H), 2.12 (1H), 2.76 (2H), 2.84 (1H), 3.07 (1H), 3.20 (1H), 4.82 (1H), 6.86-6.97 (3H), 7.01 (1H), 7.28 (2H), 7.65 (2H), 7.72 (1H), 8.23 (1H), 8.82 (1H), 10.04 (1H), 11.57 (1H) ppm.

### Examples 194-200

Compounds of examples 194-200 were prepared in analogy to example 14 from (RS)-4-[(5-fluoro-2-methoxyphenyl)amino]-6,7,8,9-tetrahydro-5H-pyrimido[4,5-b]indole-6-carboxylic acid (prepared according to example 181) as starting material and the reagent given.

### Example 194

### (RS)—N-Ethyl-4-[(5-fluoro-2-methoxyphenyl)amino]-6,7,8,9-tetrahydro-5H-pyrimido[4,5-b]indole-6-carboxamide

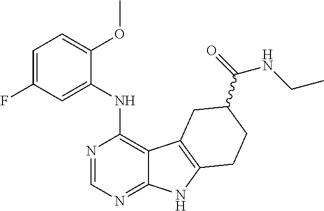 [SMILES: CCNC(=O)C1CCc2[nH]c3ncnc(Nc4cc(F)ccc4OC)c3c2C1]

Starting material: Reagent: Product:

60 mg (168 μmol) ethanamine 44.9 mg (66%)

¹H-NMR (DMSO-d6): δ=1.04 (3H), 1.79 (1H), 2.01 (1H), 2.58 (1H), 2.69 (2H), 2.91-3.19 (4H), 3.85 (3H), 6.74 (1H), 7.02 (1H), 7.78 (1H), 7.92 (1H), 8.25 (1H), 8.56 (1H), 11.57 (1H) ppm.

### Example 195

### (RS)-4-[(5-Fluoro-2-methoxyphenyl)amino]-N-(propan-2-yl)-6,7,8,9-tetrahydro-5H-pyrimido[4,5-b]indole-6-carboxamide

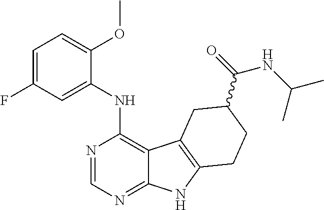 [SMILES: COc1ccc(F)cc1Nc1ncnc2[nH]c3c(c12)CC(C(=O)NC(C)C)CC3]

Starting material: Reagent: Product:

60 mg (168 μmol) propan-2-amine 57.3 mg (81%)

¹H-NMR (DMSO-d6): δ=1.07 (6H), 1.78 (1H), 1.99 (1H), 2.55 (1H), 2.69 (2H), 2.92 (1H), 3.03 (1H), 3.86 (3H), 3.88 (1H), 6.74 (1H), 7.02 (1H), 7.77 (2H), 8.26 (1H), 8.58 (1H), 11.57 (1H) ppm.

### Example 196

### (RS)-{4-[(5-Fluoro-2-methoxyphenyl)amino]-6,7,8,9-tetrahydro-5H-pyrimido[4,5-b]indol-6-yl}(4-methylpiperazin-1-yl)methanone

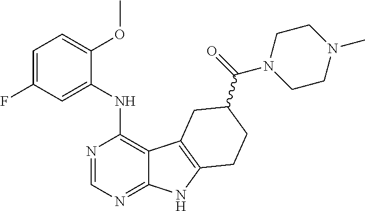 [SMILES: COc1ccc(F)cc1Nc1ncnc2[nH]c3c(c12)CC(C(=O)N1CCN(C)CC1)CC3]

Starting material: Reagent: Product:

40 mg (112 μmol) 1-methylpiperazine 24 mg (46%)

¹H-NMR (DMSO-d6): δ=1.79 (1H), 1.95 (1H), 2.17 (3H), 2.18-2.40 (4H), 2.63-2.85 (2H), 2.88-3.66 (7H), 3.87 (3H), 6.73 (1H), 7.02 (1H), 7.76 (1H), 8.25 (1H), 8.59 (1H), 11.58 (1H) ppm.

### Example 197

### (RS)—N-Cyclopropyl-4-[(5-fluoro-2-methoxyphenyl)amino]-6,7,8,9-tetrahydro-5H-pyrimido[4,5-b]indole-6-carboxamide

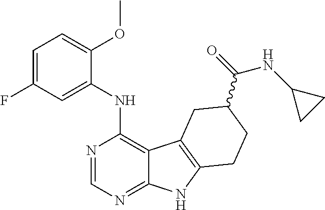 [SMILES: COc1ccc(F)cc1Nc1ncnc2[nH]c3c(c12)CC(C(=O)NC1CC1)CC3]

Starting material: Reagent: Product:

60 mg (168 μmol) cyclopropanamine 43.6 mg (62%)

¹H-NMR (DMSO-d6): δ=0.36-0.45 (2H), 0.55-0.68 (2H), 1.77 (1H), 2.00 (1H), 2.53 (1H), 2.60-2.74 (3H), 2.93 (1H), 3.01 (1H), 3.86 (3H), 6.74 (1H), 7.03 (1H), 7.77 (1H), 7.98 (1H), 8.25 (1H), 8.55 (1H), 11.56 (1H) ppm.

### Example 198

### (RS)-4-[(5-Fluoro-2-methoxyphenyl)amino]-N-(3,3,3-trifluoropropyl)-6,7,8,9-tetrahydro-5H-pyrimido[4,5-b]indole-6-carboxamide

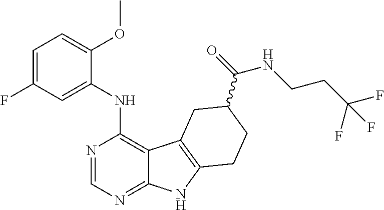 [SMILES: COc1ccc(F)cc1Nc1ncnc2[nH]c3c(c12)CC(C(=O)NCCC(F)(F)F)CC3]

Starting material: Reagent: Product:

60 mg (168 μmol) 3,3,3-trifluoropropan-1-amine 48.2 mg (60%)

¹H-NMR (DMSO-d6): δ=1.80 (1H), 2.01 (1H), 2.38-2.45 (1H), 2.62 (1H), 2.69 (2H), 2.93-3.04 (2H), 3.32-3.39 (3H), 3.85 (3H), 6.73 (1H), 7.02 (1H), 7.74 (1H), 8.19 (1H), 8.25 (1H), 8.56 (1H), 11.54 (1H) ppm.

### Example 199

### (RS)-4-[(5-Fluoro-2-methoxyphenyl)amino]-N-phenyl-6,7,8,9-tetrahydro-5H-pyrimido[4,5-b]indole-6-carboxamide

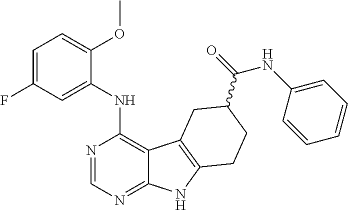 [SMILES: COc1ccc(F)cc1Nc1ncnc2[nH]c3c(c12)CC(C(=O)Nc1ccccc1)CC3]

Starting material: Reagent: Product:

34 mg (95 μmol) aniline 28.8 mg (66%)

¹H-NMR (DMSO-d6): δ=1.87 (1H), 2.16 (1H), 2.76 (2H), 2.85 (1H), 3.06 (1H), 3.19 (1H), 3.79 (3H), 6.73 (1H), 6.98-7.04 (2H), 7.29 (2H), 7.63 (2H), 7.82 (1H), 8.26 (1H), 8.53 (1H), 10.02 (1H), 11.61 (1H) ppm.

### Example 200

### (RS)—N-Benzyl-4-[(5-fluoro-2-methoxyphenyl)amino]-6,7,8,9-tetrahydro-5H-pyrimido[4,5-b]indole-6-carboxamide

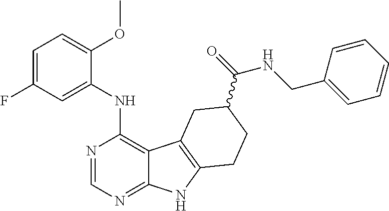 [SMILES: COc1ccc(F)cc1Nc1ncnc2[nH]c3c(c12)CC(C(=O)NCc1ccccc1)CC3]

Starting material: Reagent: Product:

60 mg (168 μmol) 1-phenylmethanamine 55.3 mg (70%)

¹H-NMR (DMSO-d6): δ=1.85 (1H), 2.06 (1H), 2.71 (3H), 2.97-3.11 (2H), 3.76 (3H), 4.26-4.38 (2H), 6.73 (1H), 7.01 (1H), 7.21 (1H), 7.24-7.33 (4H), 7.77 (1H), 8.25 (1H), 8.47 (1H), 8.56 (1H), 11.58 (1H) ppm.

### Examples 201-206

Compounds of examples 201-206 were prepared in analogy to example 14 from (RS)-4-{[2-(morpholin-4-yl)phenyl]amino}-6,7,8,9-tetrahydro-5H-pyrimido[4,5-b]indole-6-carboxylic acid (prepared according to example 186) as starting material and the reagent given.

### Example 201

### (RS)—N-Ethyl-4-{[2-(morpholin-4-yl)phenyl]amino}-6,7,8,9-tetrahydro-5H-pyrimido[4,5-b]indole-6-carboxamide

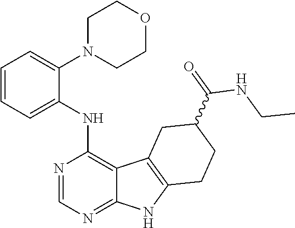 [SMILES: CCNC(=O)C1CCc2[nH]c3ncnc(Nc4ccccc4N4CCOCC4)c3c2C1]

Starting material: Reagent: Product:

42 mg (107 μmol) ethanamine 26.1 mg (55%)

¹H-NMR (DMSO-d6): δ=1.04 (3H), 1.83 (1H), 1.99 (1H), 2.56-2.86 (7H), 3.04-3.16 (3H), 3.21 (1H), 3.73 (4H), 6.96 (1H), 7.15 (1H), 7.35 (1H), 7.96 (1H), 8.23 (1H), 8.65 (1H), 8.88 (1H), 11.51 (1H) ppm.

### Example 202

### (RS)-4-{[2-(Morpholin-4-yl)phenyl]amino}-N-(propan-2-yl)-6,7,8,9-tetrahydro-5H-pyrimido[4,5-b]indole-6-carboxamide

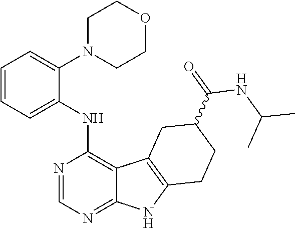 [SMILES: CC(C)NC(=O)C1CCc2[nH]c3ncnc(Nc4ccccc4N4CCOCC4)c3c2C1]

Starting material: Reagent: Product:

42 mg (107 μmol) propan-2-amine 27.9 mg (57%)

¹H-NMR (DMSO-d6): δ=1.07 (6H), 1.82 (1H), 1.98 (1H), 2.55-2.85 (7H), 3.15 (2H), 3.73 (4H), 3.83 (1H), 6.96 (1H), 7.15 (1H), 7.34 (1H), 7.81 (1H), 8.22 (1H), 8.63 (1H), 8.86 (1H), 11.51 (1H) ppm.

### Example 203

### (RS)—N-Cyclopropyl-4-{[2-(morpholin-4-yl)phenyl]amino}-6,7,8,9-tetrahydro-5H-pyrimido[4,5-b]indole-6-carboxamide

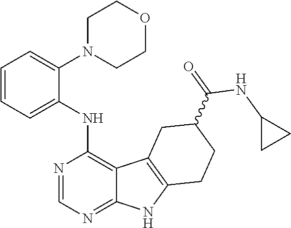 [SMILES: O=C(NC1CC1)C1CCc2[nH]c3ncnc(Nc4ccccc4N4CCOCC4)c3c2C1]

Starting material: Reagent: Product:

42 mg (107 μmol) cyclopropanamine 34.2 mg (70%)

¹H-NMR (DMSO-d6): δ=0.37 (1H), 0.45 (1H), 0.55-0.65 (2H), 1.81 (1H), 1.98 (1H), 2.51-2.71 (4H), 2.71-2.87 (4H), 3.09 (1H), 3.19 (1H), 3.68-3.85 (4H), 6.96 (1H), 7.15 (1H), 7.34 (1H), 8.04 (1H), 8.22 (1H), 8.63 (1H), 8.87 (1H), 11.52 (1H) ppm.

### Example 204

### (RS)-4-{[2-(Morpholin-4-yl)phenyl]amino}-N-(3,3,3-trifluoropropyl)-6,7,8,9-tetrahydro-5H-pyrimido[4,5-b]indole-6-carboxamide

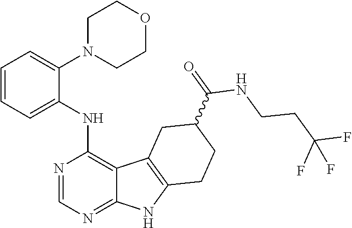 [SMILES: O=C(NCCC(F)(F)F)C1CCc2[nH]c3ncnc(Nc4ccccc4N4CCOCC4)c3c2C1]

Starting material: Reagent: Product:

42 mg (107 μmol) 3,3,3-trifluoropropan-1-amine 36.6 mg (67%)

¹H-NMR (DMSO-d6): δ=1.83 (1H), 2.01 (1H), 2.38-2.55 (2H), 2.61-2.86 (7H), 3.12 (1H), 3.17-3.40 (3H), 3.67-3.79 (4H), 6.96 (1H), 7.15 (1H), 7.34 (1H), 8.23 (1H), 8.26 (1H), 8.66 (1H), 8.88 (1H), 11.52 (1H) ppm.

### Example 205

### (RS)-4-{[2-(Morpholin-4-yl)phenyl]amino}-N-phenyl-6,7,8,9-tetrahydro-5H-pyrimido[4,5-b]indole-6-carboxamide

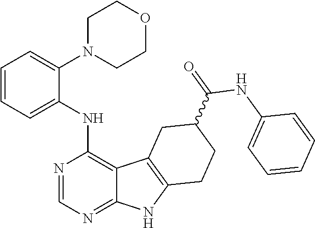 [SMILES: O=C(Nc1ccccc1)C1CCc2[nH]c3ncnc(Nc4ccccc4N4CCOCC4)c3c2C1]

Starting material: Reagent: Product:

42 mg (107 μmol) aniline 12.4 mg (24%)

¹H-NMR (DMSO-d6): δ=1.96 (1H), 2.21 (1H), 2.73-2.87 (6H), 2.91 (1H), 3.27 (1H), 3.38 (1H), 3.65-3.73 (4H), 6.99 (1H), 7.06 (1H), 7.19 (1H), 7.32 (2H), 7.36 (1H), 7.65 (2H), 8.29 (1H), 8.71 (1H), 8.88 (1H), 10.07 (1H), 11.60 (1H) ppm.

### Example 206

### (RS)—N-Benzyl-4-{[2-(morpholin-4-yl)phenyl]amino}-6,7,8,9-tetrahydro-5H-pyrimido[4,5-b]indole-6-carboxamide

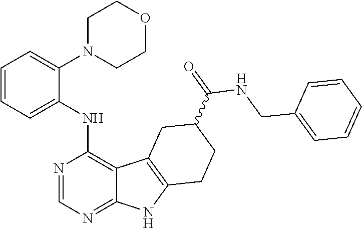 [SMILES: O=C(NCc1ccccc1)C1CCc2[nH]c3ncnc(Nc4ccccc4N4CCOCC4)c3c2C1]

Starting material: Reagent: Product:

33 mg (84 μmol) 1-phenylmethanamine 13 mg (29%)

¹H-NMR (DMSO-d6): δ=1.92 (1H), 2.11 (1H), 2.71-2.90 (7H), 3.22 (1H), 3.29 (1H), 3.78 (4H), 4.27 (1H), 4.45 (1H), 7.01 (1H), 7.20 (1H), 7.25 (1H), 7.29-7.36 (4H), 7.39 (1H), 8.27 (1H), 8.54 (1H), 8.68 (1H), 8.89 (1H), 11.57 (1H) ppm.

### Examples 207-212

Compounds of examples 207-212 were prepared in analogy to example 14 from (RS)-4-(2,3-dihydro-1H-indol-1-yl)-6,7,8,9-tetrahydro-5H-pyrimido[4,5-b]indole-6-carboxylic acid (prepared according to example 185) as starting material and the reagent given.

### Example 207

### (RS)-4-(2,3-Dihydro-1H-indol-1-yl)-N-ethyl-6,7,8,9-tetrahydro-5H-pyrimido[4,5-b]indole-6-carboxamide

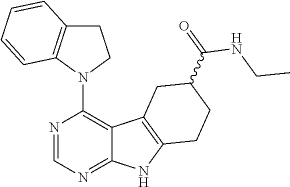 [SMILES: CCNC(=O)C1CCc2[nH]c3ncnc(N4CCc5ccccc54)c3c2C1]

Starting material: Reagent: Product:

40 mg (108 μmol) ethanamine 20.9 mg (51%)

¹H-NMR (DMSO-d6): δ=0.94 (3H), 1.79 (1H), 1.94 (1H), 2.34 (1H), 2.62-2.81 (4H), 2.92-3.15 (4H), 3.98 (1H), 4.20 (1H), 6.80 (1H), 7.00 (1H), 7.10 (1H), 7.19 (1H), 7.74 (1H), 8.23 (1H), 11.66 (1H) ppm.

### Example 208

### (RS)-4-(2,3-Dihydro-1H-indol-1-yl)-N-(propan-2-yl)-6,7,8,9-tetrahydro-5H-pyrimido[4,5-b]indole-6-carboxamide

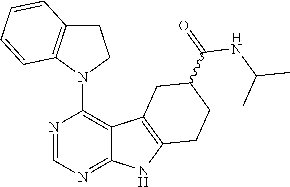 [SMILES: CC(C)NC(=O)C1CCc2[nH]c3ncnc(N4CCc5ccccc54)c3c2C1]

Starting material: Reagent: Product:

40 mg (108 μmol) propan-2-amine 19.4 mg (46%)

¹H-NMR (DMSO-d6): δ=0.97 (6H), 1.78 (1H), 1.92 (1H), 2.31 (1H), 2.60-2.81 (4H), 2.98-3.16 (2H), 3.75 (1H), 3.98 (1H), 4.19 (1H), 6.79 (1H), 7.00 (1H), 7.10 (1H), 7.19 (1H), 7.62 (1H), 8.23 (1H), 11.67 (1H) ppm.

### Example 209

### (RS)—N-Benzyl-4-(2,3-dihydro-1H-indol-1-yl)-6,7,8,9-tetrahydro-5H-pyrimido[4,5-b]indole-6-carboxamide

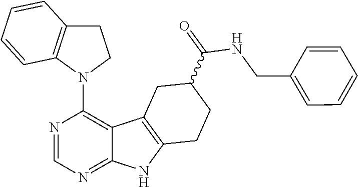 [SMILES: O=C(NCc1ccccc1)C1CCc2[nH]c3ncnc(N4CCc5ccccc54)c3c2C1]

Starting material: Reagent: Product:

40 mg (108 μmol) 1-phenylmethanamine 26.2 mg (54%)

¹H-NMR (DMSO-d6): δ=1.85 (1H), 2.00 (1H), 2.47 (1H), 2.65-2.83 (4H), 3.01-3.15 (2H), 4.00 (1H), 4.15-4.23 (3H), 6.81 (1H), 7.01 (1H), 7.09-7.27 (7H), 8.24 (1H), 8.30 (1H), 11.67 (1H) ppm.

### Example 210

### (RS)-4-(2,3-Dihydro-1H-indol-1-yl)-N-[3-(trifluoromethyl)benzyl]-6,7,8,9-tetrahydro-5H-pyrimido[4,5-b]indole-6-carboxamide

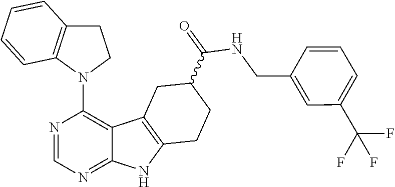 [SMILES: O=C(NCc1cccc(C(F)(F)F)c1)C1CCc2[nH]c3ncnc(N4CCc5ccccc54)c3c2C1]

Starting material: Reagent: Product:

40 mg (108 μmol) 1-[3-(trifluoromethyl)phenyl]methanamine 32.8 mg (59%)

¹H-NMR (DMSO-d6): δ=1.86 (1H), 2.00 (1H), 2.48-2.57 (1H), 2.66-2.83 (4H), 3.00-3.14 (2H), 4.00 (1H), 4.19 (1H), 4.30 (2H), 6.80 (1H), 7.00 (1H), 7.12 (1H), 7.20 (1H), 7.42-7.57 (4H), 8.23 (1H), 8.43 (1H), 11.67 (1H) ppm.

### Example 211

### (RS)-[4-(2,3-Dihydro-1H-indol-1-yl)-6,7,8,9-tetrahydro-5H-pyrimido[4,5-b]indol-6-yl](4-methylpiperazin-1-yl)methanone

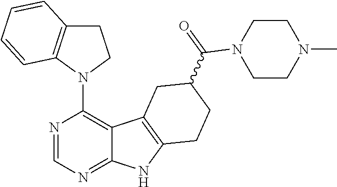 [SMILES: CN1CCN(C(=O)C2CCc3[nH]c4ncnc(N5CCc6ccccc65)c4c3C2)CC1]

Starting material: Reagent: Product:

10 mg (108 μmol) 1-methylpiperazine 14.7 mg (31%)

¹H-NMR (DMSO-d6): δ=1.80 (1H), 1.92 (1H), 2.08 (2H), 2.12 (3H), 2.23 (2H), 2.57-2.68 (2H), 2.73-2.96 (3H), 3.02-3.16 (2H), 3.33-3.51 (4H), 4.05 (1H), 4.16 (1H), 6.82 (1H), 6.98-7.08 (2H), 7.21 (1H), 8.27 (1H), 11.71 (1H) ppm.

### Example 212

### (RS)-4-(2,3-Dihydro-1H-indol-1-yl)-N-methyl-6,7,8,9-tetrahydro-5H-pyrimido[4,5-b]indole-6-carboxamide

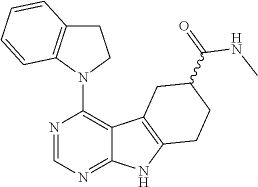 [SMILES: CNC(=O)C1CCc2[nH]c3ncnc(N4CCc5ccccc54)c3c2C1]

Starting material: Reagent: Product:

40 mg (108 μmol) methanamine 23.2 mg (59%)

¹H-NMR (DMSO-d6): δ=1.78 (1H), 1.94 (1H), 2.34 (1H), 2.51 (3H), 2.58-2.82 (4H), 3.00-3.16 (2H), 3.98 (1H), 4.19 (1H), 6.79 (1H), 7.00 (1H), 7.09 (1H), 7.19 (1H), 7.73 (1H), 8.23 (1H), 11.68 (1H) ppm.

### Examples 213-215

Compounds of examples 213-215 were prepared in analogy to example 14 from (RS)-4-{[2-methoxy-5-(trifluoromethyl)phenyl]amino}-6,7,8,9-tetrahydro-5H-pyrimido[4,5-b]indole-6-carboxylic acid (prepared according to example 103) as starting material and the reagent given.

### Example 213

### (RS)-4-{[2-Methoxy-5-(trifluoromethyl)phenyl]amino}-N-(propan-2-yl)-6,7,8,9-tetrahydro-5H-pyrimido[4,5-b]indole-6-carboxamide

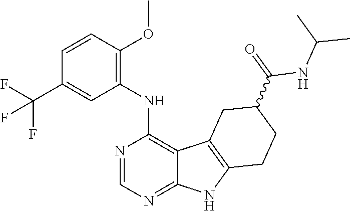 [SMILES: COc1ccc(C(F)(F)F)cc1Nc1ncnc2[nH]c3c(c12)CC(C(=O)NC(C)C)CC3]

Starting material: Reagent: Product:

50 mg (171 μmol) propan-2-amine 17.1 mg (21%)

¹H-NMR (DMSO-d6): δ=1.08 (6H), 1.78 (1H), 2.00 (1H), 2.56 (1H), 2.69 (2H), 2.95 (1H), 3.04 (1H), 3.88 (1H), 3.94 (3H), 7.21 (1H), 7.32 (1H), 7.78 (1H), 7.82 (1H), 8.26 (1H), 9.01 (1H), 11.58 (1H) ppm.

### Example 214

### (RS)-4-{[2-Methoxy-5-(trifluoromethyl)phenyl]amino}-N-phenyl-6,7,8,9-tetrahydro-5H-pyrimido[4,5-b]indole-6-carboxamide

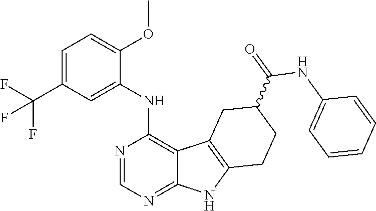 [SMILES: COc1ccc(C(F)(F)F)cc1Nc1ncnc2[nH]c3c(c12)CC(C(=O)Nc1ccccc1)CC3]

Starting material: Reagent: Product:

60 mg (148 μmol) aniline 15 mg (20%)

¹H-NMR (DMSO-d6): δ=1.88 (1H), 2.16 (1H), 2.77 (2H), 2.86 (1H), 3.08 (1H), 3.21 (1H), 3.87 (3H), 7.02 (1H), 7.19 (1H), 7.26-7.34 (3H), 7.63 (2H), 7.86 (1H), 8.26 (1H), 8.93 (1H), 10.03 (1H), 11.61 (1H) ppm.

### Example 215

### (RS)-(4-{[2-Methoxy-5-(trifluoromethyl)phenyl]amino}-6,7,8,9-tetrahydro-5H-pyrimido[4,5-b]indol-6-yl)(4-methylpiperazin-1-yl)methanone

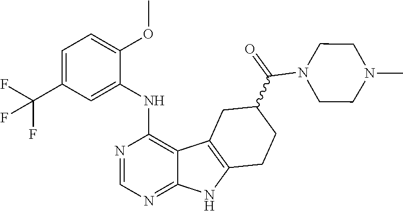 [SMILES: COc1ccc(C(F)(F)F)cc1Nc1ncnc2[nH]c3c(c12)CC(C(=O)N1CCN(C)CC1)CC3]

Starting material: Reagent: Product:

40 mg (98 μmol) 1-methylpiperazine 6.8 mg (13%)

¹H-NMR (DMSO-d6): δ=1.79 (1H), 1.95 (1H), 2.18 (3H), 2.21-2.37 (3H), 2.66-2.91 (3H), 2.92-3.05 (2H), 3.06-3.21 (1H), 3.56 (4H), 3.95 (3H), 7.21 (1H), 7.32 (1H), 7.81 (1H), 8.25 (1H), 9.01 (1H), 11.57 (1H) ppm.

### Examples 216-221

Compounds of examples 216-221 were prepared in analogy to example 14 from (RS)-4-[(6-methoxy-1H-indazol-5-yl)amino]-6,7,8,9-tetrahydro-5H-pyrimido[4,5-b]indole-6-carboxylic acid hydrochloride (1:1) (prepared according to example 184) as starting material and the reagent given.

### Example 216

### (RS)-4-[(6-Methoxy-1H-indazol-5-yl)amino]-N-(propan-2-yl)-6,7,8,9-tetrahydro-5H-pyrimido[4,5-b]indole-6-carboxamide

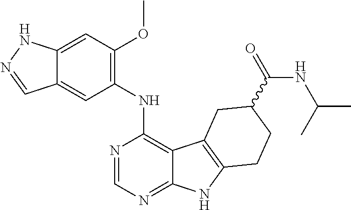 [SMILES: COc1cc2[nH]ncc2cc1Nc1ncnc2[nH]c3c(c12)CC(C(=O)NC(C)C)CC3]

Starting material: Reagent: Product:

50 mg (121 μmol) propan-2-amine 30.8 mg (58%)

¹H-NMR (DMSO-d6): δ=1.08 (6H), 1.78 (1H), 2.00 (1H), 2.56 (1H), 2.69 (2H), 2.94 (1H), 3.07 (1H), 3.90 (1H), 3.94 (3H), 7.04 (1H), 7.77 (1H), 7.78 (1H), 7.94 (1H), 8.22 (1H), 8.86 (1H), 11.46 (1H), 12.75 (1H) ppm.

### Example 217

### (RS)-4-[(6-Methoxy-1H-indazol-5-yl)amino]-N-methyl-6,7,8,9-tetrahydro-5H-pyrimido[4,5-b]indole-6-carboxamide

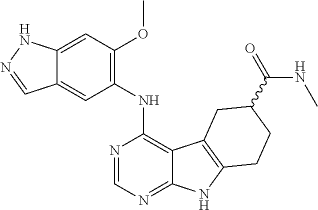 [SMILES: CNC(=O)C1CCc2[nH]c3ncnc(Nc4cc5cn[nH]c5cc4OC)c3c2C1]

Starting material: Reagent: Product:

50 mg (121 μmol) methanamine 25.7 mg (52%)

¹H-NMR (DMSO-d6): δ=1.79 (1H), 2.02 (1H), 2.55-2.73 (3H), 2.63 (3H), 2.97 (1H), 3.08 (1H), 3.93 (3H), 7.04 (1H), 7.76 (1H), 7.87 (1H), 7.94 (1H), 8.20 (1H), 8.79 (1H), 11.45 (1H), 12.76 (1H) ppm.

### Example 218

### (RS)—N-Ethyl-4-[(6-methoxy-1H-indazol-5-yl)amino]-6,7,8,9-tetrahydro-5H-pyrimido[4,5-b]indole-6-carboxamide

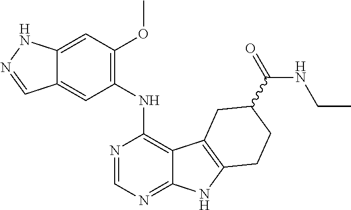 [SMILES: CCNC(=O)C1CCc2[nH]c3ncnc(Nc4cc5cn[nH]c5cc4OC)c3c2C1]

Starting material: Reagent: Product:

50 mg (121 μmol) ethanamine 38 mg (72%)

¹H-NMR (DMSO-d6): δ=1.05 (3H), 1.79 (1H), 2.01 (1H), 2.58 (1H), 2.69 (2H), 2.96 (1H), 3.04-3.18 (3H), 3.93 (3H), 7.04 (1H), 7.77 (1H), 7.86-8.00 (2H), 8.21 (1H), 8.83 (1H), 11.45 (1H), 12.75 (1H) ppm.

### Example 219

### (RS)—N-Benzyl-4-[(6-methoxy-1H-indazol-5-yl)amino]-6,7,8,9-tetrahydro-5H-pyrimido[4,5-b]indole-6-carboxamide

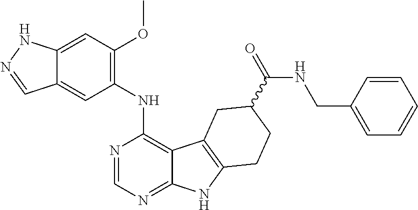 [SMILES: COc1cc2[nH]ncc2cc1Nc1ncnc2[nH]c3c(c12)CC(C(=O)NCc1ccccc1)CC3]

Starting material: Reagent: Product:

50 mg (121 μmol) 1-phenylmethanamine 31.6 mg (53%)

¹H-NMR (DMSO-d6): δ=1.85 (1H), 2.08 (1H), 2.62-2.79 (3H), 2.97-3.24 (2H), 3.84 (3H), 4.33 (2H), 7.03 (1H), 7.17-7.35 (5H), 7.76 (1H), 7.94 (1H), 8.21 (1H), 8.48 (1H), 8.83 (1H), 11.47 (1H), 12.76 (1H) ppm.

### Example 220

### (RS)-4-[(6-Methoxy-1H-indazol-5-yl)amino]-N-[3-(trifluoromethyl)benzyl]-6,7,8,9-tetrahydro-5H-pyrimido[4,5-b]indole-6-carboxamide

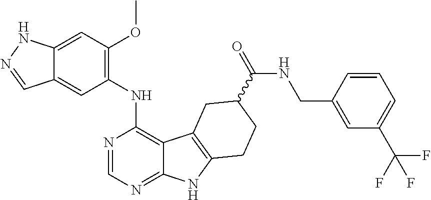 [SMILES: COc1cc2[nH]ncc2cc1Nc1ncnc2[nH]c3c(c12)CC(C(=O)NCc1cccc(C(F)(F)F)c1)CC3]

Starting material: Reagent: Product:

50 mg (121 μmol) 1-[3-(trifluoromethyl)phenyl]methanamine 36.9 mg (54%)

¹H-NMR (DMSO-d6): δ=1.86 (1H), 2.08 (1H), 2.66-2.80 (3H), 3.02 (1H), 3.13 (1H), 3.82 (3H), 4.41 (2H), 7.02 (1H), 7.54-7.65 (4H), 7.75 (1H), 7.94 (1H), 8.21 (1H), 8.59 (1H), 8.82 (1H), 11.46 (1H), 12.74 (1H) ppm.

### Example 221

### (RS)-{4-[(6-Methoxy-1H-indazol-5-yl)amino]-6,7,8,9-tetrahydro-5H-pyrimido[4,5-b]indol-6-yl}(4-methylpiperazin-1-yl)methanone

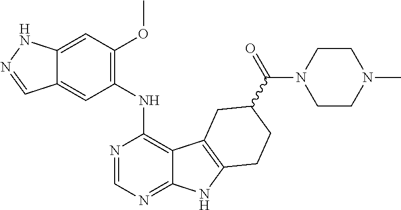 [SMILES: COc1cc2[nH]ncc2cc1Nc1ncnc2[nH]c3c(c12)CC(C(=O)N1CCN(C)CC1)CC3]

Starting material: Reagent: Product:

50 mg (121 μmol) 1-methylpiperazine 33 mg (56%)

¹H-NMR (DMSO-d6): δ=1.78 (1H), 1.95 (1H), 2.18 (3H), 2.21-2.38 (4H), 2.63-2.81 (2H), 2.94-3.04 (2H), 3.11 (1H), 3.41-3.63 (4H), 3.94 (3H), 7.04 (1H), 7.75 (1H), 7.94 (1H), 8.21 (1H), 8.85 (1H), 11.46 (1H), 12.75 (1H) ppm.

### Example 222

### (RS)-6-({6-[(4-Methylpiperazin-1-yl)carbonyl]-6,7,8,9-tetrahydro-5H-pyrimido[4,5-b]indol-4-yl}amino)-1,3-benzothiazol-2(3H)-one

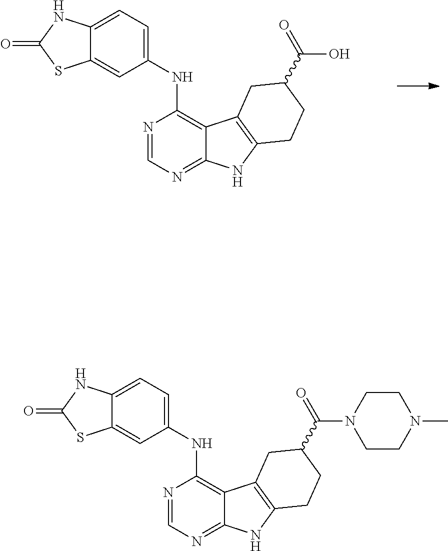 [SMILES: O=C(O)C1CCc2[nH]c3ncnc(Nc4ccc5[nH]c(=O)sc5c4)c3c2C1 ; CN1CCN(C(=O)C2CCc3[nH]c4ncnc(Nc5ccc6[nH]c(=O)sc6c5)c4c3C2)CC1]

80 mg (210 μmol) (RS)-4-[(2-oxo-2,3-dihydro-1,3-benzothiazol-6-yl)amino]-6,7,8,9-tetrahydro-5H-pyrimido[4,5-b]indole-6-carboxylic acid (prepared according to example 88) were transformed in analogy to example 14 using 1-methylpiperazine to give after working up and purification 11 mg (11%) of the title compound.

¹H-NMR (DMSO-d6): δ=1.78 (1H), 1.90 (1H), 2.15 (3H), 2.20-2.44 (4H), 2.59-3.07 (6H), 3.53 (4H), 7.01 (1H), 7.42 (1H), 7.81 (1H), 7.98 (1H), 8.05 (1H), 11.40 (1H) ppm.

### Examples 223-227

Compounds of examples 223-227 were prepared in analogy to example 1 from (RS)-4-chloro-N-isopropyl-6,7,8,9-tetrahydro-5H-pyrimido[4,5-b]indole-6-carboxamide (prepared according to example 223a) as starting material and the reagent given.

### Example 223

### (RS)-4-{[4-Fluoro-2-(propan-2-yloxy)phenyl]amino}-N-(propan-2-yl)-6,7,8,9-tetrahydro-5H-pyrimido[4,5-b]indole-6-carboxamide

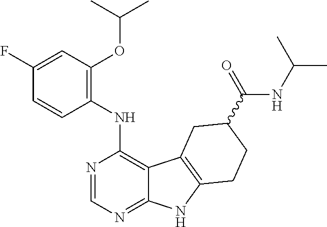 [SMILES: CC(C)NC(=O)C1CCc2[nH]c3ncnc(Nc4ccc(F)cc4OC(C)C)c3c2C1]

Starting material: Reagent: Product:

750 mg (2.56 mmol) 4-fluoro-2-isopropoxyaniline 868 mg (76%)

¹H-NMR (DMSO-d6): δ=1.06 (6H), 1.28 (6H), 1.79 (1H), 1.96 (1H), 2.55 (1H), 2.62-2.72 (2H), 2.99 (2H), 3.83 (1H), 4.74 (1H), 6.75 (1H), 7.01 (1H), 7.63 (1H), 7.77 (1H), 8.20 (1H), 8.73 (1H), 11.51 (1H) ppm.

### Example 224

### (RS)-4-[(5-Chloro-2-methoxyphenyl)amino]-N-(propan-2-yl)-6,7,8,9-tetrahydro-5H-pyrimido[4,5-b]indole-6-carboxamide

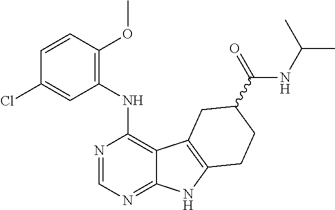 [SMILES: COc1ccc(Cl)cc1Nc1ncnc2[nH]c3c(c12)CC(C(=O)NC(C)C)CC3]

Starting material: Reagent: Product:

50 mg (171 μmol) 5-chloro-2-methoxyaniline 49.0 mg (66%)

¹H-NMR (DMSO-d6): δ=1.07 (6H), 1.78 (1H), 1.99 (1H), 2.55 (1H), 2.61-2.75 (2H), 2.92 (1H), 3.02 (1H), 3.86 (3H), 3.87 (1H), 6.97 (1H), 7.04 (1H), 7.74 (1H), 7.77 (1H), 8.26 (1H), 8.75 (1H), 11.57 (1H) ppm.

### Example 225

### (RS)-4-{[5-Chloro-2-(propan-2-yloxy)phenyl]amino}-N-(propan-2-yl)-6,7,8,9-tetrahydro-5H-pyrimido[4,5-b]indole-6-carboxamide

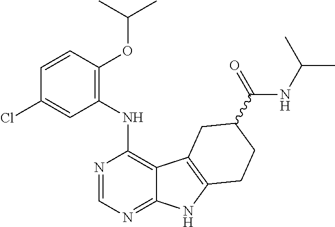 [SMILES: CC(C)NC(=O)C1CCc2[nH]c3ncnc(Nc4cc(Cl)ccc4OC(C)C)c3c2C1]

Starting material: Reagent: Product:

50 mg (171 μmol) 5-chloro-2-isopropoxyaniline 50.0 mg (63%)

¹H-NMR (DMSO-d6): δ=1.05 (6H), 1.27 (6H), 1.79 (1H), 1.96 (1H), 2.55 (1H), 2.69 (1H), 2.99 (2H), 3.40 (1H), 3.81 (1H), 4.71 (1H), 6.92 (1H), 7.06 (1H), 7.76-7.86 (2H), 8.28 (1H), 8.90 (1H), 11.62 (1H) ppm.

### Example 226

### (RS)-4-[(4,5-Dichloro-2-methoxyphenyl)amino]-N-(propan-2-yl)-6,7,8,9-tetrahydro-5H-pyrimido[4,5-b]indole-6-carboxamide

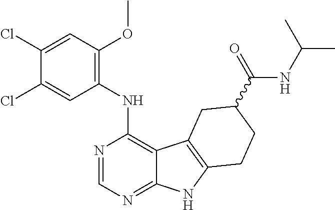 [SMILES: COc1cc(Cl)c(Cl)cc1Nc1ncnc2[nH]c3c(c12)CC(C(=O)NC(C)C)CC3]

Starting material: Reagent: Product:

50 mg (171 μmol) 4,5-dichloro-2-methoxyaniline 20.1 mg (25%)

¹H-NMR (DMSO-d6): δ=1.07 (6H), 1.77 (1H), 1.99 (1H), 2.55 (1H), 2.61-2.74 (2H), 2.91 (1H), 3.01 (1H), 3.88 (1H), 3.90 (3H), 7.29 (1H), 7.70 (1H), 7.77 (1H), 8.26 (1H), 8.90 (1H), 11.60 (1H) ppm.

### Example 227

### (RS)-4-(1H-Indazol-5-ylamino)-N-(propan-2-yl)-6,7,8,9-tetrahydro-5H-pyrimido[4,5-b]indole-6-carboxamide

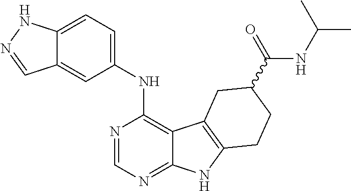 [SMILES: CC(C)NC(=O)C1CCc2[nH]c3ncnc(Nc4ccc5[nH]ncc5c4)c3c2C1]

Starting material: Reagent: Product:

50 mg (171 μmol) 1H-indazol-5-amine 15.1 mg (22%)

¹H-NMR (DMSO-d6): δ=1.05 (6H), 1.76 (1H), 1.95 (1H), 2.47 (1H), 2.59-2.73 (2H), 2.93 (1H), 3.07 (1H), 3.84 (1H), 7.43 (1H), 7.49 (1H), 7.70 (1H), 7.95-7.99 (3H), 8.04 (1H), 11.34 (1H), 12.89 (1H) ppm.

### Example 223a

### (RS)-4-Chloro-N-isopropyl-6,7,8,9-tetrahydro-5H-pyrimido[4,5-b]indole-6-carboxamide

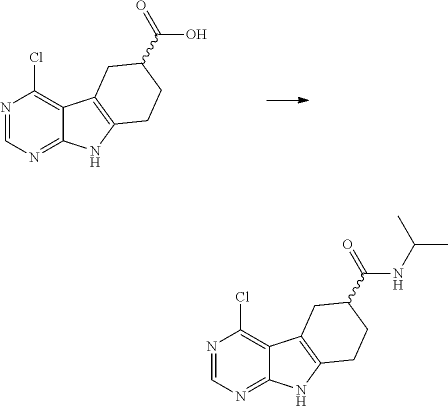 [SMILES: O=C(O)C1CCc2[nH]c3ncnc(Cl)c3c2C1 ; CC(C)NC(=O)C1CCc2[nH]c3ncnc(Cl)c3c2C1]

550 mg (2.19 mmol) (RS)-4-Chloro-6,7,8,9-tetrahydro-5H-pyrimido[4,5-b]indole-6-carboxylic acid (prepared according to intermediate example 28b) were transformed in analogy to example 14 using propan-2-amine to give after working up and purification 526.7 mg (82%) of the title compound.

### Example 228

### (RS)-Ethyl N-[(4-{[4-fluoro-2-(2-hydroxyethoxy)phenyl]amino}-6,7,8,9-tetrahydro-5H-pyrimido[4,5-b]indol-6-yl)carbonyl]glycinate

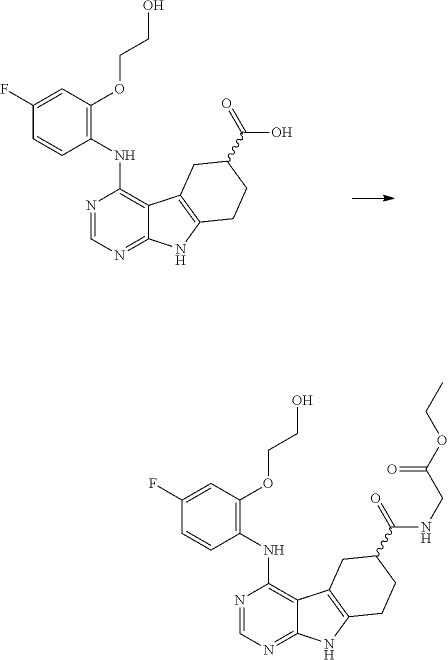 [SMILES: O=C(O)C1CCc2[nH]c3ncnc(Nc4ccc(F)cc4OCCO)c3c2C1 ; CCOC(=O)CNC(=O)C1CCc2[nH]c3ncnc(Nc4ccc(F)cc4OCCO)c3c2C1]

216 mg (559 μmol) (RS)-4-{[4-fluoro-2-(2-hydroxyethoxy)phenyl]amino}-6,7,8,9-tetrahydro-5H-pyrimido[4,5-b]indole-6-carboxylic acid (prepared according to intermediate example 182) were transformed in analogy to example 14 using ethyl glycinate to give after working up and purification 62 mg (22%) of the title compound.

¹H-NMR (DMSO-d6): δ=1.17 (3H), 1.81 (1H), 2.05 (1H), 2.61-2.79 (3H), 3.00 (1H), 3.11 (1H), 3.66-3.84 (3H), 3.92 (1H), 4.01-4.15 (4H), 4.76 (1H), 6.76 (1H), 6.98 (1H), 7.68 (1H), 8.18 (1H), 8.38 (1H), 8.63 (1H), 11.51 (1H) ppm.

### Example 229

### (RS)-11-Fluoro-1,3,4,7,8,14-hexahydro-5H-2,4-ethano-6,9-dioxa-1,14,15,17-tetraazabenzo[5,6]cyclotrideca[1,2,3-cd]inden-5-one

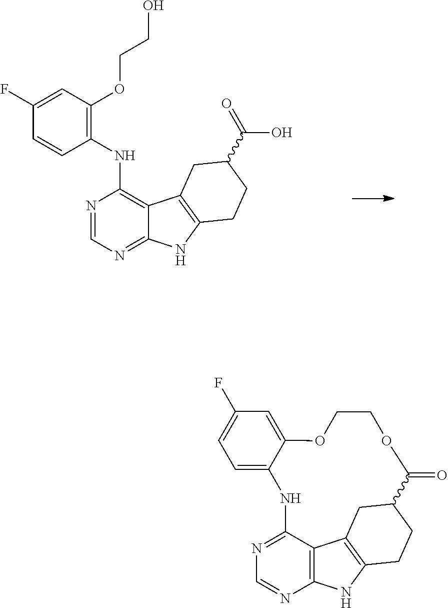 [SMILES: O=C(O)C1CCc2[nH]c3ncnc(Nc4ccc(F)cc4OCCO)c3c2C1 ; O=C1OCCOc2cc(F)ccc2Nc2ncnc3[nH]c4c(c23)CC1CC4]

To the solution of 100 mg (259 μmol) (RS)-4-{[4-fluoro-2-(2-hydroxyethoxy)phenyl]amino}-6,7,8,9-tetrahydro-5H-pyrimido[4,5-b]indole-6-carboxylic acid (prepared according to intermediate example 182) in 5 mL N,N-dimethylformamide were added at 23° C. 264 μL N-ethyl-N-isopropylpropan-2-amine triethylamine, 206 μL 2,4,6-trichlorobenzoylchloride and after 10 min 50 mL N,N-dimethylformamide. This solution was added with constant speed over a time period of about 6 h to the stirred solution of 329 mg N,N-dimethylpyridin-4-amine in 20 mL dichloromethane. After stirring overnight the solution was concentrated in vacuo and the residue purified chromatography to give 4.7 mg (5%) of the title compound.

¹H-NMR (DMSO-d6): δ=1.94 (1H), 2.24 (1H), 2.63 (1H), 2.68-2.90 (3H), 3.15 (1H), 3.76 (1H), 3.87-4.01 (2H), 4.14 (1H), 6.83 (1H), 6.96 (1H), 6.98 (1H), 7.50 (1H), 7.94 (1H), 11.34 (1H) ppm.

### Example 230

### (RS)—N-[3-(Methylsulfonyl)propyl]-4-{[6-(propan-2-yloxy)-1H-indazol-5-yl]amino}-6,7,8,9-tetrahydro-5H-pyrimido[4,5-b]indole-6-carboxamide

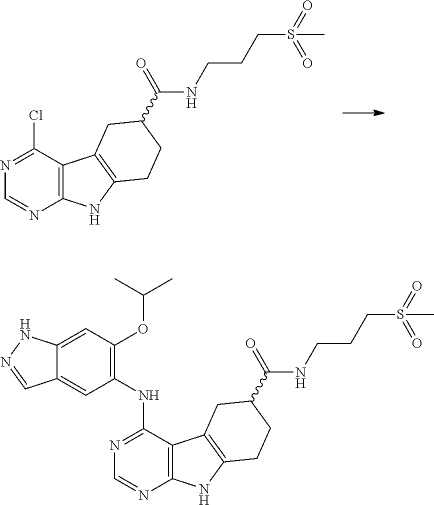 [SMILES: CS(=O)(=O)CCCNC(=O)C1CCc2[nH]c3ncnc(Cl)c3c2C1 ; CC(C)Oc1cc2[nH]ncc2cc1Nc1ncnc2[nH]c3c(c12)CC(C(=O)NCCCS(C)(=O)=O)CC3]

60 mg (162 μmol) (RS)-4-chloro-N-[3-(methylsulfonyl)propyl]-6,7,8,9-tetrahydro-5H-pyrimido[4,5-b]indole-6-carboxamide (prepared according to intermediate example 28a) were transformed in analogy to example 1 using 6-isopropoxy-1H-indazol-5-amine to give after working up and purification 58.5 mg (62%) of the title compound.

¹H-NMR (DMSO-d6): δ=1.16 (6H), 1.73-1.93 (3H), 2.03 (1H), 2.58 (1H), 2.67-2.99 (3H), 2.93 (3H), 3.04-3.26 (6H), 4.69 (1H), 7.13 (1H), 8.02 (1H), 8.05-8.19 (2H), 9.38 (1H), 12.41 (1H), 12.95 (1H) ppm.

### Example 231

### (RS)-6-Methoxy-6-(methoxymethyl)-N-[6-(propan-2-yloxy)-1H-indazol-5-yl]-6,7,8,9-tetrahydro-5H-pyrimido[4,5-b]indol-4-amine

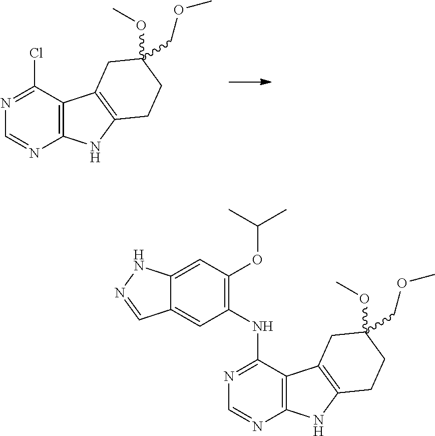

60 mg (213 μmol) (RS)-4-chloro-6-methoxy-6-(methoxymethyl)-6,7,8,9-tetrahydro-5H-pyrimido[4,5-b]indole (prepared according to intermediate example 131a) were transformed in analogy to example 1 using 6-isopropoxy-1H-indazol-5-amine to give after working up and purification 25.3 mg (26%) of the title compound.

¹H-NMR (DMSO-d6): δ=1.15 (6H), 1.80 (1H), 1.99 (1H), 2.60-2.72 (2H), 2.80 (1H), 3.03 (1H), 3.14 (3H), 3.27 (3H), 3.40 (2H), 4.69 (1H), 7.14 (1H), 8.02 (1H), 8.07 (1H), 8.12 (1H), 9.41 (1H), 12.46 (1H), 12.98 (1H) ppm.

### Example 232

### 6,6-Difluoro-N-[6-(propan-2-yloxy)-1H-indazol-5-yl]-6,7,8,9-tetrahydro-5H-pyrimido[4,5-b]indol-4-amine

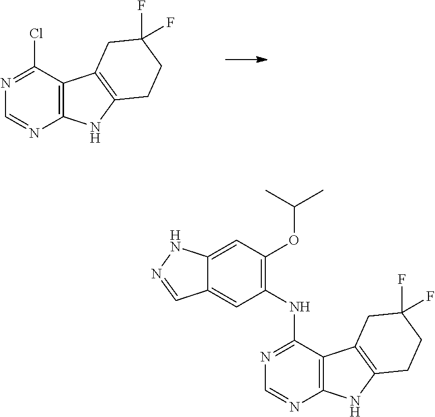 [SMILES: FC1(F)CCc2[nH]c3ncnc(Cl)c3c2C1 ; CC(C)Oc1cc2[nH]ncc2cc1Nc1ncnc2[nH]c3c(c12)CC(F)(F)CC3]

60 mg (246 μmol) 4-chloro-6,6-difluoro-6,7,8,9-tetrahydro-5H-pyrimido[4,5-b]indole (prepared according to intermediate example 9a) were transformed in analogy to example 1 using 6-isopropoxy-1H-indazol-5-amine to give after working up and purification 78.6 mg (76%) of the title compound.

¹H-NMR (DMSO-d6): δ=1.16 (6H), 2.33 (2H), 2.91 (2H), 3.48 (2H), 4.69 (1H), 7.14 (1H), 8.03 (1H), 8.09 (1H), 8.16 (1H), 9.50 (1H), 12.66 (1H), 12.99 (1H) ppm.

### Example 233

### (RS)-4-[(3,4-Dichlorophenyl)amino]-6,7,8,9-tetrahydro-5H-pyrimido[4,5-b]indole-6-carboxylic acid

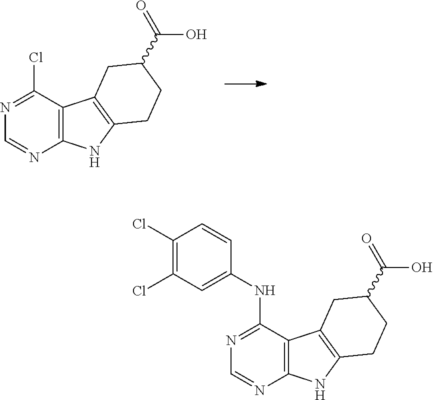 [SMILES: O=C(O)C1CCc2[nH]c3ncnc(Cl)c3c2C1 ; O=C(O)C1CCc2[nH]c3ncnc(Nc4ccc(Cl)c(Cl)c4)c3c2C1]

40 mg (99 μmol) (RS)-4-Chloro-6,7,8,9-tetrahydro-5H-pyrimido[4,5-b]indole-6-carboxylic acid (prepared according to intermediate example 28b) were transformed in analogy to example 1 using 3,4-dichloroaniline to give after working up and purification 31.7 mg (81%) of the title compound.

¹H-NMR (DMSO-d6): δ=1.81 (1H), 2.12 (1H), 2.62-2.75 (3H), 2.98 (1H), 3.22 (1H), 7.49 (1H), 7.70 (1H), 8.05 (1H), 8.18 (1H), 8.25 (1H), 11.54 (1H), 12.29 (1H) ppm.

### Example 234

### (RS)-4-{[5-Chloro-2-(propan-2-yloxy)phenyl]amino}-6,7,8,9-tetrahydro-5H-pyrimido[4,5-b]indole-6-carboxylic acid

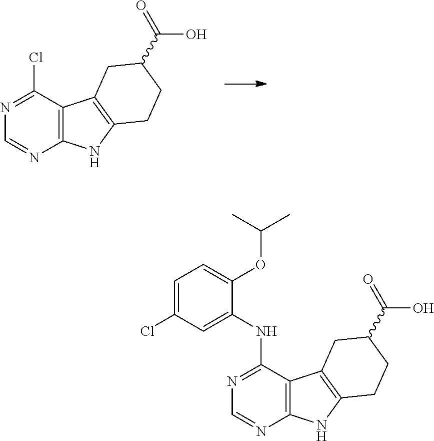 [SMILES: O=C(O)C1CCc2[nH]c3ncnc(Cl)c3c2C1 ; CC(C)Oc1ccc(Cl)cc1Nc1ncnc2[nH]c3c(c12)CC(C(=O)O)CC3]

28 mg (65 μmol) (RS)-4-Chloro-6,7,8,9-tetrahydro-5H-pyrimido[4,5-b]indole-6-carboxylic acid (prepared according to intermediate example 28b) were transformed in analogy to example 1 using 5-chloro-2-isopropoxyaniline to give after working up and purification 16.5 mg (63%) of the title compound.

¹H-NMR (DMSO-d6): δ=1.32 (6H), 1.82 (1H), 2.17 (1H), 2.68-2.82 (3H), 3.04 (1H), 3.17 (1H), 4.73 (1H), 6.93 (1H), 7.07 (1H), 7.82 (1H), 8.28 (1H), 8.92 (1H), 11.60 (1H), 12.39 (1H) ppm.

### Example 235

### (RS)-4-[(5-Chloro-2-methoxyphenyl)amino]-6,7,8,9-tetrahydro-5H-pyrimido[4,5-b]indole-6-carboxylic acid

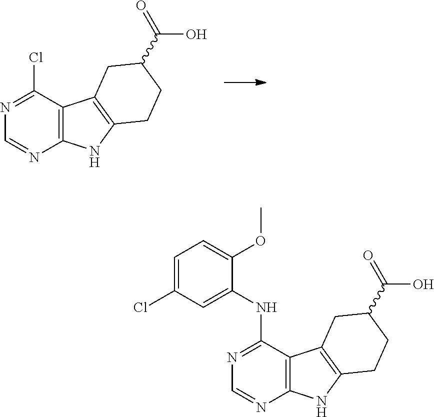 [SMILES: O=C(O)C1CCc2[nH]c3ncnc(Cl)c3c2C1 ; COc1ccc(Cl)cc1Nc1ncnc2[nH]c3c(c12)CC(C(=O)O)CC3]

12 mg (30 μmol) (RS)-4-Chloro-6,7,8,9-tetrahydro-5H-pyrimido[4,5-b]indole-6-carboxylic acid (prepared according to intermediate example 28b) were transformed in analogy to example 1 using 5-chloro-2-methoxyaniline to give after working up and purification 3.1 mg (26%) of the title compound.

¹H-NMR (DMSO-d6): δ=1.83 (1H), 2.16 (1H), 2.71 (2H), 2.79 (1H), 3.01 (1H), 3.16 (1H), 3.89 (3H), 6.98 (1H), 7.05 (1H), 7.78 (1H), 8.26 (1H), 8.73 (1H), 11.57 (1H), 12.34 (1H) ppm.

### Example 236

### 5-Chloro-6-(6,7,8,9-tetrahydro-5H-pyrimido[4,5-b]indol-4-ylamino)-1,3-benzothiazol-2(3H)-one

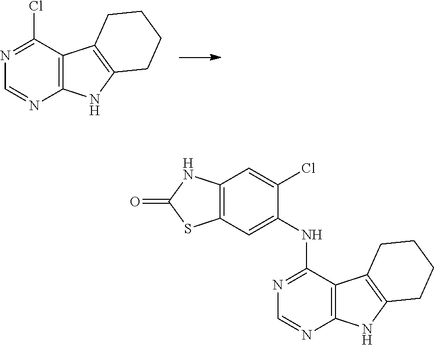 [SMILES: Clc1ncnc2[nH]c3c(c12)CCCC3 ; O=c1[nH]c2cc(Cl)c(Nc3ncnc4[nH]c5c(c34)CCCC5)cc2s1]

75 mg (361 μmol) 4-chloro-6,7,8,9-tetrahydro-5H-pyrimido[4,5-b]indole (prepared according to intermediate example 1a) were transformed in analogy to example 1 using 6-amino-5-chloro-1,3-benzothiazol-2(3H)-one to give after working up and purification 8.7 mg (6%) of the title compound.

¹H-NMR (DMSO-d6): δ=1.79 (4H), 2.62 (2H), 2.87 (2H), 7.18 (1H), 7.67 (1H), 8.07 (1H), 8.37 (1H), 11.40 (1H), 11.89 (1H) ppm.

### Example 237

### (RS)-4-[(6-Chloro-2-oxo-2,3-dihydro-1H-benzimidazol-5-yl)amino]-6,7,8,9-tetrahydro-5H-pyrimido[4,5-b]indole-6-carboxylic acid

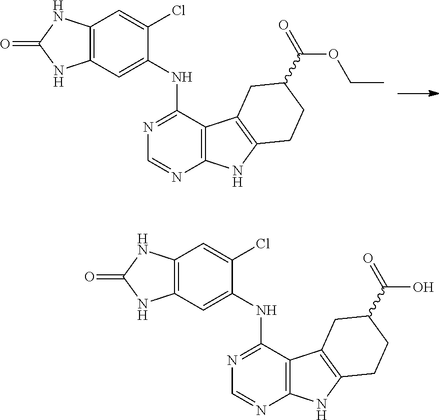 [SMILES: CCOC(=O)C1CCc2[nH]c3ncnc(Nc4cc5[nH]c(=O)[nH]c5cc4Cl)c3c2C1 ; O=C(O)C1CCc2[nH]c3ncnc(Nc4cc5[nH]c(=O)[nH]c5cc4Cl)c3c2C1]

19.2 mg (45 μmol) (RS)-ethyl 4-[(6-chloro-2-oxo-2,3-dihydro-1H-benzimidazol-5-yl)amino]-6,7,8,9-tetrahydro-5H-pyrimido[4,5-b]indole-6-carboxylate (prepared according to intermediate example 237a) were transformed in analogy to example 6 to give after working up and purification 8.8 mg (42%) of the title compound.

¹H-NMR (DMSO-d6): δ=1.82 (1H), 2.15 (1H), 2.67-2.82 (3H), 2.92 (1H), 3.19 (1H), 7.13 (1H), 7.27 (1H), 8.12 (1H), 9.56 (1H), 10.98 (1H), 12.49 (2H) ppm.

### Example 237a

### (RS)-Ethyl 4-[(6-chloro-2-oxo-2,3-dihydro-1H-benzimidazol-5-yl)amino]-6,7,8,9-tetrahydro-5H-pyrimido[4,5-b]indole-6-carboxylate

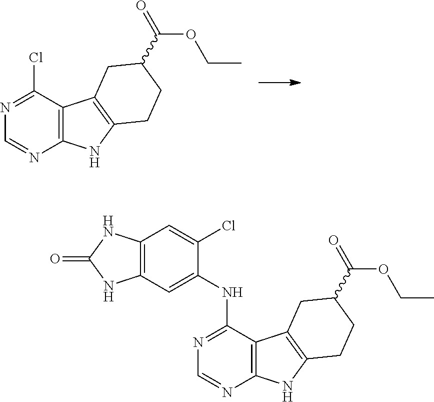 [SMILES: CCOC(=O)C1CCc2[nH]c3ncnc(Cl)c3c2C1 ; CCOC(=O)C1CCc2[nH]c3ncnc(Nc4cc5[nH]c(=O)[nH]c5cc4Cl)c3c2C1]

100 mg (357 μmol) (RS)-ethyl 4-chloro-6,7,8,9-tetrahydro-5H-pyrimido[4,5-b]indole-6-carboxylate (prepared according to intermediate example 5a) were transformed in analogy to example 1 using 5-amino-6-chloro-1,3-dihydro-2H-benzimidazol-2-one to give after working up and purification 19.2 mg (9%) of the title compound.

### Example 238

### 6-[(6,6-Difluoro-6,7,8,9-tetrahydro-5H-pyrimido[4,5-b]indol-4-yl)amino]-5-methoxy-1,3-benzothiazol-2(3H)-one

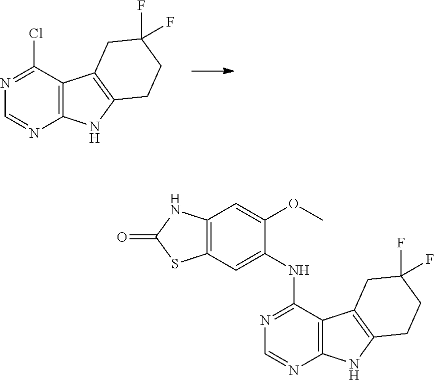 [SMILES: FC1(F)CCc2[nH]c3ncnc(Cl)c3c2C1 ; COc1cc2[nH]c(=O)sc2cc1Nc1ncnc2[nH]c3c(c12)CC(F)(F)CC3]

30 mg (123 μmol) 4-chloro-6,6-difluoro-6,7,8,9-tetrahydro-5H-pyrimido[4,5-b]indole (prepared according to intermediate example 9a) were transformed in analogy to example 1 using 6-methoxy-1H-indazol-5-amine to give after working up and purification 20.9 mg (40%) of the title compound.

¹H-NMR (DMSO-d6): δ=2.30 (2H), 2.85 (2H), 3.44 (2H), 3.84 (3H), 6.78 (1H), 7.59 (1H), 8.13 (1H), 8.33 (1H), 11.64 (1H), 11.71 (1H) ppm.

Further, the compounds of formula I of the present invention can be converted to any salt as described herein, by any method which is known to the person skilled in the art. Similarly, any salt of a compound of formula I of the present invention can be converted into the free compound, by any method which is known to the person skilled in the art.

### Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al., “Compendium of Excipients for Parenteral Formulations” PDA Journal of Pharmaceutical Science [symbol] Technology 1998, 52(5), 238-311; Strickley, R. G “Parenteral Formulations of Small Molecule Therapeutics Marketed in the United States (1999)—Part-1” PDA Journal of Pharmaceutical Science [symbol] Technology 1999, 53(6), 324-349; and Nema, S. et al., “Excipients and Their Use in Injectable Products” PDA Journal of Pharmaceutical Science [symbol] Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃)
air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate)
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)
chelating agents (examples include but are not limited to edetate disodium and edetic acid)
colourants (examples include but are not limited to FD [symbol] C Red No. 3, FD [symbol] C Red No. 20, FD [symbol] C Yellow No. 6, FD [symbol] C Blue No. 2, D [symbol] C Green No. 5, D [symbol] C Orange No. 5, D [symbol] C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples include but are not limited to gelatin and cellulose acetate phthalate)
flavourants (examples include but are not limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)
plasticizers (examples include but are not limited to diethyl phthalate and glycerol);
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, Lecithins, sorbitol monooleate, polyoxyethylene sorbitol monooleate, and polyoxyethylene stearate).

Pharmaceutical compositions according to the present invention can be illustrated as follows:

Sterile IV Solution:

A 5 mg/mL solution of the desired compound of this invention can be made using sterile, injectable water, and the pH is adjusted if necessary. The solution is diluted for administration to 1-2 mg/mL with sterile 5% dextrose and is administered as an IV infusion over about 60 minutes.

Lyophilised Powder for IV Administration:

A sterile preparation can be prepared with (i) 100-1000 mg of the desired compound of this invention as a lyophilised powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.

Intramuscular Suspension:

The following solution or suspension can be prepared, for intramuscular injection:

50 mg/mL of the desired, water-insoluble compound of this invention
5 mg/mL sodium carboxymethylcellulose
4 mg/mL TWEEN 80
9 mg/mL sodium chloride
9 mg/mL benzyl alcohol

Hard Shell Capsules:

A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules:

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets:

A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules:

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known chemotherapeutic agents or anti-cancer agents, e.g. anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, toposisomerase inhibitors, biological response modifiers, or anti-hormones.

The terms “chemotherapeutic agent” and “anti-cancer agent”, include but are not limited to 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, BAY 1000394, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, Lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, radium-223 chloride, raloxifene, raltitrexed, ranimustine, razoxane, refametinib, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

In a preferred embodiment, a compound of general formula (I) as defined herein is administered in combination with one or more inhibitors of the PI3K-AKT-mTOR pathway. Examples of inhibitors of the mammalian Target of Rapamycin (mTOR) are Afinitor, Votubia (everolimus).

Generally, the use of cytotoxic and/or cytostatic agents in combination with a compound or composition of the present invention will serve to:
(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

### Methods of Sensitizing Cells to Radiation

In a distinct embodiment of the present invention, a compound of the present invention may be used to sensitize a cell to radiation. That is, treatment of a cell with a compound of the present invention prior to radiation treatment of the cell renders the cell more susceptible to DNA damage and cell death than the cell would be in the absence of any treatment with a compound of the invention. In one aspect, the cell is treated with at least one compound of the invention.

Thus, the present invention also provides a method of killing a cell, wherein a cell is administered one or more compounds of the invention in combination with conventional radiation therapy.

The present invention also provides a method of rendering a cell more susceptible to cell death, wherein the cell is treated with one or more compounds of the invention prior to the treatment of the cell to cause or induce cell death. In one aspect, after the cell is treated with one or more compounds of the invention, the cell is treated with at least one compound, or at least one method, or a combination thereof, in order to cause DNA damage for the purpose of inhibiting the function of the normal cell or killing the cell.

In one embodiment, a cell is killed by treating the cell with at least one DNA damaging agent. That is, after treating a cell with one or more compounds of the invention to sensitize the cell to cell death, the cell is treated with at least one DNA damaging agent to kill the cell. DNA damaging agents useful in the present invention include, but are not limited to, chemotherapeutic agents (e.g., cisplatinum), ionizing radiation (X-rays, ultraviolet radiation), carcinogenic agents, and mutagenic agents.

In another embodiment, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of the invention is administered to a cell prior to the radiation or other induction of DNA damage in the cell. In another aspect of the invention, a compound of the invention is administered to a cell concomitantly with the radiation or other induction of DNA damage in the cell. In yet another aspect of the invention, a compound of the invention is administered to a cell immediately after radiation or other induction of DNA damage in the cell has begun.

In another aspect, the cell is in vitro. In another embodiment, the cell is in vivo.

As mentioned supra, the compounds of the present invention have surprisingly been found to effectively inhibit MKNK-1 and may therefore be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK-1, such as, for example, haematological tumours, solid tumours, and/or metastases thereof, e.g. Leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

In accordance with another aspect therefore, the present invention covers a compound of general formula I, or a stereoisomer, a tautomer, an N-oxide, a hydrate, a solvate, or a salt thereof, particularly a pharmaceutically acceptable salt thereof, or a mixture of same, as described and defined herein, for use in the treatment or prophylaxis of a disease, as mentioned supra.

Another particular aspect of the present invention is therefore the use of a compound of general formula I, described supra, or a stereoisomer, a tautomer, an N-oxide, a hydrate, a solvate, or a salt thereof, particularly a pharmaceutically acceptable salt thereof, or a mixture of same, for the prophylaxis or treatment of a disease.

Another particular aspect of the present invention is therefore the use of a compound of general formula I described supra for manufacturing a pharmaceutical composition for the treatment or prophylaxis of a disease.

The diseases referred to in the two preceding paragraphs are diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK-1, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Preferably, the use is in the treatment or prophylaxis of diseases, wherein the diseases are haemotological tumours, solid tumours and/or metastases thereof.

### Method of Treating Hyper-Proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus.

Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's Lymphoma, cutaneous T-cell Lymphoma, Burkitt Lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

### Methods of Treating Kinase Disorders

The present invention also provides methods for the treatment of disorders associated with aberrant mitogen extracellular kinase activity, including, but not limited to stroke, heart failure, hepatomegaly, cardiomegaly, diabetes, Alzheimer's disease, cystic fibrosis, symptoms of xenograft rejections, septic shock or asthma.

Effective amounts of compounds of the present invention can be used to treat such disorders, including those diseases (e.g., cancer) mentioned in the Background section above. Nonetheless, such cancers and other diseases can be treated with compounds of the present invention, regardless of the mechanism of action and/or the relationship between the kinase and the disorder.

The phrase “aberrant kinase activity” or “aberrant tyrosine kinase activity,” includes any abnormal expression or activity of the gene encoding the kinase or of the polypeptide it encodes. Examples of such aberrant activity, include, but are not limited to, over-expression of the gene or polypeptide; gene amplification; mutations which produce constitutively-active or hyperactive kinase activity; gene mutations, deletions, substitutions, additions, etc.

The present invention also provides for methods of inhibiting a kinase activity, especially of mitogen extracellular kinase, comprising administering an effective amount of a compound of the present invention, including salts, polymorphs, metabolites, hydrates, solvates, prodrugs (e.g.: esters) thereof, and diastereoisomeric forms thereof. Kinase activity can be inhibited in cells (e.g., in vitro), or in the cells of a mammalian subject, especially a human patient in need of treatment.

### Methods of Treating Angiogenic Disorders

The present invention also provides methods of treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death or apoptosis of such cell types.

### Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the diseases of said method are haematological tumours, solid tumour and/or metastases thereof.

The compounds of the present invention can be used in particular in therapy and prevention, i.e. prophylaxis, of tumour growth and metastases, especially in solid tumours of all indications and stages with or without pre-treatment of the tumour growth.

Methods of testing for a particular pharmacological or pharmaceutical property are well known to persons skilled in the art.

The example testing experiments described herein serve to illustrate the present invention and the invention is not limited to the examples given.

### Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

### MKNK1 Kinase Assay

MKNK1-inhibitory activity of compounds of the present invention was quantified employing the MKNK1 TR-FRET assay as described in the following paragraphs.

A recombinant fusion protein of Glutathione-S-Transferase (GST, N-terminally) and human full-length MKNK1 (amino acids 1-424 and T344D of accession number BAA 19885.1), expressed in insect cells using baculovirus expression system and purified via glutathione sepharose affinity chromatography, was purchased from Carna Biosciences (product no 02-145) and used as enzyme. As substrate for the kinase reaction the biotinylated peptide biotin-Ahx-IKKRKLTRRKSLKG (C-terminus in amide form) was used which can be purchased e.g. form the company Biosyntan (Berlin-Buch, Germany).

For the assay 50 nL of a 100fold concentrated solution of the test compound in DMSO was pipetted into a black low volume 384well microtiter plate (Greiner Bio-One, Frickenhausen, Germany), 2 μL of a solution of MKNK1 in aqueous assay buffer [50 mM HEPES pH 7.5, 5 mM MgCl₂, 1.0 mM dithiothreitol, 0.005% (v/v) Nonidet-P40 (Sigma)] was added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (0.1 μM=>final conc. in the 5 μL assay volume is 0.06 μM) in assay buffer and the resulting mixture was incubated for a reaction time of 45 min at 22° C. The concentration of MKNK1 was adjusted depending of the activity of the enzyme lot and was chosen appropriate to have the assay in the linear range, typical concentrations were in the range of 0.05 μg/mL. The reaction was stopped by the addition of 5 μL of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 Labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm were measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100fold concentrated solutions in DMSO by serial 1:3.4 dilutions) in duplicate values for each concentration and IC50 values were calculated by a 4 parameter fit using an inhouse software.

### MKNK1 Kinase High ATP Assay

MKNK1-inhibitory activity at high ATP of compounds of the present invention after their preincubation with MKNK1 was quantified employing the TR-FRET-based MKNK1 high ATP assay as described in the following paragraphs.

A recombinant fusion protein of Glutathione-S-Transferase (GST, N-terminally) and human full-length MKNK1 (amino acids 1-424 and T344D of accession number BAA 19885.1), expressed in insect cells using baculovirus expression system and purified via glutathione sepharose affinity chromatography, was purchased from Carna Biosciences (product no 02-145) and used as enzyme. As substrate for the kinase reaction the biotinylated peptide biotin-Ahx-IKKRKLTRRKSLKG (C-terminus in amide form) was used, which can be purchased e.g. from the company Biosyntan (Berlin-Buch, Germany).

For the assay 50 nL of a 100fold concentrated solution of the test compound in DMSO was pipetted into a black low volume 384well microtiter plate (Greiner Bio-One, Frickenhausen, Germany), 2 μL of a solution of MKNK1 in aqueous assay buffer [50 mM HEPES pH 7.5, 5 mM MgCl₂, 1.0 mM dithiothreitol, 0.005% (v/v) Nonidet-P40 (Sigma)] was added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 3.3 mM=>final conc. in the 5 μL assay volume is 2 mM) and substrate (0.1 μM=>final conc. in the 5 μL assay volume is 0.06 μM) in assay buffer and the resulting mixture was incubated for a reaction time of 30 min at 22° C. The concentration of MKNK1 was adjusted depending of the activity of the enzyme lot and was chosen appropriate to have the assay in the linear range, typical concentrations were in the range of 0.003 μg/mL. The reaction was stopped by the addition of 5 μL of a solution of TR-FRET detection reagents (5 nM streptavidine-XL665 [Cisbio Bioassays, Codolet, France] and 1 nM anti-ribosomal protein 56 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm were measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100fold concentrated solutions in DMSO by serial dilutions, the exact concentrations may vary depending on the pipettor used) in duplicate values for each concentration and IC50 values were calculated by a 4 parameter fit using an inhouse software. Data are presented in Table 1.

TABLE 1

| | MKNK1 |
|---|---|
| | IC50 |
| Example | [nM] |
| 1 | 43 |
| 2 | 27 |
| 3 | 45 |
| 4 | 258 |
| 5 | 18 |
| 6 | 89 |
| 7 | 20 |
| 8 | 2940 |
| 9 | 85 |
| 10 | 24 |
| 11 | 42 |
| 14 | 8 |
| 15 | 19 |
| 16 | 15 |
| 17 | 46 |
| 18 | 24 |
| 20 | 6 |
| 21 | 12 |
| 22 | 313 |
| 24 | 367 |
| 25 | 381 |
| 26 | 1740 |
| 27 | 51 |
| 28 | 8 |
| 29 | 14 |
| 30 | 52 |
| 31 | 2750 |
| 32 | 746 |
| 33 | 394 |
| 34 | 306 |
| 35 | 9660 |
| 36 | 3170 |
| 37 | 145 |
| 38 | 10600 |
| 39 | 59 |
| 40 | 1160 |
| 41 | 9330 |
| 42 | 615 |
| 43 | 782 |
| 44 | 6 |
| 45 | 82 |
| 46 | 2240 |
| 47 | 1310 |
| 48 | 2840 |
| 49 | 231 |
| 50 | 6410 |
| 51 | 370 |
| 52 | 904 |
| 53 | 335 |
| 54 | 3080 |
| 55 | 511 |
| 56 | 226 |
| 57 | n.s. |
| 58 | 1170 |
| 59 | 124 |
| 60 | 1360 |
| 61 | 17 |
| 62 | 677 |
| 64 | 19700 |
| 65 | 13300 |
| 66 | 43 |
| 67 | 28 |
| 68 | 6160 |
| 69 | 507 |
| 70 | 48 |
| 71 | 10800 |
| 73 | 4140 |
| 74 | 1160 |
| 75 | 7670 |
| 77 | 3630 |
| 78 | 24 |
| 79 | 58 |
| 80 | 494 |
| 81 | 1270 |
| 82 | 10500 |
| 83 | 62 |
| 84 | 82 |
| 85 | 192 |
| 86 | 220 |
| 103 | 400 |
| 108 | 126 |
| 109 | 20000 |
| 110 | 12200 |
| 111 | 1100 |
| 112 | 6 |
| 113 | 683 |
| 114 | 1490 |
| 115 | 1790 |
| 116 | 20000 |
| 117 | 58 |
| 118 | 20000 |
| 119 | 20000 |
| 120 | 8350 |
| 121 | 20000 |
| 122 | 4290 |
| 123 | 1550 |
| 124 | 1740 |
| 125 | 104 |
| 126 | 5 |
| 127 | 6270 |
| 128 | 313 |
| 129 | 4050 |
| 130 | 89 |
| 131 | 5700 |
| 132 | 503 |
| 133 | 205 |
| 134 | 1650 |
| 135 | 167 |
| 136 | 48 |
| 137 | 773 |
| 138 | 358 |
| 139 | 197 |
| 140 | 339 |
| 141 | 6 |
| 142 | 38 |
| 143 | 407 |
| 144 | 20000 |
| 145 | 20000 |
| 146 | 1130 |
| 147 | 5920 |
| 148 | 1730 |
| 149 | 10 |
| 150 | 12 |
| 151 | 190 |
| 152 | 113 |
| 153 | 193 |
| 154 | 469 |
| 155 | 504 |
| 156 | 20000 |
| 157 | 444 |
| 158 | 20000 |
| 159 | 36 |
| 160 | 37 |
| 161 | 35 |
| 162 | 120 |
| 163 | 4 |
| 164 | 225 |
| 165 | 86 |
| 166 | 45 |
| 167 | 70 |
| 168 | 803 |
| 169 | 2700 |
| 170 | 1900 |
| 171 | 6090 |
| 172 | 751 |
| 173 | 3600 |
| 174 | 706 |
| 175 | 359 |
| 176 | 174 |
| 177 | 17000 |
| 178 | 215 |
| 179 | 13 |
| 180 | 2890 |
| 181 | 1100 |
| 182 | 251 |
| 183 | 406 |
| 184 | 26 |
| 185 | 8990 |
| 186 | 911 |
| 187 | 123 |
| 188 | 710 |
| 189 | 84 |
| 190 | 88 |
| 191 | 171 |
| 192 | 181 |
| 193 | 2890 |
| 194 | 159 |
| 195 | 123 |
| 196 | 115 |
| 197 | 188 |
| 198 | 249 |
| 199 | 20000 |
| 200 | 100 |
| 201 | 215 |
| 202 | 267 |
| 203 | 175 |
| 204 | nd |
| 205 | 242 |
| 206 | 77 |
| 207 | 3140 |
| 208 | 2750 |
| 209 | 450 |
| 210 | 20000 |
| 211 | 3410 |
| 212 | 910 |
| 213 | 103 |
| 214 | 3400 |
| 215 | 93 |
| 216 | 5 |
| 217 | 10 |
| 218 | 6 |
| 219 | 1 |
| 220 | 3 |
| 221 | 4 |
| 222 | 25 |
| 223 | 53 |
| 224 | 40 |
| 225 | 1040 |
| 226 | 19 |
| 227 | 24 |
| 228 | 51 |
| 229 | 3540 |
| 230 | 1 |
| 231 | 50 |
| 232 | 5 |
| 233 | 149 |
| 234 | 288 |
| 235 | 159 |
| 236 | 55 |
| 237 | 12300 |
| 238 | 10 |

### Mnk2 Kinase High ATP Assay

Mnk2-inhibitory activity at high ATP of compounds of the present invention after their preincubation with Mnk2 was quantified employing the TR-FRET-based Mnk2 high ATP assay as described in the following paragraphs.

A recombinant fusion protein of Glutathione-S-Transferase (GST, N-terminally) and human full-length Mnk2 (Genbank accession number NP_{—}060042.2), expressed in insect cells using baculovirus expression system, purified via glutathione sepharose affinity chromatography, and activated in vitro with MAPK12, was purchased from Invitrogen (product no PV5608) and used as enzyme. As substrate for the kinase reaction the biotinylated peptide biotin-Ahx-IKKRKLTRRKSLKG (C-terminus in amide form) was used which can be purchased e.g. form the company Biosyntan (Berlin-Buch, Germany).

For the assay 50 nl of a 100fold concentrated solution of the test compound in DMSO was pipetted into a black low volume 384well microtiter plate (Greiner Bio-One, Frickenhausen, Germany), 2 μl of a solution of Mnk2 in aqueous assay buffer [50 mM HEPES pH 7.5, 5 mM MgCl₂, 1.0 mM dithiothreitol, 0.005% (v/v) Nonidet-P40 (G-Biosciences, St. Louis, USA)] was added and the mixture was incubated for 15 min at 22° C. to allow pre-binding of the test compounds to the enzyme before the start of the kinase reaction. Then the kinase reaction was started by the addition of 3 μl of a solution of adenosine-tri-phosphate (ATP, 3.3 mM=>final conc. in the 5 μl assay volume is 2 mM) and substrate (0.1 μM=>final conc. in the 5 μl assay volume is 0.06 μM) in assay buffer and the resulting mixture was incubated for a reaction time of 30 min at 22° C. The concentration of Mnk2 was adjusted depending of the activity of the enzyme lot and was chosen appropriate to have the assay in the linear range, typical concentrations were in the range of 0.0045 μg/mL. The reaction was stopped by the addition of 5 μl of a solution of TR-FRET detection reagents (5 nM streptavidine-XL665 [Cisbio Bioassays, Codolet, France] and 1 nM anti-ribosomal protein 56 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL665. Therefore, the fluorescence emissions at 620 nm and 665 nm after excitation at 350 nm were measured in a TR-FRET reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100fold concentrated solutions in DMSO by serial dilutions, the exact concentrations may vary depending on the pipettor used) in duplicate values for each concentration and IC₅₀ values were calculated by a 4 parameter fit using an inhouse software.

### EGFR Kinase Assay

EGFR inhibitory activity of compounds of the present invention can be quantified employing the TR-FRET based EGFR assay as described in the following paragraphs. Epidermal Growth Factor Receptor (EGFR) affinity purified from human carcinoma A431 cells (Sigma-Aldrich, #E3641) was used as kinase. As substrate for the kinase reaction the biotinylated peptide biotin-Ahx-AEEEEYFELVAKKK (C-terminus in amid form) is used which can be purchased e.g. form the company Biosynthan GmbH (Berlin-Buch, Germany).

For the assay 50 nL of a 100fold concentrated solution of the test compound in DMSO is pipetted into a black low volume 384well microtiter plate (Greiner Bio-One, Frickenhausen, Germany), 2 μL of a solution of EGFR in aqueous assay [50 mM Hepes/HCl pH 7.0, 1 mM MgCl₂, 5 mM MnCl₂, 0.5 mM activated sodium ortho-vanadate, 0.005% (v/v) Tween-20] are added and the mixture was incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (1.67 μM=>final conc. in the 5 μL assay volume is 1 μM) in assay buffer and the resulting mixture is incubated for a reaction time of 30 min at 22° C. The concentration of EGFR is adjusted depending of the activity of the enzyme lot and is chosen appropriate to have the assay in the linear range, typical concentration are in the range of 3 U/ml. The reaction is stopped by the addition of 5 μl of a solution of HTRF detection reagents (0.1 μM streptavidine-XL665 [Cis Biointernational] and 1 nM PT66-Tb-Chelate, an terbium-chelate labelled anti-phospho-tyrosine antibody from Cis Biointernational [instead of the PT66-Tb-chelate PT66-Eu-Cryptate from Perkin Elmer can also be used]) in an aqueous EDTA-solution (80 mM EDTA, 0.2% (w/v) bovine serum albumin in 50 mM HEPES pH 7.5).

The resulting mixture is incubated 1 h at 22° C. to allow the binding of the biotinylated phosphorylated peptide to the streptavidine-XL665 and the PT66-Eu-Chelate. Subsequently the amount of phosphorylated substrate is evaluated by measurement of the resonance energy transfer from the PT66-Eu-Chelate to the streptavidine-XL665. Therefore, the fluorescence emissions at 620 nm and 665 nm after excitation at 337 nm are measured in a HTRF reader, e.g. a Pherastar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100fold concentrated solutions in DMSO by serial dilutions, the exact concentrations may vary depending on the pipettor used) in duplicate values for each concentration and IC₅₀ values were calculated by a 4 parameter fit using an inhouse software.

### CDK2/CycE Kinase Assay

CDK2/CycE inhibitory activity of compounds of the present invention can be quantified employing the CDK2/CycE TR-FRET assay as described in the following paragraphs.

Recombinant fusion proteins of GST and human CDK2 and of GST and human CycE, expressed in insect cells (Sf9) and purified by Glutathion-Sepharose affinity chromatography, can be purchased from ProQinase GmbH (Freiburg, Germany). As substrate for the kinase reaction biotinylated peptide biotin-Ttds-YISPLKSPYKISEG (C-terminus in amid form) can be used which can be purchased e.g. from the company JERINI peptide technologies (Berlin, Germany).

For the assay 50 nL of a 100fold concentrated solution of the test compound in DMSO is pipetted into a black low volume 384well microtiter plate (Greiner Bio-One, Frickenhausen, Germany), 2 μL of a solution of CDK2/CycE in aqueous assay buffer [50 mM Tris/HCl pH 8.0, 10 mM MgCl₂, 1.0 mM dithiothreitol, 0.1 mM sodium ortho-vanadate, 0.01% (v/v) Nonidet-P40 (Sigma)] are added and the mixture is incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (1.25 μM=>final conc. in the 5 μL assay volume is 0.75 μM) in assay buffer and the resulting mixture is incubated for a reaction time of 25 min at 22° C. The concentration of CDK2/CycE is adjusted depending of the activity of the enzyme lot and is chosen appropriate to have the assay in the linear range, typical concentrations are in the range of 130 ng/ml. The reaction is stopped by the addition of 5 μL of a solution of TR-FRET detection reagents (0.2 μM streptavidine-XL665 [Cisbio Bioassays, Codolet, France] and 1 nM anti-RB (pSer807/pSer811)-antibody from BD Pharmingen [#558389] and 1.2 nM LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]) in an aqueous EDTA-solution (100 mM EDTA, 0.2% (w/v) bovine serum albumin in 100 mM HEPES/NaOH pH 7.0).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Eu-chelate to the streptavidine-XL. Therefore, the fluorescence emissions at 620 nm and 665 nm after excitation at 350 nm is measured in a TR-FRET reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100fold concentrated solutions in DMSO by serial 1:3.4 dilutions) in duplicate values for each concentration and IC50 values are calculated by a 4 parameter fit using an inhouse software.

### PDGFRβ Kinase Assay

PDGFRβ inhibitory activity of compounds of the present invention can be quantified employing the PDGFRβ HTRF assay as described in the following paragraphs.

As kinase, a GST-His fusion protein containing a C-terminal fragment of human PDGFRβ (amino acids 561-1106, expressed in insect cells [SF9] and purified by affinity chromatography, purchased from Proqinase [Freiburg i.Brsg., Germany] is used. As substrate for the kinase reaction the biotinylated poly-Glu,Tyr (4:1) copolymer (#61GT0BLA) from Cis Biointernational (Marcoule, France) is used.

For the assay 50 nL of a 100fold concentrated solution of the test compound in DMSO is pipetted into a black low volume 384well microtiter plate (Greiner Bio-One, Frickenhausen, Germany), 2 μL of a solution of PDGFRβ in aqueous assay buffer [50 mM HEPES/NaOH pH 7.5, 10 mM MgCl₂, 2.5 mM dithiothreitol, 0.01% (v/v) Triton-X100 (Sigma)] are added and the mixture was incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (2.27 μg/ml=>final conc. in the 5 μL assay volume is 1.36 μg/ml [−30 nM]) in assay buffer and the resulting mixture is incubated for a reaction time of 25 min at 22° C. The concentration of PDGFRβ in the assay is adjusted depending of the activity of the enzyme lot and is chosen appropriate to have the assay in the linear range, typical enzyme concentrations are in the range of about 125 pg/μL (final conc. in the 5 μL assay volume). The reaction is stopped by the addition of 5 μL of a solution of HTRF detection reagents (200 nM streptavidine-XLent [Cis Biointernational] and 1.4 nM PT66-Eu-Chelate, an europium-chelate labelled anti-phospho-tyrosine antibody from Perkin Elmer [instead of the PT66-Eu-chelate PT66-Tb-Cryptate from Cis Biointernational can also be used]) in an aqueous EDTA-solution (100 mM EDTA, 0.2% (w/v) bovine serum albumin in 50 mM HEPES/NaOH pH 7.5).

The resulting mixture is incubated 1 h at 22° C. to allow the binding of the biotinylated phosphorylated peptide to the streptavidine-XLent and the PT66-Eu-Chelate. Subsequently the amount of phosphorylated substrate is evaluated by measurement of the resonance energy transfer from the PT66-Eu-Chelate to the streptavidine-XLent. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100fold conc. stock solutions by serial 1:3 dilutions) in duplicate values for each concentration and IC₅₀ values are calculated by a 4 parameter fit using an inhouse software.

### Fyn Kinase Assay

C-terminally His6-tagged human recombinant kinase domain of the human T-Fyn expressed in baculovirus infected insect cells (purchased from Invitrogen, P3042) is used as kinase. As substrate for the kinase reaction the biotinylated peptide biotin-KVEKIGEGTYGVV (C-terminus in amid form) is used which can be purchased e.g. form the company Biosynthan GmbH (Berlin-Buch, Germany).

For the assay 50 nL of a 100fold concentrated solution of the test compound in DMSO is pipetted into a black low volume 384well microtiter plate (Greiner Bio-One, Frickenhausen, Germany), 2 μL of a solution of T-Fyn in aqueous assay buffer [25 mM Tris/HCl pH 7.2, 25 mM MgCl₂, 2 mM dithiothreitol, 0.1% (w/v) bovine serum albumin, 0.03% (v/v) Nonidet-P40 (Sigma)]. are added and the mixture is incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (2 μM=>final conc. in the 5 μL assay volume is 1.2 μM) in assay buffer and the resulting mixture is incubated for a reaction time of 60 min at 22° C. The concentration of Fyn is adjusted depending of the activity of the enzyme lot and is chosen appropriate to have the assay in the linear range, typical concentration was 0.13 nM. The reaction is stopped by the addition of 5 μL of a solution of HTRF detection reagents (0.2 μM streptavidine-XL [Cisbio Bioassays, Codolet, France) and 0.66 nM PT66-Eu-Chelate, an europium-chelate labelled anti-phospho-tyrosine antibody from Perkin Elmer [instead of the PT66-Eu-chelate PT66-Tb-Cryptate from Cisbio Bioassays can also be used]) in an aqueous EDTA-solution (125 mM EDTA, 0.2% (w/v) bovine serum albumin in 50 mM HEPES/NaOH pH 7.0).

The resulting mixture is incubated 1 h at 22° C. to allow the binding of the biotinylated phosphorylated peptide to the streptavidine-XL and the PT66-Eu-Chelate. Subsequently the amount of phosphorylated substrate is evaluated by measurement of the resonance energy transfer from the PT66-Eu-Chelate to the streptavidine-XL. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compounds are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100fold conc. stock solutions by serial 1:3 dilutions) in duplicate values for each concentration and IC₅₀ values are calculated by a 4 parameter fit using an inhouse software.

### Flt4 Kinase Assay

Flt4 inhibitory activity of compounds of the present invention can be quantified employing the Flt4 TR-FRET assay as described in the following paragraphs.

As kinase, a GST-His fusion protein containing a C-terminal fragment of human Flt4 (amino acids 799-1298, expressed in insect cells [SF9] and purified by affinity chromatography, purchased from Proqinase [Freiburg i.Brsg., Germany] is used. As substrate for the kinase reaction the biotinylated peptide Biotin-Ahx-GGEEEEYFELVKKKK (C-terminus in amide form, purchased from Biosyntan, Berlin-Buch, Germany) is used.

For the assay 50 nL of a 100fold concentrated solution of the test compound in DMSO was pipetted into a black low volume 384well microtiter plate (Greiner Bio-One, Frickenhausen, Germany), 2 μL of a solution of Flt4 in aqueous assay buffer [25 mM HEPES pH 7.5, 10 mM MgCl₂, 2 mM dithiothreitol, 0.01% (v/v) Triton-X100 (Sigma), 0.5 mM EGTA, and 5 mM β-phospho-glycerol] are added and the mixture is incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (1.67 μM=>final conc. in the 5 μL assay volume is 1 μM) in assay buffer and the resulting mixture is incubated for a reaction time of 45 min at 22° C. The concentration of Flt4 in the assay is adjusted depending of the activity of the enzyme lot and was chosen appropriate to have the assay in the linear range, typical enzyme concentrations are in the range of about 120 pg/μL (final conc. in the 5 μL assay volume). The reaction is stopped by the addition of 5 μL of a solution of HTRF detection reagents (200 nM streptavidine-XL665 [Cis Biointernational] and 1 nM PT66-Tb-Cryptate, an terbium-cryptate labelled anti-phospho-tyrosine antibody from Cisbio Bioassays (Codolet, France) in an aqueous EDTA-solution (50 mM EDTA, 0.2% (w/v) bovine serum albumin in 50 mM HEPES pH 7.5).

The resulting mixture is incubated 1 h at 22° C. to allow the binding of the biotinylated phosphorylated peptide to the streptavidine-XL665 and the PT66-Tb-Cryptate. Subsequently the amount of phosphorylated substrate is evaluated by measurement of the resonance energy transfer from the PT66-Tb-Cryptate to the streptavidine-XL665. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100fold conc. stock solutions by serial 1:3 dilutions) in duplicate values for each concentration and IC₅₀ values are calculated by a 4 parameter fit using an inhouse software.

### TrkA Kinase Assay

TrkA inhibitory activity of compounds of the present invention can be quantified employing the TrkA HTRF assay as described in the following paragraphs.

As kinase, a GST-His fusion protein containing a C-terminal fragment of human TrkA (amino acids 443-796, expressed in insect cells [SF9] and purified by affinity chromatography, purchased from Proqinase [Freiburg i.Brsg., Germany] is used. As substrate for the kinase reaction the biotinylated poly-Glu,Tyr (4:1) copolymer (#61GT0BLA) from Cis Biointernational (Marcoule, France) is used.

For the assay 50 nL of a 100fold concentrated solution of the test compound in DMSO is pipetted into a black low volume 384well microtiter plate (Greiner Bio-One, Frickenhausen, Germany), 2 μL of a solution of TrkA in aqueous assay buffer [8 mM MOPS/HCl pH 7.0, 10 mM MgCl₂, 1 mM dithiothreitol, 0.01% (v/v) NP-40 (Sigma), 0.2 mM EDTA] are added and the mixture was incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (2.27 μg/ml=>final conc. in the 5 μL assay volume is 1.36 μg/ml [−30 nM]) in assay buffer and the resulting mixture is incubated for a reaction time of 60 min at 22° C. The concentration of TrkA in the assay is adjusted depending of the activity of the enzyme lot and is chosen appropriate to have the assay in the linear range, typical enzyme concentrations are in the range of about 20 pg/μL (final conc. in the 5 μL assay volume). The reaction is stopped by the addition of 5 μL of a solution of HTRF detection reagents (30 nM streptavidine-XL665 [Cis Biointernational] and 1.4 nM PT66-Eu-Chelate, an europium-chelate labelled anti-phospho-tyrosine antibody from Perkin Elmer [instead of the PT66-Eu-chelate PT66-Tb-Cryptate from Cis Biointernational can also be used]) in an aqueous EDTA-solution (100 mM EDTA, 0.2% (w/v) bovine serum albumin in 50 mM HEPES/NaOH pH 7.5).

The resulting mixture is incubated 1 h at 22° C. to allow the binding of the biotinylated phosphorylated peptide to the streptavidine-XL665 and the PT66-Eu-Chelate. Subsequently the amount of phosphorylated substrate is evaluated by measurement of the resonance energy transfer from the PT66-Eu-Chelate to the streptavidine-XL665. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100fold conc. stock solutions by serial 1:3 dilutions) in duplicate values for each concentration and IC₅₀ values are calculated by a 4 parameter fit using an inhouse software.

AlphaScreen SureFire eIF4E Ser209 Phosphorylation Assay

The AlphaScreen SureFire eIF4E Ser209 phosphorylation assay can be used to measure the phosphorylation of endogenous eIF4E in cellular lysates. The AlphaScreen SureFire technology allows the detection of phosphorylated proteins in cellular lysates. In this assay, sandwich antibody complexes, which are only formed in the presence of the analyte (p-eIF4E Ser209), are captured by AlphaScreen donor and acceptor beads, bringing them into close proximity. The excitation of the donor bead provokes the release of singlet oxygen molecules that triggers a cascade of energy transfer in the Acceptor beads, resulting in the emission of light at 520-620 nm.

Surefire EIF4e Alphascreen in A549 Cells with 20% FCS Stimulation

For the assay the AlphaScreen SureFire p-eIF4E Ser209 10K Assay Kit and the AlphaScreen ProteinA Kit (for 10K assay points) both from Perkin Elmer are used.

On day one 50,000 A549 cells are plated in a 96-well plate in 100 μL per well in growth medium (DMEM/Hams' F12 with stable Glutamin, 10% FCS) and incubated at 37° C. After attachment of the cells, medium is changed to starving medium (DMEM, 0.1% FCS, without Glucose, with Glutamin, supplemented with 5 g/L Maltose). On day two, test compounds are serially diluted in 50 μL starving medium with a final DMSO concentration of 1% and are added to A549 cells in test plates at a final concentration range from as high 10 μM to as low 10 nM depending on the activities of the tested compounds. Treated cells are incubated at 37° C. for 2 h. 37 ul FCS is added to the wells (=final FCS concentration 20%) for 20 min. Then medium is removed and cells are lysed by adding 50 μL Lysis buffer. Plates are then agitated on a plate shaker for 10 min. After 10 min Lysis time, 4 μL of the lysate is transferred to a 384well plate (Proxiplate from Perkin Elmer) and 5 μL Reaction Buffer plus Activation Buffer mix containing AlphaScreen Acceptor beads is added. Plates are sealed with TopSeal-A adhesive film, gently agitated on a plate shaker for 2 hours at room temperature. Afterwards 2 μL Dilution buffer with AlphaScreen Donor beads are added under subdued light and plates are sealed again with TopSeal-A adhesive film and covered with foil. Incubation takes place for further 2 h gently agitation at room temperature. Plates are then measured in an EnVision reader (Perkin Elmer) with the AlphaScreen program. Each data point (compound dilution) is measured as triplicate.

The IC₅₀ values are determined by means of a 4-parameter fit using the company's own software.

### Proliferation Assays

The tumor cell proliferation assay which can be used to test the compounds of the present invention involves a readout called Cell Titer-Glow® Luminescent Cell Viability Assay developed by Promega® (B. A. Cunningham, “A Growing Issue: Cell Proliferation Assays, Modern kits ease quantification of cell growth”, The Scientist 2001, 15(13), 26; S. P. Crouch et al., “The use of ATP bioluminescence as a measure of cell proliferation and cytotoxicity”, Journal of Immunological Methods 1993, 160, 81-88), that measures inhibition of cell proliferation. Generation of a luminescent signal corresponds to the amount of ATP present, which is directly proportional to the number of metabolically active (proliferating) cells.

### In Vitro Tumor Cell Proliferation Assay:

Cultivated tumour cells (MOLM-13 (human acute myeloid leukemia cells obtained from DSMZ #ACC 554), JJN-3 (human plasma cell leukemia cells obtained from DSMZ #ACC 541), Ramos (RA1) (human Burkitt's Lymphoma cells obtained from ATCC #CRL-159)) are plated at a density of 2,500 cells/well (JJN-3), 3,000 cells/well (MOLM-13), 4,000 cells/well (Ramos (RA1)), in a 96-well multititer plate (Costar 3603 black/clear bottom) in 100 μL of their respective growth medium supplemented with 10% fetal calf serum. After 24 hours, the cells of one plate (zero-point plate) are measured for viability. Therefore, 70 μL/well CTG solution (Promega Cell Titer Glo solution (catalog #G755B and G756B)) is added to zero-point plate. The plates are mixed for two minutes on orbital shaker to ensure cell lysis and incubated for ten minutes at room temperature in the dark to stabilize luminescence signal. The samples are read on a VICTOR 3 plate reader. In parallel, serially test compounds are diluted in growth medium, and 50 μL of 3× dilutions/well are pipetted into the test plates (final concentrations: 0 μM, as well as in the range of 0.001-30 μM). The final concentration of the solvent dimethyl sulfoxide is 0.3-0.4%. The cells are incubated for 3 days in the presence of test substances. 105 μL/well CTG solution (Promega Cell Titer Glo solution (catalog #G755B and G756B)) is added to the test wells. The plates are mixed for 2 minutes on an orbital shaker to ensure cell lysis and incubated for 10 min at room temperature in the dark to stabilize luminescence signal. The samples are read on a VICTOR 3 plate reader. The change of cell number, in percent, is calculated by normalization of the measured values to the extinction values of the zero-point plate (=0%) and the extinction of the untreated (0 μm) cells (=100%). The IC₅₀ values (inhibitory concentration at 50% of maximal effect) are determined by means of a 4 parameter fit using the company's own software.

### Overview Cell Lines for Proliferation Assays

| | | Cell | |
|---|---|---|---|
| Cell line | Origin | number/well | Culture Medium |
| MOLM-13 | human | 3000 | RPMI 1640 with stable |
| (obtained | acute | | Glutamin with 10% |
| from DSMZ | myeloid | | Fetal Bovine Serum |
| # ACC 554) | leukemia | | |
| JJN-3 | human | 2500 | 45% Dulbecco's Modified |
| (obtained | plasma | | Eagle Medium with stable |
| from DSMZ | cell | | Glutamin, 45% Iscove's |
| # ACC 541) | leukemia | | Modified Dulbecco's Media |
| | | | with stable Glutamin and |
| | | | 10% Fetal Bovine Serum |
| Ramos (RA1) | human | 4000 | RPMI 1640 media with stable |
| (obtained from | Burkitt's | | Glutamin with 10% Fetal |
| ATCC # | lymphoma | | Bovine Serum |
| CRL-159) | | | |

### Kinase Selectivity Profiling

Often, kinase inhibitors show inhibitory action with respect to different kinases. In order to prevent undesirable side effects, the selectivity of a kinase inhibitor should be high. The selectivity can be determined e.g. by a target profiling in which the selectivity of compounds against various kinases is tested e.g. by Merck Millipore in a service called KinaseProfiler.

The compounds of the present invention are characterized by a high selectivity with respect to MKNK.

Thus, the compounds of the present invention effectively inhibit MKNK1 and/or MKNK2 and are therefore suitable for the treatment or prophylaxis of diseases of uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK1 and/or MKNK2, more particularly in which the diseases of uncontrolled cell growth, proliferation and/or survival, inappropriate cellular immune responses, or inappropriate cellular inflammatory responses are haemotological tumours, solid tumours and/or metastases thereof, e.g. Leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

1. A compound of general formula I: 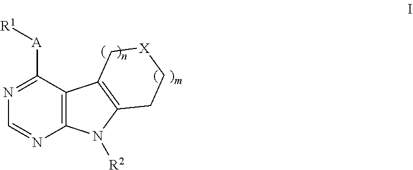
in which:
A represents —O—, —S—, —S(═O)—, —S(═O)₂—, —S(═O)(NR³)— or —NR³—;
X represents —O—, —S—, —S(═O)—, —S(═O)₂—, —S(═O)(NR^{4a})—,
—NR^{4a}—, —C(O—C₁-C₆-alkyl)₂-, —C(O—CH₂—CH₂—O)—, —C(O—CH₂—CH₂—CH₂—O)—, —C(O—CH₂—C(CH₃)₂—CH₂—O)—, —C(═O)—, —C(═O)NR^{4a}—, —NR^{4a}C(═O)—, —C(═O)O—, —OC(═O)—, —C(H)(OR^{4a})—, —C(R^{4a})(OR^{4b})—, —C═NR^{4a}—, —C(H)NR^{4a}R^{4b}—, —C(R^{5a})(R^{5b})—, —C(═CR^{6a}R^{6b})— or —CH(CHR^{6a}R^{6b})—;
R¹ represents an aryl- or heteroaryl- group; wherein said aryl- or heteroaryl- group is optionally substituted, identically or differently, with 1, 2, 3, 4 or 5 R⁷ groups;
R² represents a hydrogen atom, or a C₁-C₆-alkyl- or C₃-C₆-cycloalkyl- group; wherein said C₁-C₆-alkyl- or C₃-C₆-cycloalkyl- group is optionally substituted, identically or differently, with 1, 2, 3 or 4 groups selected from:
halogen, —OH, —CN, —C₁-C₆-alkyl, C₁-C₆-alkoxy-;
R³ represents a hydrogen atom, or a C₁-C₆-alkyl- or C₃-C₆-cycloalkyl- group; wherein said C₁-C₆-alkyl- or C₃-C₆-cycloalkyl- group is optionally substituted, identically or differently, with 1, 2, 3 or 4 groups selected from:
halogen, —OH, —CN, —C₁-C₆-alkyl, C₁-C₆-alkoxy-;
or
A=—NR³—; and R¹ and R³, together with the nitrogen atom they are attached to, represent a 3- to 7-membered heterocycloalkyl- or benzo fused 3- to 7-membered heterocycloalkyl- group; wherein said group is optionally substituted, identically or differently, with 1, 2, 3 or 4 R⁷ groups;
R^{4a}, R^{4b}
represent, independently from each other, a hydrogen atom, or a C₁-C₆-alkyl-, —(CH₂)ₚ—C₂-C₆-alkenyl, —(CH₂)_{q}—C₄-C₈-cycloalkenyl, (CH₂)ₚ—C₂-C₆-alkynyl, —(CH₂)_{q}—C₃-C₆-cycloalkyl,
—(CH₂)_{q}-(3- to 7-membered heterocycloalkyl),
—(CH₂)_{q}-(4- to 8-membered heterocycloalkenyl), aryl-, aryl-C₁-C₆-alkyl-, heteroaryl-, heteroaryl-C₁-C₆-alkyl-, halo-C₆-C₆-alkyl-, R^{8a}(R^{8b})N—C₁-C₆-alkyl-, halo-C₁-C₆-alkoxy-C₁-C₆-alkyl-, —C(═O)R⁸, —C(═O)N(R^{8a}R^{8b}), —C(═O)O—R⁸, —S(═O)R⁸, —S(═O)₂R⁸, —S(═O)(═NR^{8a})R^{8b} or —S(═O)₂N(R^{8a})R^{8b} group;
said C₁-C₆-alkyl-, —(CH₂)ₚ—C₂-C₆-alkenyl, —(CH₂)_{q}—C₄-C₈-cycloalkenyl, —(CH₂)ₚ—C₂-C₆-alkynyl, —(CH₂)_{q}—C₃-C₆-cycloalkyl,
—(CH₂)_{q}-(3- to 7-membered heterocycloalkyl),
—(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl- or halo-C₁-C₆-alkoxy-C₁-C₆-alkyl- group being optionally substituted, identically or differently, with 1, 2, 3, 4 or 5 R⁷ groups;
R^{5a}, R^{5b}
represent, independently from each other, a hydrogen atom or a halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
—(CH₂)_{q}-(3- to 7-membered heterocycloalkyl),
—(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, C₁-C₆-alkoxy-, C₁-C₆-alkoxy-C₁-C₆-alkyl-, halo-C₁-C₆-alkoxy-C₁-C₆-alkyl-, —C(═O)R⁸, —C(═O)N(R^{8a}R^{8b}), —C(═O)O—R⁸, —S(═O)R⁸, —S(═O)₂R⁸, —S(═O)(═NR^{8a})R^{8b} or —S(═O)₂N(R^{8a})R^{8b} group;
or
R^{5a} and R^{5b} together
form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- or —O—(C₂-C₆-alkylene)-O— group;
said C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
—(CH₂)_{q}-(3- to 7-membered heterocycloalkyl),
—(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- or —O—(C₂-C₆-alkylene)-O— group being optionally substituted, identically or differently, with 1, 2, 3, 4 or 5 R⁷ groups;
R^{6a}, R^{6b}
represent, independently from each other, a hydrogen atom or halogen atom, or a C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
—(CH₂)_{q}-(3- to 7-membered heterocycloalkyl),
—(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C(═O)R⁸, —C(═O)N(R^{8a}R^{8b}) or —C(═O)O—R⁸ group;
or
R^{6a} and R^{6b} together
form a —C₁-C₆-alkylene-, —(CH₂)_{q}—C₂-C₆-alkenylene-, halo-C₁-C₆-alkylene-, —(C₁-C₃-alkylene)-Q-(C₁-C₃-alkylene)- group;
said C₁-C₆-alkyl-, —(CH₂)_{q}—C₂-C₆-alkenyl, —(CH₂)_{q}—C₄-C₈-cycloalkenyl, —(CH₂)_{q}—C₂-C₆-alkynyl, —(CH₂)_{q}—C₃-C₆-cycloalkyl,
—(CH₂)_{q}-(3- to 7-membered heterocycloalkyl),
—(CH₂)_{q}-(4- to 8-membered heterocycloalkenyl), aryl-, aryl-C₁-C₆-alkyl-, heteroaryl-, heteroaryl-C₁-C₆-alkyl-, halo-C₁-C₆-alkyl-, halo-C₁-C₆-alkoxy-C₁-C₆-alkyl-, —C₁-C₆-alkylene-, —(CH₂)_{q}—C₂-C₆-alkenylene-, halo-C₁-C₆-alkylene- or —(C₁-C₃-alkylene)-Q-(C₁-C₃-alkylene)- group being optionally substituted, identically or differently, with 1, 2, 3, 4 or 5 R⁷ groups;
R⁷ represents a halogen atom, or a HO—, —CN, C₁-C₆-alkoxy-, C₁-C₆-alkyl-, halo-C₁-C₆-alkyl-, R^{8a}(R^{8b})N—C₁-C₆-alkyl-, halo-C₁-C₆-alkoxy-, HO—C₁-C₆-alkyl-, HO—C₁-C₆-alkoxy-, C₁-C₆-alkoxy-C₁-C₆-alkyl-, halo-C₁-C₆-alkoxy-C₁-C₆-alkyl-, C₂-C₆-alkenyl-, C₂-C₆-alkynyl-, 3- to 7-membered heterocycloalkyl-, aryl-, heteroaryl-, —C(═O)R⁸, —C(═O)N(R^{8a})R^{8b}, —C(═O)O—R⁸, —N(R^{8a})R^{8b}, —NO₂, —N(R^{8a})C(═O)R^{8b}, —N(R^{8c})C(═O)N(R^{8a})R^{8b}, —N(R^{8a})C(═O)OR^{8b}, —N(R^{8a})S(═O)R^{8b}, —N(R^{8a})S(═O)₂R^{8b}, —N═S(═O)(R^{8a})R^{8b}, —OR⁸, —O(C═O)R⁸, —O(C═O)N(R^{8a})R^{8b},
—O(C═O)OR⁸, —SR⁸, —S(═O)R⁸, —S(═O)N(R^{8a})R^{8b}, —S(═O)₂R⁸, —S(═O)₂OR⁸, —S(═O)₂N(R^{8a})R^{8b}, —S(═O)(═NR^{8c})R⁸ or —P(═O)(R^{8a})(OR^{8b}) group;
wherein said aryl- or heteroaryl- group is optionally substituted, identically or differently, with 1, 2 or 3 C₁-C₆-alkyl groups;
or
when 2 R⁷ groups are present ortho- to each other on an aryl ring, said 2 R⁷ groups together form a bridge:
*CH═N—N(H)*, *O(CH₂)₂O*, *O(CH₂)O*, *O(CF₂)O*, *C(═O)OCH₂*, *OC(═O)C(R^{8a})═C(R^{8b})*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═S)S*, *N(R^{8a})C(═O)C(R^{8b})═C(R^{8c})*, *NHC(═O)NH*, *S(═O)ₓCH₂CH₂*, *CH₂S(═O)ₓCH₂*, *N(H)C(═O)—C(═O)N(H)*, *(CH₂)ₜ*; wherein each * represents the point of attachment to said aryl ring;
R⁸, R^{8a}, R^{8b}, R^{8c}
represent, independently from each other, a hydrogen atom, or a C₁-C₆-alkyl-, C₃-C₆-cycloalkyl-, C₃-C₆-cycloalkyl-(CH₂)—, C₁-C₆-alkyl-aryl-, C₂-C₆-alkenyl-, 3- to 7-membered heterocycloalkyl-, aryl-, heteroaryl-, aryl-C₁-C₆-alkyl-, or heteroaryl-C₁-C₆-alkyl- group;
said C₁-C₆-alkyl-, C₃-C₆-cycloalkyl-, C₁-C₆-alkyl-aryl-, C₂-C₆-alkenyl-, 3- to 7-membered heterocycloalkyl-, aryl-, heteroaryl-, aryl-C₁-C₆-alkyl- or heteroaryl-C₁-C₆-alkyl- group being optionally substituted, identically or differently, with 1, 2, 3, 4 or 5 R¹⁰ groups;
or
R^{8a} and R^{8b} together form a C₁-C₆-alkylene- or halo-C₁-C₆-alkylene- group;
Q represents a —O—, —S—, —S(═O)—, —S(═O)₂—, —S(═O)(NR⁹)—, —N(R⁹)—, —C(═O)—, —C(═O)—O— or —C(═O)—N(R⁹)— group;
R⁹, R^{9a}, R^{9b}
represent, independently from each other, a hydrogen atom, or a C₁-C₆-alkyl-, C₃-C₆-cycloalkyl-, C₁-C₆-alkyl-aryl-, aryl- or heteroaryl- group; wherein said C₁-C₆-alkyl-, C₃-C₆-cycloalkyl-, C₁-C₆-alkyl-aryl-, aryl- or heteroaryl- group is optionally substituted, identically or differently, with 1, 2, 3, or 4 groups selected from: halogen, —OH, —CN, —C₁-C₆-alkyl, C₁-C₆-alkoxy-;
R¹⁰ represents a halogen atom or a group selected from:
C₁-C₆-alkoxy-, C₁-C₆-alkyl-, halo-C₁-C₆-alkyl-, halo-C₁-C₆-alkoxy-, —CN, —OH, HO—C₁-C₆-alkyl-, —S(═O)_{X}(C₁-C₆-alkyl), —S(═O)_{X}(aryl), —S(═O)_{X}(C₁-C₆-alkyl-aryl),
—S(═O)N(R⁹)(C₁-C₆-alkyl), —N(R^{9a})(R^{9b}), —C(═O)R⁹, —C(═O)OR⁹, —C(═O)N(R^{9a})(R^{9b});
m is an integer of 0, 1, 2, or 3;
n is an integer of 0, 1, 2, or 3;
p is an integer of 1, 2, 3, 4 or 5;
q is an integer of 0, 1, 2, 3, 4 or 5;
t is an integer of 3, 4, 5 or 6;
x is an integer of 0, 1 or 2
or a stereoisomer, a tautomer, an N-oxide, a hydrate, a solvate, or a salt thereof, or a mixture of same.

2. The compound of claim 1, wherein
A represents —O—, —S—, —S(═O)—, —S(═O)₂— or —NR³—;
or a stereoisomer, a tautomer, an N-oxide, a hydrate, a solvate, or a salt thereof, or a mixture of same.

3. The compound of claim 1, wherein
X represents a group selected from:
—(CH₂)—, —(CF₂)—, —C(H)(C(═O)R⁸)—, —C(H)(C(═O)N(R^{8a}R^{8b}))—, —C(H)(C(═O)O—R⁸)—;
or a stereoisomer, a tautomer, an N-oxide, a hydrate, a solvate, or a salt thereof, or a mixture of same.

4. The compound of claim 1, wherein
m is an integer of 1;
n is an integer of 1;
or a stereoisomer, a tautomer, an N-oxide, a hydrate, a solvate, or a salt thereof, or a mixture of same.

5. The compound of claim 1, wherein
R¹ represents 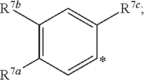
wherein * represents the point of attachment of the group to the rest of the molecule;
wherein R^{7a} and R^{7b} represent, independently from each other, a hydrogen atom or a halogen atom, or a group selected from:
—CN, C₁-C₆-alkoxy-, C₁-C₆-alkyl-, halo-C₁-C₆-alkyl-, halo-C₁-C₆-alkoxy-, C₁-C₆-alkoxy-C₁-C₆-alkyl-, halo-C₁-C₆-alkoxy-C₁-C₆-alkyl-;
or
R^{7a} and R^{7b} together form a bridge:
*CH═N—N(H)*, *O(CH₂)₂O*, *O(CF₂)O*, *C(═O)OCH₂*, *CH₂C(R^{8a})(R^{8b})O*, *C(═O)N(R^{8a})CH₂*, *N(R^{8a})C(═O)CH₂O*, *N(R^{8a})C(═O)S*, *N(R^{8a})C(═S)S*, *N(R^{8a})C(═O)C(R^{8b})═C(R^{8c})*, *S(═O)ₓCH₂CH₂*, *CH₂S(═O)ₓCH₂*,
*N(H)C(═O)—C(═O)N(H)*, *(CH₂)ₜ*; wherein each * represents the point of attachment to the phenyl ring;
wherein R^{7c} represents a hydrogen atom, a halogen atom or a group selected from: 3- to 7-membered heterocycloalkyl-, C₁-C₆-alkoxy-, halo-C₁-C₆-alkoxy-, HO—C₁-C₆-alkoxy-, —OR⁸; in which R⁸ represents a C₃-C₆-cycloalkyl-, C₃-C₆-cycloalkyl-(CH₂)— or 3- to 7-membered heterocycloalkyl- group;
or a stereoisomer, a tautomer, an N-oxide, a hydrate, a solvate, or a salt thereof, or a mixture of same.

6. The compound of claim 1, selected from the group consisting of:
N-(4-fluoro-2-isopropoxyphenyl)-6,7,8,9-tetrahydro-5H-pyrimido[4,5-b]indol-4-amine,
N-(1H-indazol-5-yl)-6,7,8,9-tetrahydro-5H-pyrimido[4,5-b]indol-4-amine,
N-(1H-indazol-5-yl)-5,6,7,8,9,10-hexahydrocyclohepta[4,5]pyrrolo[2,3-d]pyrimidin-4-amine,
N-(4-fluoro-2-isopropoxyphenyl)-5,6,7,8,9,10-hexahydrocyclohepta[4,5]pyrrolo[2,3-d]pyrimidin-4-amine,
(RS)-Ethyl 4-(1H-indazol-5-ylamino)-6,7,8,9-tetrahydro-5H-pyrimido[4,5-b]indole-6-carboxylate,
(RS)-4-(1H-indazol-5-ylamino)-6,7,8,9-tetrahydro-5H-pyrimido[4,5-b]indole-6-carboxylic acid,
(RS)—N-ethyl-4-(1H-indazol-5-ylamino)-6,7,8,9-tetrahydro-5H-pyrimido[4,5-b]indole-6-carboxamide,
6-benzyl-N-(4-fluoro-2-isopropoxyphenyl)-6,7,8,9-tetrahydro-5H-pyrido[3′,4′:4,5]pyrrolo[2,3-d]pyrimidin-4-amine,
6,6-difluoro-N-(4-fluoro-2-isopropoxyphenyl)-6,7,8,9-tetrahydro-5H-pyrimido[4,5-b]indol-4-amine,
6,6-difluoro-N-(1H-indazol-5-yl)-6,7,8,9-tetrahydro-5H-pyrimido[4,5-b]indol-4-amine,
N-(4-fluoro-2-isopropoxyphenyl)-5,7,8,9-tetrahydrothiopyrano[3′,4′:4,5]pyrrolo[2,3-d]pyrimidin-4-amine,
N-(4,5-dichloro-2-methoxyphenyl)-5,6,7,8,9,10-hexahydrocyclohepta[4,5]pyrrolo[2,3-d]pyrimidin-4-amine,
(RS)-ethyl 4-[(4,5-dichloro-2-methoxyphenyl)amino]-6,7,8,9-tetrahydro-5H-pyrimido[4,5-b]indole-6-carboxylate,
(RS)-4-(1H-indazol-5-ylamino)-N-[3-(methylsulfonyl)propyl]-6,7,8,9-tetrahydro-5H-pyrimido[4,5-b]indole-6-carboxamide,
(RS)—N-cyclopropyl-4-(1H-indazol-5-ylamino)-6,7,8,9-tetrahydro-5H-pyrimido[4,5-b]indole-6-carboxamide,
(RS)-4-(1H-indazol-5-ylamino)-N-phenyl-6,7,8,9-tetrahydro-5H-pyrimido[4,5-b]indole-6-carboxamide,
(RS)-(3,3-difluoroazetidin-1-yl)[4-(1H-indazol-5-ylamino)-6,7,8,9-tetrahydro-5H-pyrimido[4,5-b]indol-6-yl]methanone,
(RS)-4-[(4,5-dichloro-2-methoxyphenyl)amino]-6,7,8,9-tetrahydro-5H-pyrimido[4,5-b]indole-6-carboxylic acid,
N-(4,5-dichloro-2-methoxyphenyl)-6,6-difluoro-6,7,8,9-tetrahydro-5H-pyrimido[4,5-b]indol-4-amine,
(RS)—N-cyclopropyl-4-[(4,5-dichloro-2-methoxyphenyl)amino]-6,7,8,9-tetrahydro-5H-pyrimido[4,5-b]indole-6-carboxamide,
(RS)-4-[(4,5-dichloro-2-methoxyphenyl)amino]-N-ethyl-6,7,8,9-tetrahydro-5H-pyrimido[4,5-b]indole-6-carboxamide,
(RS)-4-[(4,5-dichloro-2-methoxyphenyl)amino]-N-phenyl-6,7,8,9-tetrahydro-5H-pyrimido[4,5-b]indole-6-carboxamide,
N-(4,5-dichloro-2-methoxyphenyl)-6,7,8,9-tetrahydro-5H-pyrimido[4,5-b]indol-4-amine,
6-benzyl-N-(1H-indazol-5-yl)-6,7,8,9-tetrahydro-5H-pyrido[3′,4′:4,5]pyrrolo[2,3-d]pyrimidin-4-amine,
(RS)-4-[(4-fluoro-2-isopropoxyphenyl)amino]-9-methyl-N-[3-(methylsulfonyl)propyl]-6,7,8,9-tetrahydro-5H-pyrimido[4,5-b]indole-6-carboxamide,
(RS)-4-(1H-indazol-5-ylamino)-9-methyl-N-[3-(methylsulfonyl)propyl]-6,7,8,9-tetrahydro-5H-pyrimido[4,5-b]indole-6-carboxamide,
N-(1H-indazol-5-yl)-5′,7′,8′,9′-tetrahydrospiro[1,3-dioxolane-2,6′-pyrimido[4,5-b]indol]-4′-amine,
(RS)-4-{[4-fluoro-2-(propan-2-yloxy)phenyl]amino}-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-[(2-oxo-2,3-dihydro-1,3-benzothiazol-6-yl)amino]-6,7,8,9-tetrahydro-5H-pyrimido[4,5-b]indole-6-carboxamide,
(RS)—N-[3-(methylsulfonyl)propyl]-4-{[2-(morpholin-4-yl)phenyl]amino}-6,7,8,9-tetrahydro-5H-pyrimido[4,5-b]indole-6-carboxamide,
(RS)-4-{[4-(difluoromethoxy)phenyl]amino}-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-{[2-(trifluoromethoxy)phenyl]amino}-6,7,8,9-tetrahydro-5H-pyrimido[4,5-b]indole-6-carboxamide,
(RS)-4-[(4-methyl-2-oxo-2H-chromen-7-yl)amino]-N-[3-(methylsulfonyl)propyl]-6,7,8,9-tetrahydro-5H-pyrimido[4,5-b]indole-6-carboxamide,
(RS)-4-[(4-fluoro-3-methoxyphenyl)amino]-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-{[4-(propan-2-yloxy)phenyl]amino}-6,7,8,9-tetrahydro-5H-pyrimido[4,5-b]indole-6-carboxamide,
(RS)-4-[(2,2-difluoro-1,3-benzodioxol-5-yl)amino]-N-[3-(methylsulfonyl)propyl]-6,7,8,9-tetrahydro-5H-pyrimido[4,5-b]indole-6-carboxamide,
(RS)-4-[(4-fluoro-2-methoxyphenyl)amino]-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-{[4-(pyrrolidin-1-ylcarbonyl)phenyl]amino}-6,7,8,9-tetrahydro-5H-pyrimido[4,5-b]indole-6-carboxamide,
(RS)-4-[(5-fluoro-2-methoxyphenyl)amino]-N-[3-(methylsulfonyl)propyl]-6,7,8,9-tetrahydro-5H-pyrimido[4,5-b]indole-6-carboxamide,
(RS)-4-[(2,2-dioxido-1,3-dihydro-2-benzothiophen-5-yl)amino]-N-[3-(methylsulfonyl)propyl]-6,7,8,9-tetrahydro-5H-pyrimido[4,5-b]indole-6-carboxamide,
(RS)-4-{[4-(methylsulfamoyl)phenyl]amino}-N-[3-(methylsulfonyl)propyl]-6,7,8,9-tetrahydro-5H-pyrimido[4,5-b]indole-6-carboxamide,
(RS)-4-[(2,5-dimethoxyphenyl)amino]-N-[3-(methylsulfonyl)propyl]-6,7,8,9-tetrahydro-5H-pyrimido[4,5-b]indole-6-carboxamide,
(RS)-4-{[4-fluoro-3-(trifluoromethoxy)phenyl]amino}-N-[3-(methylsulfonyl)propyl]-6,7,8,9-tetrahydro-5H-pyrimido[4,5-b]indole-6-carboxamide,
(RS)-4-[(4,5-dichloro-2-methoxyphenyl)amino]-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-[(1-oxo-1,3-dihydro-2-benzofuran-5-yl)amino]-6,7,8,9-tetrahydro-5H-pyrimido[4,5-b]indole-6-carboxamide,
(RS)—N-[3-(methylsulfonyl)propyl]-4-[(3-oxo-1,3-dihydro-2-benzofuran-5-yl)amino]-6,7,8,9-tetrahydro-5H-pyrimido[4,5-b]indole-6-carboxamide,
(RS)—N-[3-(methylsulfonyl)propyl]-4-{[3-(1H-tetrazol-1-yl)phenyl]amino}-6,7,8,9-tetrahydro-5H-pyrimido[4,5-b]indole-6-carboxamide,
(RS)—N-[3-(methylsulfonyl)propyl]-4-[(4-sulfamoylphenyl)amino]-6,7,8,9-tetrahydro-5H-pyrimido[4,5-b]indole-6-carboxamide,
(RS)-4-(1,3-benzothiazol-6-ylamino)-N-[3-(methylsulfonyl)propyl]-6,7,8,9-tetrahydro-5H-pyrimido[4,5-b]indole-6-carboxamide,
(RS)-4-[(2-methyl-1,3-benzothiazol-5-yl)amino]-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-[(3-oxo-3,4-dihydro-2H-1,4-benzoxazin-7-yl)amino]-6,7,8,9-tetrahydro-5H-pyrimido[4,5-b]indole-6-carboxamide,
(RS)-4-[(1,1-dioxido-2,3-dihydro-1-benzothiophen-5-yl)amino]-N-[3-(methylsulfonyl)propyl]-6,7,8,9-tetrahydro-5H-pyrimido[4,5-b]indole-6-carboxamide,
(RS)-4-[(4-acetylphenyl)amino]-N-[3-(methylsulfonyl)propyl]-6,7,8,9-tetrahydro-5H-pyrimido[4,5-b]indole-6-carboxamide,
(RS)-4-[(2-hydroxy-4-methylphenyl)amino]-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-{[3-(1,3-oxazol-5-yl)phenyl]amino}-6,7,8,9-tetrahydro-5H-pyrimido[4,5-b]indole-6-carboxamide,
(RS)-4-(1H-indazol-5-yloxy)-N-[3-(methylsulfonyl)propyl]-6,7,8,9-tetrahydro-5H-pyrimido[4,5-b]indole-6-carboxamide,
(RS)-4-(4-fluoro-2-methoxyphenoxy)-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-(5,6,7,8-tetrahydronaphthalen-1-ylamino)-6,7,8,9-tetrahydro-5H-pyrimido[4,5-b]indole-6-carboxamide,
(RS)-2-methyl-5-[(6-{[3-(methylsulfonyl)propyl]carbamoyl}-6,7,8,9-tetrahydro-5H-pyrimido[4,5-b]indol-4-yl)amino]benzenesulfonic acid,
(RS)-4-[(2,2-difluoro-1,3-benzodioxol-4-yl)amino]-N-[3-(methylsulfonyl)propyl]-6,7,8,9-tetrahydro-5H-pyrimido[4,5-b]indole-6-carboxamide,
(RS)-4-[(6-fluoro-1H-indazol-5-yl)amino]-N-[3-(methylsulfonyl)propyl]-6,7,8,9-tetrahydro-5H-pyrimido[4,5-b]indole-6-carboxamide,
(RS)-4-{[2-fluoro-5-(trifluoromethoxy)phenyl]amino}-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-{[4-(morpholin-4-yl)phenyl]amino}-6,7,8,9-tetrahydro-5H-pyrimido[4,5-b]indole-6-carboxamide,
(RS)-methyl methyl {4-[(6-{[3-(methylsulfonyl)propyl]carbamoyl}-6,7,8,9-tetrahydro-5H-pyrimido[4,5-b]indol-4-yl)amino]phenyl}phosphinate,
(RS)-4-{[7-(methylsulfanyl)-2,3-dihydro-1,4-benzodioxin-6-yl]amino}-N-[3-(methylsulfonyl)propyl]-6,7,8,9-tetrahydro-5H-pyrimido[4,5-b]indole-6-carboxamide,
N-(1H-indazol-5-yl)-5,7,8,9-tetrahydrothiopyrano[3′,4′:4,5]pyrrolo[2,3-d]pyrimidin-4-amine,
(RS)-4-{[2-methoxy-5-(trifluoromethyl)phenyl]amino}-N-[3-(methylsulfonyl)propyl]-6,7,8,9-tetrahydro-5H-pyrimido[4,5-b]indole-6-carboxamide,
(RS)-4-[(5-methoxy-2-methyl-1,3-benzothiazol-6-yl)amino]-N-[3-(methylsulfonyl)propyl]-6,7,8,9-tetrahydro-5H-pyrimido[4,5-b]indole-6-carboxamide,
N-(1H-indazol-5-yl)-5,6,7,8-tetrahydrocyclopenta[4,5]pyrrolo[2,3-d]pyrimidin-4-amine,
N-(4-fluoro-2-isopropoxyphenyl)-5,6,7,8-tetrahydrocyclopenta[4,5]pyrrolo[2,3-d]pyrimidin-4-amine,
(RS)-4-{[2-methoxy-4-(morpholin-4-yl)phenyl]amino}-N-[3-(methylsulfonyl)propyl]-6,7,8,9-tetrahydro-5H-pyrimido[4,5-b]indole-6-carboxamide,
(RS)-4-[(2,6-dimethoxyphenyl)amino]-N-[3-(methylsulfonyl)propyl]-6,7,8,9-tetrahydro-5H-pyrimido[4,5-b]indole-6-carboxamide,
(RS)-4-[(2,4-dimethoxyphenyl)amino]-N-[3-(methylsulfonyl)propyl]-6,7,8,9-tetrahydro-5H-pyrimido[4,5-b]indole-6-carboxamide,
(RS)-4-[(2,3-dimethoxyphenyl)amino]-N-[3-(methylsulfonyl)propyl]-6,7,8,9-tetrahydro-5H-pyrimido[4,5-b]indole-6-carboxamide,
(RS)-4-[(2-methoxy-6-methylphenyl)amino]-N-[3-(methylsulfonyl)propyl]-6,7,8,9-tetrahydro-5H-pyrimido[4,5-b]indole-6-carboxamide,
(RS)-4-{[2-methoxy-4-(4-methylpiperazin-1-yl)phenyl]amino}-N-[3-(methylsulfonyl)propyl]-6,7,8,9-tetrahydro-5H-pyrimido[4,5-b]indole-6-carboxamide,
(RS)-4-[(2,3-difluorophenyl)amino]-N-[3-(methylsulfonyl)propyl]-6,7,8,9-tetrahydro-5H-pyrimido[4,5-b]indole-6-carboxamide,
(RS)-4-{[2-(cyclopentyloxy)phenyl]amino}-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-{[2-(tetrahydro-2H-pyran-4-yloxy)phenyl]amino}-6,7,8,9-tetrahydro-5H-pyrimido[4,5-b]indole-6-carboxamide,
(RS)-4-[(2,2-dimethyl-2,3-dihydro-1-benzofuran-7-yl)amino]-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-[(1-oxo-2,3-dihydro-1H-isoindol-4-yl)amino]-6,7,8,9-tetrahydro-5H-pyrimido[4,5-b]indole-6-carboxamide,
(RS)-4-[(8-fluoro-2-oxo-1,2-dihydroquinolin-5-yl)amino]-N-[3-(methylsulfonyl)propyl]-6,7,8,9-tetrahydro-5H-pyrimido[4,5-b]indole-6-carboxamide,
N-(1H-indazol-5-yl)-5,7,8,9-tetrahydrothiopyrano[3′,4′:4,5]pyrrolo[2,3-d]pyrimidin-4-amine 6,6-dioxide,
N-(4-fluoro-2-isopropoxyphenyl)-5,7,8,9-tetrahydrothiopyrano[3′,4′:4,5]pyrrolo[2,3-d]pyrimidin-4-amine 6,6-dioxide,
(RS)—N-(1H-indazol-5-yl)-5,7,8,9-tetrahydrothiopyrano[3′,4′:4,5]pyrrolo[2,3-d]pyrimidin-4-amine 6-oxide,
(RS)—N-(4-fluoro-2-isopropoxyphenyl)-5,7,8,9-tetrahydrothiopyrano[3′,4′:4,5]pyrrolo[2,3-d]pyrimidin-4-amine 6-oxide,
(RS)-ethyl 4-[(2-oxo-2,3-dihydro-1,3-benzothiazol-6-yl)amino]-6,7,8,9-tetrahydro-5H-pyrimido[4,5-b]indole-6-carboxylate,
(RS)-4-[(2-oxo-2,3-dihydro-1,3-benzothiazol-6-yl)amino]-6,7,8,9-tetrahydro-5H-pyrimido[4,5-b]indole-6-carboxylic acid,
(RS)—N,N-dimethyl-4-[(2-oxo-2,3-dihydro-1,3-benzothiazol-6-yl)amino]-6,7,8,9-tetrahydro-5H-pyrimido[4,5-b]indole-6-carboxamide,
(RS)-4-[(2-oxo-2,3-dihydro-1,3-benzothiazol-6-yl)amino]-N-[3-(trifluoromethyl)benzyl]-6,7,8,9-tetrahydro-5H-pyrimido[4,5-b]indole-6-carboxamide,
(RS)—N-(3-fluorobenzyl)-4-[(2-oxo-2,3-dihydro-1,3-benzothiazol-6-yl)amino]-6,7,8,9-tetrahydro-5H-pyrimido[4,5-b]indole-6-carboxamide,
(RS)—N-benzyl-4-[(2-oxo-2,3-dihydro-1,3-benzothiazol-6-yl)amino]-6,7,8,9-tetrahydro-5H-pyrimido[4,5-b]indole-6-carboxamide,
(RS)-4-[(2-oxo-2,3-dihydro-1,3-benzothiazol-6-yl)amino]-N-phenyl-6,7,8,9-tetrahydro-5H-pyrimido[4,5-b]indole-6-carboxamide,
(RS)-4-[(2-oxo-2,3-dihydro-1,3-benzothiazol-6-yl)amino]-N-(3,3,3-trifluoropropyl)-6,7,8,9-tetrahydro-5H-pyrimido[4,5-b]indole-6-carboxamide,
(RS)-4-[(2-oxo-2,3-dihydro-1,3-benzothiazol-6-yl)amino]-N-(2,2,2-trifluoroethyl)-6,7,8,9-tetrahydro-5H-pyrimido[4,5-b]indole-6-carboxamide,
(RS)—N-(cyclopropylmethyl)-4-[(2-oxo-2,3-dihydro-1,3-benzothiazol-6-yl)amino]-6,7,8,9-tetrahydro-5H-pyrimido[4,5-b]indole-6-carboxamide,
(RS)—N-isobutyl-4-[(2-oxo-2,3-dihydro-1,3-benzothiazol-6-yl)amino]-6,7,8,9-tetrahydro-5H-pyrimido[4,5-b]indole-6-carboxamide,
(RS)—N-isopropyl-4-[(2-oxo-2,3-dihydro-1,3-benzothiazol-6-yl)amino]-6,7,8,9-tetrahydro-5H-pyrimido[4,5-b]indole-6-carboxamide,
(RS)—N-ethyl-4-[(2-oxo-2,3-dihydro-1,3-benzothiazol-6-yl)amino]-6,7,8,9-tetrahydro-5H-pyrimido[4,5-b]indole-6-carboxamide,
(RS)—N-methyl-4-[(2-oxo-2,3-dihydro-1,3-benzothiazol-6-yl)amino]-6,7,8,9-tetrahydro-5H-pyrimido[4,5-b]indole-6-carboxamide,
(RS)-ethyl 4-{[2-methoxy-5-(trifluoromethyl)phenyl]amino}-6,7,8,9-tetrahydro-5H-pyrimido[4,5-b]indole-6-carboxylate,
(RS)-4-[(4-chloro-2-methoxybenzyl)amino]-N-[3-(methylsulfonyl)propyl]-6,7,8,9-tetrahydro-5H-pyrimido[4,5-b]indole-6-carboxamide,
(RS)-4-{[2-methoxy-5-(trifluoromethyl)phenyl]amino}-6,7,8,9-tetrahydro-5H-pyrimido[4,5-b]indole-6-carboxylic acid,
(RS)—N-cyclopropyl-4-{[2-methoxy-5-(trifluoromethyl)phenyl]amino}-6,7,8,9-tetrahydro-5H-pyrimido[4,5-b]indole-6-carboxamide,
(RS)—N-ethyl-4-{[2-methoxy-5-(trifluoromethyl)phenyl]amino}-6,7,8,9-tetrahydro-5H-pyrimido[4,5-b]indole-6-carboxamide,
(RS)-4-{[2-methoxy-5-(trifluoromethyl)phenyl]amino}-N-methyl-6,7,8,9-tetrahydro-5H-pyrimido[4,5-b]indole-6-carboxamide,
N-(5-fluoro-2-methoxyphenyl)-6,7,8,9-tetrahydro-5H-pyrimido[4,5-b]indol-4-amine 4-fluoro-5-nitro-N-(6,7,8,9-tetrahydro-5H-pyrimido[4,5-b]indol-4-yl)benzene-1,2-diamine,
6-(6,7,8,9-tetrahydro-5H-pyrimido[4,5-b]indol-4-ylamino)-1,4-dihydroquinoxaline-2,3-dione,
1-(6,7,8,9-tetrahydro-5H-pyrimido[4,5-b]indol-4-yl)-2,3-dihydro-1H-indol-6-amine,
N-(6-methoxy-1H-indazol-5-yl)-6,7,8,9-tetrahydro-5H-pyrimido[4,5-b]indol-4-amine,
1-(6,7,8,9-tetrahydro-5H-pyrimido[4,5-b]indol-4-yl)-2,3-dihydro-1H-indol-5-amine,
N-(3,4-dichlorophenyl)-6,7,8,9-tetrahydro-5H-pyrimido[4,5-b]indol-4-amine,
5-(6,7,8,9-tetrahydro-5H-pyrimido[4,5-b]indol-4-ylamino)-1,3-dihydro-2H-benzimidazol-2-one,
N-[2-(cyclopentyloxy)phenyl]-6,7,8,9-tetrahydro-5H-pyrimido[4,5-b]indol-4-amine,
6-(6,7,8,9-tetrahydro-5H-pyrimido[4,5-b]indol-4-ylamino)-1,3-benzothiazol-2(3H)-one,
N-[2-(morpholin-4-yl)phenyl]-6,7,8,9-tetrahydro-5H-pyrimido[4,5-b]indol-4-amine,
N-[2-methoxy-5-(trifluoromethyl)phenyl]-6,7,8,9-tetrahydro-5H-pyrimido[4,5-b]indol-4-amine,
4-(2,3-dihydro-1H-indol-1-yl)-6,7,8,9-tetrahydro-5H-pyrimido[4,5-b]indole,
4-(6-nitro-2,3-dihydro-1H-indol-1-yl)-6,7,8,9-tetrahydro-5H-pyrimido[4,5-b]indole hydrochloride (1:1),
4-(5-chloro-2,3-dihydro-1H-indol-1-yl)-6,7,8,9-tetrahydro-5H-pyrimido[4,5-b]indole hydrochloride (1:1),
4-(5-nitro-2,3-dihydro-1H-indol-1-yl)-6,7,8,9-tetrahydro-5H-pyrimido[4,5-b]indole,
N-[5-chloro-2-(propan-2-yloxy)phenyl]-6,7,8,9-tetrahydro-5H-pyrimido[4,5-b]indol-4-amine,
N-(5-chloro-2-methoxyphenyl)-6,7,8,9-tetrahydro-5H-pyrimido[4,5-b]indol-4-amine,
N-[6-(propan-2-yloxy)-1H-indazol-5-yl]-6,7,8,9-tetrahydro-5H-pyrimido[4,5-b]indol-4-amine hydrochloride (1:1),
N-[5-bromo-2-(propan-2-yloxy)phenyl]-6,7,8,9-tetrahydro-5H-pyrimido[4,5-b]indol-4-amine,
N-(5-bromo-2-methoxyphenyl)-6,7,8,9-tetrahydro-5H-pyrimido[4,5-b]indol-4-amine,
N-[3-methoxy-4-(6,7,8,9-tetrahydro-5H-pyrimido[4,5-b]indol-4-ylamino)phenyl]methanesulfonamide,
N-[2-(tetrahydro-2H-pyran-4-yloxy)phenyl]-6,7,8,9-tetrahydro-5H-pyrimido[4,5-b]indol-4-amine,
(RS)-5-chloro-6-{[6-methoxy-6-(methoxymethyl)-6,7,8,9-tetrahydro-5H-pyrimido[4,5-b]indol-4-yl]amino}-1,3-dihydro-2H-benzimidazol-2-one,
(RS)—N-[5-chloro-2-(propan-2-yloxy)phenyl]-6-methoxy-6-(methoxymethyl)-6,7,8,9-tetrahydro-5H-pyrimido[4,5-b]indol-4-amine,
(RS)—N-(5-chloro-2-methoxyphenyl)-6-methoxy-6-(methoxymethyl)-6,7,8,9-tetrahydro-5H-pyrimido[4,5-b]indol-4-amine,
(RS)—N-[2-(cyclopentyloxy)-4-fluorophenyl]-6-methoxy-6-(methoxymethyl)-6,7,8,9-tetrahydro-5H-pyrimido[4,5-b]indol-4-amine,
(RS)—N-(1H-indazol-5-yl)-6-methoxy-6-(methoxymethyl)-6,7,8,9-tetrahydro-5H-pyrimido[4,5-b]indol-4-amine,
(RS)-6-methoxy-N-(6-methoxy-1H-indazol-5-yl)-6-(methoxymethyl)-6,7,8,9-tetrahydro-5H-pyrimido[4,5-b]indol-4-amine,
(RS)—N-[4-fluoro-2-(propan-2-yloxy)phenyl]-6-methoxy-6-(methoxymethyl)-6,7,8,9-tetrahydro-5H-pyrimido[4,5-b]indol-4-amine,
(RS)-6-{[6-methoxy-6-(methoxymethyl)-6,7,8,9-tetrahydro-5H-pyrimido[4,5-b]indol-4-yl]amino}-1,3-benzothiazol-2(3H)-one,
(RS)—N-(6-fluoro-1H-indazol-5-yl)-6-methoxy-6-(methoxymethyl)-6,7,8,9-tetrahydro-5H-pyrimido[4,5-b]indol-4-amine,
(RS)—N-(4,5-dichloro-2-methoxyphenyl)-6-methoxy-6-(methoxymethyl)-6,7,8,9-tetrahydro-5H-pyrimido[4,5-b]indol-4-amine,
6,6-difluoro-N-(6-methoxy-1H-indazol-5-yl)-6,7,8,9-tetrahydro-5H-pyrimido[4,5-b]indol-4-amine,
6-[(6,6-difluoro-6,7,8,9-tetrahydro-5H-pyrimido[4,5-b]indol-4-yl)amino]-1,3-benzothiazol-2(3H)-one,
N-[5-chloro-2-(propan-2-yloxy)phenyl]-6,6-difluoro-6,7,8,9-tetrahydro-5H-pyrimido[4,5-b]indol-4-amine,
N-(5-chloro-2-methoxyphenyl)-6,6-difluoro-6,7,8,9-tetrahydro-5H-pyrimido[4,5-b]indol-4-amine,
6,6-difluoro-N-[2-methoxy-5-(trifluoromethyl)phenyl]-6,7,8,9-tetrahydro-5H-pyrimido[4,5-b]indol-4-amine,
(RS)-4-(1H-indazol-5-ylsulfanyl)-N-[3-(methylsulfonyl)propyl]-6,7,8,9-tetrahydro-5H-pyrimido[4,5-b]indole-6-carboxamide,
(RS)-4-[(4-fluoro-2-methoxyphenyl)sulfanyl]-N-[3-(methylsulfonyl)propyl]-6,7,8,9-tetrahydro-5H-pyrimido[4,5-b]indole-6-carboxamide,
(RS)-4-[(5-chloro-2-methoxyphenyl)sulfanyl]-N-[3-(methylsulfonyl)propyl]-6,7,8,9-tetrahydro-5H-pyrimido[4,5-b]indole-6-carboxamide,
(RS)-4-[(5-chloro-2-methoxyphenyl)amino]-N-[3-(methylsulfonyl)propyl]-6,7,8,9-tetrahydro-5H-pyrimido[4,5-b]indole-6-carboxamide,
(RS)-4-{[5-chloro-2-(propan-2-yloxy)phenyl]amino}-N-[3-(methylsulfonyl)propyl]-6,7,8,9-tetrahydro-5H-pyrimido[4,5-b]indole-6-carboxamide,
(RS)-4-{[2-(cyclopropylmethoxy)phenyl]amino}-N-[3-(methylsulfonyl)propyl]-6,7,8,9-tetrahydro-5H-pyrimido[4,5-b]indole-6-carboxamide,
(RS)-4-{[2-(2,2-difluoroethoxy)phenyl]amino}-N-[3-(methylsulfonyl)propyl]-6,7,8,9-tetrahydro-5H-pyrimido[4,5-b]indole-6-carboxamide,
(RS)-4-{[2-(2-methylpropoxy)phenyl]amino}-N-[3-(methylsulfonyl)propyl]-6,7,8,9-tetrahydro-5H-pyrimido[4,5-b]indole-6-carboxamide,
(RS)-4-{[2-(difluoromethoxy)phenyl]amino}-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-[(2-thioxo-2,3-dihydro-1,3-benzothiazol-6-yl)amino]-6,7,8,9-tetrahydro-5H-pyrimido[4,5-b]indole-6-carboxamide,
(RS)-4-{[2-(difluoromethoxy)-4-methylphenyl]amino}-N-[3-(methylsulfonyl)propyl]-6,7,8,9-tetrahydro-5H-pyrimido[4,5-b]indole-6-carboxamide,
(RS)-4-[(5-chloro-4-fluoro-2-methoxyphenyl)amino]-N-[3-(methylsulfonyl)propyl]-6,7,8,9-tetrahydro-5H-pyrimido[4,5-b]indole-6-carboxamide,
(RS)-4-{[5-(dimethylamino)-2-methoxyphenyl]amino}-N-[3-(methylsulfonyl)propyl]-6,7,8,9-tetrahydro-5H-pyrimido[4,5-b]indole-6-carboxamide,
(RS)-4-{[4-fluoro-2-(2-hydroxyethoxy)phenyl]amino}-N-[3-(methylsulfonyl)propyl]-6,7,8,9-tetrahydro-5H-pyrimido[4,5-b]indole-6-carboxamide,
(RS)-4-[(5-fluoro-2-propoxyphenyl)amino]-N-[3-(methylsulfonyl)propyl]-6,7,8,9-tetrahydro-5H-pyrimido[4,5-b]indole-6-carboxamide,
(RS)-4-[(4,5-difluoro-2-methoxyphenyl)amino]-N-[3-(methylsulfonyl)propyl]-6,7,8,9-tetrahydro-5H-pyrimido[4,5-b]indole-6-carboxamide,
(RS)-4-[(4-chloro-2-methoxy-5-methylphenyl)amino]-N-[3-(methylsulfonyl)propyl]-6,7,8,9-tetrahydro-5H-pyrimido[4,5-b]indole-6-carboxamide,
(RS)-4-[(6-methoxy-1H-indazol-5-yl)amino]-N-[3-(methylsulfonyl)propyl]-6,7,8,9-tetrahydro-5H-pyrimido[4,5-b]indole-6-carboxamide,
(RS)-4-[(6-methoxy-2-oxo-2,3-dihydro-1H-benzimidazol-5-yl)amino]-N-[3-(methylsulfonyl)propyl]-6,7,8,9-tetrahydro-5H-pyrimido[4,5-b]indole-6-carboxamide,
(RS)-4-[(4-chloro-2-ethoxyphenyl)amino]-N-[3-(methylsulfonyl)propyl]-6,7,8,9-tetrahydro-5H-pyrimido[4,5-b]indole-6-carboxamide,
(RS)-4-[(3,4-dichlorophenyl)amino]-N-[3-(methylsulfonyl)propyl]-6,7,8,9-tetrahydro-5H-pyrimido[4,5-b]indole-6-carboxamide,
(RS)—N-[3-(methylsulfonyl)propyl]-4-{[2-(tetrahydrofuran-3-yloxy)phenyl]amino}-6,7,8,9-tetrahydro-5H-pyrimido[4,5-b]indole-6-carboxamide,
(RS)-4-(2,3-dihydro-1H-indol-1-yl)-N-[3-(methylsulfonyl)propyl]-6,7,8,9-tetrahydro-5H-pyrimido[4,5-b]indole-6-carboxamide,
(RS)-4-[(2-methyl-1H-benzimidazol-4-yl)amino]-N-[3-(methylsulfonyl)propyl]-6,7,8,9-tetrahydro-5H-pyrimido[4,5-b]indole-6-carboxamide,
(RS)-ethyl 4-{[2-(cyclopentyloxy)phenyl]amino}-6,7,8,9-tetrahydro-5H-pyrimido[4,5-b]indole-6-carboxylate,
(RS)-ethyl 4-[(5-fluoro-2-methoxyphenyl)amino]-6,7,8,9-tetrahydro-5H-pyrimido[4,5-b]indole-6-carboxylate,
(RS)-ethyl 4-{[2-(morpholin-4-yl)phenyl]amino}-6,7,8,9-tetrahydro-5H-pyrimido[4,5-b]indole-6-carboxylate,
(RS)-ethyl 4-(2,3-dihydro-1H-indol-1-yl)-6,7,8,9-tetrahydro-5H-pyrimido[4,5-b]indole-6-carboxylate,
(RS)-ethyl 4-{[4-fluoro-2-(propan-2-yloxy)phenyl]amino}-6,7,8,9-tetrahydro-5H-pyrimido[4,5-b]indole-6-carboxylate,
(RS)-ethyl 4-[(5-chloro-2-methoxyphenyl)amino]-6,7,8,9-tetrahydro-5H-pyrimido[4,5-b]indole-6-carboxylate,
(RS)-ethyl 4-{[4-fluoro-2-(2-hydroxyethoxy)phenyl]amino}-6,7,8,9-tetrahydro-5H-pyrimido[4,5-b]indole-6-carboxylate,
(RS)-ethyl 4-{[5-chloro-2-(propan-2-yloxy)phenyl]amino}-6,7,8,9-tetrahydro-5H-pyrimido[4,5-b]indole-6-carboxylate,
(RS)-ethyl 4-[(3,4-dichlorophenyl)amino]-6,7,8,9-tetrahydro-5H-pyrimido[4,5-b]indole-6-carboxylate,
(RS)-ethyl 4-[(6-methoxy-1H-indazol-5-yl)amino]-6,7,8,9-tetrahydro-5H-pyrimido[4,5-b]indole-6-carboxylate,
(RS)-4-{[2-(cyclopentyloxy)phenyl]amino}-6,7,8,9-tetrahydro-5H-pyrimido[4,5-b]indole-6-carboxylic acid,
(RS)-4-[(5-fluoro-2-methoxyphenyl)amino]-6,7,8,9-tetrahydro-5H-pyrimido[4,5-b]indole-6-carboxylic acid,
(RS)-4-{[4-fluoro-2-(2-hydroxyethoxy)phenyl]amino}-6,7,8,9-tetrahydro-5H-pyrimido[4,5-b]indole-6-carboxylic acid,
(RS)-4-{[4-fluoro-2-(propan-2-yloxy)phenyl]amino}-6,7,8,9-tetrahydro-5H-pyrimido[4,5-b]indole-6-carboxylic acid,
(RS)-4-[(6-methoxy-1H-indazol-5-yl)amino]-6,7,8,9-tetrahydro-5H-pyrimido[4,5-b]indole-6-carboxylic acid hydrochloride (1:1),
(RS)-4-(2,3-dihydro-1H-indol-1-yl)-6,7,8,9-tetrahydro-5H-pyrimido[4,5-b]indole-6-carboxylic acid,
(RS)-4-{[2-(morpholin-4-yl)phenyl]amino}-6,7,8,9-tetrahydro-5H-pyrimido[4,5-b]indole-6-carboxylic acid,
(RS)—N-[(4-{[4-fluoro-2-(2-hydroxyethoxy)phenyl]amino}-6,7,8,9-tetrahydro-5H-pyrimido[4,5-b]indol-6-yl)carbonyl]glycine,
(RS)-4-{[2-(cyclopentyloxy)phenyl]amino}-N,N-dimethyl-6,7,8,9-tetrahydro-5H-pyrimido[4,5-b]indole-6-carboxamide,
(RS)-4-{[2-(cyclopentyloxy)phenyl]amino}-N-methyl-6,7,8,9-tetrahydro-5H-pyrimido[4,5-b]indole-6-carboxamide,
(RS)-4-{[2-(cyclopentyloxy)phenyl]amino}-N-ethyl-6,7,8,9-tetrahydro-5H-pyrimido[4,5-b]indole-6-carboxamide,
(RS)-4-{[2-(cyclopentyloxy)phenyl]amino}-N-cyclopropyl-6,7,8,9-tetrahydro-5H-pyrimido[4,5-b]indole-6-carboxamide,
(RS)-(4-{[2-(cyclopentyloxy)phenyl]amino}-6,7,8,9-tetrahydro-5H-pyrimido[4,5-b]indol-6-yl)(4-methylpiperazin-1-yl)methanone,
(RS)-4-{[2-(cyclopentyloxy)phenyl]amino}-N-phenyl-6,7,8,9-tetrahydro-5H-pyrimido[4,5-b]indole-6-carboxamide,
(RS)—N-ethyl-4-[(5-fluoro-2-methoxyphenyl)amino]-6,7,8,9-tetrahydro-5H-pyrimido[4,5-b]indole-6-carboxamide,
(RS)-4-[(5-fluoro-2-methoxyphenyl)amino]-Nropan-2-yl)-6,7,8,9-tetrahydro-5H-pyrimido[4,5-b]indole-6-carboxamide,
(RS)-{4-[(5-fluoro-2-methoxyphenyl)amino]-6,7,8,9-tetrahydro-5H-pyrimido[4,5-b]indol-6-yl}(4-methylpiperazin-1-yl)methanone,
(RS)—N-cyclopropyl-4-[(5-fluoro-2-methoxyphenyl)amino]-6,7,8,9-tetrahydro-5H-pyrimido[4,5-b]indole-6-carboxamide,
(RS)-4-[(5-fluoro-2-methoxyphenyl)amino]-N-(3,3,3-trifluoropropyl)-6,7,8,9-tetrahydro-5H-pyrimido[4,5-b]indole-6-carboxamide,
(RS)-4-[(5-fluoro-2-methoxyphenyl)amino]-N-phenyl-6,7,8,9-tetrahydro-5H-pyrimido[4,5-b]indole-6-carboxamide,
(RS)—N-benzyl-4-[(5-fluoro-2-methoxyphenyl)amino]-6,7,8,9-tetrahydro-5H-pyrimido[4,5-b]indole-6-carboxamide,
(RS)—N-ethyl-4-{[2-(morpholin-4-yl)phenyl]amino}-6,7,8,9-tetrahydro-5H-pyrimido[4,5-b]indole-6-carboxamide,
(RS)-4-{[2-(morpholin-4-yl)phenyl]amino}-N-(propan-2-yl)-6,7,8,9-tetrahydro-5H-pyrimido[4,5-b]indole-6-carboxamide,
(RS)—N-cyclopropyl-4-{[2-(morpholin-4-yl)phenyl]amino}-6,7,8,9-tetrahydro-5H-pyrimido[4,5-b]indole-6-carboxamide,
(RS)-4-{[2-(morpholin-4-yl)phenyl]amino}-N-(3,3,3-trifluoropropyl)-6,7,8,9-tetrahydro-5H-pyrimido[4,5-b]indole-6-carboxamide,
(RS)-4-{[2-(morpholin-4-yl)phenyl]amino}-N-phenyl-6,7,8,9-tetrahydro-5H-pyrimido[4,5-b]indole-6-carboxamide,
(RS)—N-benzyl-4-{[2-(morpholin-4-yl)phenyl]amino}-6,7,8,9-tetrahydro-5H-pyrimido[4,5-b]indole-6-carboxamide,
(RS)-4-(2,3-dihydro-1H-indol-1-yl)-N-ethyl-6,7,8,9-tetrahydro-5H-pyrimido[4,5-b]indole-6-carboxamide,
(RS)-4-(2,3-dihydro-1H-indol-1-yl)-N-(propan-2-yl)-6,7,8,9-tetrahydro-5H-pyrimido[4,5-b]indole-6-carboxamide,
(RS)—N-benzyl-4-(2,3-dihydro-1H-indol-1-yl)-6,7,8,9-tetrahydro-5H-pyrimido[4,5-b]indole-6-carboxamide,
(RS)-4-(2,3-dihydro-1H-indol-1-yl)-N-[3-(trifluoromethyl)benzyl]-6,7,8,9-tetrahydro-5H-pyrimido[4,5-b]indole-6-carboxamide,
(RS)-[4-(2,3-dihydro-1H-indol-1-yl)-6,7,8,9-tetrahydro-5H-pyrimido[4,5-b]indol-6-yl](4-methylpiperazin-1-yl)methanone,
(RS)-4-(2,3-dihydro-1H-indol-1-yl)-N-methyl-6,7,8,9-tetrahydro-5H-pyrimido[4,5-b]indole-6-carboxamide,
(RS)-4-{[2-methoxy-5-(trifluoromethyl)phenyl]amino}-N-(propan-2-yl)-6,7,8,9-tetrahydro-5H-pyrimido[4,5-b]indole-6-carboxamide,
(RS)-4-{[2-methoxy-5-(trifluoromethyl)phenyl]amino}-N-phenyl-6,7,8,9-tetrahydro-5H-pyrimido[4,5-b]indole-6-carboxamide,
(RS)-(4-{[2-methoxy-5-(trifluoromethyl)phenyl]amino}-6,7,8,9-tetrahydro-5H-pyrimido[4,5-b]indol-6-yl)(4-methylpiperazin-1-yl)methanone,
(RS)-4-[(6-methoxy-1H-indazol-5-yl)amino]-Nropan-2-yl)-6,7,8,9-tetrahydro-5H-pyrimido[4,5-b]indole-6-carboxamide,
(RS)-4-[(6-methoxy-1H-indazol-5-yl)amino]-N-methyl-6,7,8,9-tetrahydro-5H-pyrimido[4,5-b]indole-6-carboxamide,
(RS)—N-ethyl-4-[(6-methoxy-1H-indazol-5-yl)amino]-6,7,8,9-tetrahydro-5H-pyrimido[4,5-b]indole-6-carboxamide,
(RS)—N-benzyl-4-[(6-methoxy-1H-indazol-5-yl)amino]-6,7,8,9-tetrahydro-5H-pyrimido[4,5-b]indole-6-carboxamide,
(RS)-4-[(6-methoxy-1H-indazol-5-yl)amino]-N-[3-(trifluoromethyl)benzyl]-6,7,8,9-tetrahydro-5H-pyrimido[4,5-b]indole-6-carboxamide,
(RS)-{4-[(6-methoxy-1H-indazol-5-yl)amino]-6,7,8,9-tetrahydro-5H-pyrimido[4,5-b]indol-6-yl}(4-methylpiperazin-1-yl)methanone,
(RS)-6-({6-[(4-methylpiperazin-1-yl)carbonyl]-6,7,8,9-tetrahydro-5H-pyrimido[4,5-b]indol-4-yl}amino)-1,3-benzothiazol-2(3H)-one,
(RS)-4-{[4-fluoro-2-(propan-2-yloxy)phenyl]amino}-N-(propan-2-yl)-6,7,8,9-tetrahydro-5H-pyrimido[4,5-b]indole-6-carboxamide,
(RS)-4-[(5-chloro-2-methoxyphenyl)amino]-Nropan-2-yl)-6,7,8,9-tetrahydro-5H-pyrimido[4,5-b]indole-6-carboxamide,
(RS)-4-{[5-chloro-2-(propan-2-yloxy)phenyl]amino}-N-(propan-2-yl)-6,7,8,9-tetrahydro-5H-pyrimido[4,5-b]indole-6-carboxamide,
(RS)-4-[(4,5-dichloro-2-methoxyphenyl)amino]-N-(propan-2-yl)-6,7,8,9-tetrahydro-5H-pyrimido[4,5-b]indole-6-carboxamide,
(RS)-4-(1H-indazol-5-ylamino)-N-(propan-2-yl)-6,7,8,9-tetrahydro-5H-pyrimido[4,5-b]indole-6-carboxamide,
(RS)-ethyl N-[(4-{[4-fluoro-2-(2-hydroxyethoxy)phenyl]amino}-6,7,8,9-tetrahydro-5H-pyrimido[4,5-b]indol-6-yl)carbonyl]glycinate,
(RS)-11-fluoro-1,3,4,7,8,14-hexahydro-5H-2,4-ethano-6,9-dioxa-1,14,15,17-tetraazabenzo[5,6]cyclotrideca[1,2,3-cd]inden-5-one,
(RS)—N-[3-(methylsulfonyl)propyl]-4-{[6-(propan-2-yloxy)-1H-indazol-5-yl]amino}-6,7,8,9-tetrahydro-5H-pyrimido[4,5-b]indole-6-carboxamide,
(RS)-6-methoxy-6-(methoxymethyl)-N-[6-(propan-2-yloxy)-1H-indazol-5-yl]-6,7,8,9-tetrahydro-5H-pyrimido[4,5-b]indol-4-amine,
6,6-difluoro-N-[6-(propan-2-yloxy)-1H-indazol-5-yl]-6,7,8,9-tetrahydro-5H-pyrimido[4,5-b]indol-4-amine,
(RS)-4-[(3,4-dichlorophenyl)amino]-6,7,8,9-tetrahydro-5H-pyrimido[4,5-b]indole-6-carboxylic acid,
(RS)-4-{[5-chloro-2-(propan-2-yloxy)phenyl]amino}-6,7,8,9-tetrahydro-5H-pyrimido[4,5-b]indole-6-carboxylic acid,
(RS)-4-[(5-chloro-2-methoxyphenyl)amino]-6,7,8,9-tetrahydro-5H-pyrimido[4,5-b]indole-6-carboxylic acid,
5-chloro-6-(6,7,8,9-tetrahydro-5H-pyrimido[4,5-b]indol-4-ylamino)-1,3-benzothiazol-2(3H)-one,
(RS)-4-[(6-chloro-2-oxo-2,3-dihydro-1H-benzimidazol-5-yl)amino]-6,7,8,9-tetrahydro-5H-pyrimido[4,5-b]indole-6-carboxylic acid,
6-[(6,6-difluoro-6,7,8,9-tetrahydro-5H-pyrimido[4,5-b]indol-4-yl)amino]-5-methoxy-1,3-benzothiazol-2(3H)-one,
or a stereoisomer, a tautomer, an N-oxide, a hydrate, a solvate, or a salt thereof, or a mixture of same.

7. A method of preparing a compound of general formula I according to claim 1, comprising reacting an intermediate compound of general formula II: 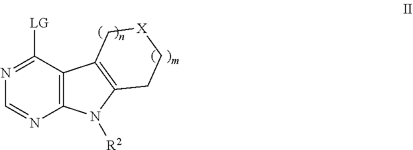
in which R², X, m, and n are as defined in claim 1, and LG represents a leaving group;
with a compound of general formula IIa:
R¹-A′ IIa
in which A′ represents a HO— or a HS— or a HNR³— group, and R¹ and R³ are as defined in claim 1;
thus providing a compound of general formula I: 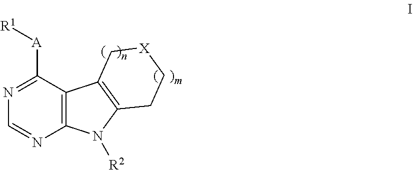
in which R¹, R², X, m, and n are as defined in claim 1.

8. (canceled)

9. A pharmaceutical composition comprising a compound of general formula I, or a stereoisomer, a tautomer, an N-oxide, a hydrate, a solvate, or a salt thereof, particularly a pharmaceutically acceptable salt thereof, or a mixture of same, according to claim 1, and a pharmaceutically acceptable diluent or carrier.

10. A pharmaceutical combination comprising:
one or more first active ingredients selected from a compound of general formula I according to claim 1, and
one or more second active ingredients selected from chemotherapeutic anti-cancer agents and target-specific anti-cancer agents.

11. (canceled)

12. (canceled)

13. A method of treatment of uncontrolled cell growth, proliferation and/or survival, an inappropriate cellular immune response, or an inappropriate cellular inflammatory response, particularly in which the uncontrolled cell growth, proliferation and/or survival, inappropriate cellular immune response, or inappropriate cellular inflammatory response is mediated by the MKNK-1 pathway, more particularly in which the disease of uncontrolled cell growth, proliferation and/or survival, inappropriate cellular immune response, or inappropriate cellular inflammatory response is a haematological tumour, a solid tumour and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof comprising administering and effective amount of a compound of claim 1 to a mammal in need thereof.

14. A compound of general formula II: 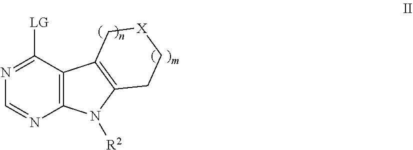
in which R², X, m, and n are as defined in claims 1, and LG represents a leaving group.

15. (canceled)
